(19) United States
(12) Patent Application Publication
(10) Pub. No.: US 2026/0354018 A1
(43) Pub. Date: Nov. 5, 2026

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si (KR)

(72) Inventors: Eunjeong CHOI, Suwon-si (KR); Dosik KIM, Suwon-si (KR); Eunhye AN, Suwon-si (KR); Jieun LEE, Suwon-si (KR); Dongjin JANG, Suwon-si (KR)

(21) Appl. No.: 19/639,854

(22) Filed: Apr. 6, 2026

(30) Foreign Application Priority Data
Apr. 22, 2025 (KR) 10-2025-0052218

(51) Int. Cl. H10K 85/60; C07B 59/00; C07D 401/14; C07D 403/10; C07D 403/14; C07D 405/14; C07D 409/14; C07D 487/04; C09K 11/02; H10K 50/11

(52) U.S. Cl. CPC H10K 85/6572; C07B 59/00; C07D 401/14; C07D 403/10; C07D 403/14; C07D 405/14; C07D 409/14; C07D 487/04; C09K 11/02; H10K 85/654; H10K 50/11

(57) ABSTRACT

A compound, a composition including the compound, an organic optoelectronic device including the compound or composition, and a display device including the organic optoelectronic device, the compound being represented by Chemical Formula 1:

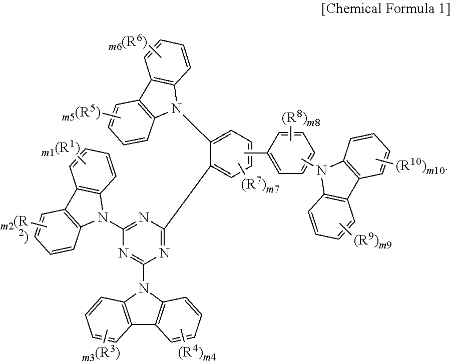

### CROSS-REFERENCE TO RELATED APPLICATION

This application claims priority to and the benefit of Korean Patent Application No. 10-2025-0052218 filed with the Korean Intellectual Property Office on Apr. 22, 2025, the entire contents of which are incorporated herein by reference.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (e.g., an organic optoelectronic diode) is a device capable of converting electrical energy and optical energy to each other.

Organic optoelectronic devices may be divided into two types according to a principle of operation. One is a photoelectric device that generates electrical energy by separating excitons formed by light energy into electrons and holes, and transferring the electrons and holes to different electrodes, respectively, and the other is light emitting device that generates light energy from electrical energy by supplying voltage or current to the electrodes.

Examples of organic optoelectronic devices may include an organic photoelectric device, an organic light emitting diode, an organic solar cell, or an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode may be influenced by an organic material between electrodes.

### SUMMARY

Embodiments are directed to a compound for an organic optoelectronic device represented by Chemical Formula 1:

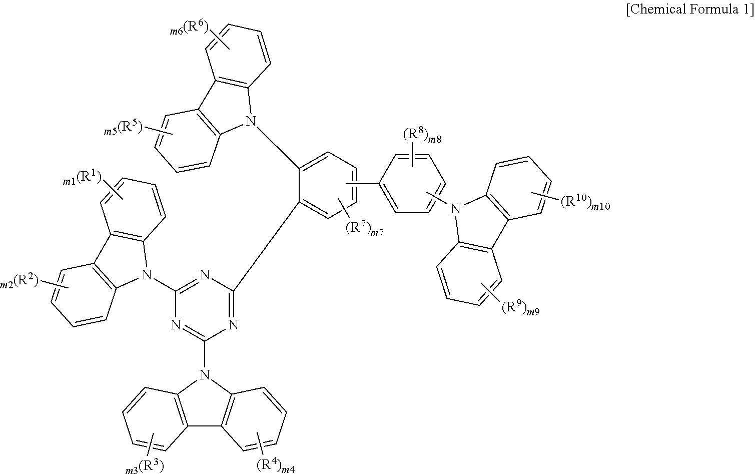

wherein, in Chemical Formula 1, R¹ to R¹⁰ are each independently hydrogen, deuterium, a substituted or unsubstituted C1 to C20 alkyl group, a substituted or unsubstituted C3 to C20 cycloalkyl group, a substituted or unsubstituted phenyl group, a substituted or unsubstituted biphenyl group, or a substituted or unsubstituted terphenyl group; m1 to m6, and m8 to m10 are each independently an integer of 1 to 4; and m7 is an integer of 1 to 3, and when m1 to m10 are 2 or more, each of R¹ to R¹⁰ may be the same or different from each other.

The embodiments may be realized by providing a composition for an organic optoelectronic device, the composition including a first compound and a second compound, wherein the first compound is the compound for the organic optoelectronic device according to an embodiment, and the second compound is represented by Chemical Formula 2, a combination of Chemical Formula 3 and Chemical Formula 4, or Chemical Formula 5:

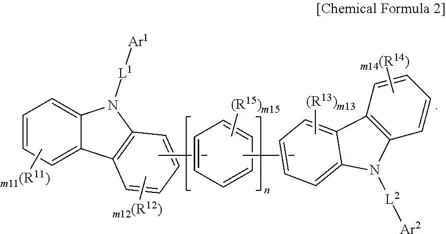

In Chemical Formula 2, R¹¹ to R¹⁵ may each independently be hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group; Ar¹ and Ar² may each independently be a substituted or unsubstituted C6 to C20 aryl group or a substituted or unsubstituted C2 to C30 heterocyclic group; L¹ and L² may each independently be a single bond or a substituted or unsubstituted C6 to C20 arylene group; m11, m14, and m15 may each independently be an integer of 1 to 4; and m12 and m13 may each independently be an integer of 1 to 3; n may be an integer of 0 to 2; and when m11 to m15 are 2 or more, each of R¹¹ to R¹⁵ may be the same or different from each other.

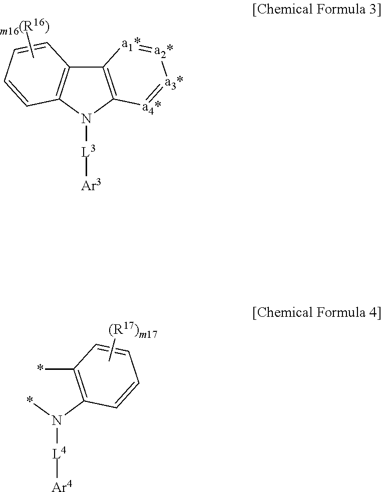

In Chemical Formula 3 and Chemical Formula 4, two adjacent ones of a₁* to a₄* of Chemical Formula 3 may each independently be linking carbons linked at * of Chemical Formula 4, the remaining two of a₁* to a₄* of Chemical Formula 3, not linked at * of Chemical Formula 4, may each independently be C-L^{a}-R^{a}; L^{a}, L³, and L⁴ may each independently be a single bond or a substituted or unsubstituted C6 to C20 arylene group; R^{a}, R¹⁶, and R¹⁷ may each independently be hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group; Ar³ and Ar⁴ may each independently be a substituted or unsubstituted C6 to C20 aryl group or a substituted or unsubstituted C2 to C30 heterocyclic group; and m16 and m17 may each independently be an integer of 1 to 4. When m16 and m17 are 2 or more, each of R¹⁶ and R¹⁷ may be the same or different from each other.

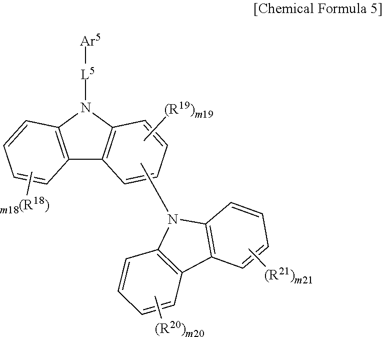

In Chemical Formula 5, L⁵ may be a single bond or a substituted or unsubstituted C6 to C20 arylene group, R¹⁸ to R²¹ may each independently be hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group; Ar⁵ may be a substituted or unsubstituted C6 to C20 aryl group or a substituted or unsubstituted C2 to C30 heterocyclic group; m18, m20, and m21 may each independently be an integer of 1 to 4; and m19 may be an integer of 1 to 3, and when m18 to m21 are 2 or more, each of R¹⁸ to R²¹ may be the same or different from each other.

The embodiments may be realized by providing an organic optoelectronic device including an anode and a cathode facing each other, and at least one organic layer between the anode and the cathode, wherein the organic layer includes the compound for an organic optoelectronic device according to an embodiment.

The embodiments may be realized by providing an organic optoelectronic device including an anode and a cathode facing each other, and at least one organic layer between the anode and the cathode, wherein the organic layer includes the composition for an organic optoelectronic device according to an embodiment.

The embodiments may be realized by providing a display device including the organic optoelectronic device according to an embodiment.

### BRIEF DESCRIPTION OF THE DRAWINGS

Features will become apparent to those of skill in the art by describing in detail exemplary embodiments with reference to the attached drawings, in which:

FIG. 1 is a cross-sectional view illustrating an organic light emitting diode according to some example embodiments; and

FIG. 2 is a cross-sectional view illustrating an organic light emitting diode according to some example embodiments.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing figure, the dimensions of layers and regions may be exaggerated for clarity of illustration. It will also be understood that when a layer or element is referred to as being “on” another layer or substrate, it can be directly on the other layer or substrate, or intervening layers may also be present. Further, it will be understood that when a layer is referred to as being “under” another layer, it can be directly under, and one or more intervening layers may also be present. In addition, it will also be understood that when a layer is referred to as being “between” two layers, it can be the only layer between the two layers, or one or more intervening layers may also be present. Like reference numerals refer to like elements throughout.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present embodiments, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In a specific example of the present embodiments, the “substituted” refers to replacement of at least one hydrogen of a substituent or a compound by deuterium, a C1 to C20 alkyl group, a C1 to C5 alkylsilyl group, a C6 to C20 aryl group, a C2 to C20 heteroaryl group, or a cyano group. In a specific example of the present embodiments, the “substituted” refers to replacement of at least one hydrogen of a substituent or a compound by deuterium, a C1 to C5 alkyl group, a C1 to C5 alkylsilyl group, a C6 to C18 aryl group, a C2 to C18 heteroaryl group, or a cyano group. In a specific example of the present embodiments, the “substituted” refers to replacement of at least one hydrogen of a substituent or a compound by deuterium, a cyano group, a methyl group, an ethyl group, an n-propyl group, an iso-propyl group, an n-butyl group, an iso-butyl group, a sec-butyl group, a tert-butyl group, a trimethylsilyl group, a phenyl group, a biphenyl group, a terphenyl group, or a naphthyl group.

As used herein, “unsubstituted” refers to non-replacement of a hydrogen atom by another substituent and remaining of the hydrogen atom.

As used herein, “hydrogen (—H) substituted” may also include “deuterium substituted (-D)” or “tritium substituted (-T).” For example, any hydrogen in any compound described herein may be protium, deuterium, or tritium (e.g., based on natural or artificial substitution).

As used herein, the term “or” is not an exclusive term, e.g., “A or B” would include A, B, or A and B.

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and all elements of the hydrocarbon aromatic moiety have p-orbitals which form conjugation, for example, a phenyl group, a naphthyl group, and the like, two or more hydrocarbon aromatic moieties may be linked by a sigma bond and may be, for example, a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof.

As used herein, hole characteristics refer to an ability to donate an electron to form a hole when an electric field is applied. A hole formed in the anode may be easily injected into the light emitting layer and transported in the light emitting layer due to conductive characteristics according to a highest occupied molecular orbital (HOMO) level.

In addition, electronic characteristics refer to an ability to accept an electron when an electric field is applied. Electrons formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to example embodiments is described.

The compound for the organic optoelectronic device according to some example embodiments is represented by Chemical Formula 1.

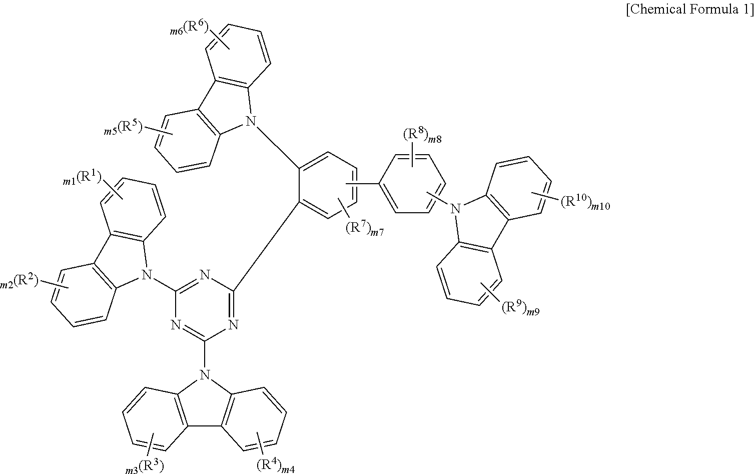

In Chemical Formula 1, R¹ to R¹⁰ may each independently be, e.g., hydrogen, deuterium, a substituted or unsubstituted C1 to C20 alkyl group, a substituted or unsubstituted C3 to C20 cycloalkyl group, a substituted or unsubstituted phenyl group, a substituted or unsubstituted biphenyl group, or a substituted or unsubstituted terphenyl group.

m1 to m6, and m8 to m10 may each independently be, e.g., an integer of 1 to 4.

m7 may be, e.g., an integer of 1 to 3.

In an implementation, m1 may be, e.g., 2, 3, or 4, and each R¹ may be the same or different from each other.

In an implementation, m2 may be, e.g., 2, 3, or 4, and each R² may be the same or different from each other.

In an implementation, m3 may be, e.g., 2, 3, or 4, and each R³ may be the same or different from each other.

In an implementation, m4 may be, e.g., 2, 3, or 4, and each R⁴ may be the same or different from each other.

In an implementation, m5 may be, e.g., 2, 3, or 4, and each R⁵ may be the same or different from each other.

In an implementation, m6 may be, e.g., 2, 3, or 4, and each R⁶ may be the same or different from each other.

In an implementation, m7 may be, e.g., 2 or 3, and each R⁷ may be the same or different from each other.

In an implementation, m8 may be, e.g., 2, 3, or 4, and each R⁸ may be the same or different from each other.

In an implementation, m9 may be, e.g., 2, 3, or 4, and each R⁹ may be the same or different from each other.

In an implementation, m10 may be, e.g., 2, 3, or 4, and each R¹⁰ may be the same or different from each other.

In an implementation, a LUMO phore (LUMO part of the molecule) may be expanded, thereby improving efficiency and life-span, by including a triazine substituted with a 9-carbazole linked via an ortho-phenylene, and including a phenyl group that is further substituted with carbazole with respect to the ortho-phenylene to which the 9-carbazole is linked.

In an implementation, by linking carbazole through an ortho-phenylene linker, it may be possible to construct a material having shallow LUMO and HOMO values overall, and the process temperature may be significantly lowered to increase thermal stability.

In an implementation, the efficiency characteristics and life-span characteristics may be further improved by including a 9-carbazolyl group directly linked to the triazine as another substituent of the triazine.

In an implementation, Chemical Formula 1 may be, e.g., represented by one of Chemical Formula 1-1 to Chemical Formula 1-4.

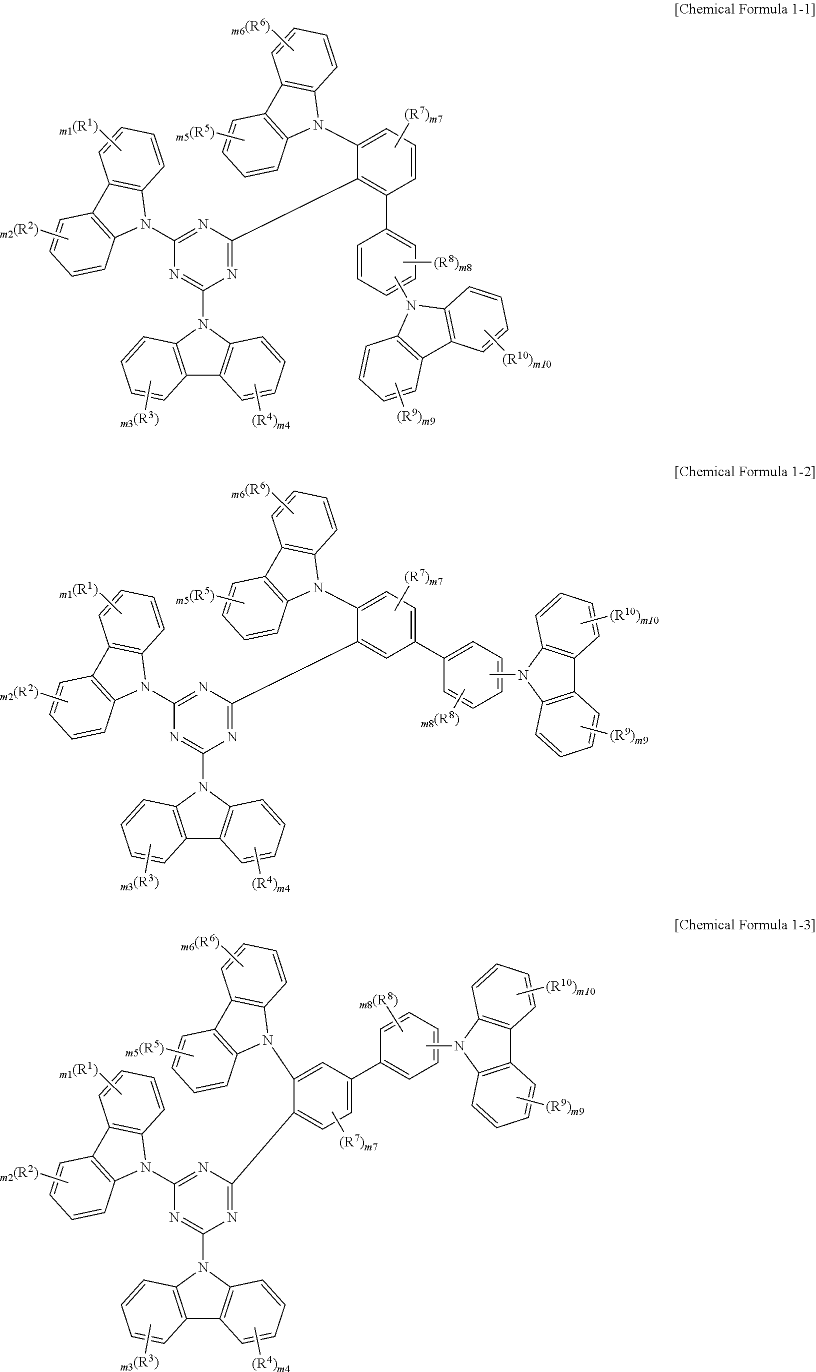

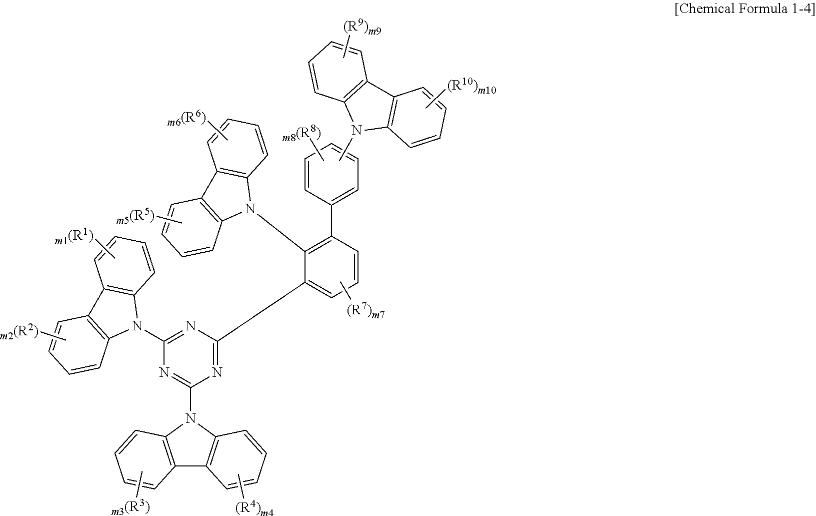

In Chemical Formula 1-1 to Chemical Formula 1-4, R¹ to R¹⁰ and m1 to m10 may be, e.g., defined the same as those of Chemical Formula 1.

In an implementation, Chemical Formula 1-1 may be, e.g., represented by one of Chemical Formula 1-1a, Chemical Formula 1-1b, and Chemical Formula 1-1c.

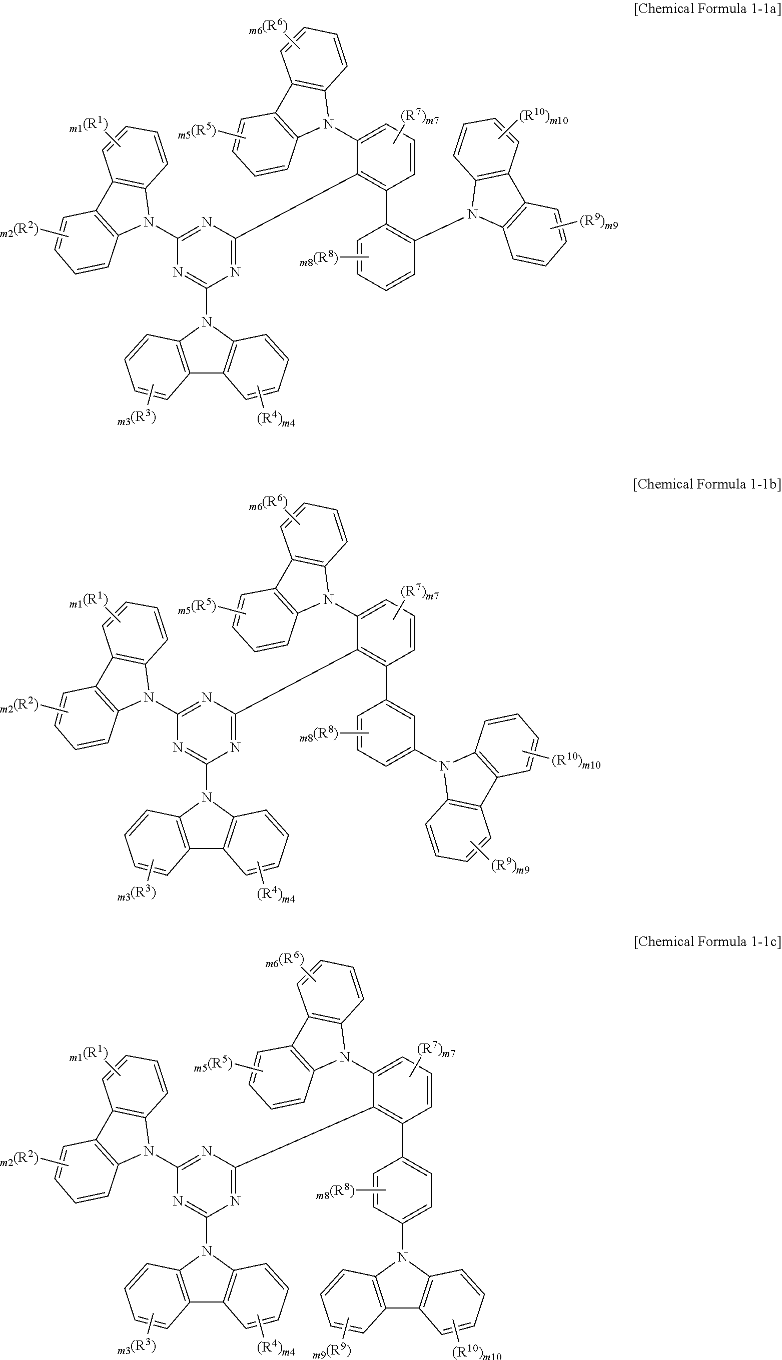

In Chemical Formula 1-1a, Chemical Formula 1-1b, and Chemical Formula 1-1c, R¹ to R¹⁰ and m1 to m10 may be, e.g., defined the same as those of Chemical Formula 1.

In an implementation, Chemical Formula 1-2 may be, e.g., represented by one of Chemical Formula 1-2a, Chemical Formula 1-2b, and Chemical Formula 1-2c.

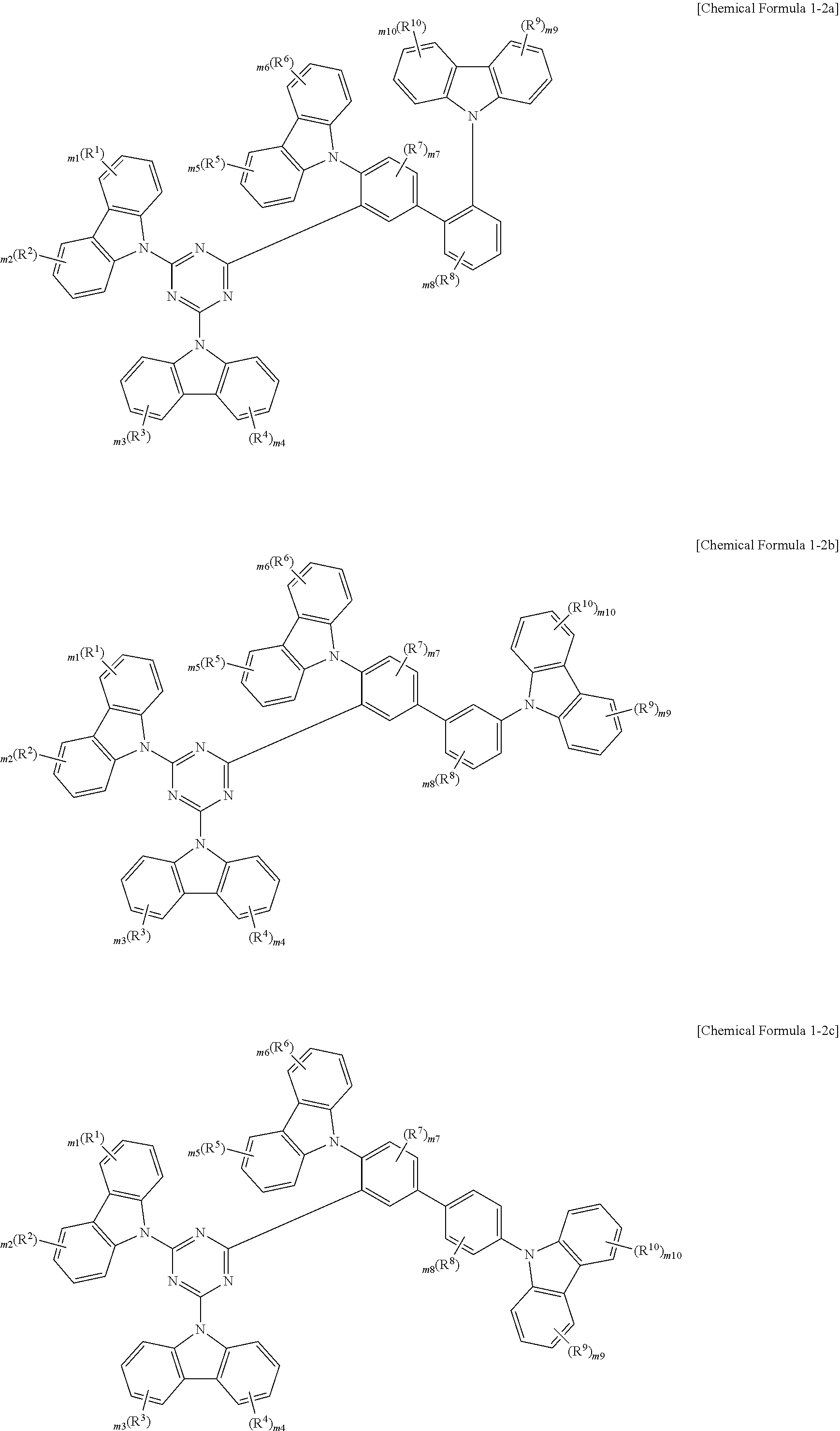

In Chemical Formula 1-2a, Chemical Formula 1-2b, and Chemical Formula 1-2c, R¹ to R¹⁰ and m1 to m10 may be, e.g., defined the same as those of Chemical Formula 1.

In an implementation, Chemical Formula 1-3 may be, e.g., represented by one of Chemical Formula 1-3a, Chemical Formula 1-3b, and Chemical Formula 1-3c.

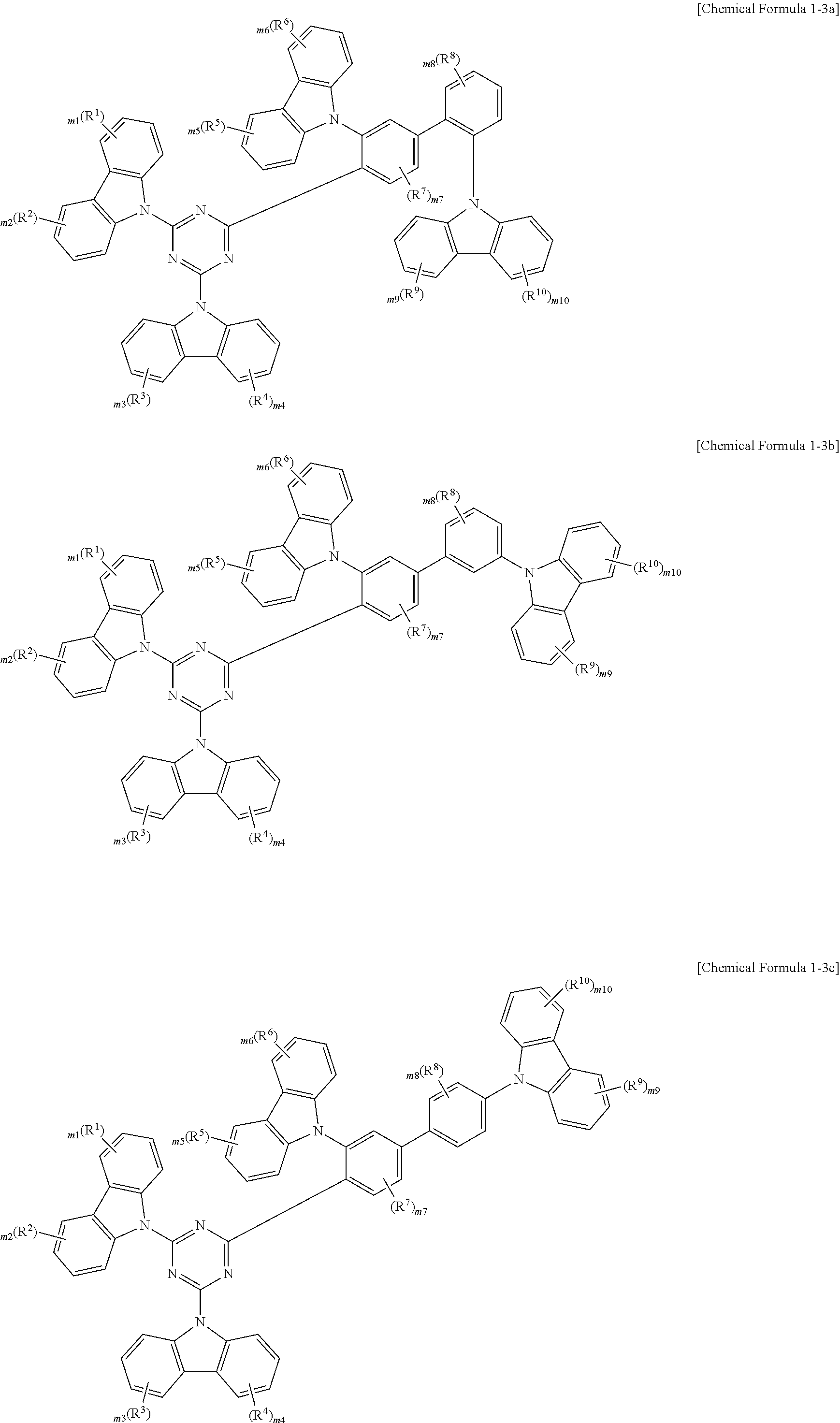

In Chemical Formula 1-3, R¹ to R¹⁰ and m1 to m10 may be, e.g., defined the same as those of Chemical Formula 1.

In an implementation, Chemical Formula 1-4 may be, e.g., represented by one of Chemical Formula 1-4a, Chemical Formula 1-4b, and Chemical Formula 1-4c.

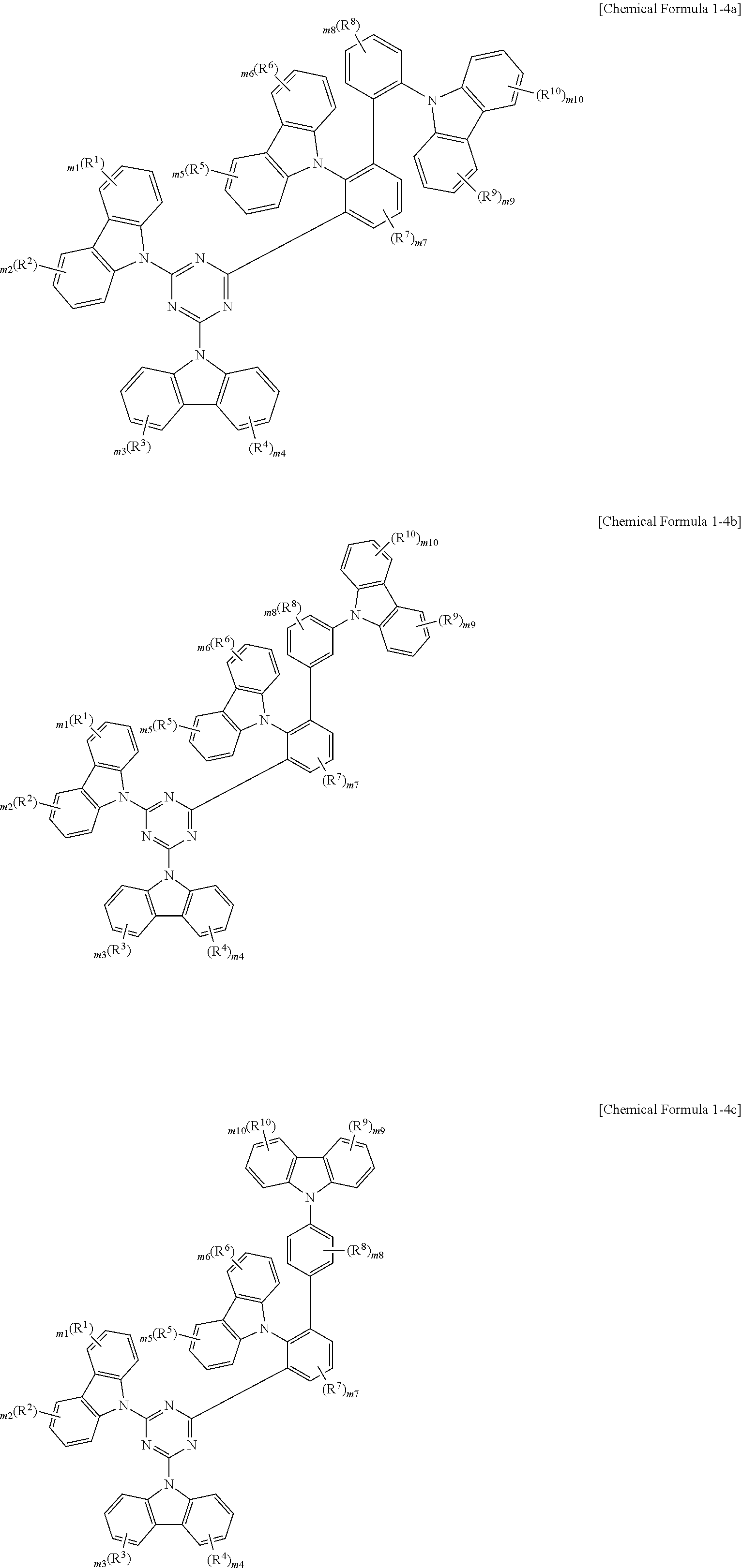

In Chemical Formula 1-4a, Chemical Formula 1-4b, and Chemical Formula 1-4c, R¹ to R¹⁰ and m1 to m10 may be, e.g., defined the same as those of Chemical Formula 1.

In an implementation, Chemical Formula 1 may be, e.g., represented by one of Chemical Formula 1-2, Chemical Formula 1-3, and Chemical Formula 1-4.

In an implementation, Chemical Formula 1 may be, e.g., represented by one of Chemical Formula 1-2a, Chemical Formula 1-3a, and Chemical Formula 1-4a.

In an implementation, Chemical Formula 1 may be, e.g., represented by Chemical Formula 1-2a or Chemical Formula 1-4a.

In an implementation, R¹ to R¹⁰ may each independently be, e.g., hydrogen, deuterium, a substituted or unsubstituted C1 to C10 alkyl group, a substituted or unsubstituted C3 to C10 cycloalkyl group, a substituted or unsubstituted phenyl group, or a substituted or unsubstituted biphenyl group.

In an implementation, R¹ to R¹⁰ may each independently be, e.g., hydrogen, deuterium, a substituted or unsubstituted methyl group, a substituted or unsubstituted ethyl group, a substituted or unsubstituted propyl group, a substituted or unsubstituted butyl group, a substituted or unsubstituted tert-butyl group, a substituted or unsubstituted cyclohexyl group, or a substituted or unsubstituted phenyl group.

In an implementation, the compound represented by Chemical Formula 1 may be, e.g., a compound of Group 1.

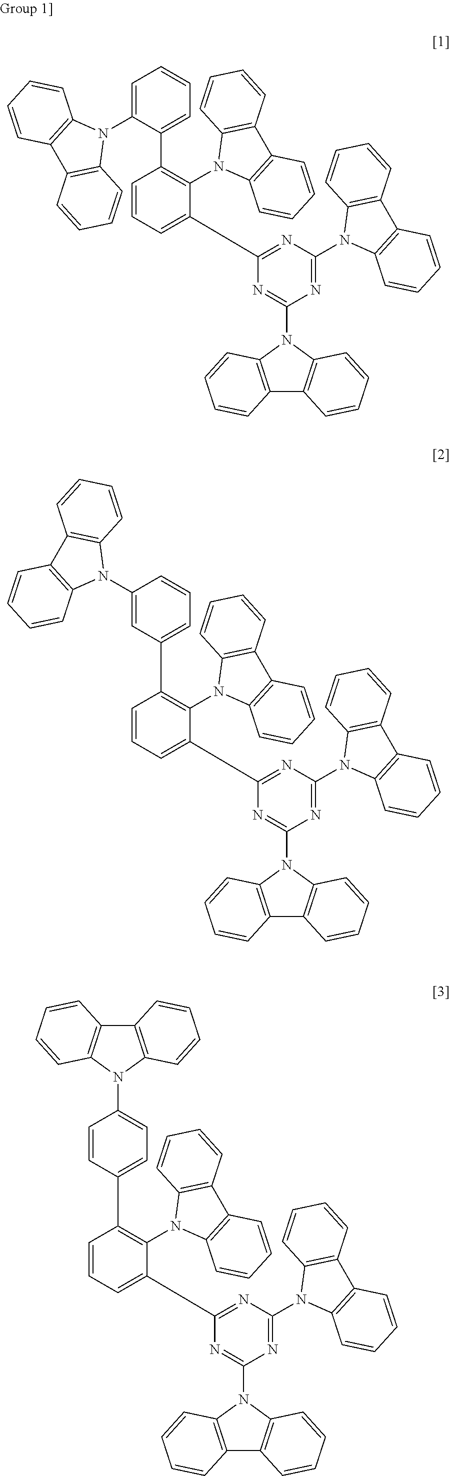 [SMILES: c1ccc(-n2c3ccccc3c3ccccc32)c(-c2cccc(-c3nc(-n4c5ccccc5c5ccccc54)nc(-n4c5ccccc5c5ccccc54)n3)c2-n2c3ccccc3c3ccccc32)c1 ; c1cc(-c2cccc(-c3nc(-n4c5ccccc5c5ccccc54)nc(-n4c5ccccc5c5ccccc54)n3)c2-n2c3ccccc3c3ccccc32)cc(-n2c3ccccc3c3ccccc32)c1 ; c1cc(-c2ccc(-n3c4ccccc4c4ccccc43)cc2)c(-n2c3ccccc3c3ccccc32)c(-c2nc(-n3c4ccccc4c4ccccc43)nc(-n3c4ccccc4c4ccccc43)n2)c1]

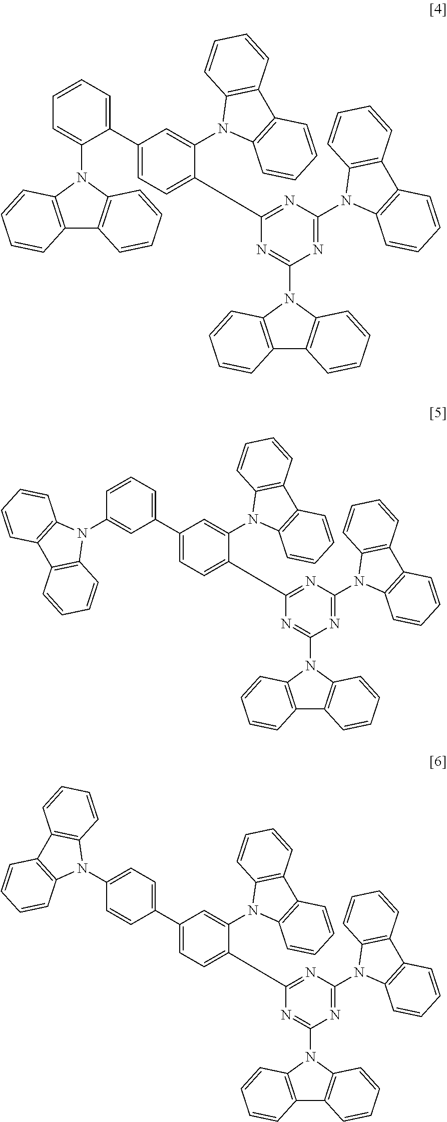 [SMILES: c1ccc(-n2c3ccccc3c3ccccc32)c(-c2ccc(-c3nc(-n4c5ccccc5c5ccccc54)nc(-n4c5ccccc5c5ccccc54)n3)c(-n3c4ccccc4c4ccccc43)c2)c1 ; c1cc(-c2ccc(-c3nc(-n4c5ccccc5c5ccccc54)nc(-n4c5ccccc5c5ccccc54)n3)c(-n3c4ccccc4c4ccccc43)c2)cc(-n2c3ccccc3c3ccccc32)c1 ; c1ccc2c(c1)c1ccccc1n2-c1ccc(-c2ccc(-c3nc(-n4c5ccccc5c5ccccc54)nc(-n4c5ccccc5c5ccccc54)n3)c(-n3c4ccccc4c4ccccc43)c2)cc1]

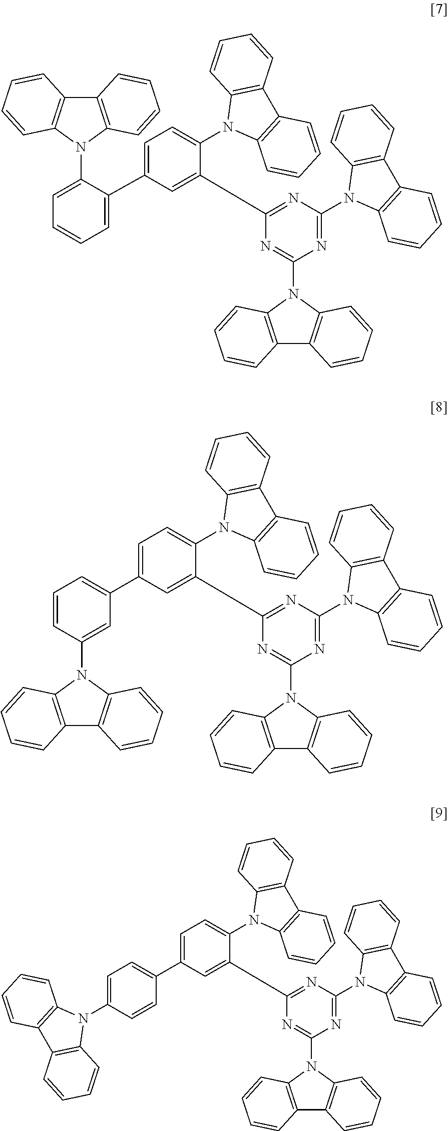 [SMILES: c1ccc(-n2c3ccccc3c3ccccc32)c(-c2ccc(-n3c4ccccc4c4ccccc43)c(-c3nc(-n4c5ccccc5c5ccccc54)nc(-n4c5ccccc5c5ccccc54)n3)c2)c1 ; c1cc(-c2ccc(-n3c4ccccc4c4ccccc43)c(-c3nc(-n4c5ccccc5c5ccccc54)nc(-n4c5ccccc5c5ccccc54)n3)c2)cc(-n2c3ccccc3c3ccccc32)c1 ; c1ccc2c(c1)c1ccccc1n2-c1ccc(-c2ccc(-n3c4ccccc4c4ccccc43)c(-c3nc(-n4c5ccccc5c5ccccc54)nc(-n4c5ccccc5c5ccccc54)n3)c2)cc1]

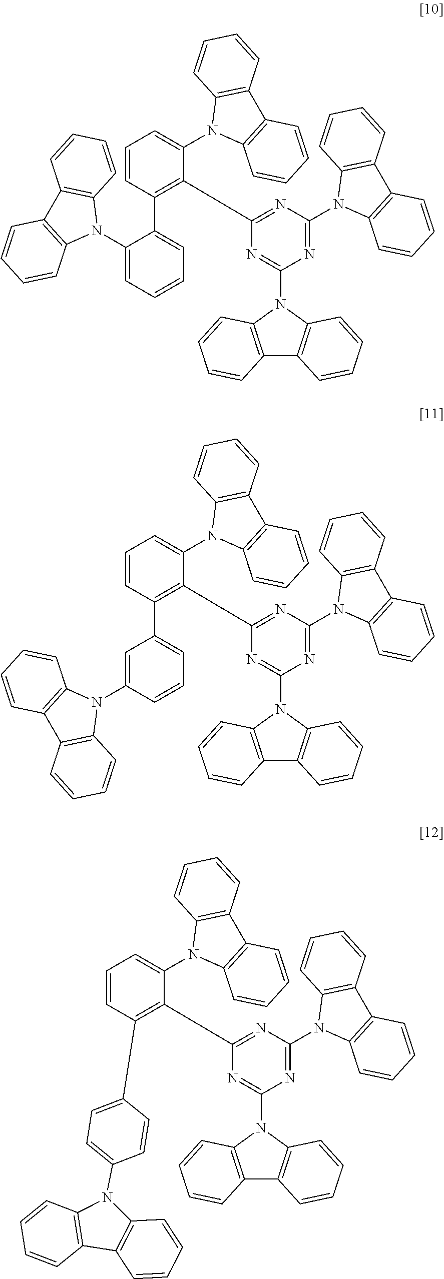 [SMILES: c1ccc(-n2c3ccccc3c3ccccc32)c(-c2cccc(-n3c4ccccc4c4ccccc43)c2-c2nc(-n3c4ccccc4c4ccccc43)nc(-n3c4ccccc4c4ccccc43)n2)c1 ; c1cc(-c2cccc(-n3c4ccccc4c4ccccc43)c2-c2nc(-n3c4ccccc4c4ccccc43)nc(-n3c4ccccc4c4ccccc43)n2)cc(-n2c3ccccc3c3ccccc32)c1 ; c1cc(-c2ccc(-n3c4ccccc4c4ccccc43)cc2)c(-c2nc(-n3c4ccccc4c4ccccc43)nc(-n3c4ccccc4c4ccccc43)n2)c(-n2c3ccccc3c3ccccc32)c1]

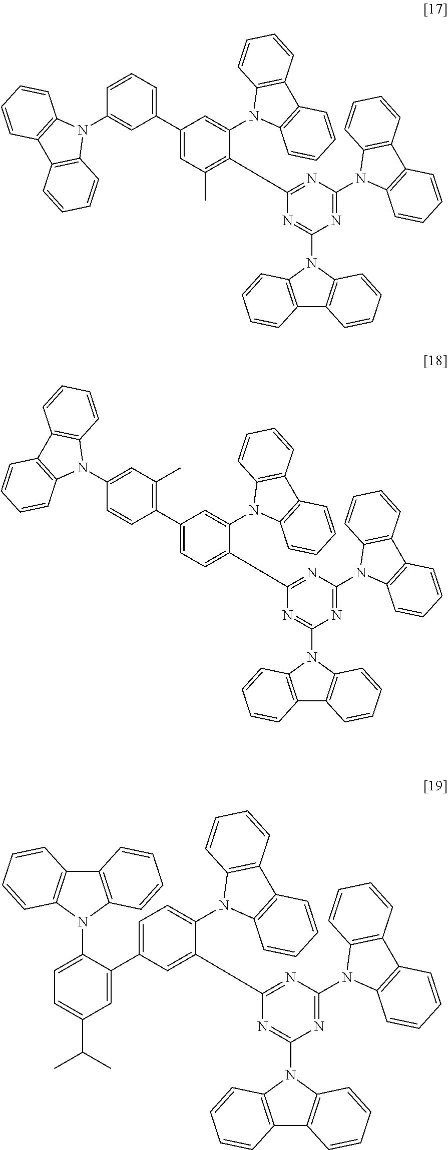 [SMILES: Cc1cc(-c2cccc(-n3c4ccccc4c4ccccc43)c2)cc(-n2c3ccccc3c3ccccc32)c1-c1nc(-n2c3ccccc3c3ccccc32)nc(-n2c3ccccc3c3ccccc32)n1 ; Cc1cc(-n2c3ccccc3c3ccccc32)ccc1-c1ccc(-c2nc(-n3c4ccccc4c4ccccc43)nc(-n3c4ccccc4c4ccccc43)n2)c(-n2c3ccccc3c3ccccc32)c1 ; CC(C)c1ccc(-n2c3ccccc3c3ccccc32)c(-c2ccc(-n3c4ccccc4c4ccccc43)c(-c3nc(-n4c5ccccc5c5ccccc54)nc(-n4c5ccccc5c5ccccc54)n3)c2)c1]

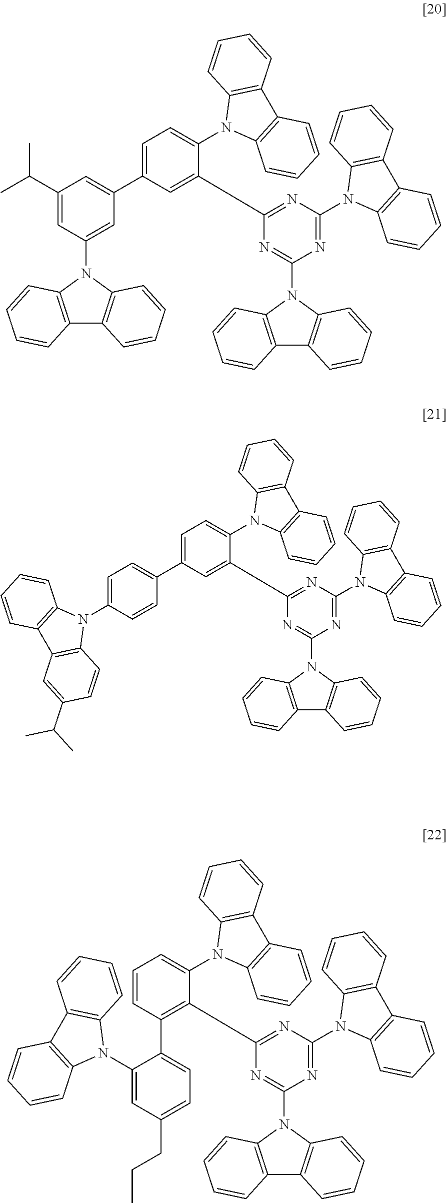 [SMILES: CC(C)c1cc(-c2ccc(-n3c4ccccc4c4ccccc43)c(-c3nc(-n4c5ccccc5c5ccccc54)nc(-n4c5ccccc5c5ccccc54)n3)c2)cc(-n2c3ccccc3c3ccccc32)c1 ; CC(C)c1ccc2c(c1)c1ccccc1n2-c1ccc(-c2ccc(-n3c4ccccc4c4ccccc43)c(-c3nc(-n4c5ccccc5c5ccccc54)nc(-n4c5ccccc5c5ccccc54)n3)c2)cc1 ; CCCc1ccc(-c2cccc(-n3c4ccccc4c4ccccc43)c2-c2nc(-n3c4ccccc4c4ccccc43)nc(-n3c4ccccc4c4ccccc43)n2)c(-n2c3ccccc3c3ccccc32)c1]

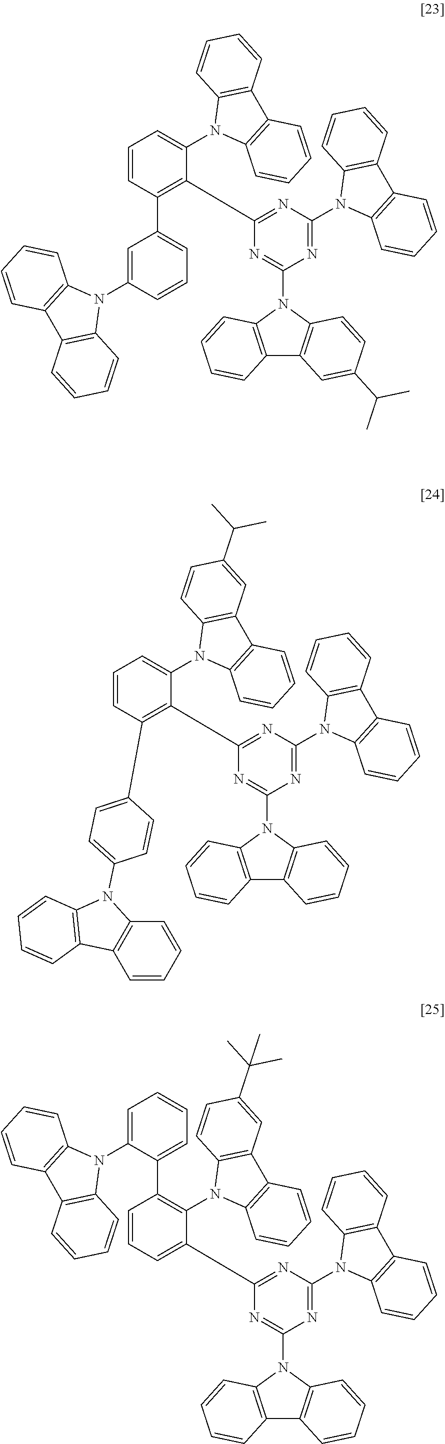 [SMILES: CC(C)c1ccc2c(c1)c1ccccc1n2-c1nc(-c2c(-c3cccc(-n4c5ccccc5c5ccccc54)c3)cccc2-n2c3ccccc3c3ccccc32)nc(-n2c3ccccc3c3ccccc32)n1 ; CC(C)c1ccc2c(c1)c1ccccc1n2-c1cccc(-c2ccc(-n3c4ccccc4c4ccccc43)cc2)c1-c1nc(-n2c3ccccc3c3ccccc32)nc(-n2c3ccccc3c3ccccc32)n1 ; CC(C)(C)c1ccc2c(c1)c1ccccc1n2-c1c(-c2nc(-n3c4ccccc4c4ccccc43)nc(-n3c4ccccc4c4ccccc43)n2)cccc1-c1ccccc1-n1c2ccccc2c2ccccc21]

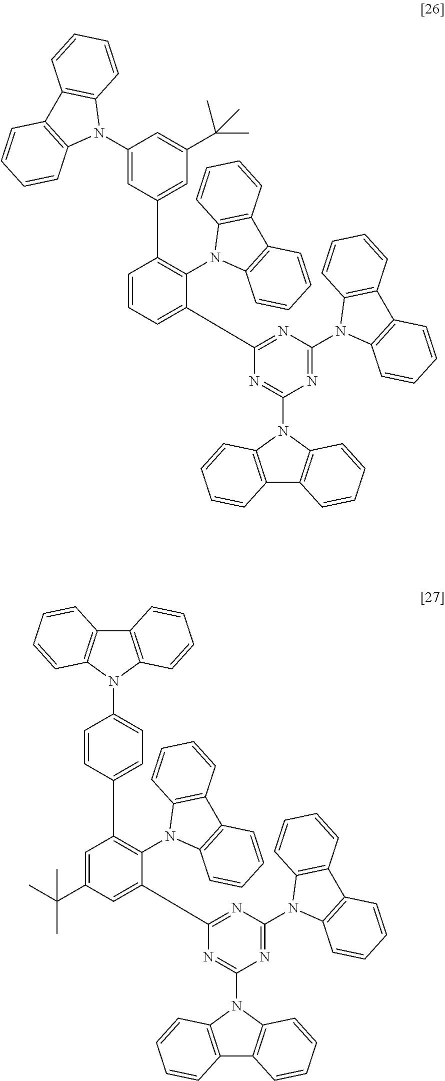 [SMILES: CC(C)(C)c1cc(-c2cccc(-c3nc(-n4c5ccccc5c5ccccc54)nc(-n4c5ccccc5c5ccccc54)n3)c2-n2c3ccccc3c3ccccc32)cc(-n2c3ccccc3c3ccccc32)c1 ; CC(C)(C)c1cc(-c2ccc(-n3c4ccccc4c4ccccc43)cc2)c(-n2c3ccccc3c3ccccc32)c(-c2nc(-n3c4ccccc4c4ccccc43)nc(-n3c4ccccc4c4ccccc43)n2)c1]

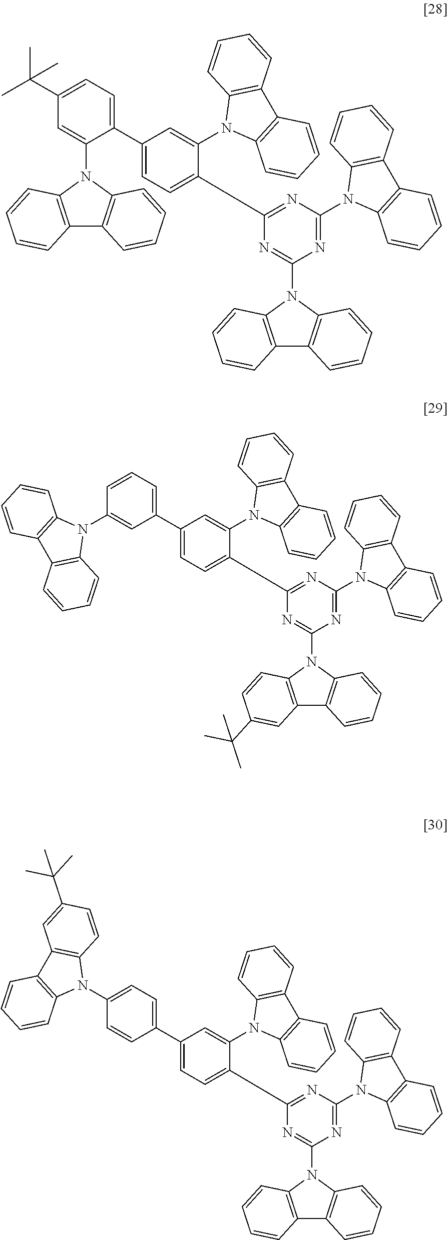 [SMILES: CC(C)(C)c1ccc(-c2ccc(-c3nc(-n4c5ccccc5c5ccccc54)nc(-n4c5ccccc5c5ccccc54)n3)c(-n3c4ccccc4c4ccccc43)c2)c(-n2c3ccccc3c3ccccc32)c1 ; CC(C)(C)c1ccc2c(c1)c1ccccc1n2-c1nc(-c2ccc(-c3cccc(-n4c5ccccc5c5ccccc54)c3)cc2-n2c3ccccc3c3ccccc32)nc(-n2c3ccccc3c3ccccc32)n1 ; CC(C)(C)c1ccc2c(c1)c1ccccc1n2-c1ccc(-c2ccc(-c3nc(-n4c5ccccc5c5ccccc54)nc(-n4c5ccccc5c5ccccc54)n3)c(-n3c4ccccc4c4ccccc43)c2)cc1]

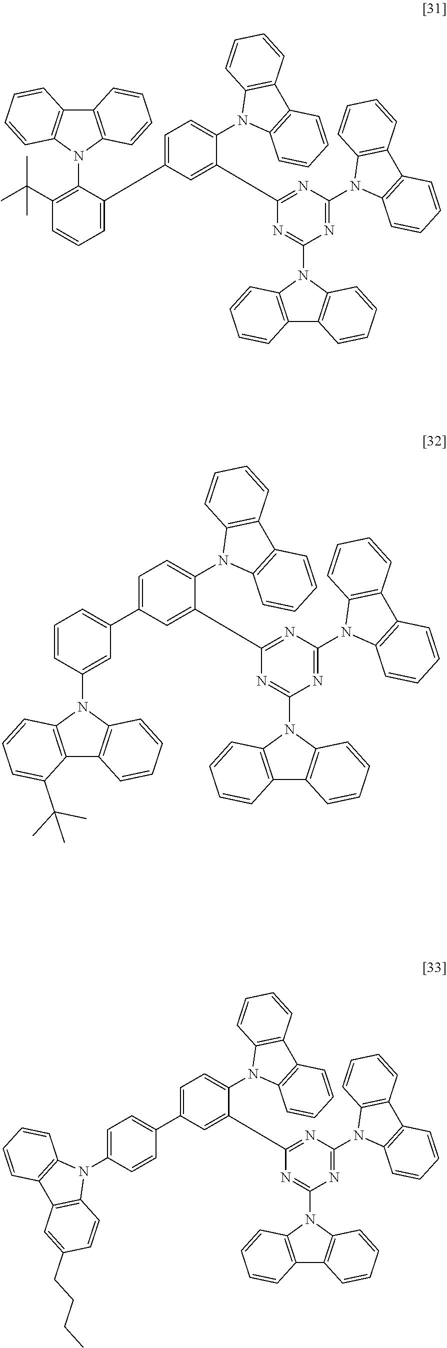 [SMILES: CC(C)(C)c1cccc(-c2ccc(-n3c4ccccc4c4ccccc43)c(-c3nc(-n4c5ccccc5c5ccccc54)nc(-n4c5ccccc5c5ccccc54)n3)c2)c1-n1c2ccccc2c2ccccc21 ; CC(C)(C)c1cccc2c1c1ccccc1n2-c1cccc(-c2ccc(-n3c4ccccc4c4ccccc43)c(-c3nc(-n4c5ccccc5c5ccccc54)nc(-n4c5ccccc5c5ccccc54)n3)c2)c1 ; CCCCc1ccc2c(c1)c1ccccc1n2-c1ccc(-c2ccc(-n3c4ccccc4c4ccccc43)c(-c3nc(-n4c5ccccc5c5ccccc54)nc(-n4c5ccccc5c5ccccc54)n3)c2)cc1]

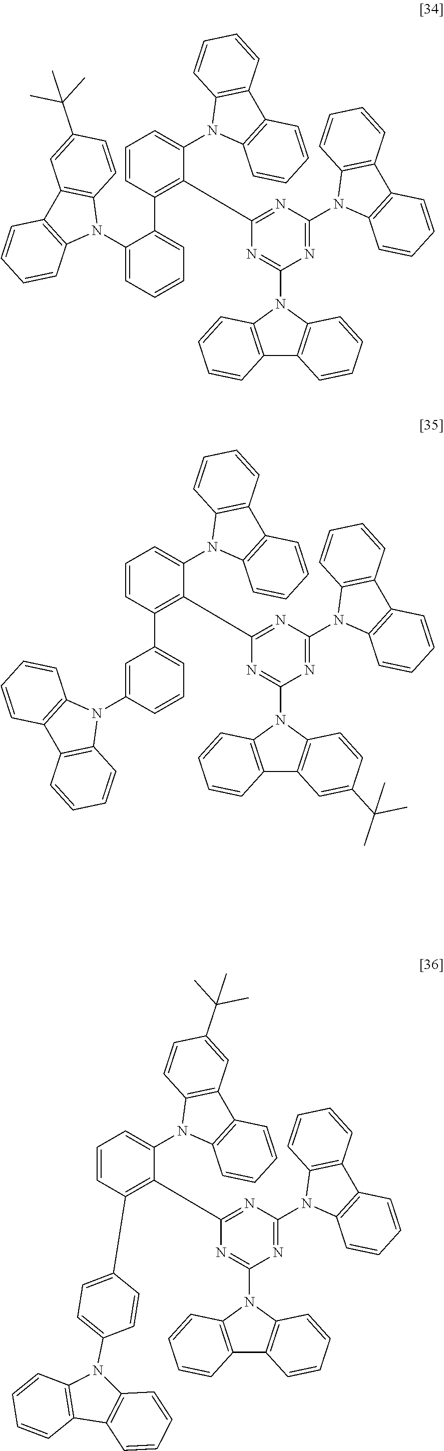 [SMILES: CC(C)(C)c1ccc2c(c1)c1ccccc1n2-c1ccccc1-c1cccc(-n2c3ccccc3c3ccccc32)c1-c1nc(-n2c3ccccc3c3ccccc32)nc(-n2c3ccccc3c3ccccc32)n1 ; CC(C)(C)c1ccc2c(c1)c1ccccc1n2-c1nc(-c2c(-c3cccc(-n4c5ccccc5c5ccccc54)c3)cccc2-n2c3ccccc3c3ccccc32)nc(-n2c3ccccc3c3ccccc32)n1 ; CC(C)(C)c1ccc2c(c1)c1ccccc1n2-c1cccc(-c2ccc(-n3c4ccccc4c4ccccc43)cc2)c1-c1nc(-n2c3ccccc3c3ccccc32)nc(-n2c3ccccc3c3ccccc32)n1]

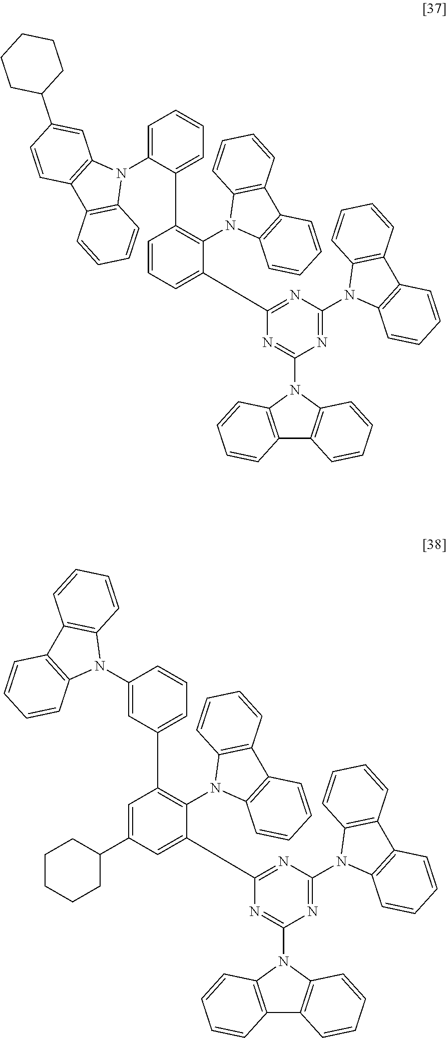 [SMILES: c1ccc(-n2c3ccccc3c3ccc(C4CCCCC4)cc32)c(-c2cccc(-c3nc(-n4c5ccccc5c5ccccc54)nc(-n4c5ccccc5c5ccccc54)n3)c2-n2c3ccccc3c3ccccc32)c1 ; c1cc(-c2cc(C3CCCCC3)cc(-c3nc(-n4c5ccccc5c5ccccc54)nc(-n4c5ccccc5c5ccccc54)n3)c2-n2c3ccccc3c3ccccc32)cc(-n2c3ccccc3c3ccccc32)c1]

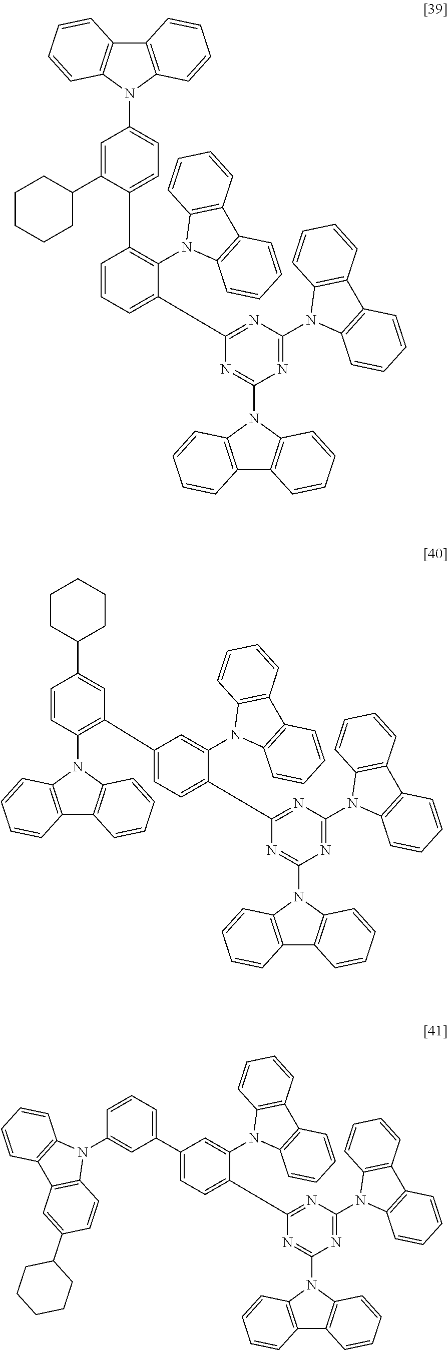 [SMILES: c1cc(-c2nc(-n3c4ccccc4c4ccccc43)nc(-n3c4ccccc4c4ccccc43)n2)c(-n2c3ccccc3c3ccccc32)c(-c2ccc(-n3c4ccccc4c4ccccc43)cc2C2CCCCC2)c1 ; c1ccc2c(c1)c1ccccc1n2-c1nc(-c2ccc(-c3cc(C4CCCCC4)ccc3-n3c4ccccc4c4ccccc43)cc2-n2c3ccccc3c3ccccc32)nc(-n2c3ccccc3c3ccccc32)n1 ; c1cc(-c2ccc(-c3nc(-n4c5ccccc5c5ccccc54)nc(-n4c5ccccc5c5ccccc54)n3)c(-n3c4ccccc4c4ccccc43)c2)cc(-n2c3ccccc3c3cc(C4CCCCC4)ccc32)c1]

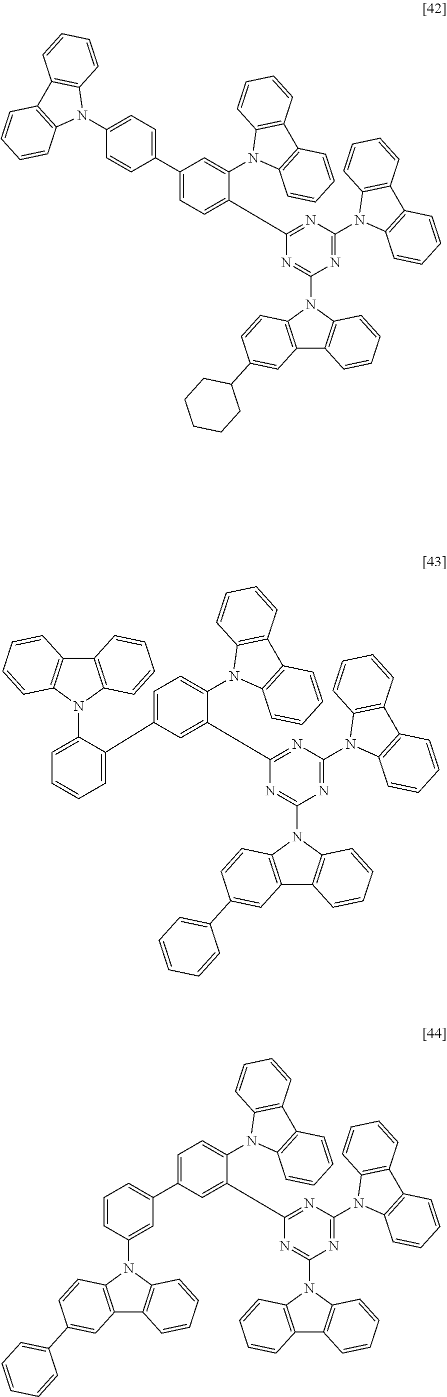

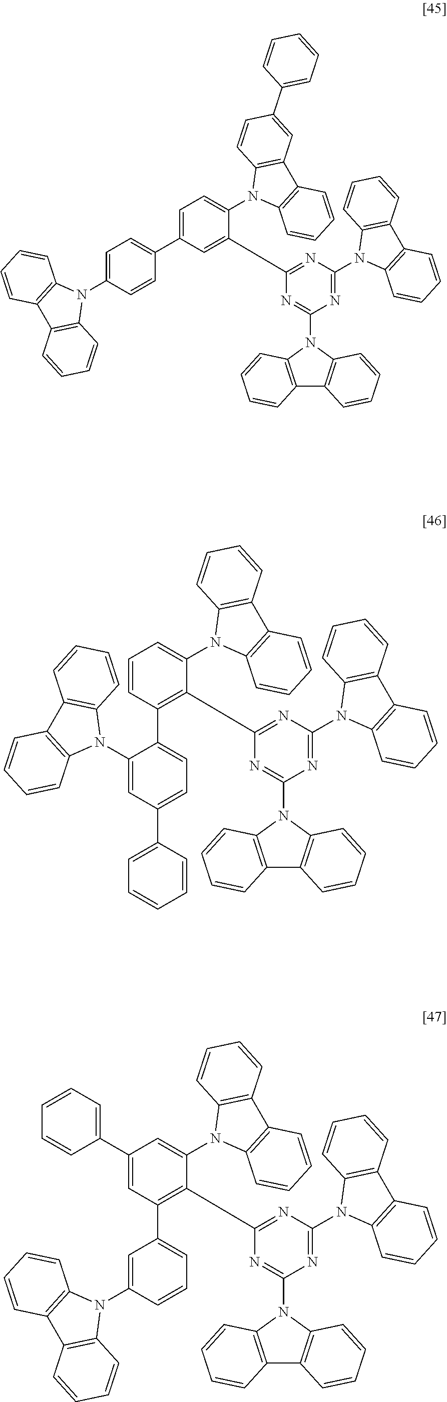

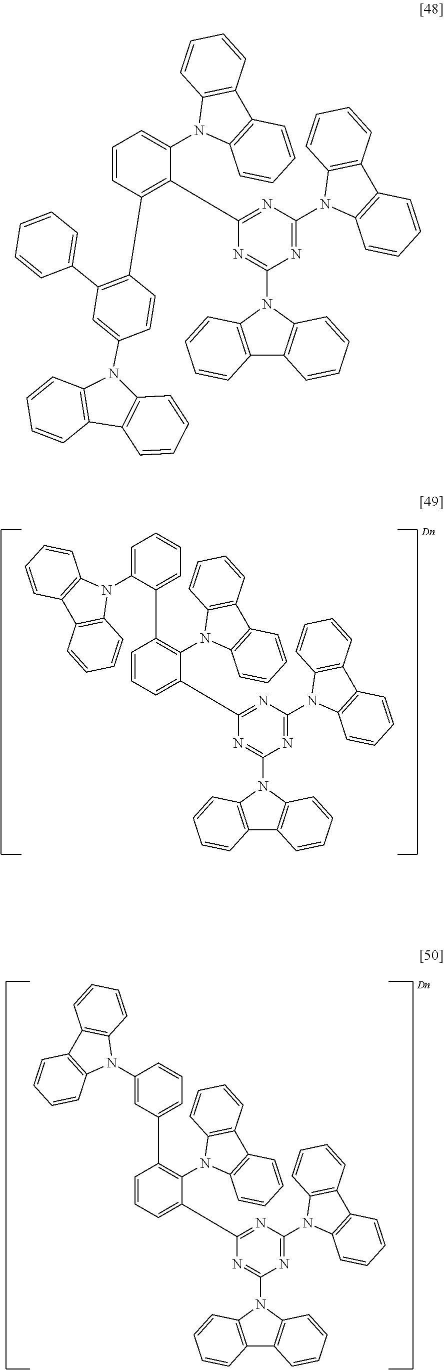

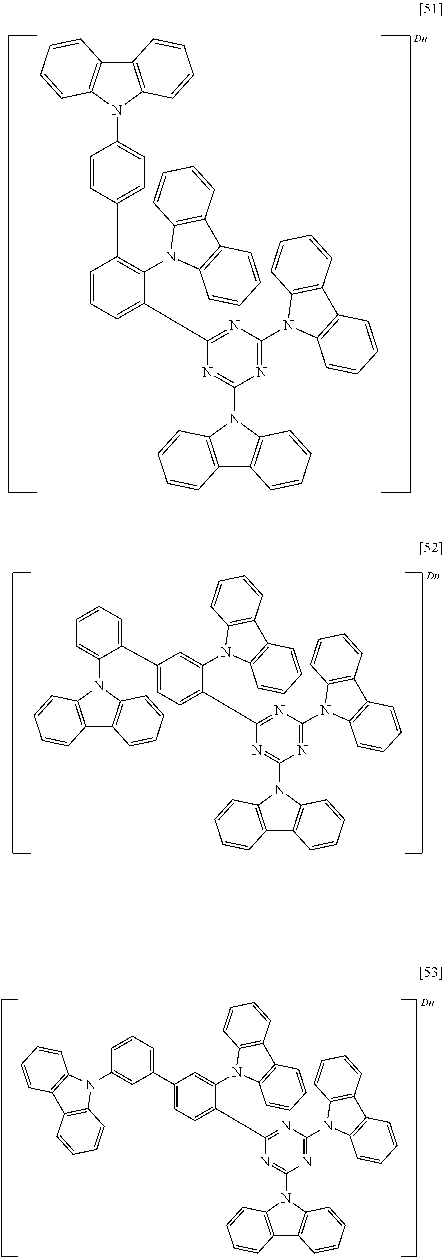

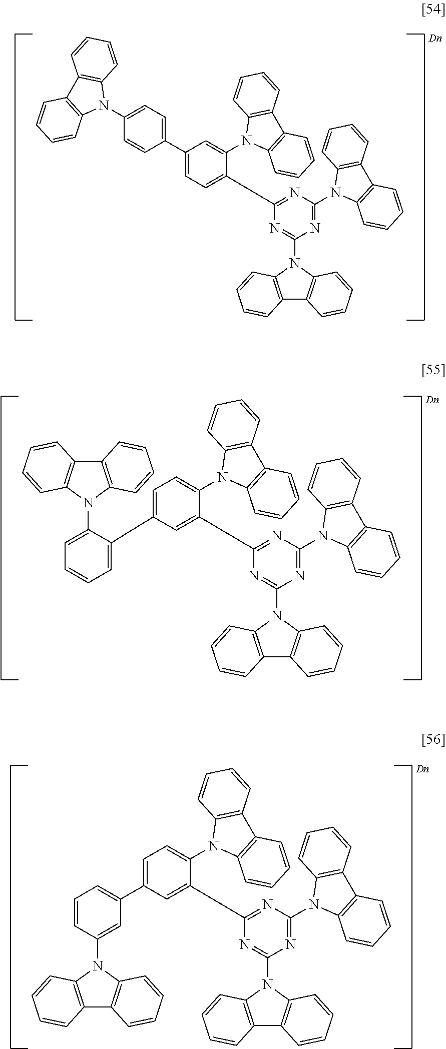

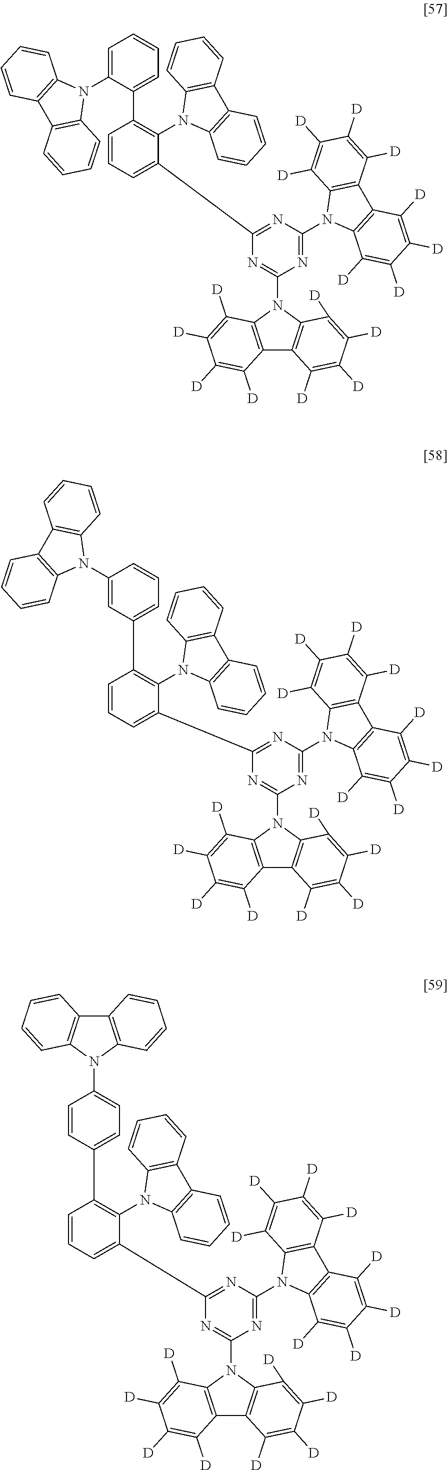

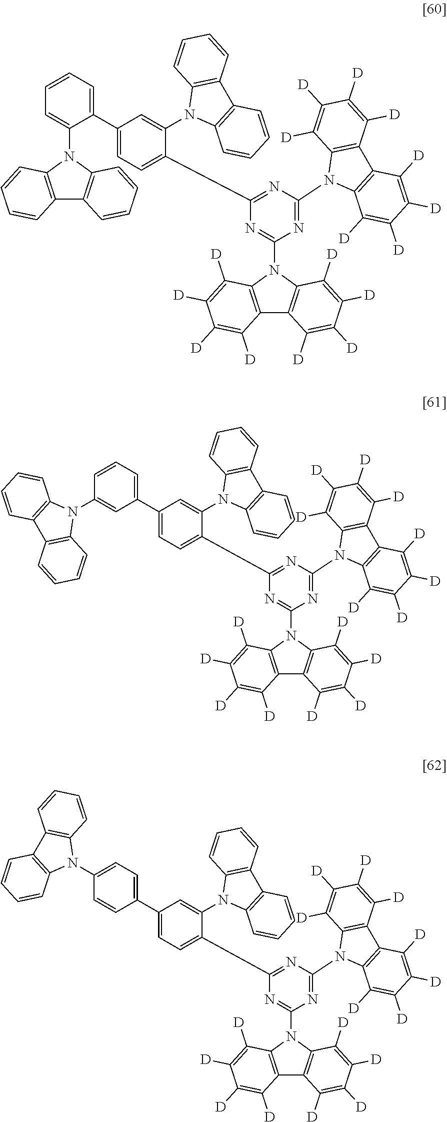

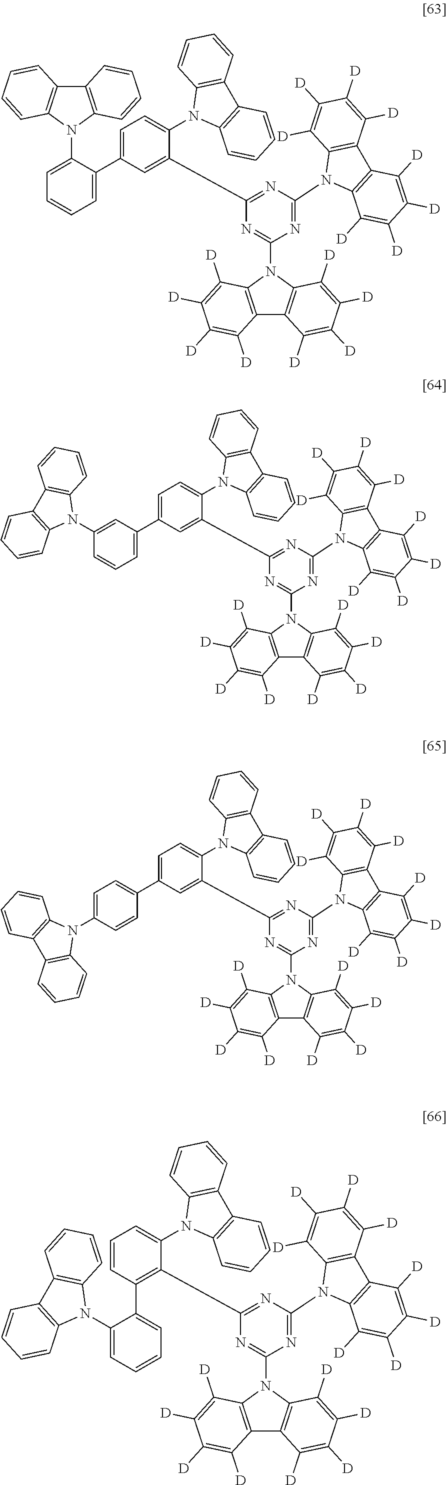

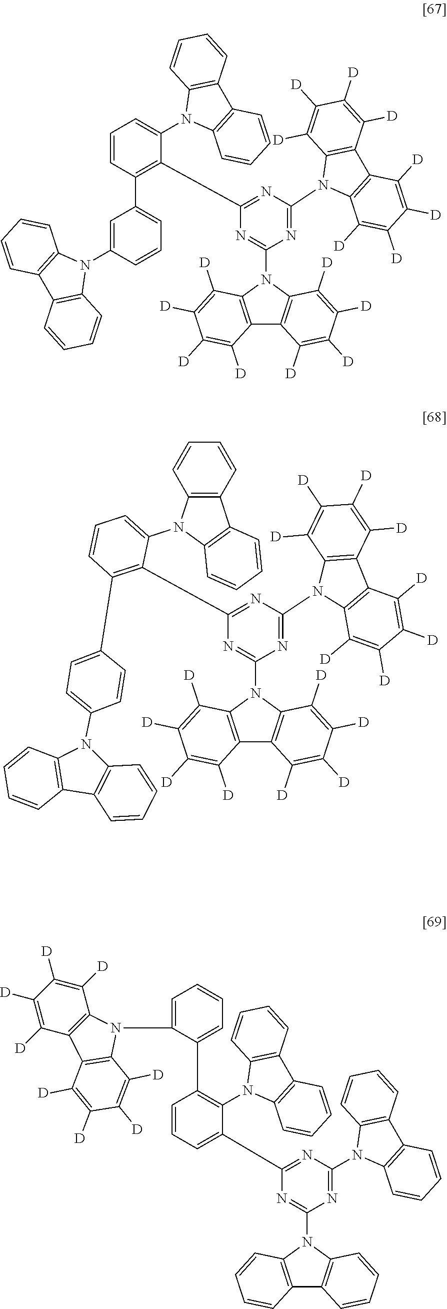

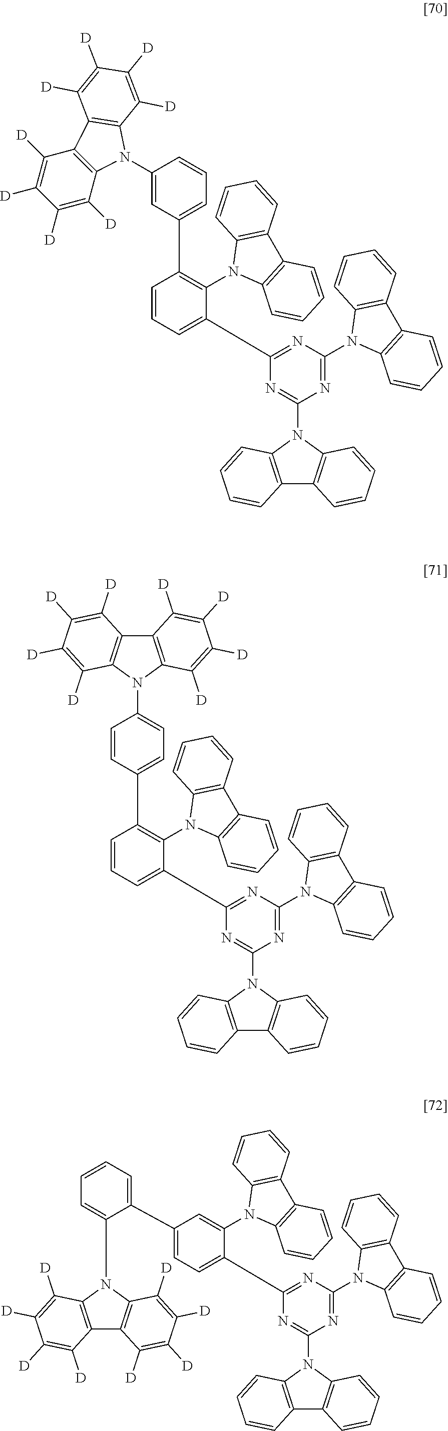 [SMILES: [2H]c1c([2H])c([2H])c2c(c1[2H])c1c([2H])c([2H])c([2H])c([2H])c1n2-c1cccc(-c2cccc(-c3nc(-n4c5ccccc5c5ccccc54)nc(-n4c5ccccc5c5ccccc54)n3)c2-n2c3ccccc3c3ccccc32)c1 ; [2H]c1c([2H])c([2H])c2c(c1[2H])c1c([2H])c([2H])c([2H])c([2H])c1n2-c1ccc(-c2cccc(-c3nc(-n4c5ccccc5c5ccccc54)nc(-n4c5ccccc5c5ccccc54)n3)c2-n2c3ccccc3c3ccccc32)cc1 ; [2H]c1c([2H])c([2H])c2c(c1[2H])c1c([2H])c([2H])c([2H])c([2H])c1n2-c1ccccc1-c1ccc(-c2nc(-n3c4ccccc4c4ccccc43)nc(-n3c4ccccc4c4ccccc43)n2)c(-n2c3ccccc3c3ccccc32)c1]

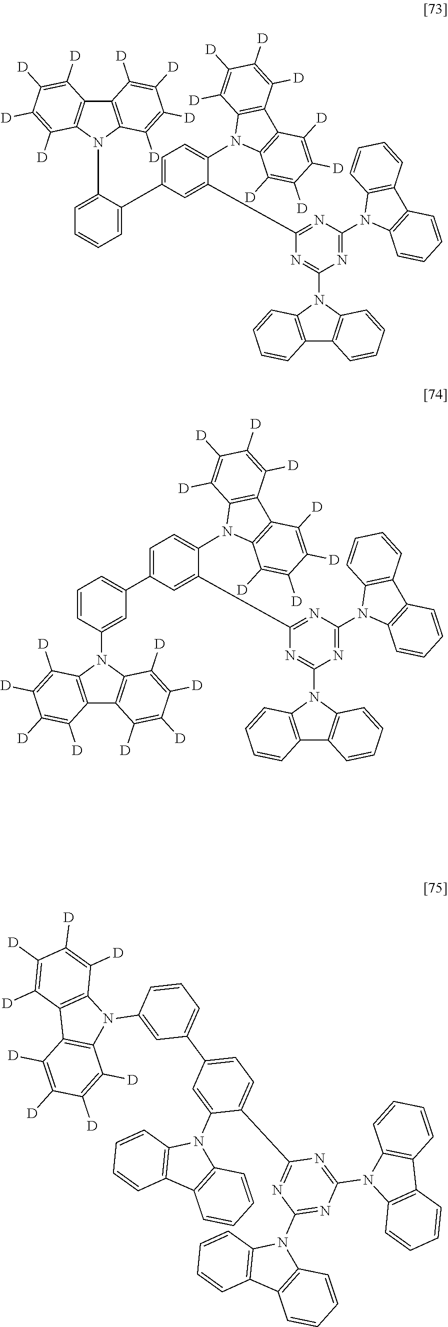

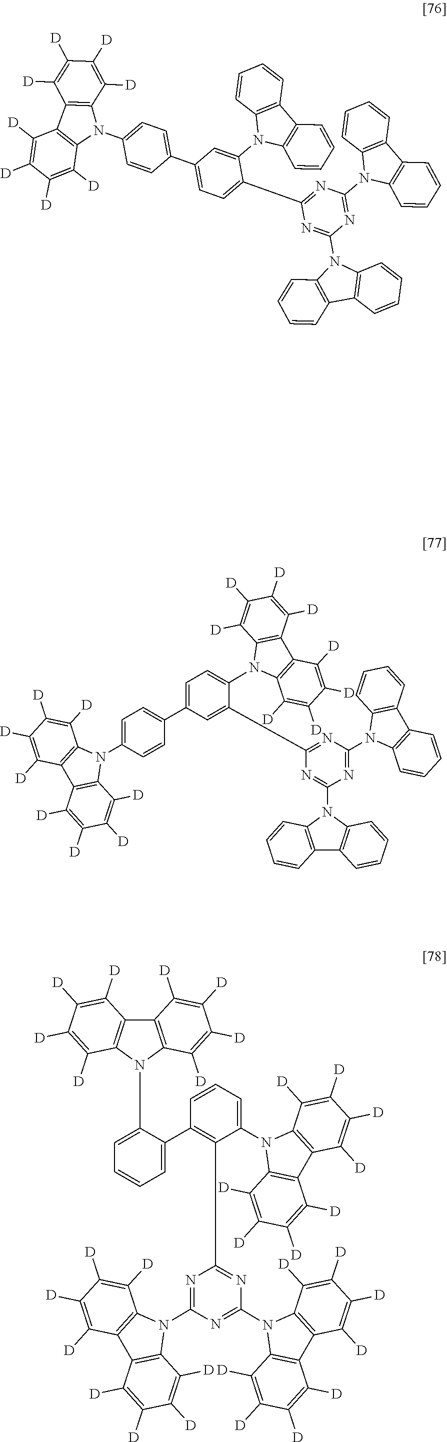

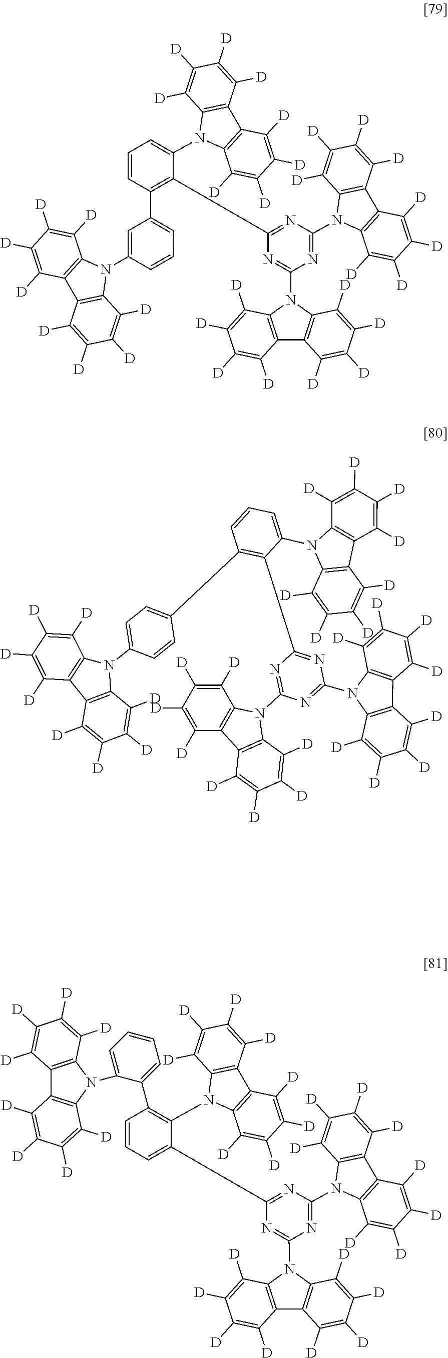

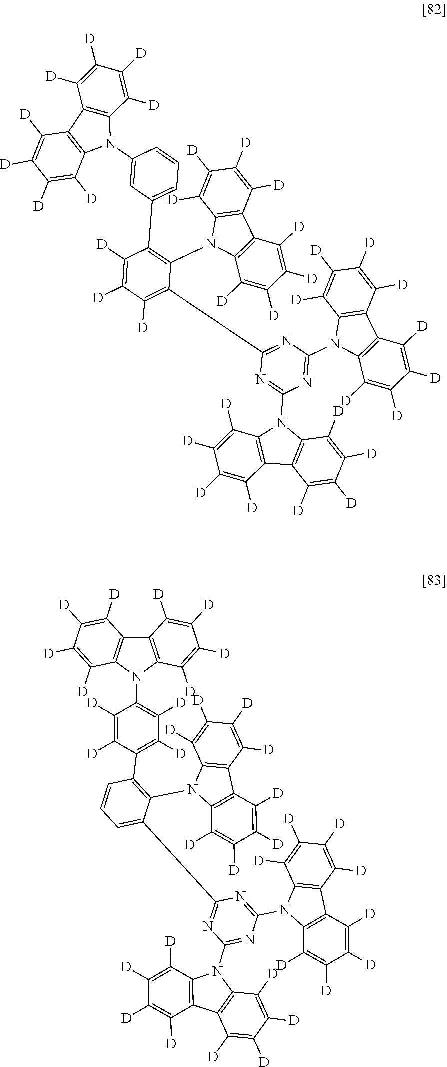

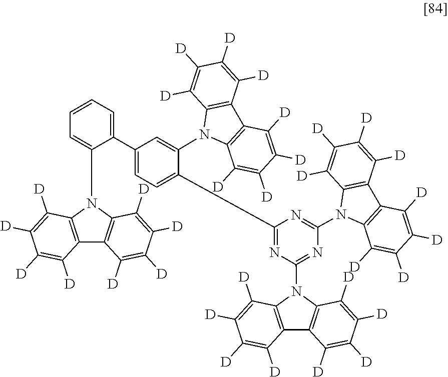 [SMILES: [2H]c1c([2H])c([2H])c2c(c1[2H])c1c([2H])c([2H])c([2H])c([2H])c1n2-c1nc(-c2ccc(-c3ccccc3-n3c4c([2H])c([2H])c([2H])c([2H])c4c4c([2H])c([2H])c([2H])c([2H])c43)cc2-n2c3c([2H])c([2H])c([2H])c([2H])c3c3c([2H])c([2H])c([2H])c([2H])c32)nc(-n2c3c([2H])c([2H])c([2H])c([2H])c3c3c([2H])c([2H])c([2H])c([2H])c32)n1]

Dn refers to the number of deuterium atoms substituted, and n is an integer between 1 and the maximum number of substitutions possible.

In an implementation, the compound represented by Chemical Formula 1 may be, e.g., a compound of Group 1-1.

### Group 1-1

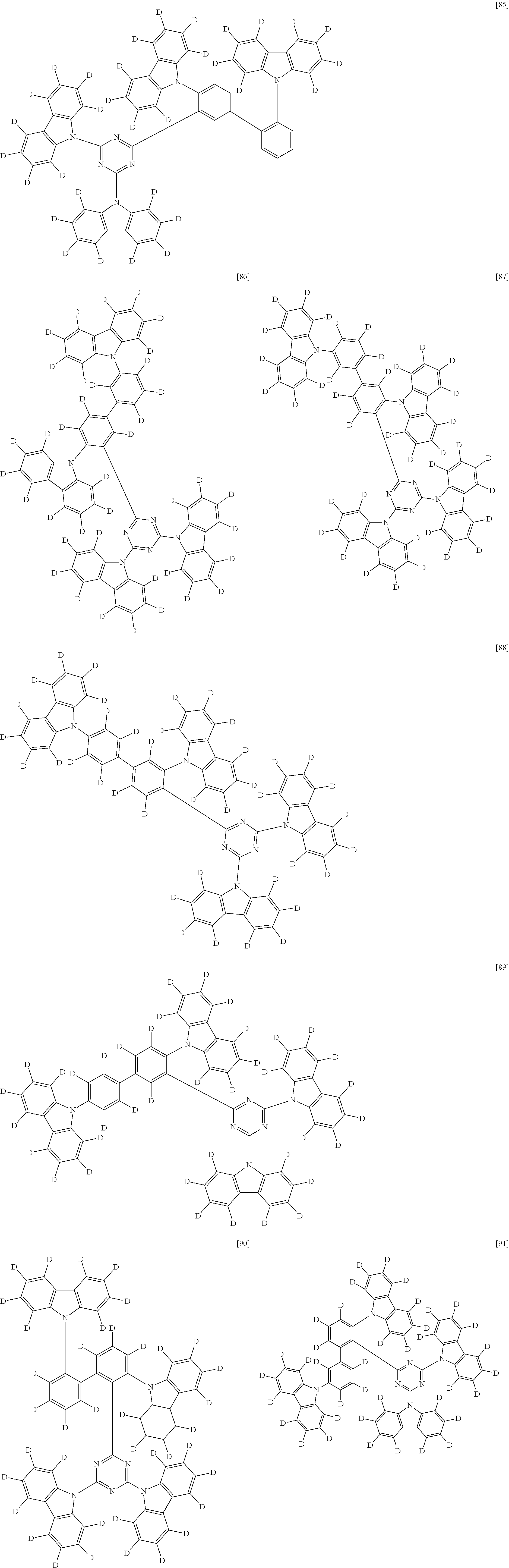

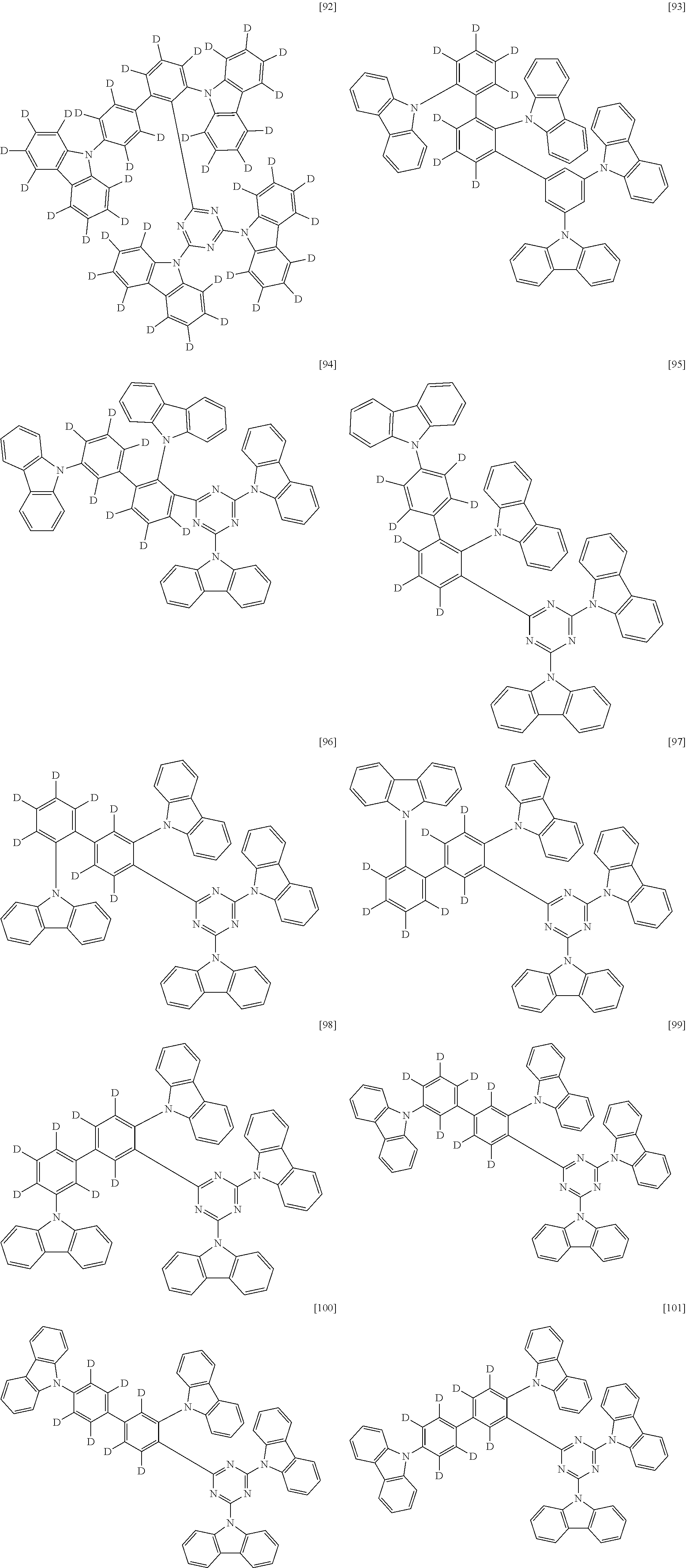

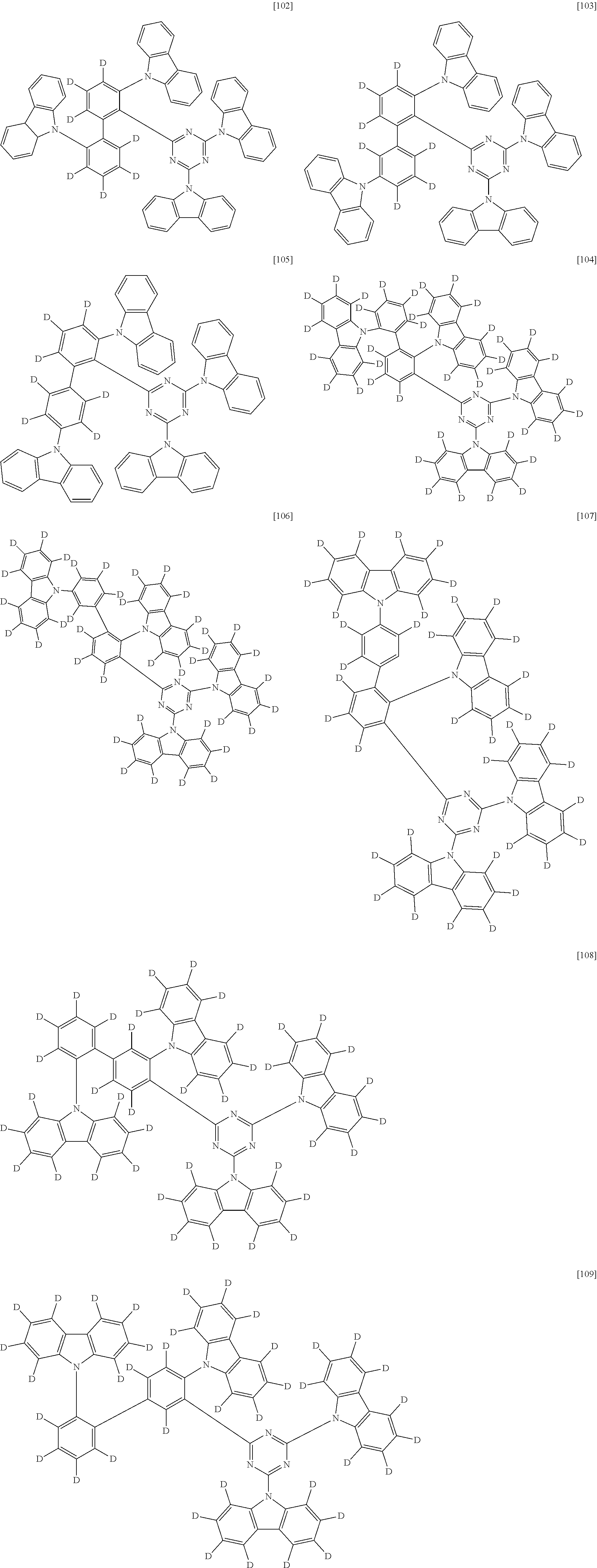

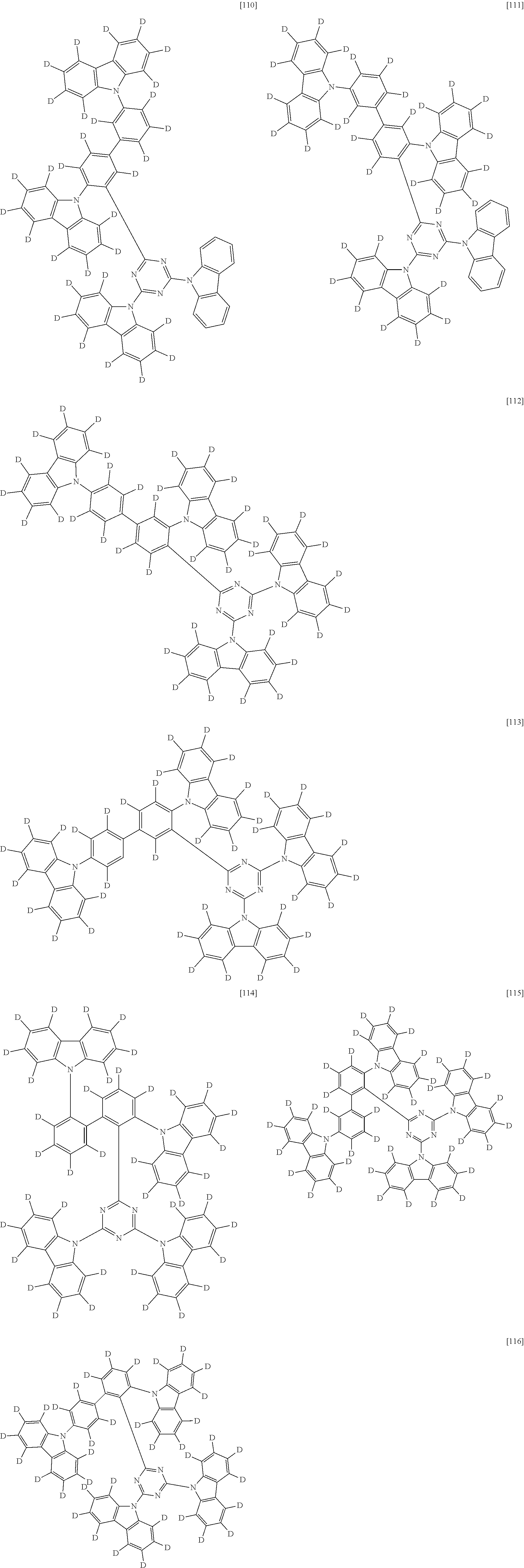

According to some example embodiments, a composition for an organic optoelectronic device includes, e.g., a first compound and a second compound, wherein the first compound may be the aforementioned compound for the organic optoelectronic device, and the second compound may be a compound represented by Chemical Formula 2, a combination of Chemical Formula 3 and Chemical Formula 4, or Chemical Formula 5.

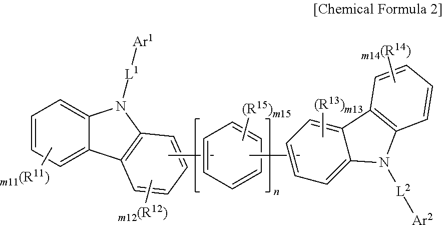

In Chemical Formula 2, R¹¹ to R¹⁵ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar¹ and Ar² may each independently be or include, e.g., a substituted or unsubstituted C6 to C20 aryl group or a substituted or unsubstituted C2 to C30 heterocyclic group.

L¹ and L² may each independently be or include, e.g., a single bond or a substituted or unsubstituted C6 to C20 arylene group.

m11, m14, and m15 may each independently be, e.g., an integer of 1 to 4.

m12 and m13 may each independently be, e.g., an integer of 1 to 3.

n may be, e.g., an integer 0 to 2.

In an implementation, m11 may be, e.g., 2, 3, or 4, and each R¹¹ may be the same or different from each other.

In an implementation, m12 may be, e.g., 2 or 3, and each R¹² may be the same or different from each other.

In an implementation, m13 may be, e.g., 2 or 3, and each R¹³ may be the same or different from each other.

In an implementation, m14 may be, e.g., 2, 3, or 4, and each R¹⁴ may be the same or different from each other.

In an implementation, m15 may be, e.g., 2, 3, or 4, and each R¹⁵ may be the same or different from each other.

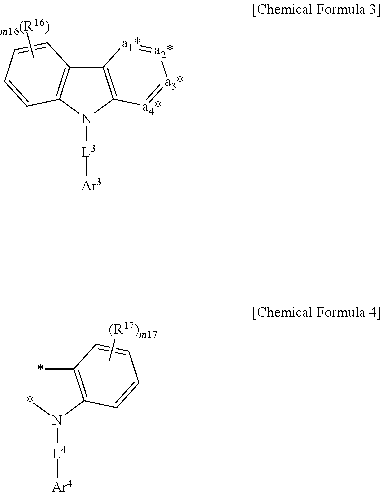

In Chemical Formula 3 and Chemical Formula 4, two adjacent ones of a₁* to a₄* of Chemical Formula 3 may each independently be linking carbons linked at * of Chemical Formula 4, the remaining two of a₁* to a₄* of Chemical Formula 3 not linked at * of Chemical Formula 4 may each independently be, e.g., C-L^{a}-R^{a}.

L^{a}, L³, and L⁴ may each independently be or include, e.g., a single bond or a substituted or unsubstituted C6 to C20 arylene group.

R^{a}, R¹⁶ and R¹⁷ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar³ and Ar⁴ may each independently be or include, e.g., a substituted or unsubstituted C6 to C20 aryl group or a substituted or unsubstituted C2 to C30 heterocyclic group.

m16 and m17 may each independently be, e.g., an integer of 1 to 4.

In an implementation, m16 may be, e.g., 2, 3, or 4, and each R¹⁶ may be the same or different from each other.

In an implementation, m17 may be, e.g., 2, 3, or 4, and each R¹⁷ may be the same or different from each other.

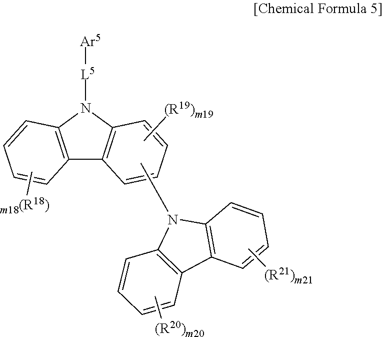

In Chemical Formula 5, L⁵ may be or include, e.g., a single bond or a substituted or unsubstituted C6 to C20 arylene group.

R¹⁸ to R²¹ may each independently be or include, e.g., hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

Ar⁵ may be or include, e.g., a substituted or unsubstituted C6 to C20 aryl group or a substituted or unsubstituted C2 to C30 heterocyclic group.

m18, m20, and m21 may each independently be, e.g., an integer of 1 to 4.

m19 may be, e.g., an integer of 1 to 3.

In an implementation, m18 may be, e.g., 2, 3, or 4, and each R¹⁸ may be the same or different from each other.

In an implementation, m19 may be, e.g., 2 or 3, and each R¹⁹ may be the same or different from each other.

In an implementation, m20 may be, e.g., 2, 3, or 4, and each R²⁰ may be the same or different from each other.

In an implementation, m21 may be, e.g., 2, 3, or 4, and each R²¹ may be the same or different from each other.

The second compound may be used in a light emitting layer together with the first compound to improve luminous efficiency and life-span characteristics by increasing charge mobility and enhancing stability.

In an implementation, in Chemical Formula 2, Ar¹ and Ar² may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, L¹ and L² may each independently be, e.g., a single bond, a substituted or unsubstituted phenylene group, or a substituted or unsubstituted biphenylene group.

In an implementation, R¹¹ to R¹⁵ may each independently be, e.g., hydrogen, deuterium, or a substituted or unsubstituted C6 to C12 aryl group.

n may be, e.g., 0 or 1.

In an implementation, in Chemical Formula 2, “substituted” may mean, e.g. that at least one hydrogen is substituted with deuterium, a C1 to C4 alkyl group, a C6 to C18 aryl group, or a C2 to C30 heteroaryl group.

In an implementation, in Chemical Formula 2, Ar¹ and Ar² may each independently be, e.g., a substituted or unsubstituted carbazolyl group, a substituted or unsubstituted dibenzothiophenyl group, a substituted or unsubstituted dibenzofuranyl group, or a substituted or unsubstituted fluorenyl group.

In an implementation, Chemical Formula 2 may be represented by, e.g., one of Chemical Formula 2-1 to Chemical Formula 2-15.

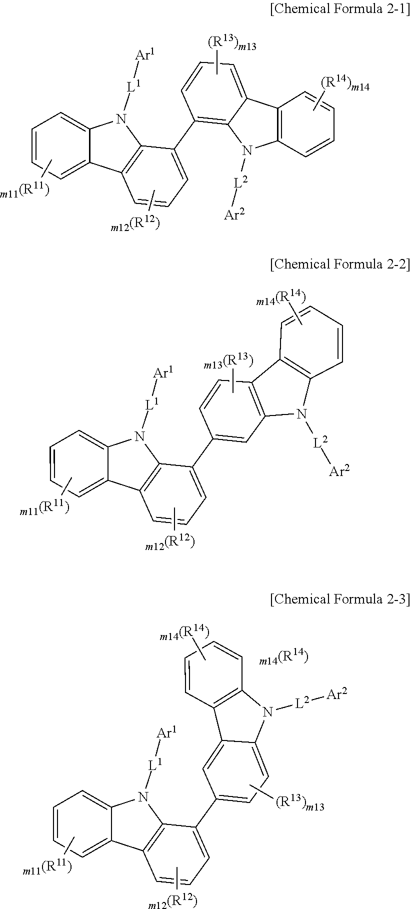

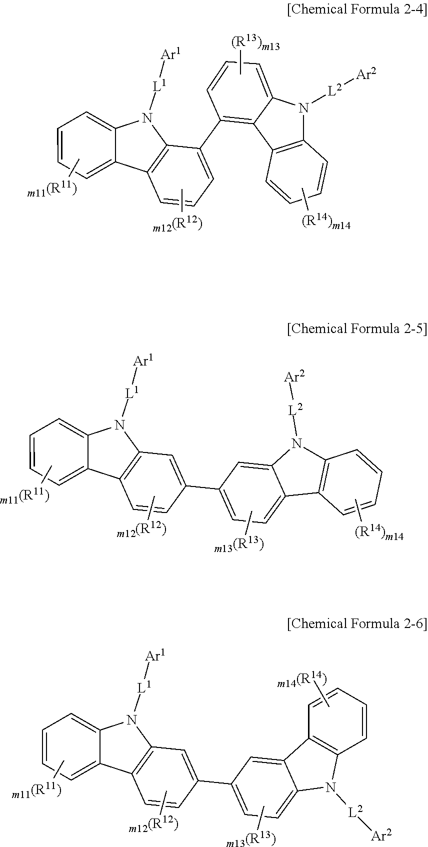

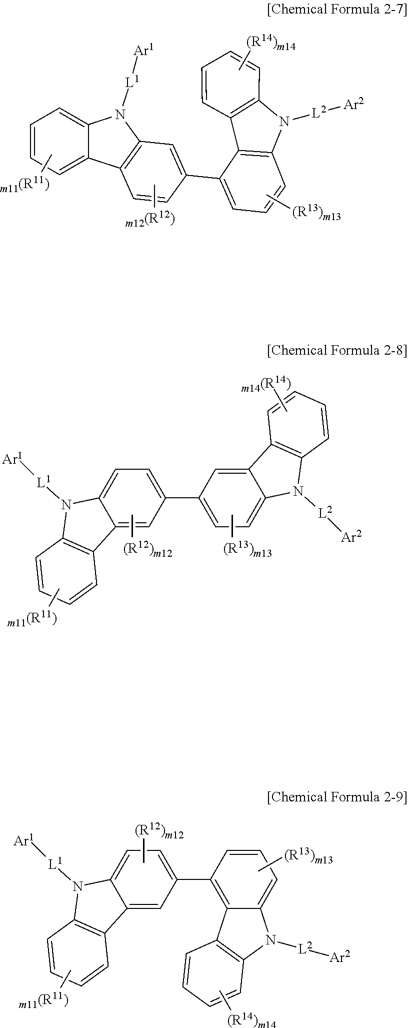

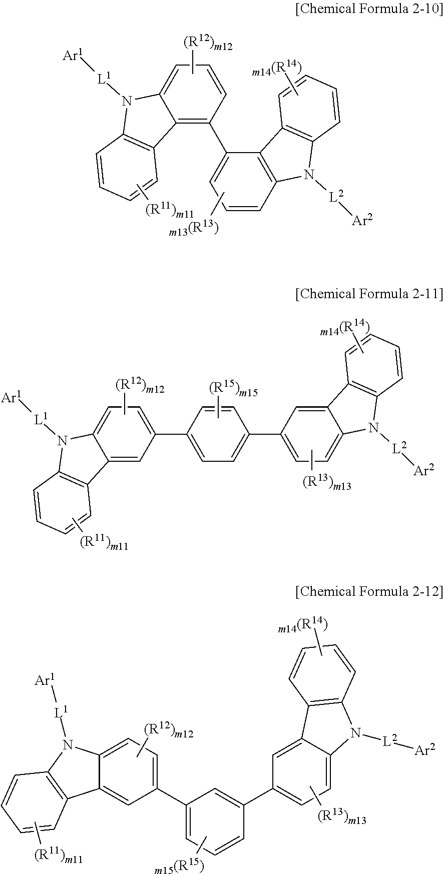

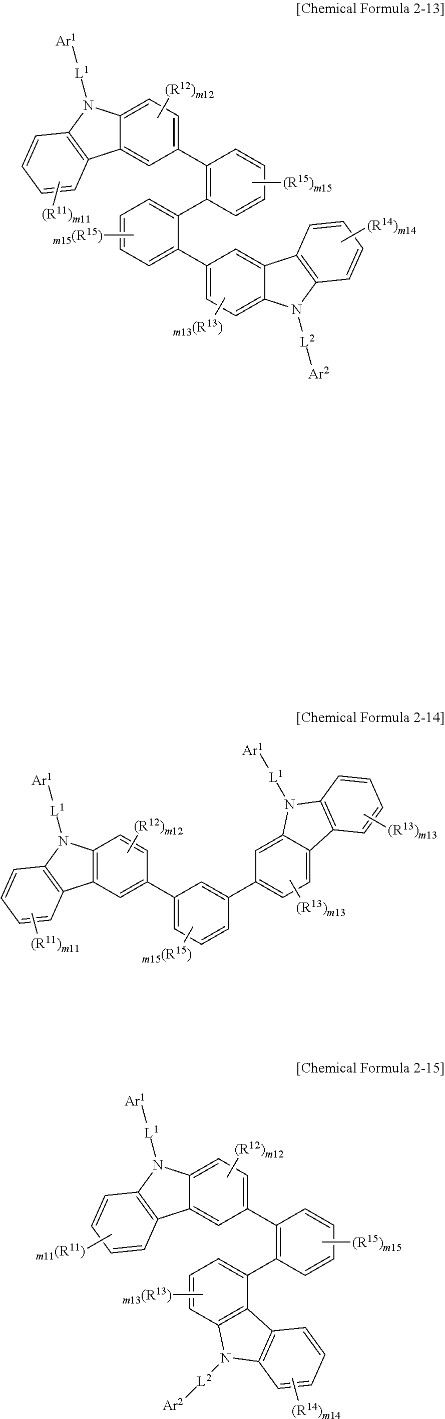

In Chemical Formula 2-1 to Chemical Formula 2-15, R¹¹ to R¹⁵ may each independently be, e.g., hydrogen, deuterium, or a substituted or unsubstituted C6 to C12 aryl group, and moieties *-L¹-Ar¹ and *-L²-Ar² may each independently be, e.g., a moiety of Group II.

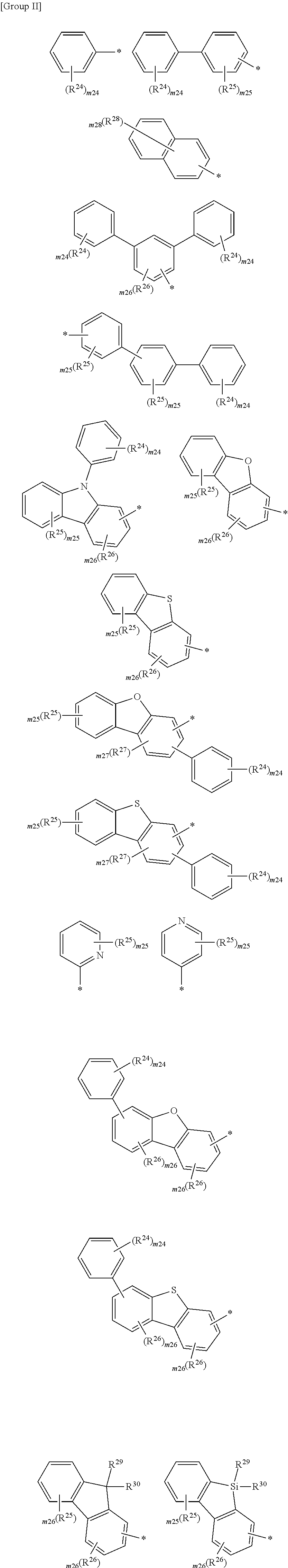

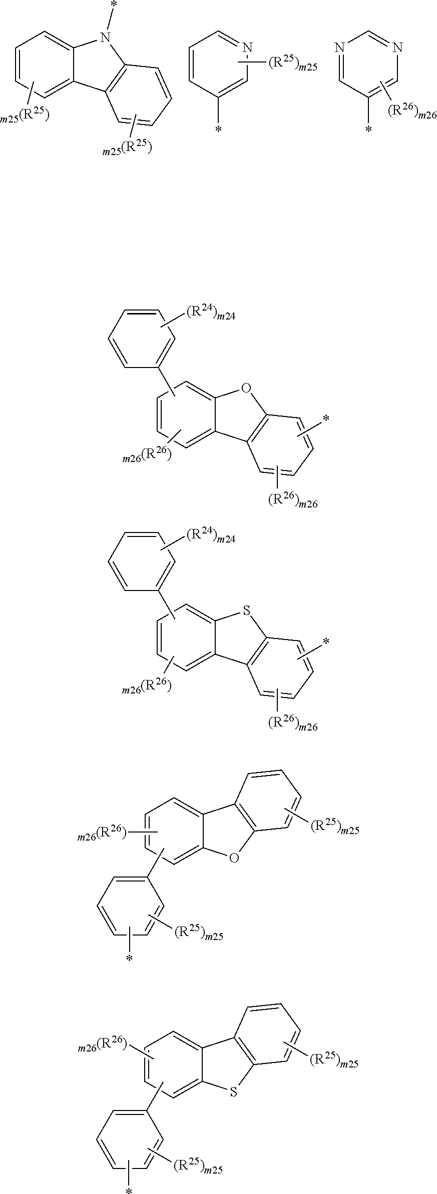

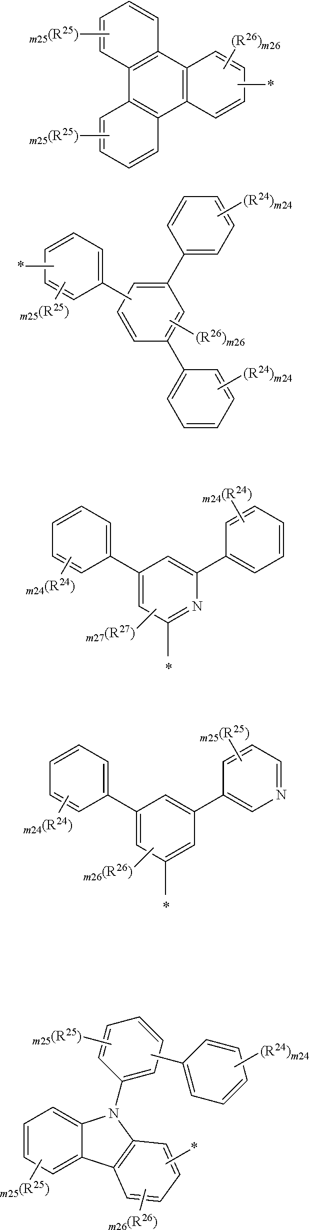

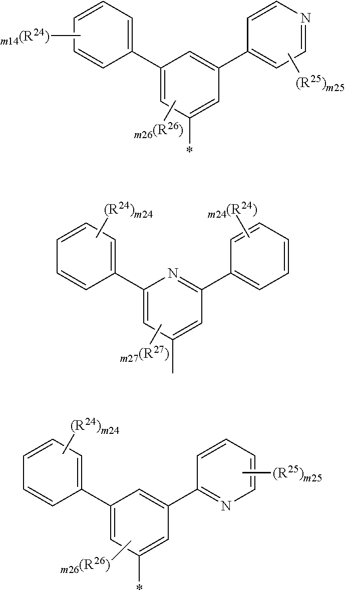

In Group II, R²⁴ to R³⁰ may each independently be, e.g., hydrogen, deuterium, a cyano group, a substituted or unsubstituted C1 to C10 alkyl group, or a substituted or unsubstituted C6 to C12 aryl group.

m24 may be, e.g., an integer of 1 to 5.

m25 may be, e.g., an integer of 1 to 4.

m26 may be, e.g., an integer of 1 to 3.

m27 may be, e.g., an integer of 1 or 2.

m28 may be, e.g., an integer of 1 to 7.

* is a linking point.

In an implementation, m24 may be, e.g., 2, 3, 4, or 5, each R²⁴ may be the same or different from each other.

In an implementation, m25 may be, e.g., 2, 3, or 4, each R²⁵ may be the same or different from each other.

In an implementation, m26 may be, e.g., 2 or 3, and R²⁶ may be the same or different from each other.

In an implementation, m27 may be, e.g., 2, and each R²⁷ may be the same or different from each other.

In an implementation, m28 may be, e.g., 2, 3, 4, 5, 6, or 7, and each R²⁸ may be the same or different from each other.

The combination of Chemical Formula 3 and Chemical Formula 4 may be, e.g., represented by one of Chemical Formula 3A, Chemical Formula 3B, Chemical Formula 3C, Chemical Formula 3D, and Chemical Formula 3E.+

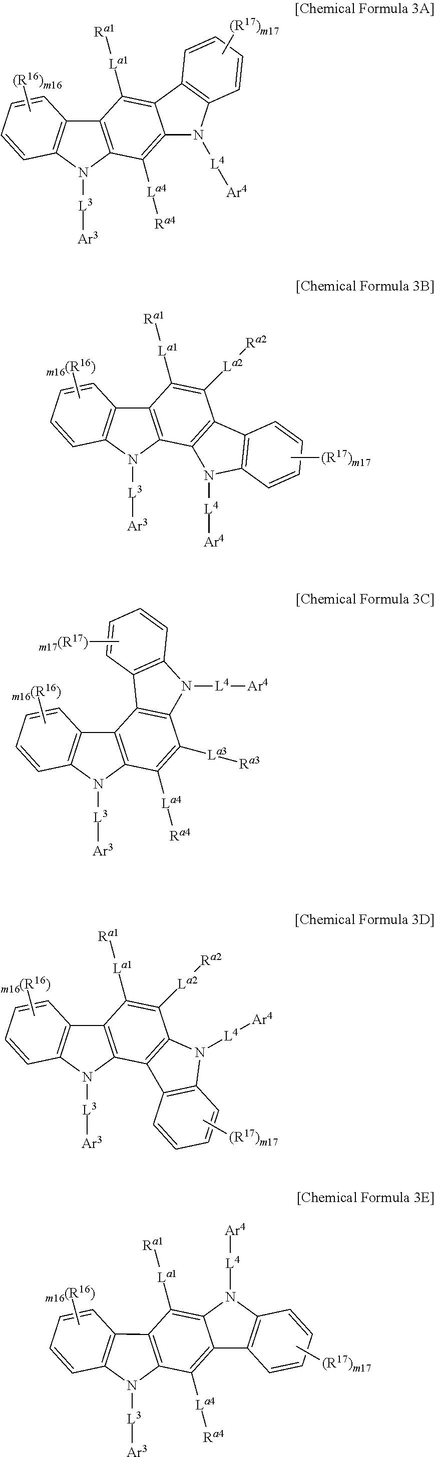

In Chemical Formula 3A to Chemical Formula 3E, L³, L⁴, Ar³, Ar⁴, R¹⁶, R¹⁷, m16 and m17 are the same as those described above,

L^{a1} to L^{a4} may be defined the same as L³ and L⁴ described above.

R^{a1} to R^{a4} may be defined the same as R¹⁶ and R¹⁷ described above.

In an implementation, in Chemical Formula 3 and Chemical Formula 4, Ar³ and Ar⁴ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

R^{a1} to R^{a4}, R¹⁶ and R¹⁷ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 3 and Chemical Formula 4, moieties *-L³-Ar³ and *-L⁴-Ar⁴ may each be, e.g., a moiety of Group II.

In an implementation, R^{a1} to R^{a4}, R¹⁶, and R¹⁷ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, R^{a1} to R^{a4}, R¹⁶, and R¹⁷ may each independently be, e.g., hydrogen, deuterium, a cyano group, or a substituted or unsubstituted phenyl group.

In an implementation, R^{a1} to R^{a4}, R¹⁶, and R¹⁷ may each independently be, e.g., hydrogen, deuterium, or a substituted or unsubstituted phenyl group.

Chemical Formula 5 may be, e.g., represented by one of Chemical Formula 5-1 to Chemical Formula 5-4.

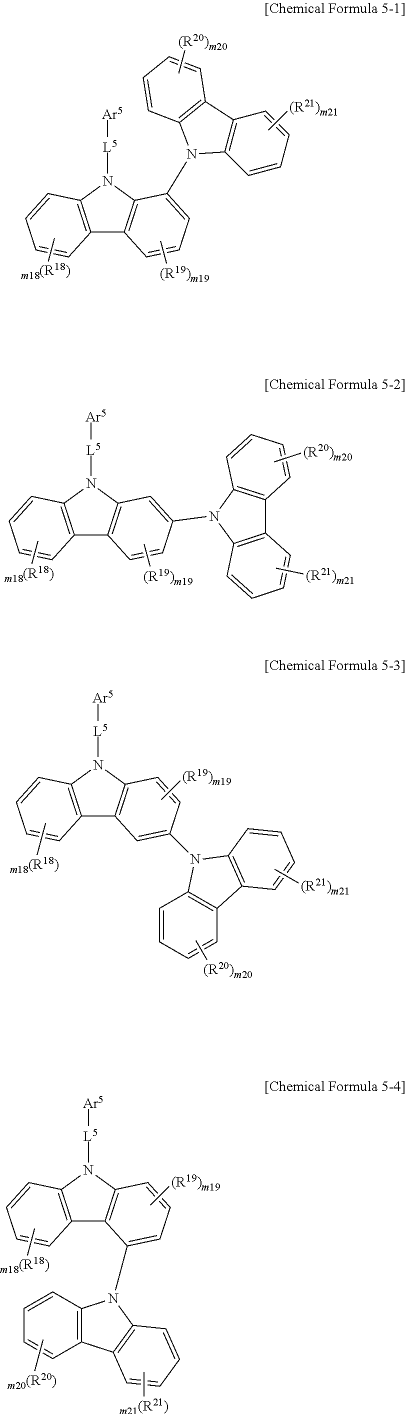

In Chemical Formula 5-1 to Chemical Formula 5-4, L⁵, Ar⁵, R¹⁸ to R²¹, and m18 to m21 may be defined the same as those described above.

In an implementation, in Chemical Formula 5, Ar⁵ may be, e.g., a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group.

R¹⁸ to R²¹ may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 5, moiety *-L⁵-Ar⁵ may be, e.g., a moiety of Group II.

In an implementation, R¹⁸ to R²¹ may each independently be, e.g., hydrogen, deuterium, a cyano group, or a substituted or unsubstituted phenyl group.

Chemical Formula 2 may be, e.g., represented by Chemical Formula 2-8, and in Chemical Formula 2-8, Ar¹ and Ar² may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, L¹ and L² may each independently be, e.g., a single bond or a substituted or unsubstituted C6 to C20 arylene group, R⁹ to R¹² may each independently be, e.g., hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 2-8, R¹¹ to R¹⁴ may each independently be, e.g., hydrogen, deuterium, or a substituted or unsubstituted C6 to C12 aryl group, and moieties *-L¹-Ar¹ and *-L²-Ar² may each independently be, e.g., a moiety of Group II.

In an implementation, the second compound may be, e.g., represented by Chemical Formula 3C, and in Chemical Formula 3C, L^{a3} and L^{a4} may each be, e.g., a single bond, L³ and L⁴ may each independently be, e.g., a single bond or a substituted or unsubstituted C6 to C12 arylene group, R¹⁶, R¹⁷, R^{a3}, and R^{a4} may each be, e.g., hydrogen, deuterium, or a phenyl group, and Ar³ and Ar⁴ may each independently be, e.g., a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an implementation, in Chemical Formula 3C, L^{a3} and L^{a4} may be, e.g., single bonds, R¹⁶, R¹⁷, R³, and R^{a4} may each independently be, e.g., hydrogen, deuterium, or a C6 to C12 aryl group, and moieties *-L³-Ar³ and *-L⁴-Ar⁴ may each independently be, e.g., a moiety of Group II.

In an implementation, the second compound for an optoelectronic device may be, e.g., a compound of Group 2.

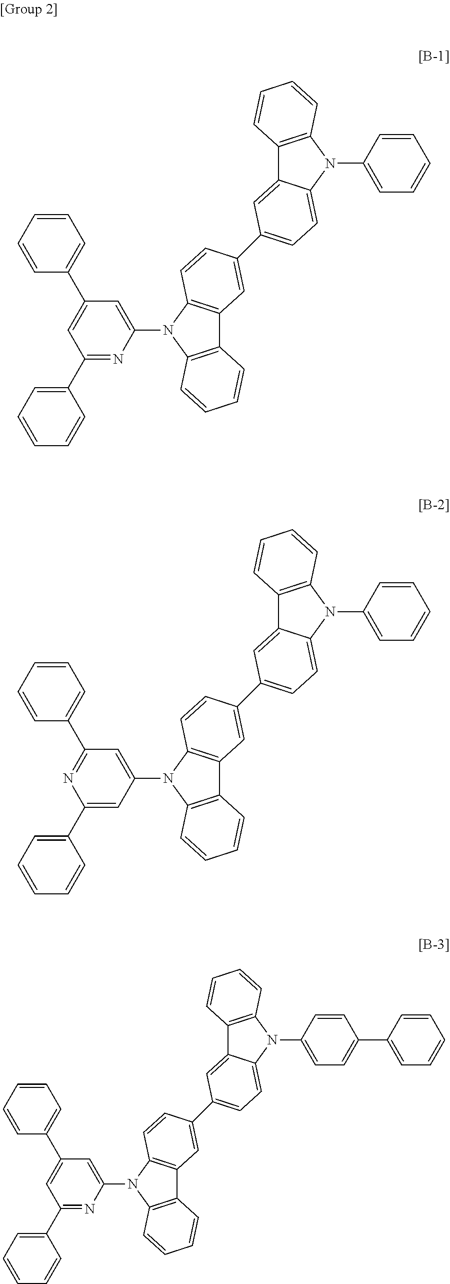 [SMILES: c1ccc(-c2cc(-c3ccccc3)nc(-n3c4ccccc4c4cc(-c5ccc6c(c5)c5ccccc5n6-c5ccccc5)ccc43)c2)cc1 ; c1ccc(-c2cc(-n3c4ccccc4c4cc(-c5ccc6c(c5)c5ccccc5n6-c5ccccc5)ccc43)cc(-c3ccccc3)n2)cc1 ; c1ccc(-c2ccc(-n3c4ccccc4c4cc(-c5ccc6c(c5)c5ccccc5n6-c5cc(-c6ccccc6)cc(-c6ccccc6)n5)ccc43)cc2)cc1]

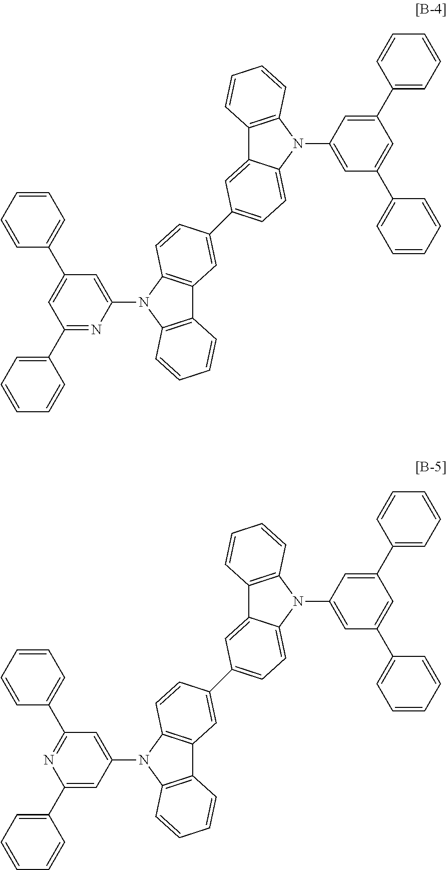 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)cc(-n3c4ccccc4c4cc(-c5ccc6c(c5)c5ccccc5n6-c5cc(-c6ccccc6)nc(-c6ccccc6)c5)ccc43)c2)cc1]

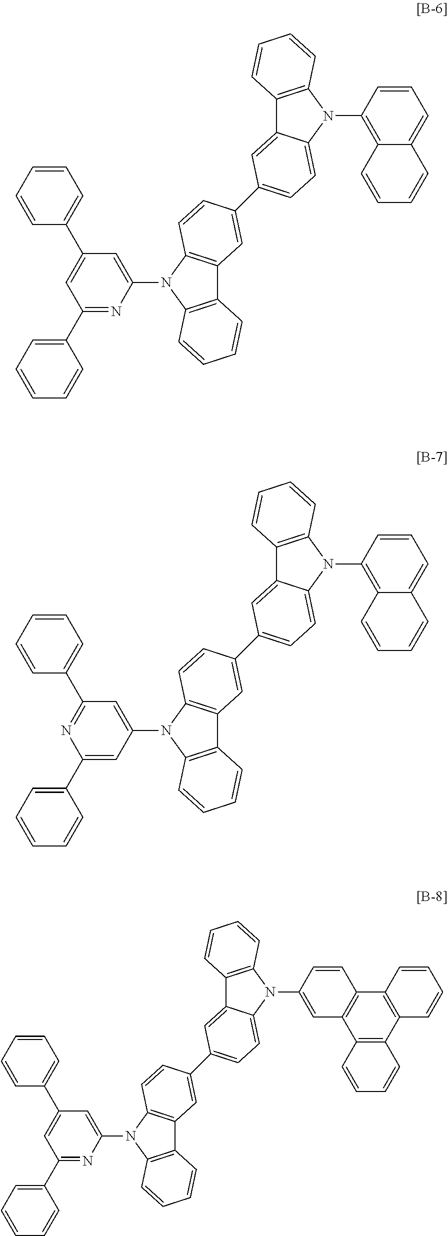 [SMILES: c1ccc(-c2cc(-c3ccccc3)nc(-n3c4ccccc4c4cc(-c5ccc6c(c5)c5ccccc5n6-c5cccc6ccccc56)ccc43)c2)cc1 ; c1ccc(-c2cc(-n3c4ccccc4c4cc(-c5ccc6c(c5)c5ccccc5n6-c5cccc6ccccc56)ccc43)cc(-c3ccccc3)n2)cc1 ; c1ccc(-c2cc(-c3ccccc3)nc(-n3c4ccccc4c4cc(-c5ccc6c(c5)c5ccccc5n6-c5ccc6c7ccccc7c7ccccc7c6c5)ccc43)c2)cc1]

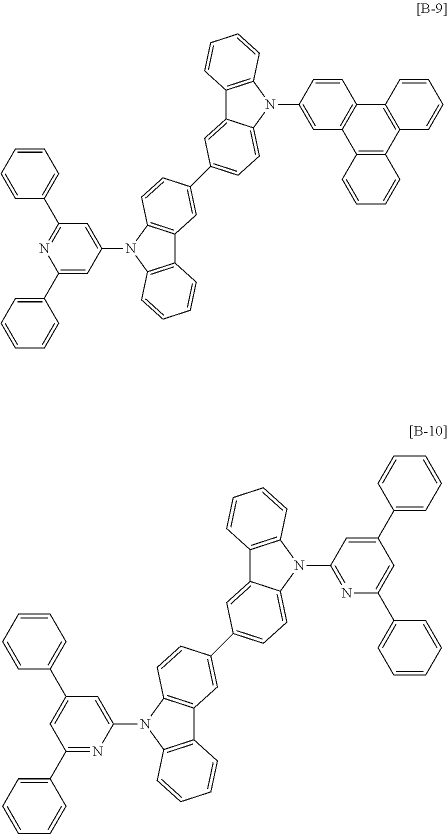 [SMILES: c1ccc(-c2cc(-n3c4ccccc4c4cc(-c5ccc6c(c5)c5ccccc5n6-c5ccc6c7ccccc7c7ccccc7c6c5)ccc43)cc(-c3ccccc3)n2)cc1 ; c1ccc(-c2cc(-c3ccccc3)nc(-n3c4ccccc4c4cc(-c5ccc6c(c5)c5ccccc5n6-c5cc(-c6ccccc6)cc(-c6ccccc6)n5)ccc43)c2)cc1]

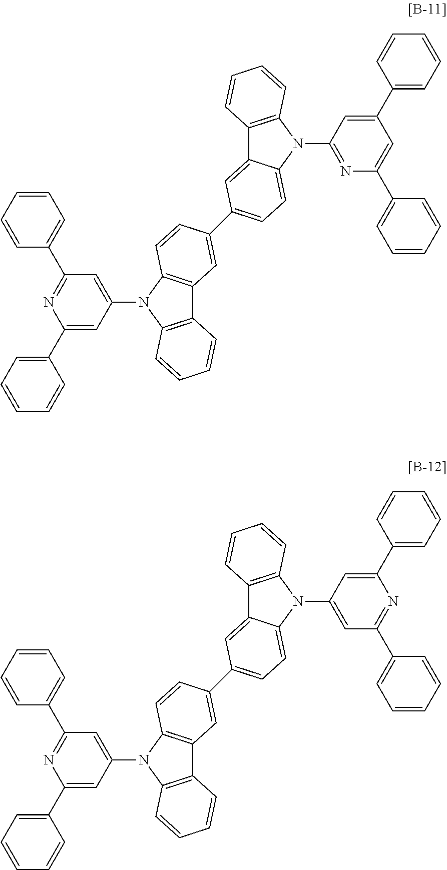 [SMILES: c1ccc(-c2cc(-c3ccccc3)nc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-n3c4ccccc4c4cc(-c5ccc6c(c5)c5ccccc5n6-c5cc(-c6ccccc6)nc(-c6ccccc6)c5)ccc43)cc(-c3ccccc3)n2)cc1]

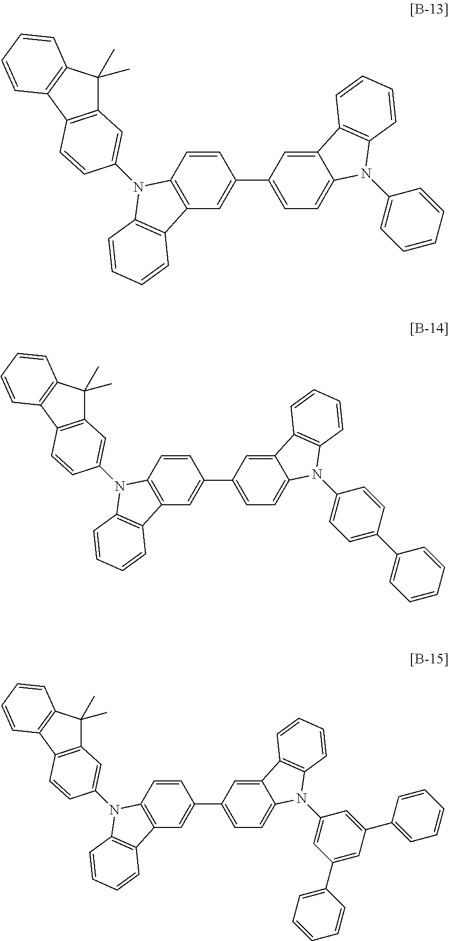 [SMILES: CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21]

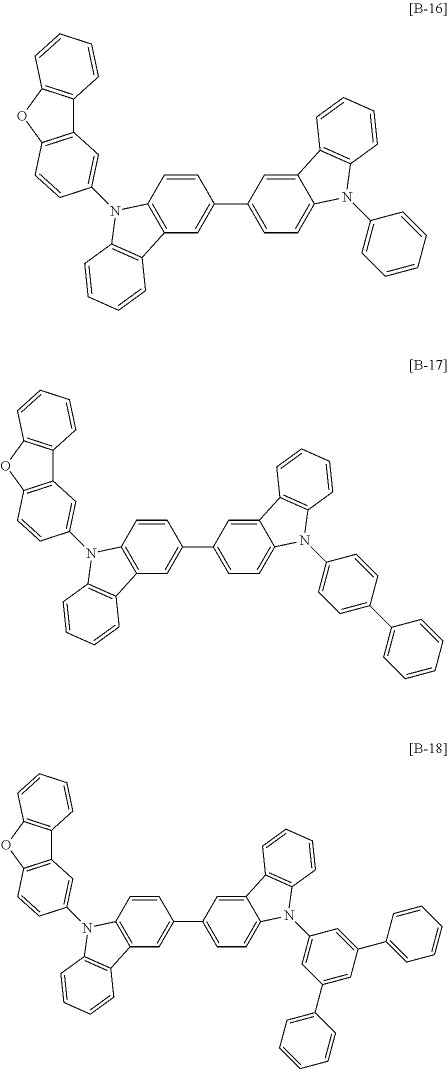 [SMILES: c1ccc(-n2c3ccccc3c3cc(-c4ccc5c(c4)c4ccccc4n5-c4ccc5oc6ccccc6c5c4)ccc32)cc1 ; c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6oc7ccccc7c6c5)ccc43)c2)cc1]

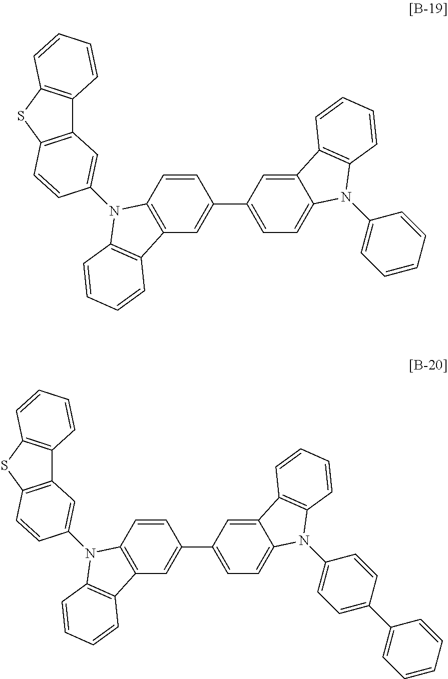 [SMILES: c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1]

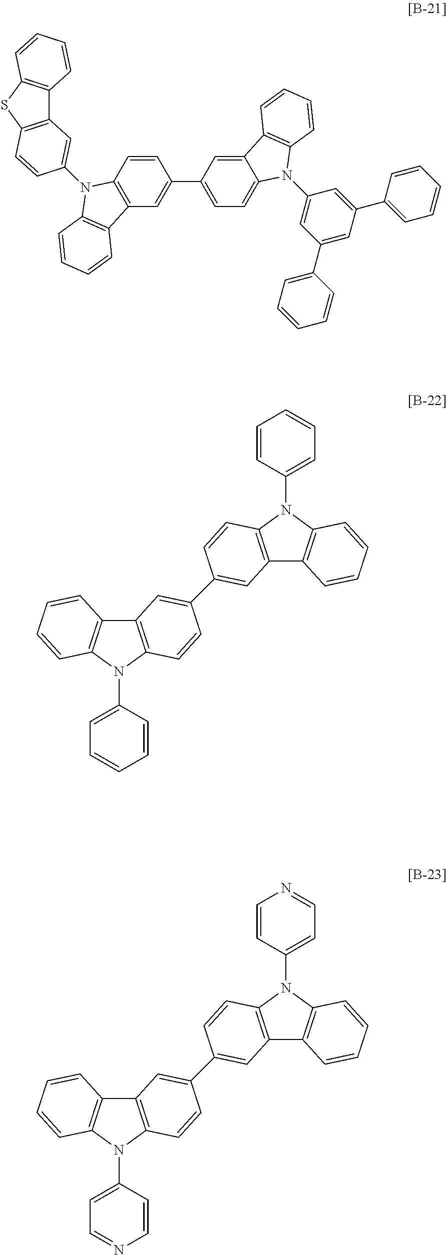 [SMILES: c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccccc4)ccc32)cc1 ; c1ccc2c(c1)c1cc(-c3ccc4c(c3)c3ccccc3n4-c3ccncc3)ccc1n2-c1ccncc1]

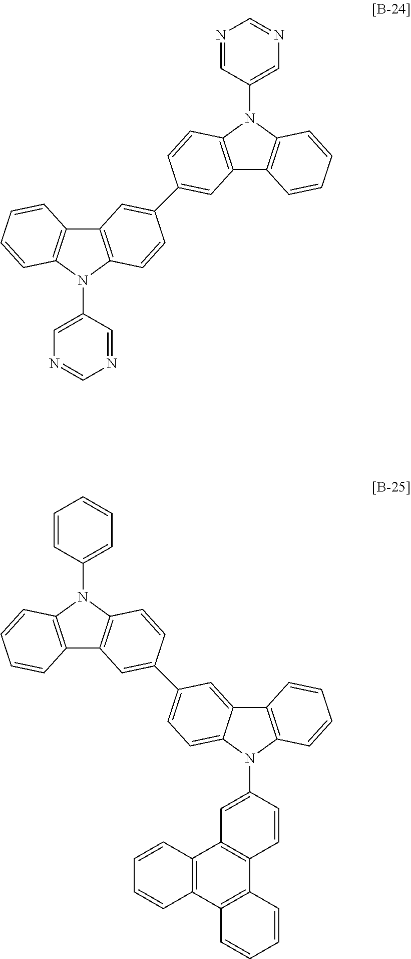 [SMILES: c1ccc2c(c1)c1cc(-c3ccc4c(c3)c3ccccc3n4-c3cncnc3)ccc1n2-c1cncnc1 ; c1ccc(-n2c3ccccc3c3cc(-c4ccc5c(c4)c4ccccc4n5-c4ccc5c6ccccc6c6ccccc6c5c4)ccc32)cc1]

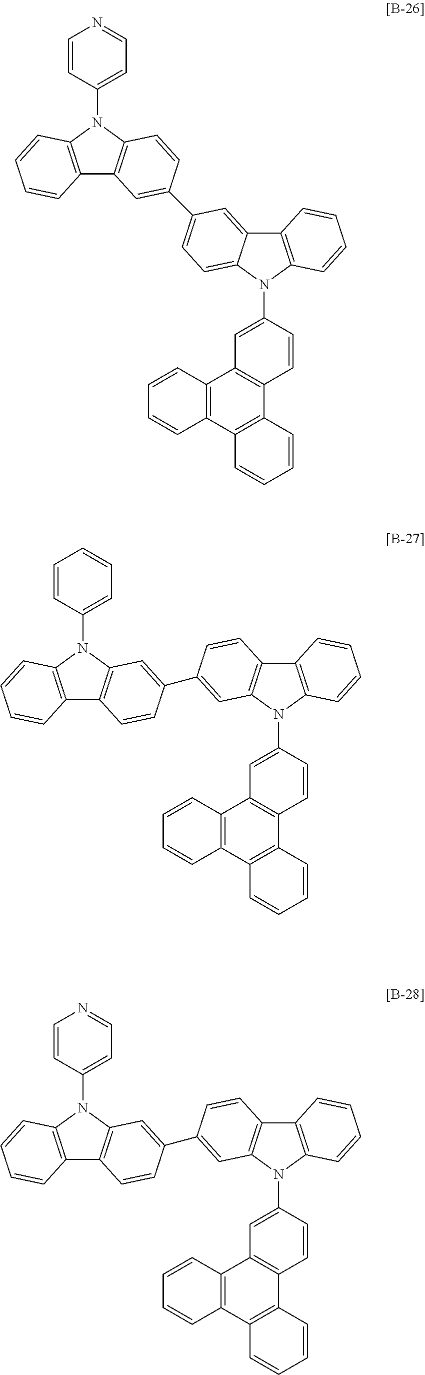 [SMILES: c1ccc2c(c1)c1ccccc1c1cc(-n3c4ccccc4c4cc(-c5ccc6c(c5)c5ccccc5n6-c5ccncc5)ccc43)ccc21 ; c1ccc(-n2c3ccccc3c3ccc(-c4ccc5c6ccccc6n(-c6ccc7c8ccccc8c8ccccc8c7c6)c5c4)cc32)cc1 ; c1ccc2c(c1)c1ccccc1c1cc(-n3c4ccccc4c4ccc(-c5ccc6c7ccccc7n(-c7ccncc7)c6c5)cc43)ccc21]

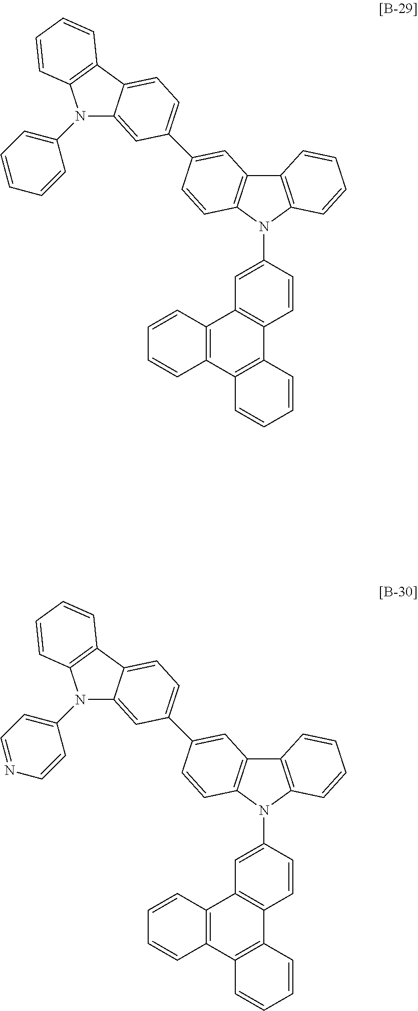 [SMILES: c1ccc(-n2c3ccccc3c3ccc(-c4ccc5c(c4)c4ccccc4n5-c4ccc5c6ccccc6c6ccccc6c5c4)cc32)cc1 ; c1ccc2c(c1)c1ccccc1c1cc(-n3c4ccccc4c4cc(-c5ccc6c7ccccc7n(-c7ccncc7)c6c5)ccc43)ccc21]

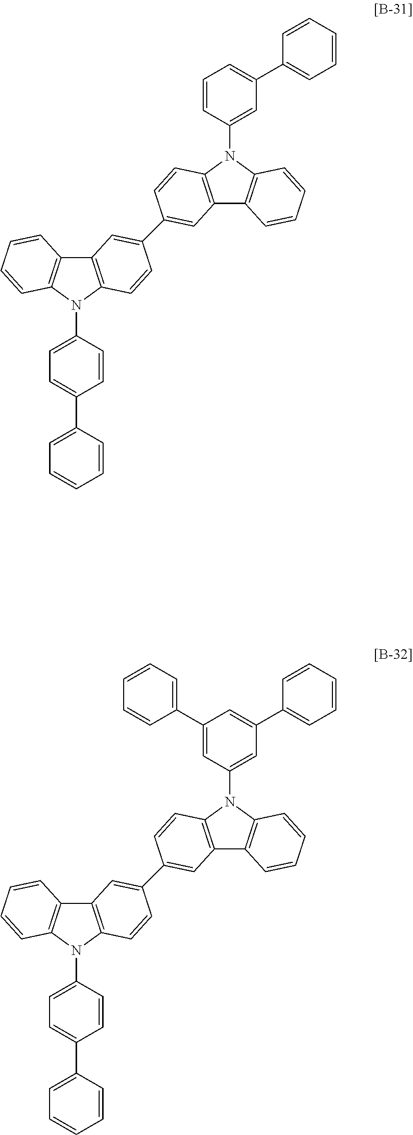 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

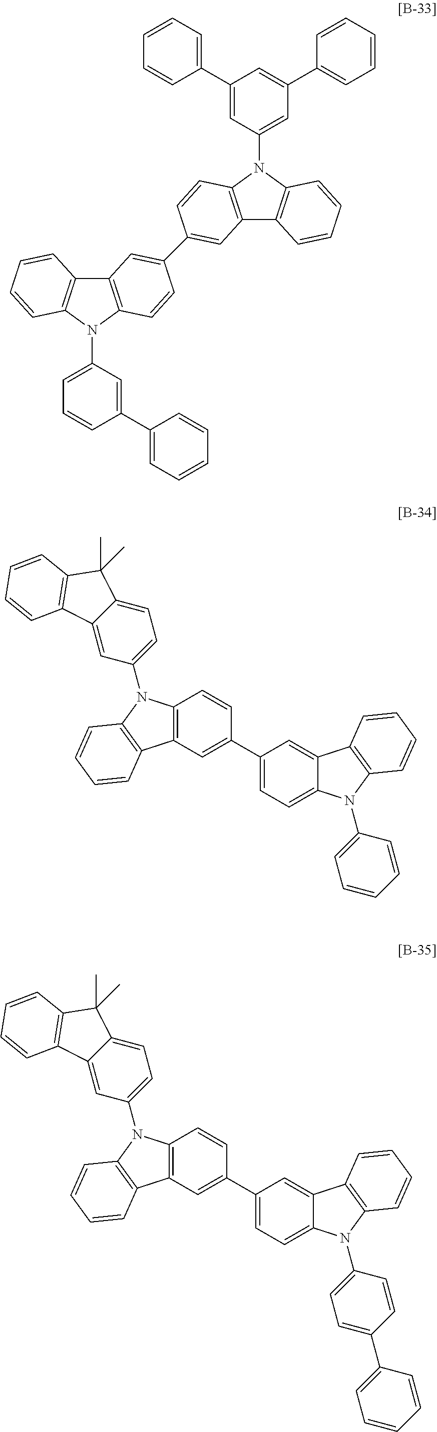 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; CC1(C)c2ccccc2-c2cc(-n3c4ccccc4c4cc(-c5ccc6c(c5)c5ccccc5n6-c5ccccc5)ccc43)ccc21 ; CC1(C)c2ccccc2-c2cc(-n3c4ccccc4c4cc(-c5ccc6c(c5)c5ccccc5n6-c5ccc(-c6ccccc6)cc5)ccc43)ccc21]

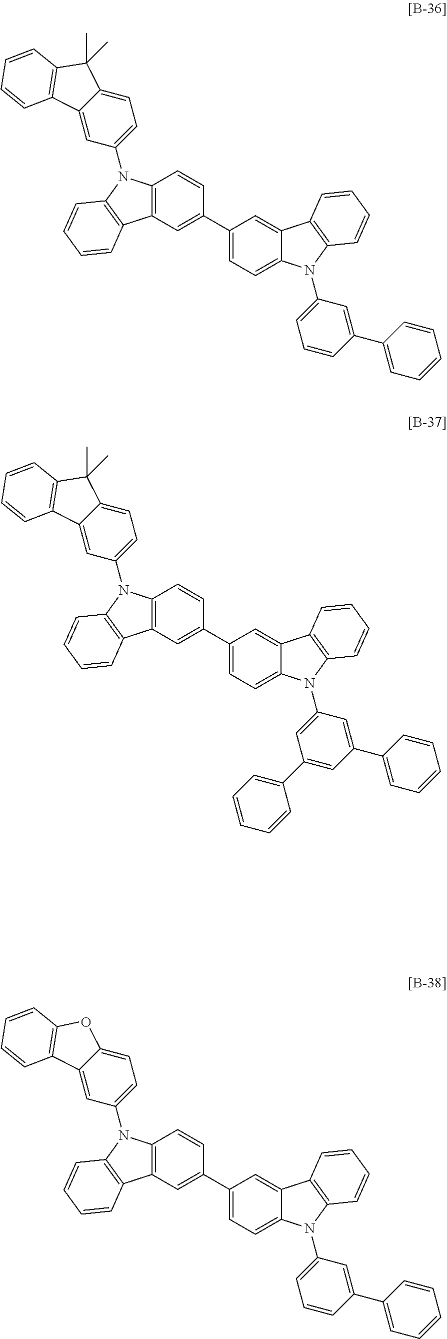 [SMILES: CC1(C)c2ccccc2-c2cc(-n3c4ccccc4c4cc(-c5ccc6c(c5)c5ccccc5n6-c5cccc(-c6ccccc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccc6)c5)ccc43)ccc21 ; c1ccc(-c2cccc(-n3c4ccccc4c4cc(-c5ccc6c(c5)c5ccccc5n6-c5ccc6oc7ccccc7c6c5)ccc43)c2)cc1]

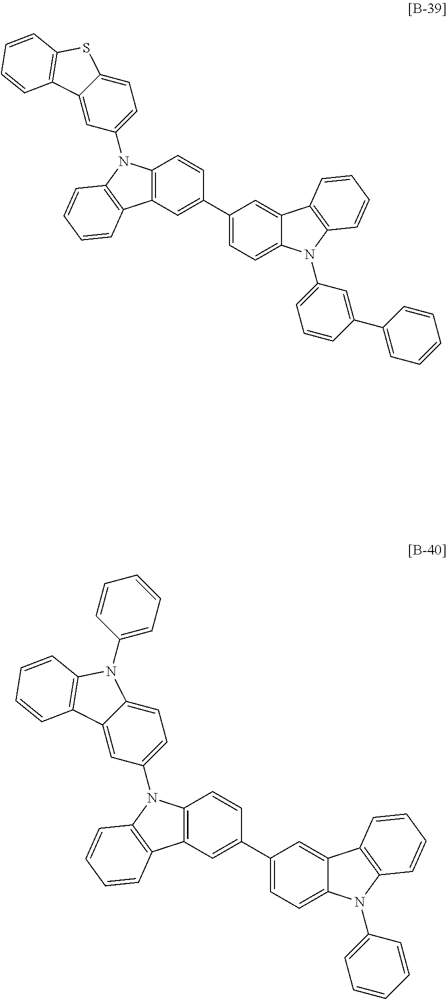 [SMILES: c1ccc(-c2cccc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccc5c(c4)c4ccccc4n5-c4ccccc4)ccc32)cc1]

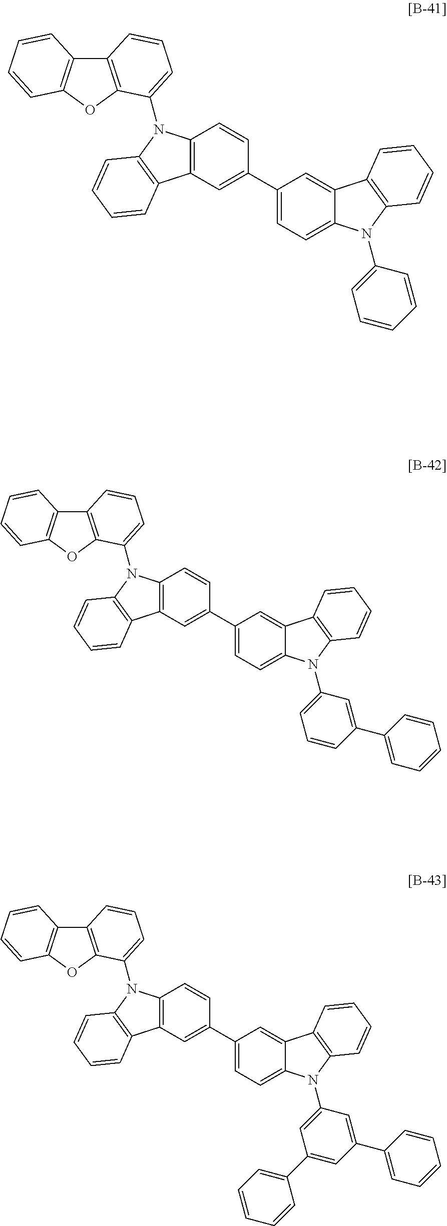 [SMILES: c1ccc(-n2c3ccccc3c3cc(-c4ccc5c(c4)c4ccccc4n5-c4cccc5c4oc4ccccc45)ccc32)cc1 ; c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5oc5ccccc56)ccc43)c2)cc1]

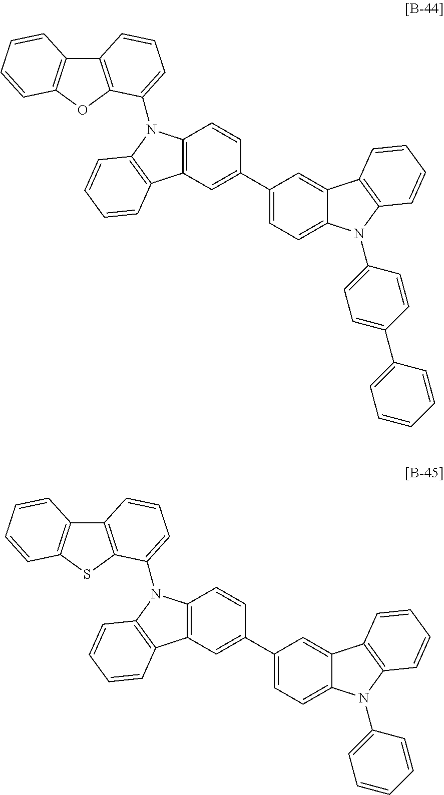 [SMILES: c1ccc(-c2ccc(-n3c4ccccc4c4cc(-c5ccc6c(c5)c5ccccc5n6-c5cccc6c5oc5ccccc56)ccc43)cc2)cc1 ; c1ccc(-n2c3ccccc3c3cc(-c4ccc5c(c4)c4ccccc4n5-c4cccc5c4sc4ccccc45)ccc32)cc1]

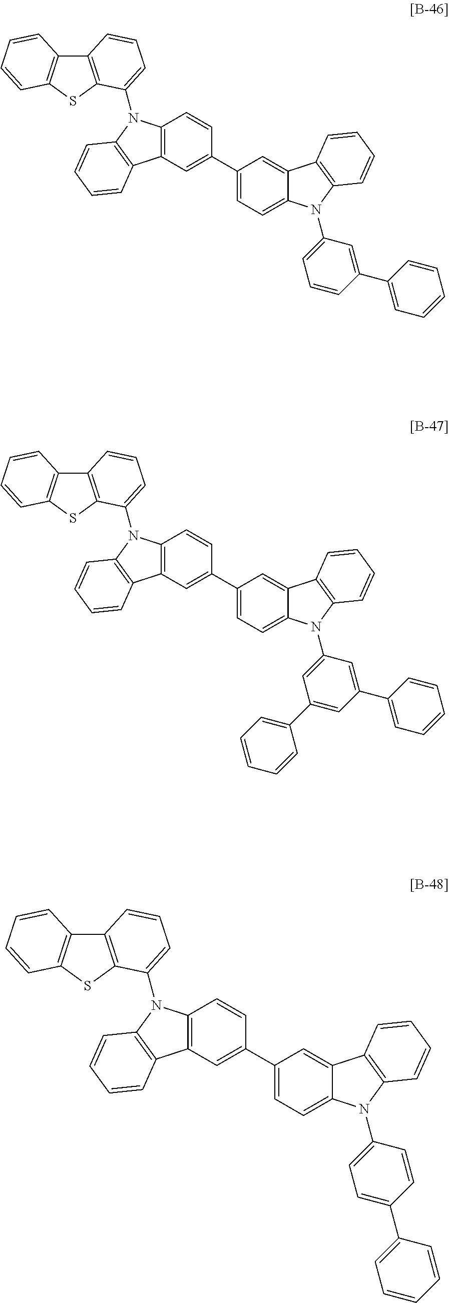 [SMILES: c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1 ; c1ccc(-c2ccc(-n3c4ccccc4c4cc(-c5ccc6c(c5)c5ccccc5n6-c5cccc6c5sc5ccccc56)ccc43)cc2)cc1]

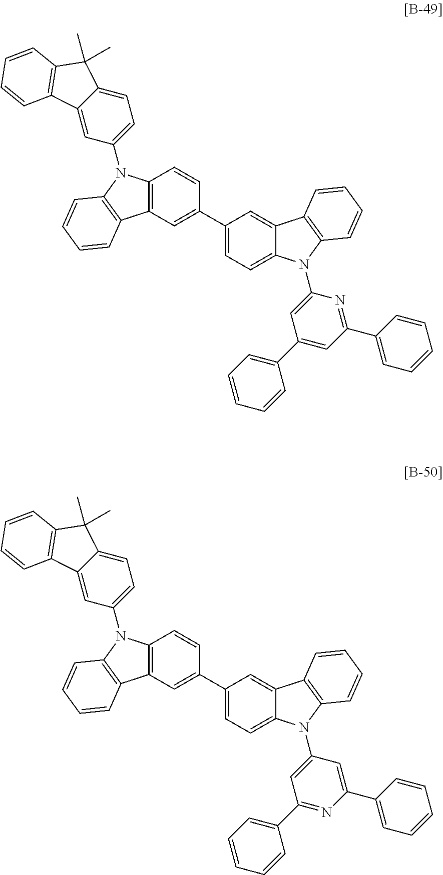 [SMILES: CC1(C)c2ccccc2-c2cc(-n3c4ccccc4c4cc(-c5ccc6c(c5)c5ccccc5n6-c5cc(-c6ccccc6)cc(-c6ccccc6)n5)ccc43)ccc21 ; CC1(C)c2ccccc2-c2cc(-n3c4ccccc4c4cc(-c5ccc6c(c5)c5ccccc5n6-c5cc(-c6ccccc6)nc(-c6ccccc6)c5)ccc43)ccc21]

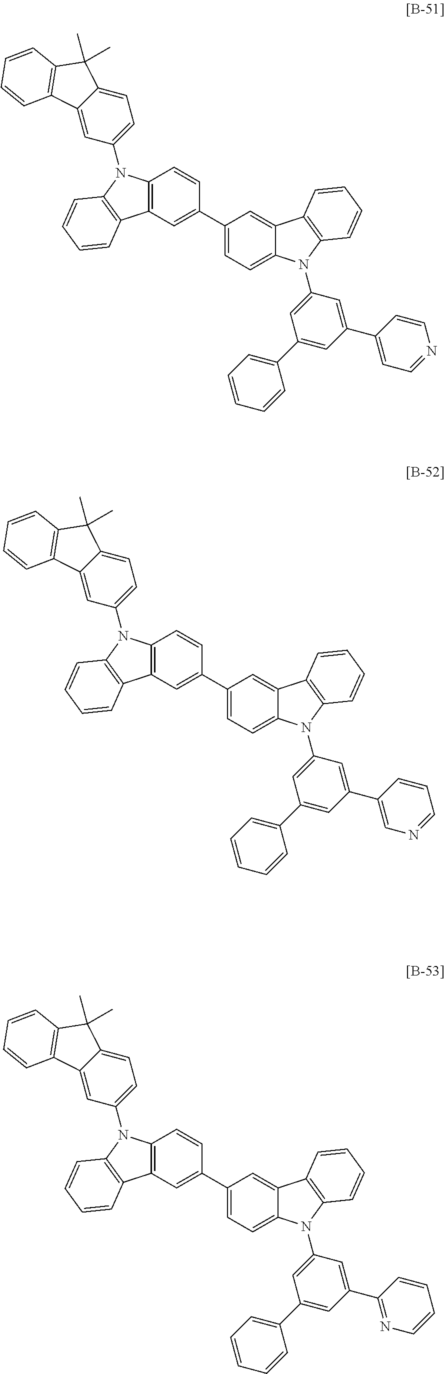 [SMILES: CC1(C)c2ccccc2-c2cc(-n3c4ccccc4c4cc(-c5ccc6c(c5)c5ccccc5n6-c5cc(-c6ccccc6)cc(-c6ccncc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6cccnc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccn6)c5)ccc43)ccc21]

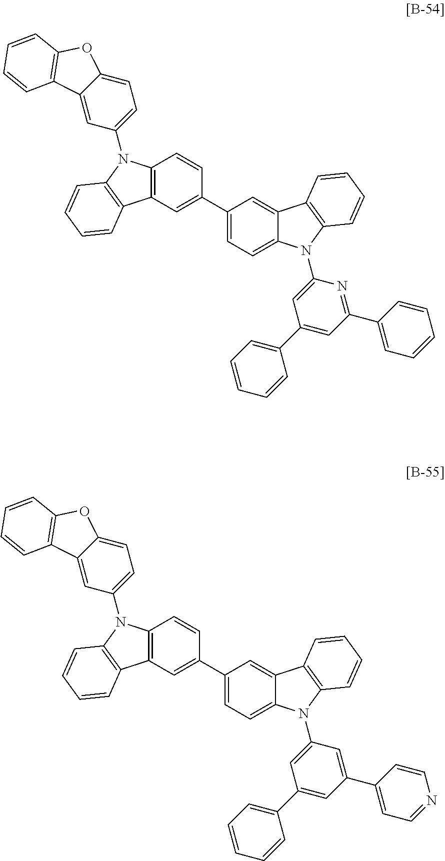 [SMILES: c1ccc(-c2cc(-c3ccccc3)nc(-n3c4ccccc4c4cc(-c5ccc6c(c5)c5ccccc5n6-c5ccc6oc7ccccc7c6c5)ccc43)c2)cc1 ; c1ccc(-c2cc(-c3ccncc3)cc(-n3c4ccccc4c4cc(-c5ccc6c(c5)c5ccccc5n6-c5ccc6oc7ccccc7c6c5)ccc43)c2)cc1]

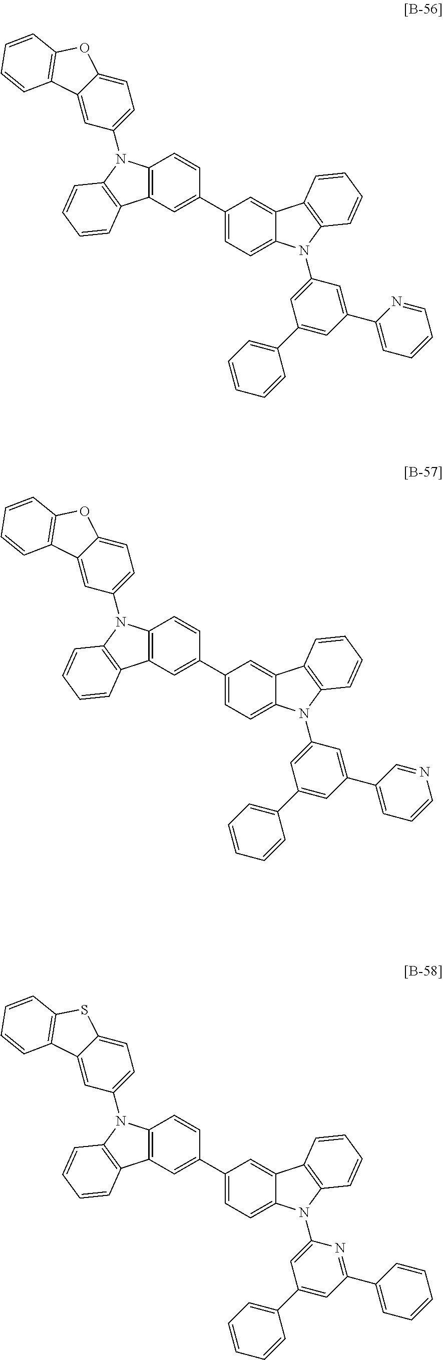 [SMILES: c1ccc(-c2cc(-c3ccccn3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3cccnc3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3ccccc3)nc(-n3c4ccccc4c4cc(-c5ccc6c(c5)c5ccccc5n6-c5ccc6sc7ccccc7c6c5)ccc43)c2)cc1]

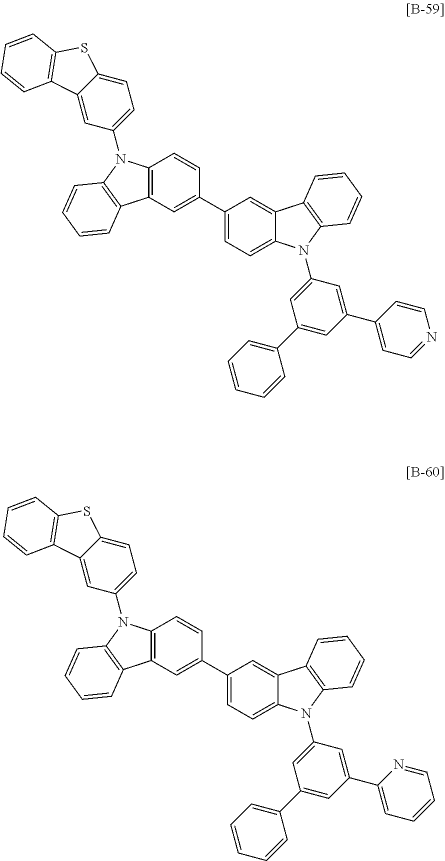 [SMILES: c1ccc(-c2cc(-c3ccncc3)cc(-n3c4ccccc4c4cc(-c5ccc6c(c5)c5ccccc5n6-c5ccc6sc7ccccc7c6c5)ccc43)c2)cc1 ; c1ccc(-c2cc(-c3ccccn3)cc(-n3c4ccccc4c4cc(-c5ccc6c(c5)c5ccccc5n6-c5ccc6sc7ccccc7c6c5)ccc43)c2)cc1]

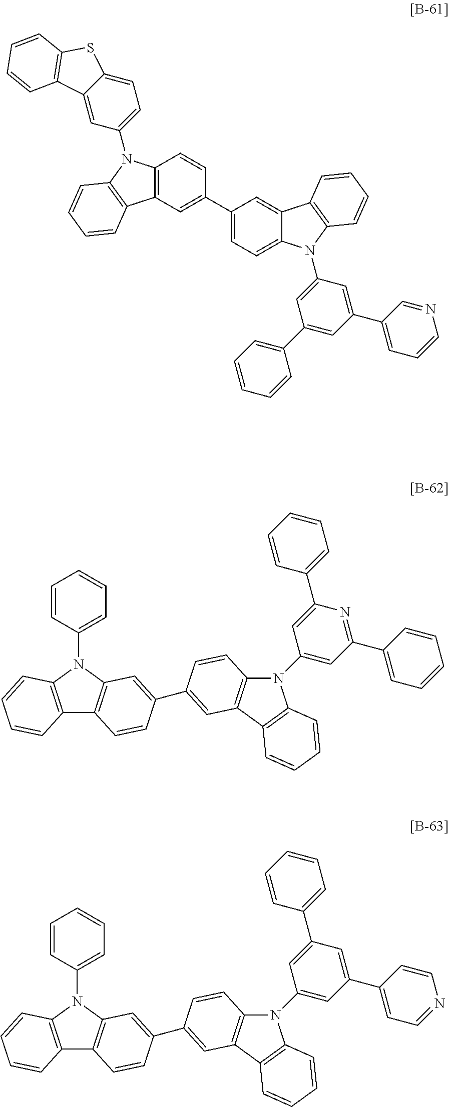 [SMILES: c1ccc(-c2cc(-c3cccnc3)cc(-n3c4ccccc4c4cc(-c5ccc6c(c5)c5ccccc5n6-c5ccc6sc7ccccc7c6c5)ccc43)c2)cc1 ; c1ccc(-c2cc(-n3c4ccccc4c4cc(-c5ccc6c7ccccc7n(-c7ccccc7)c6c5)ccc43)cc(-c3ccccc3)n2)cc1 ; c1ccc(-c2cc(-c3ccncc3)cc(-n3c4ccccc4c4cc(-c5ccc6c7ccccc7n(-c7ccccc7)c6c5)ccc43)c2)cc1]

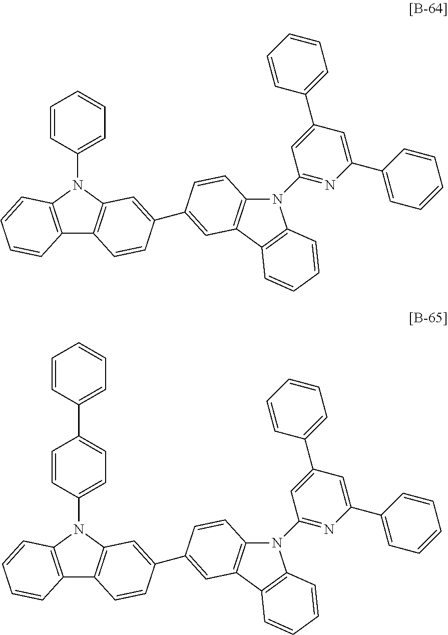 [SMILES: c1ccc(-c2cc(-c3ccccc3)nc(-n3c4ccccc4c4cc(-c5ccc6c7ccccc7n(-c7ccccc7)c6c5)ccc43)c2)cc1 ; c1ccc(-c2ccc(-n3c4ccccc4c4ccc(-c5ccc6c(c5)c5ccccc5n6-c5cc(-c6ccccc6)cc(-c6ccccc6)n5)cc43)cc2)cc1]

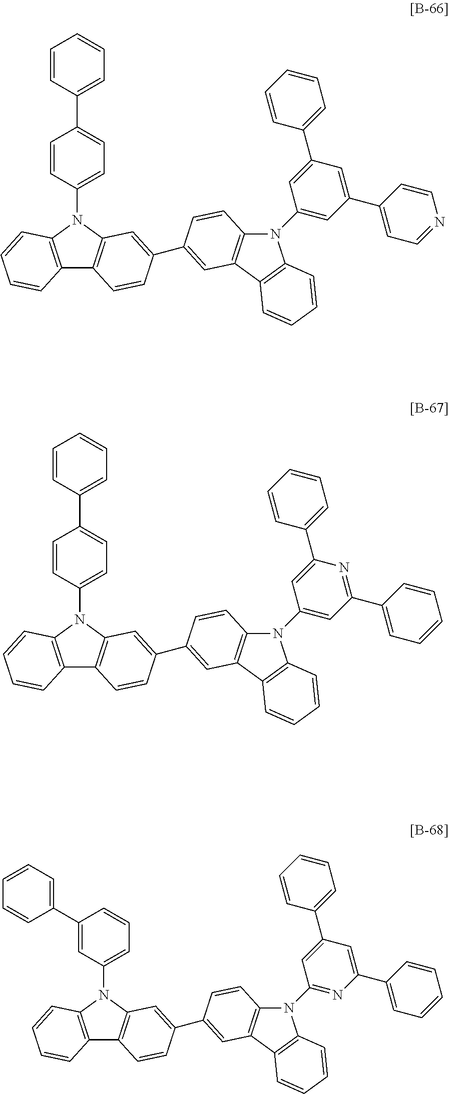 [SMILES: c1ccc(-c2ccc(-n3c4ccccc4c4ccc(-c5ccc6c(c5)c5ccccc5n6-c5cc(-c6ccccc6)cc(-c6ccncc6)c5)cc43)cc2)cc1 ; c1ccc(-c2ccc(-n3c4ccccc4c4ccc(-c5ccc6c(c5)c5ccccc5n6-c5cc(-c6ccccc6)nc(-c6ccccc6)c5)cc43)cc2)cc1 ; c1ccc(-c2cccc(-n3c4ccccc4c4ccc(-c5ccc6c(c5)c5ccccc5n6-c5cc(-c6ccccc6)cc(-c6ccccc6)n5)cc43)c2)cc1]

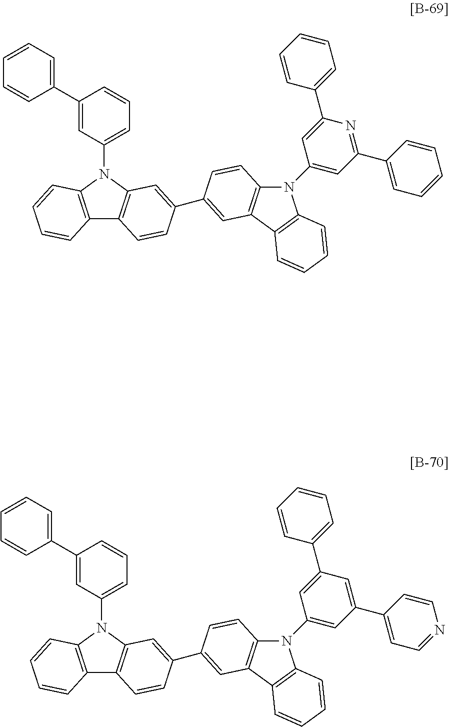 [SMILES: c1ccc(-c2cccc(-n3c4ccccc4c4ccc(-c5ccc6c(c5)c5ccccc5n6-c5cc(-c6ccccc6)nc(-c6ccccc6)c5)cc43)c2)cc1 ; c1ccc(-c2cccc(-n3c4ccccc4c4ccc(-c5ccc6c(c5)c5ccccc5n6-c5cc(-c6ccccc6)cc(-c6ccncc6)c5)cc43)c2)cc1]

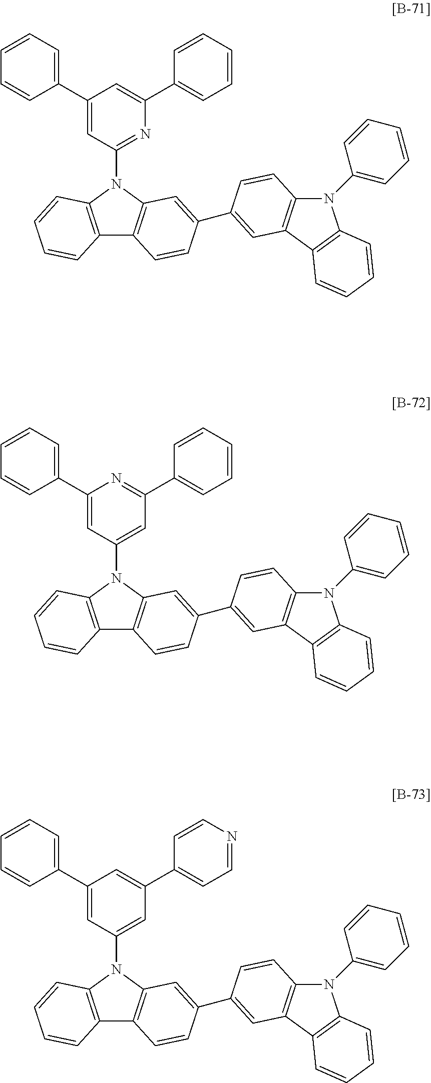 [SMILES: c1ccc(-c2cc(-c3ccccc3)nc(-n3c4ccccc4c4ccc(-c5ccc6c(c5)c5ccccc5n6-c5ccccc5)cc43)c2)cc1 ; c1ccc(-c2cc(-n3c4ccccc4c4ccc(-c5ccc6c(c5)c5ccccc5n6-c5ccccc5)cc43)cc(-c3ccccc3)n2)cc1 ; c1ccc(-c2cc(-c3ccncc3)cc(-n3c4ccccc4c4ccc(-c5ccc6c(c5)c5ccccc5n6-c5ccccc5)cc43)c2)cc1]

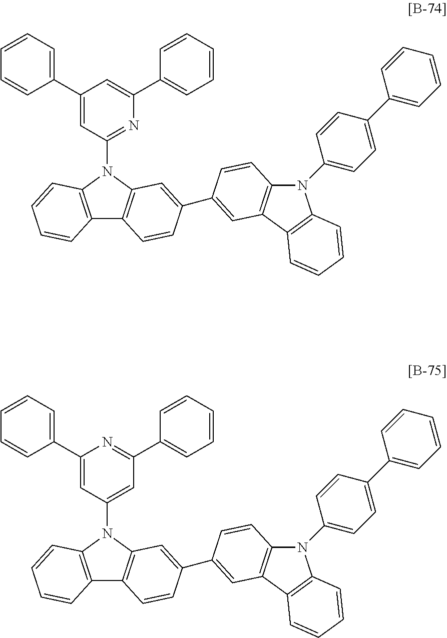 [SMILES: c1ccc(-c2ccc(-n3c4ccccc4c4cc(-c5ccc6c7ccccc7n(-c7cc(-c8ccccc8)cc(-c8ccccc8)n7)c6c5)ccc43)cc2)cc1 ; c1ccc(-c2ccc(-n3c4ccccc4c4cc(-c5ccc6c7ccccc7n(-c7cc(-c8ccccc8)nc(-c8ccccc8)c7)c6c5)ccc43)cc2)cc1]

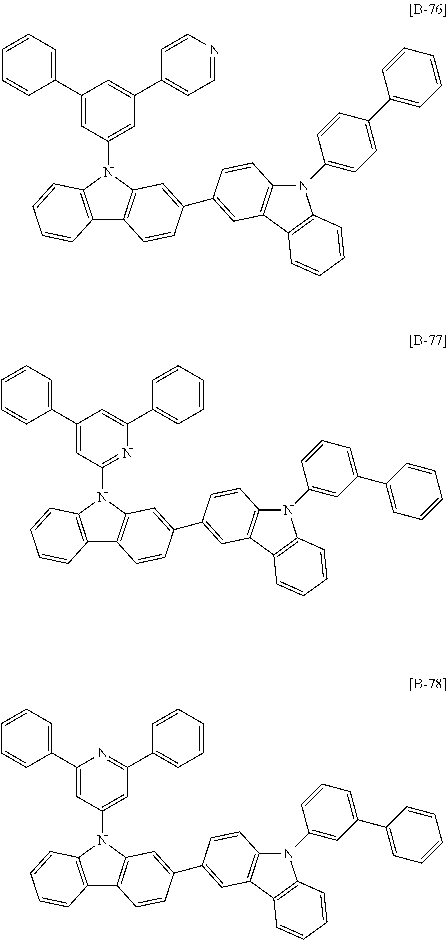 [SMILES: c1ccc(-c2ccc(-n3c4ccccc4c4cc(-c5ccc6c7ccccc7n(-c7cc(-c8ccccc8)cc(-c8ccncc8)c7)c6c5)ccc43)cc2)cc1 ; c1ccc(-c2cccc(-n3c4ccccc4c4cc(-c5ccc6c7ccccc7n(-c7cc(-c8ccccc8)cc(-c8ccccc8)n7)c6c5)ccc43)c2)cc1 ; c1ccc(-c2cccc(-n3c4ccccc4c4cc(-c5ccc6c7ccccc7n(-c7cc(-c8ccccc8)nc(-c8ccccc8)c7)c6c5)ccc43)c2)cc1]

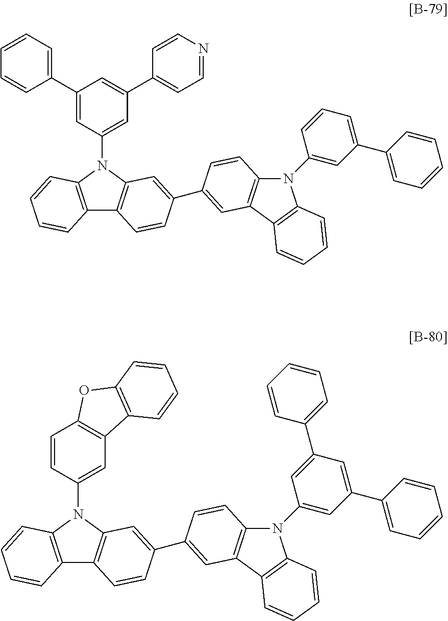 [SMILES: c1ccc(-c2cccc(-n3c4ccccc4c4cc(-c5ccc6c7ccccc7n(-c7cc(-c8ccccc8)cc(-c8ccncc8)c7)c6c5)ccc43)c2)cc1 ; c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1]

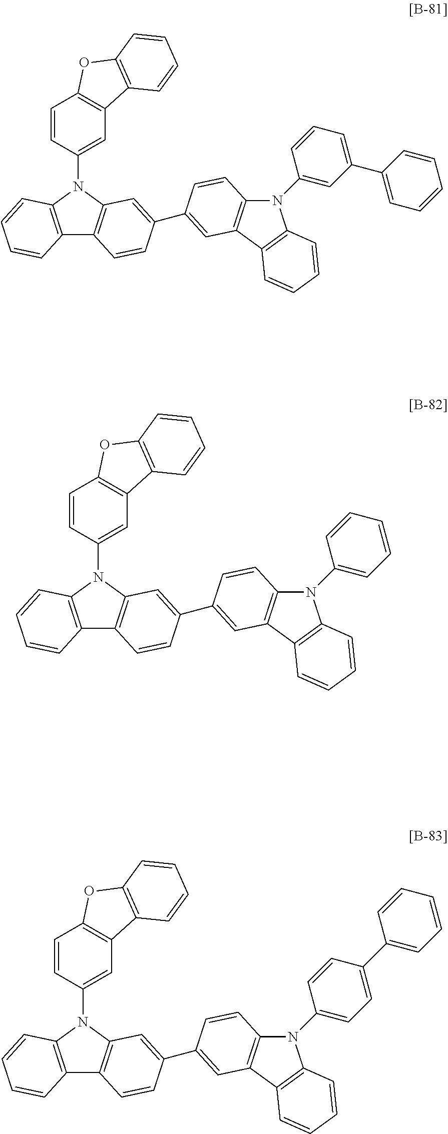 [SMILES: c1ccc(-c2cccc(-n3c4ccccc4c4cc(-c5ccc6c7ccccc7n(-c7ccc8oc9ccccc9c8c7)c6c5)ccc43)c2)cc1 ; c1ccc(-n2c3ccccc3c3cc(-c4ccc5c6ccccc6n(-c6ccc7oc8ccccc8c7c6)c5c4)ccc32)cc1 ; c1ccc(-c2ccc(-n3c4ccccc4c4cc(-c5ccc6c7ccccc7n(-c7ccc8oc9ccccc9c8c7)c6c5)ccc43)cc2)cc1]

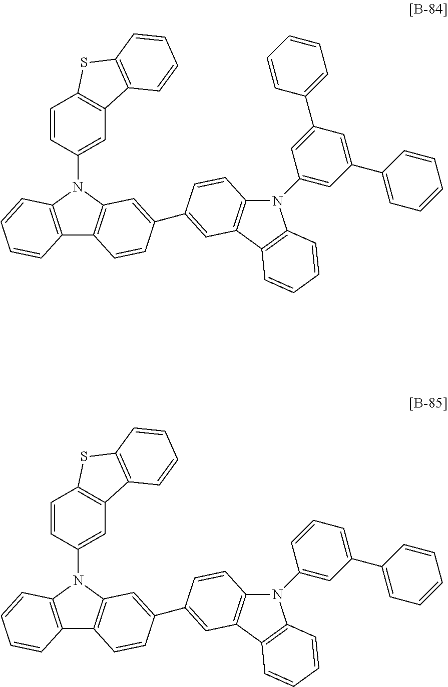 [SMILES: c1ccc(-c2cc(-c3ccccc3)cc(-n3c4ccccc4c4cc(-c5ccc6c7ccccc7n(-c7ccc8sc9ccccc9c8c7)c6c5)ccc43)c2)cc1 ; c1ccc(-c2cccc(-n3c4ccccc4c4cc(-c5ccc6c7ccccc7n(-c7ccc8sc9ccccc9c8c7)c6c5)ccc43)c2)cc1]

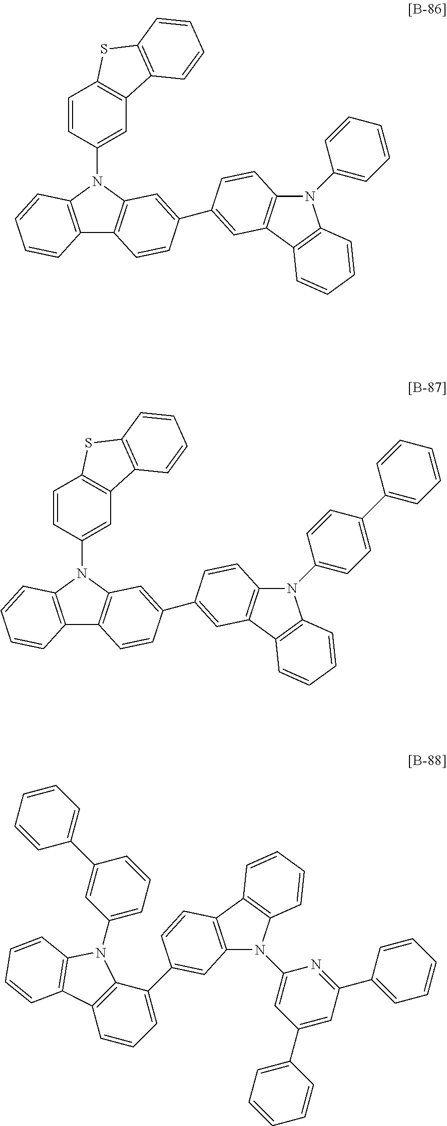 [SMILES: c1ccc(-n2c3ccccc3c3cc(-c4ccc5c6ccccc6n(-c6ccc7sc8ccccc8c7c6)c5c4)ccc32)cc1 ; c1ccc(-c2ccc(-n3c4ccccc4c4cc(-c5ccc6c7ccccc7n(-c7ccc8sc9ccccc9c8c7)c6c5)ccc43)cc2)cc1 ; c1ccc(-c2cccc(-n3c4ccccc4c4cccc(-c5ccc6c7ccccc7n(-c7cc(-c8ccccc8)cc(-c8ccccc8)n7)c6c5)c43)c2)cc1]

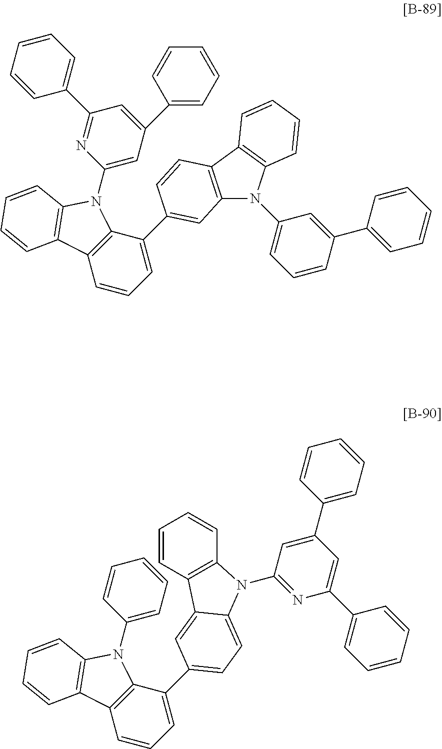 [SMILES: c1ccc(-c2cccc(-n3c4ccccc4c4ccc(-c5cccc6c7ccccc7n(-c7cc(-c8ccccc8)cc(-c8ccccc8)n7)c56)cc43)c2)cc1 ; c1ccc(-c2cc(-c3ccccc3)nc(-n3c4ccccc4c4cc(-c5cccc6c7ccccc7n(-c7ccccc7)c56)ccc43)c2)cc1]

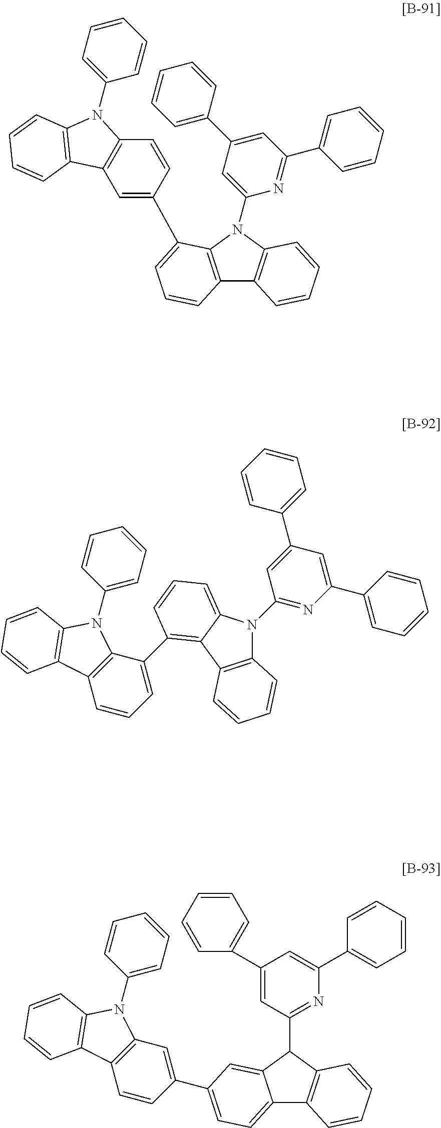 [SMILES: c1ccc(-c2cc(-c3ccccc3)nc(-n3c4ccccc4c4cccc(-c5ccc6c(c5)c5ccccc5n6-c5ccccc5)c43)c2)cc1 ; c1ccc(-c2cc(-c3ccccc3)nc(-n3c4ccccc4c4c(-c5cccc6c7ccccc7n(-c7ccccc7)c56)cccc43)c2)cc1 ; c1ccc(-c2cc(-c3ccccc3)nc(C3c4ccccc4-c4ccc(-c5ccc6c7ccccc7n(-c7ccccc7)c6c5)cc43)c2)cc1]

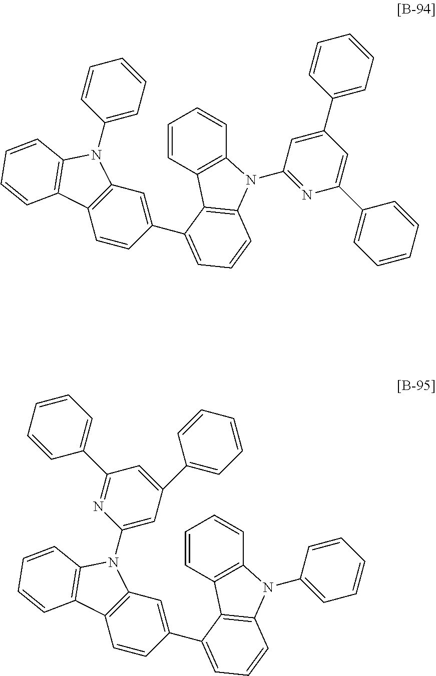 [SMILES: c1ccc(-c2cc(-c3ccccc3)nc(-n3c4ccccc4c4c(-c5ccc6c7ccccc7n(-c7ccccc7)c6c5)cccc43)c2)cc1 ; c1ccc(-c2cc(-c3ccccc3)nc(-n3c4ccccc4c4ccc(-c5cccc6c5c5ccccc5n6-c5ccccc5)cc43)c2)cc1]

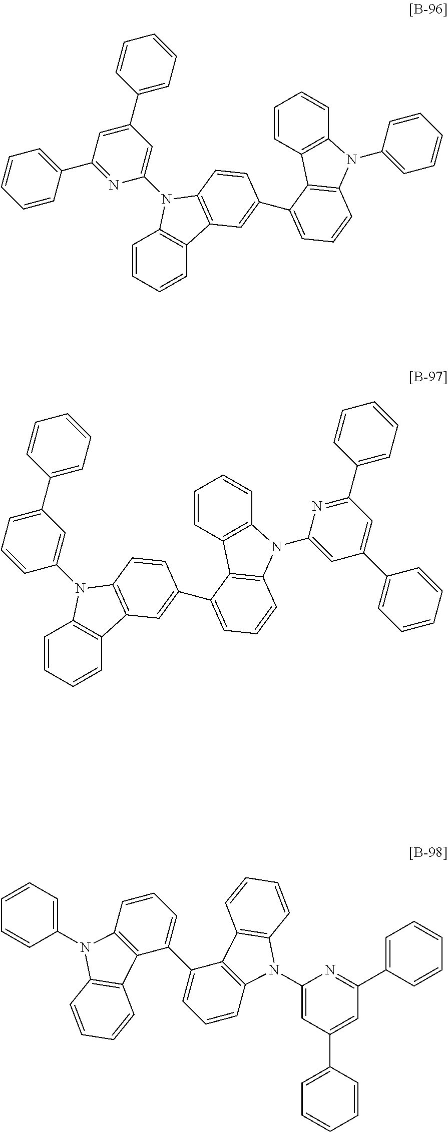 [SMILES: c1ccc(-c2cc(-c3ccccc3)nc(-n3c4ccccc4c4cc(-c5cccc6c5c5ccccc5n6-c5ccccc5)ccc43)c2)cc1 ; c1ccc(-c2cccc(-n3c4ccccc4c4cc(-c5cccc6c5c5ccccc5n6-c5cc(-c6ccccc6)cc(-c6ccccc6)n5)ccc43)c2)cc1 ; c1ccc(-c2cc(-c3ccccc3)nc(-n3c4ccccc4c4c(-c5cccc6c5c5ccccc5n6-c5ccccc5)cccc43)c2)cc1]

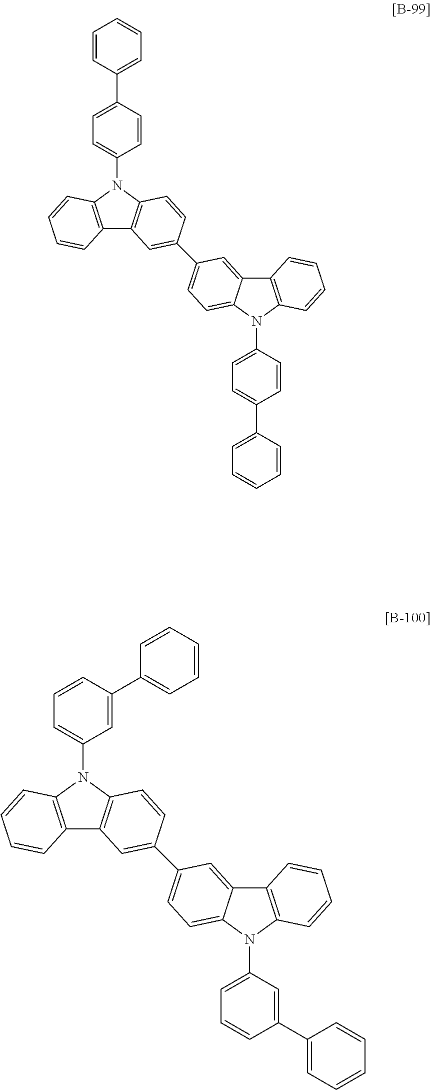 [SMILES: c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

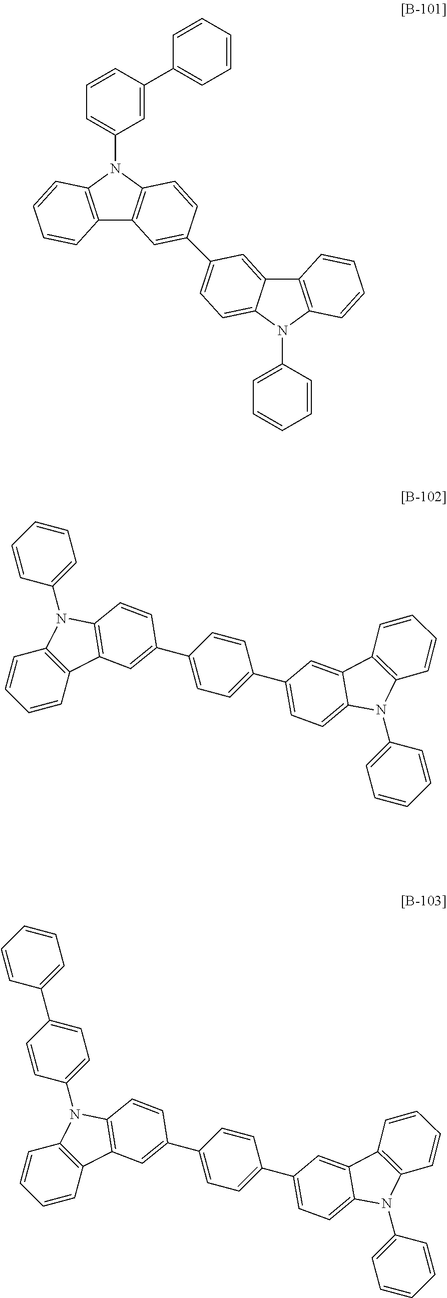 [SMILES: c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc(-c5ccc6c(c5)c5ccccc5n6-c5ccccc5)cc4)ccc32)cc1 ; c1ccc(-c2ccc(-n3c4ccccc4c4cc(-c5ccc(-c6ccc7c(c6)c6ccccc6n7-c6ccccc6)cc5)ccc43)cc2)cc1]

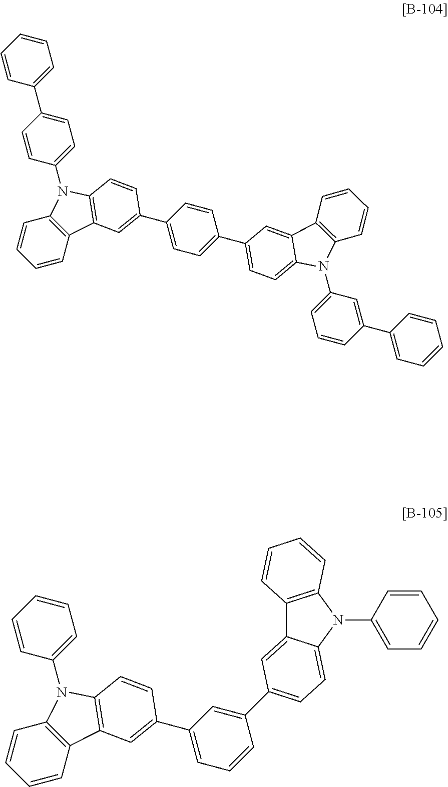 [SMILES: c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1 ; c1ccc(-n2c3ccccc3c3cc(-c4cccc(-c5ccc6c(c5)c5ccccc5n6-c5ccccc5)c4)ccc32)cc1]

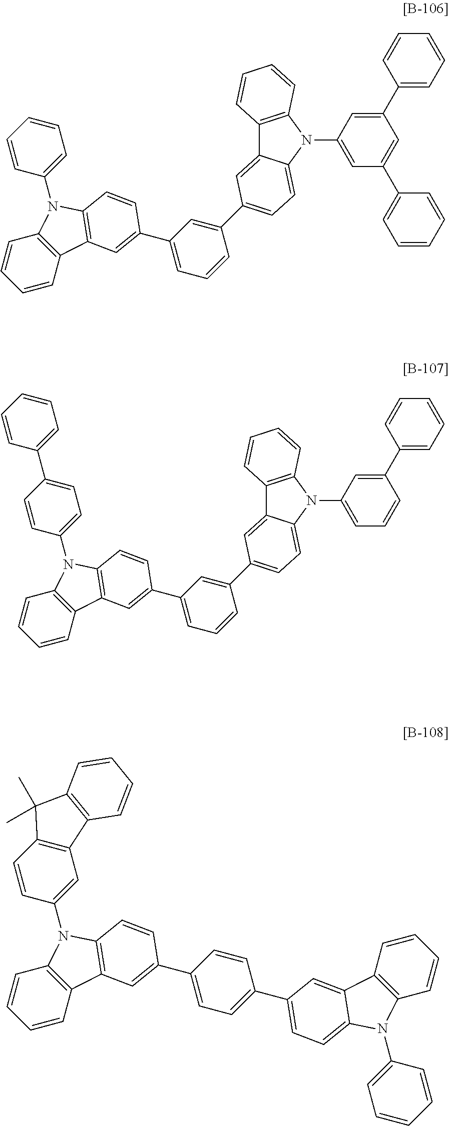 [SMILES: c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1 ; c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1 ; CC1(C)c2ccccc2-c2cc(-n3c4ccccc4c4cc(-c5ccc(-c6ccc7c(c6)c6ccccc6n7-c6ccccc6)cc5)ccc43)ccc21]

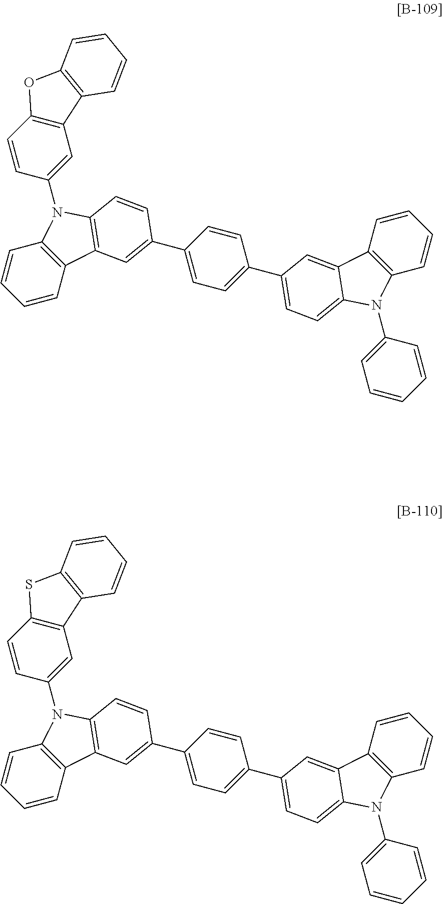 [SMILES: c1ccc(-n2c3ccccc3c3cc(-c4ccc(-c5ccc6c(c5)c5ccccc5n6-c5ccc6oc7ccccc7c6c5)cc4)ccc32)cc1 ; c1ccc(-n2c3ccccc3c3cc(-c4ccc(-c5ccc6c(c5)c5ccccc5n6-c5ccc6sc7ccccc7c6c5)cc4)ccc32)cc1]

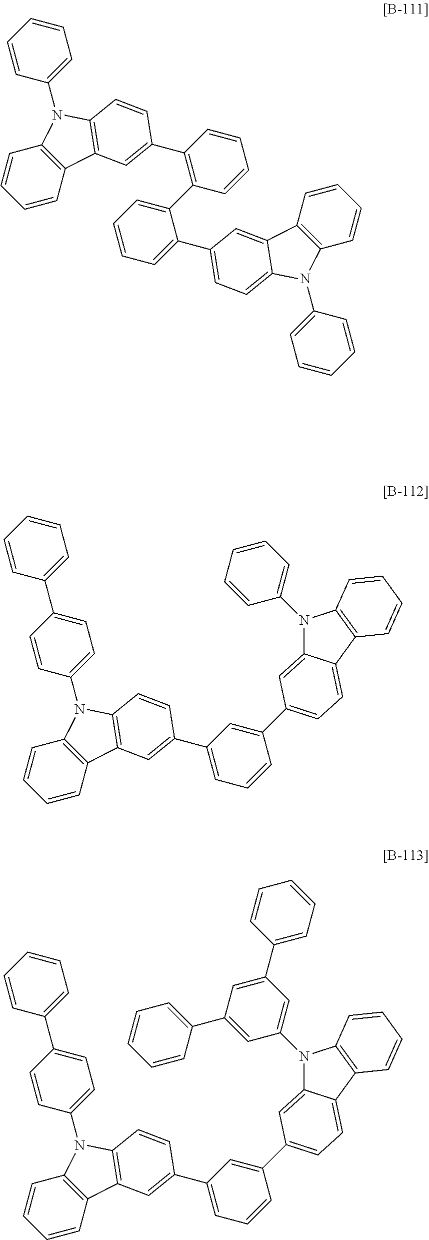 [SMILES: c1ccc(-n2c3ccccc3c3cc(-c4ccccc4-c4ccccc4-c4ccc5c(c4)c4ccccc4n5-c4ccccc4)ccc32)cc1 ; c1ccc(-c2ccc(-n3c4ccccc4c4cc(-c5cccc(-c6ccc7c8ccccc8n(-c8ccccc8)c7c6)c5)ccc43)cc2)cc1 ; c1ccc(-c2ccc(-n3c4ccccc4c4cc(-c5cccc(-c6ccc7c8ccccc8n(-c8cc(-c9ccccc9)cc(-c9ccccc9)c8)c7c6)c5)ccc43)cc2)cc1]

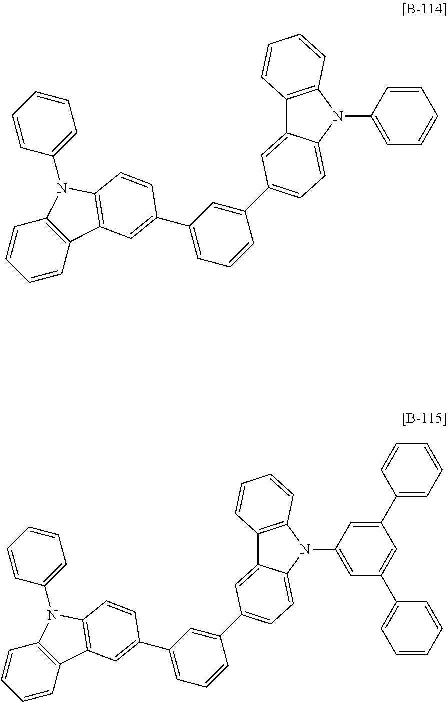 [SMILES: c1ccc(-n2c3ccccc3c3cc(-c4cccc(-c5ccc6c(c5)c5ccccc5n6-c5ccccc5)c4)ccc32)cc1 ; c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1]

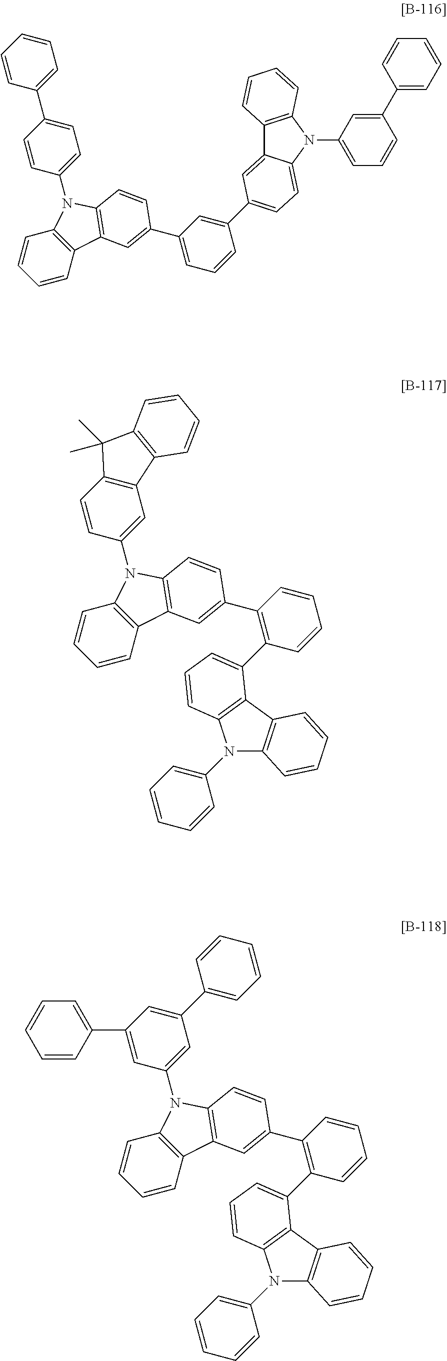 [SMILES: c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1 ; CC1(C)c2ccccc2-c2cc(-n3c4ccccc4c4cc(-c5ccccc5-c5cccc6c5c5ccccc5n6-c5ccccc5)ccc43)ccc21 ; c1ccc(-c2cc(-c3ccccc3)cc(-n3c4ccccc4c4cc(-c5ccccc5-c5cccc6c5c5ccccc5n6-c5ccccc5)ccc43)c2)cc1]

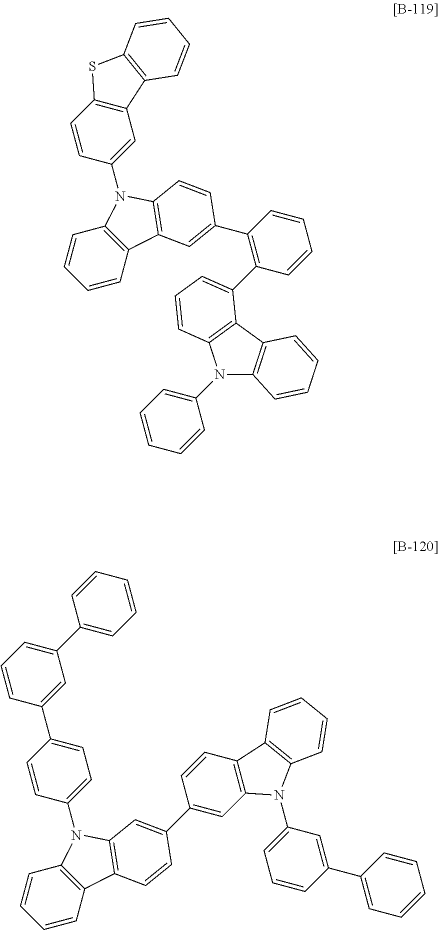 [SMILES: c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1]

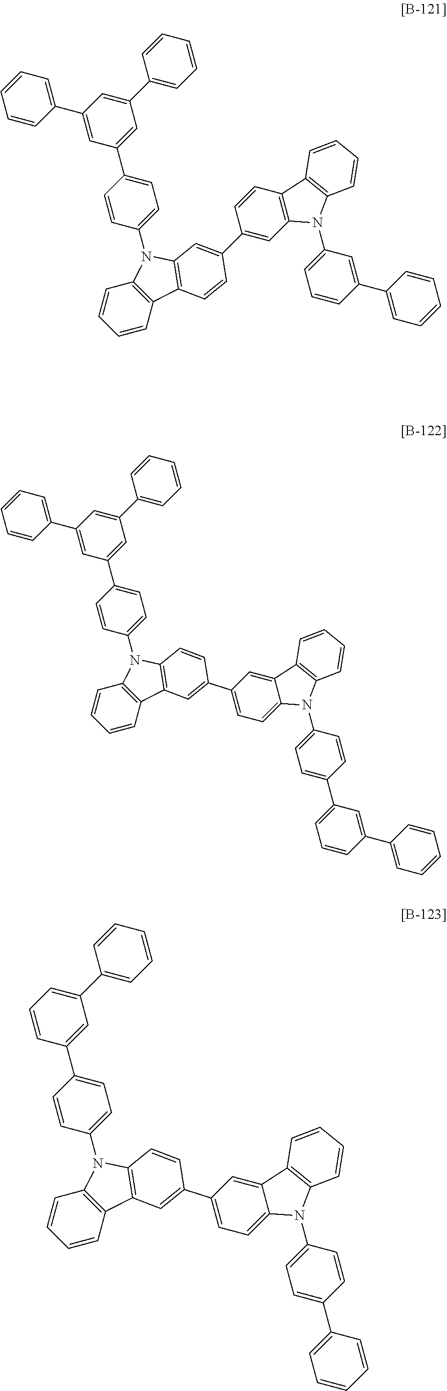 [SMILES: c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cccc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1 ; c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1]

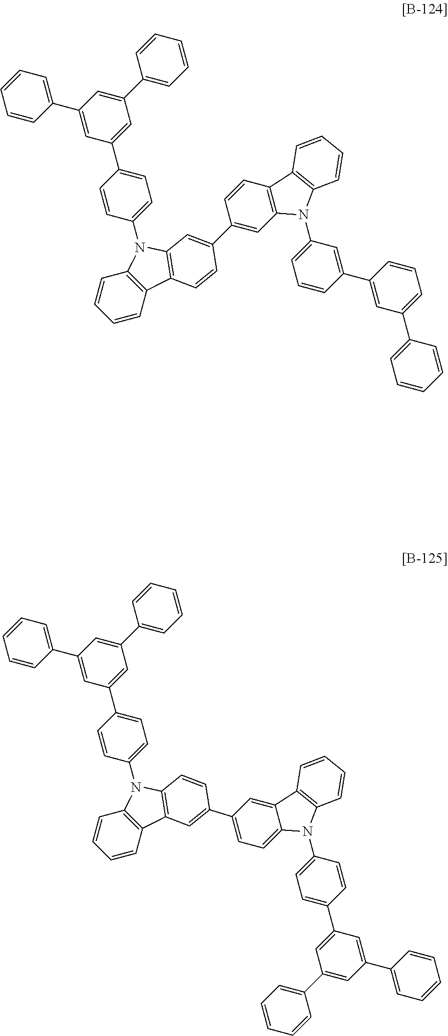 [SMILES: c1ccc(-c2cccc(-c3cccc(-n4c5ccccc5c5ccc(-c6ccc7c8ccccc8n(-c8ccc(-c9cc(-c%10ccccc%10)cc(-c%10ccccc%10)c9)cc8)c7c6)cc54)c3)c2)cc1 ; c1ccc(-c2cc(-c3ccccc3)cc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1]

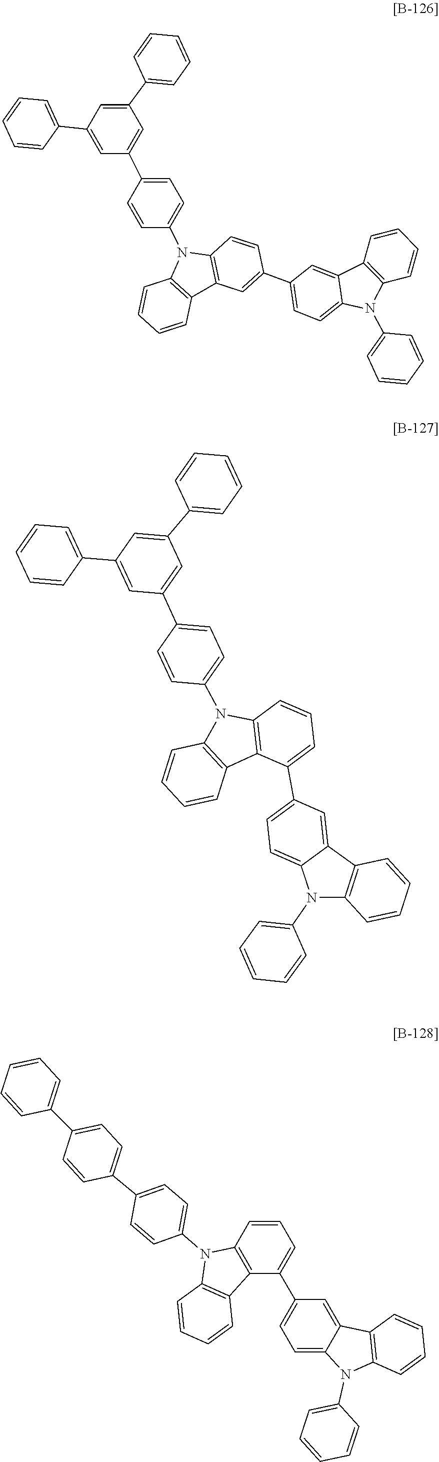 [SMILES: c1ccc(-c2cc(-c3ccccc3)cc(-c3ccc(-n4c5ccccc5c5cc(-c6ccc7c(c6)c6ccccc6n7-c6ccccc6)ccc54)cc3)c2)cc1 ; c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1 ; c1ccc(-c2ccc(-c3ccc(-n4c5ccccc5c5c(-c6ccc7c(c6)c6ccccc6n7-c6ccccc6)cccc54)cc3)cc2)cc1]

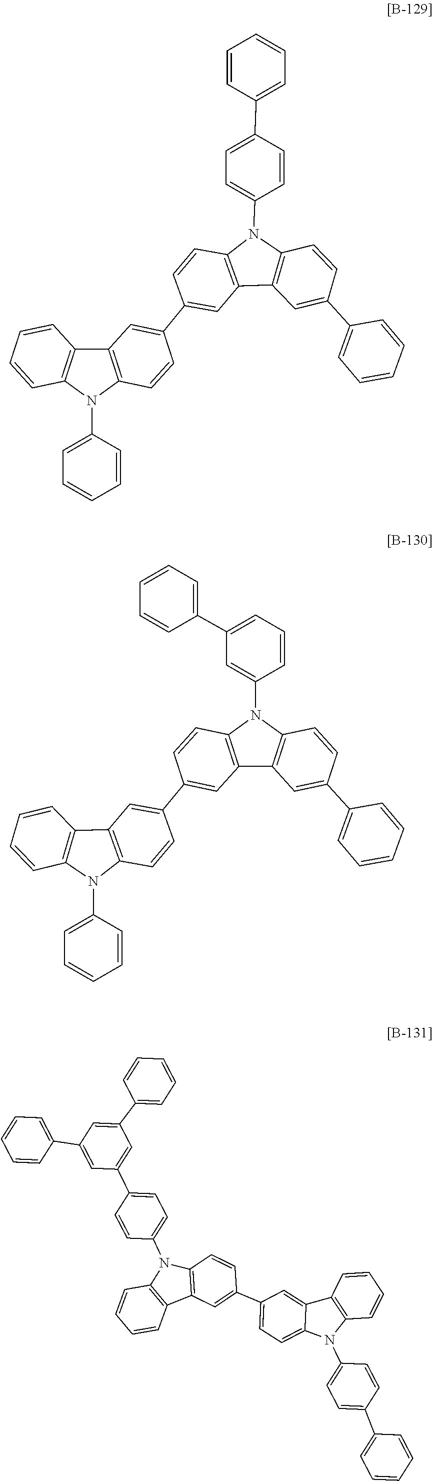 [SMILES: c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-c2cccc(-n3c4ccc(-c5ccccc5)cc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1]

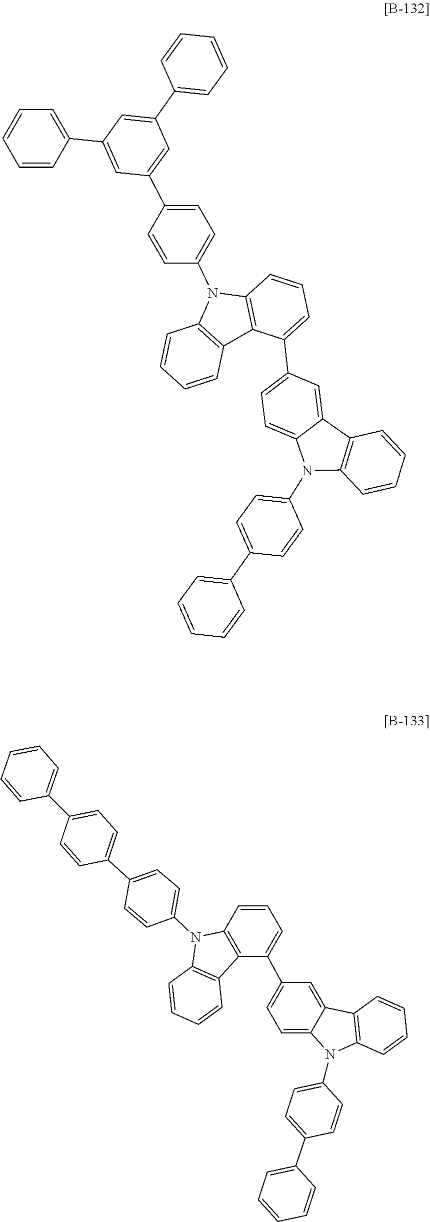 [SMILES: c1ccc(-c2ccc(-n3c4ccccc4c4cc(-c5cccc6c5c5ccccc5n6-c5ccc(-c6cc(-c7ccccc7)cc(-c7ccccc7)c6)cc5)ccc43)cc2)cc1 ; c1ccc(-c2ccc(-c3ccc(-n4c5ccccc5c5c(-c6ccc7c(c6)c6ccccc6n7-c6ccc(-c7ccccc7)cc6)cccc54)cc3)cc2)cc1]

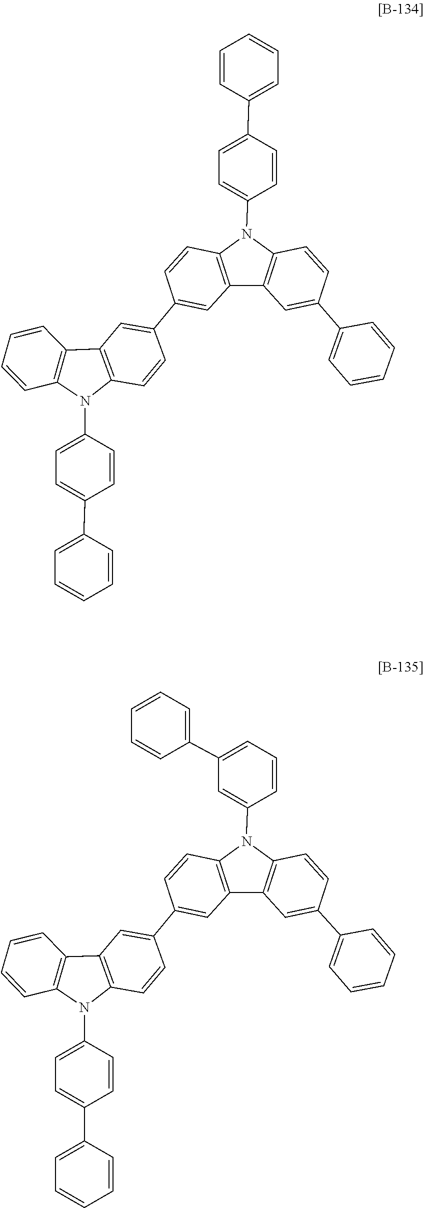 [SMILES: c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1 ; c1ccc(-c2ccc(-n3c4ccccc4c4cc(-c5ccc6c(c5)c5cc(-c7ccccc7)ccc5n6-c5cccc(-c6ccccc6)c5)ccc43)cc2)cc1]

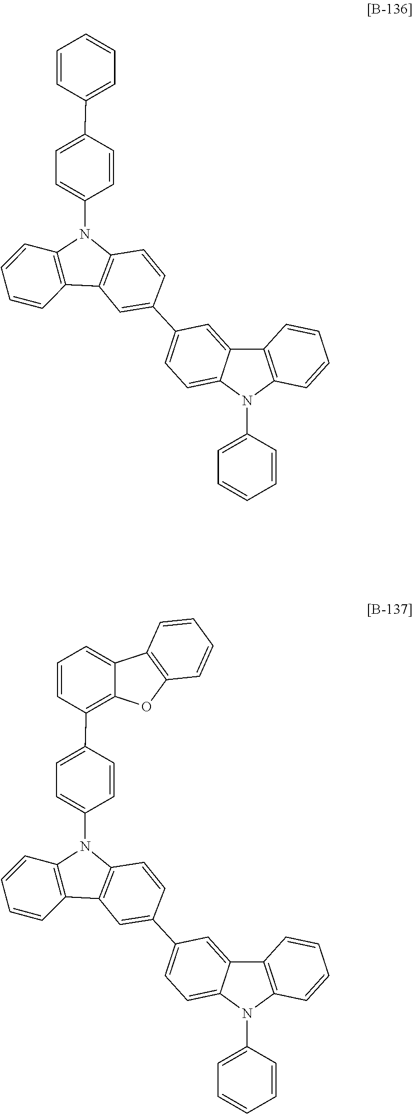 [SMILES: c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1]

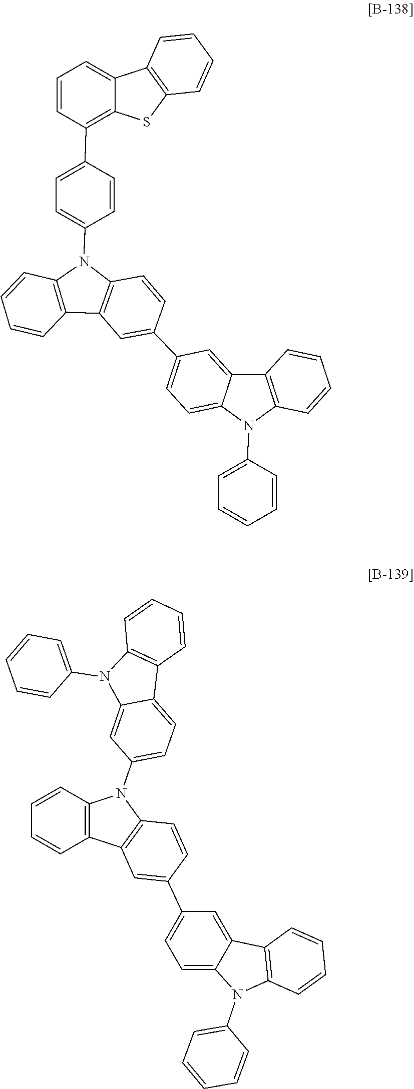 [SMILES: c1ccc(-n2c3ccccc3c3cc(-c4ccc5c(c4)c4ccccc4n5-c4ccc(-c5cccc6c5sc5ccccc56)cc4)ccc32)cc1 ; c1ccc(-n2c3ccccc3c3cc(-c4ccc5c(c4)c4ccccc4n5-c4ccc5c6ccccc6n(-c6ccccc6)c5c4)ccc32)cc1]

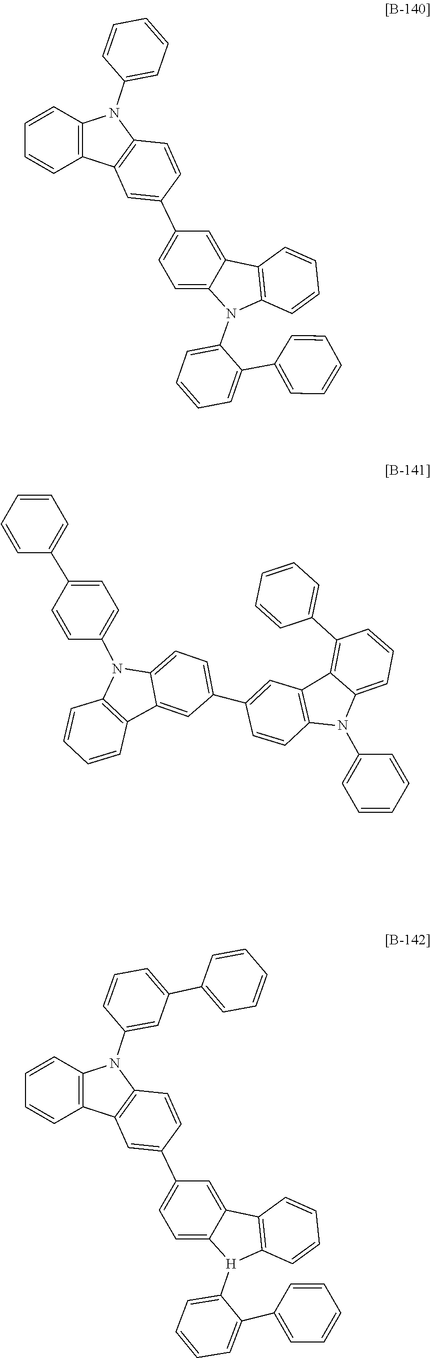

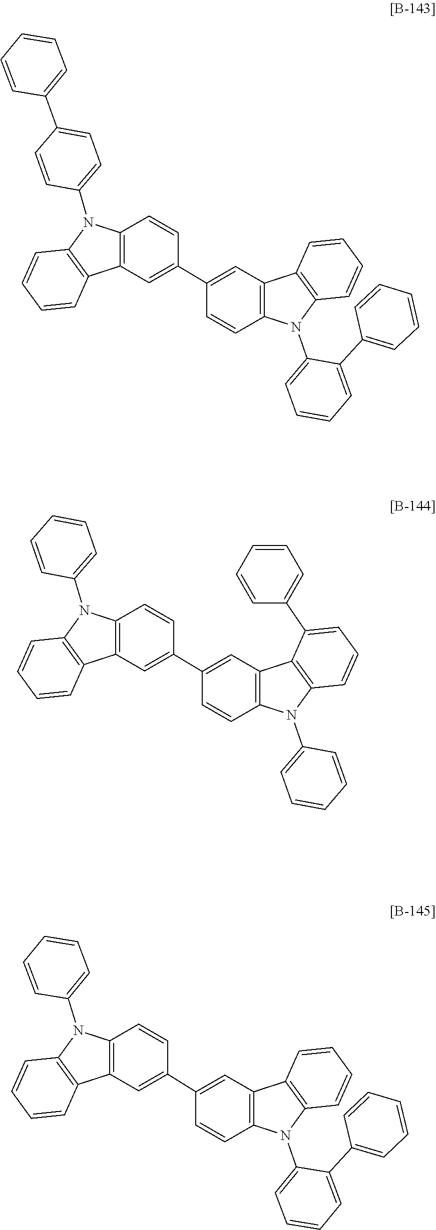 [SMILES: c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc3c2c2cc(-c4ccc5c(c4)c4ccccc4n5-c4ccccc4)ccc2n3-c2ccccc2)cc1 ; c1ccc(-c2ccccc2-n2c3ccccc3c3cc(-c4ccc5c(c4)c4ccccc4n5-c4ccccc4)ccc32)cc1]

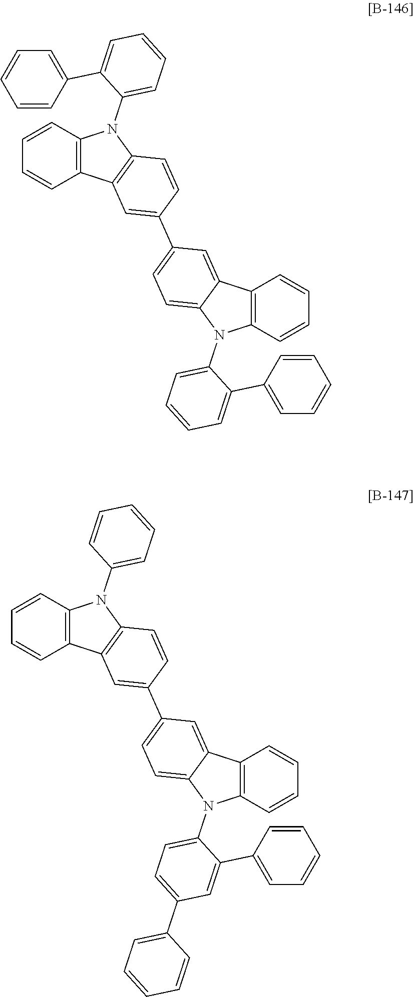 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2ccc(-n3c4ccccc4c4cc(-c5ccc6c(c5)c5ccccc5n6-c5ccccc5)ccc43)c(-c3ccccc3)c2)cc1]

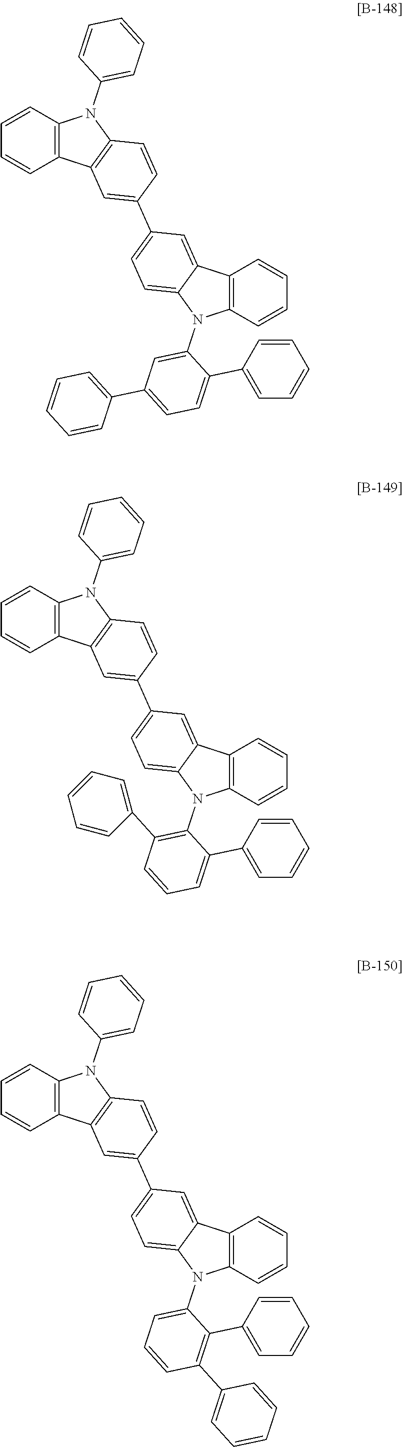 [SMILES: c1ccc(-c2ccc(-c3ccccc3)c(-n3c4ccccc4c4cc(-c5ccc6c(c5)c5ccccc5n6-c5ccccc5)ccc43)c2)cc1 ; c1ccc(-c2cccc(-c3ccccc3)c2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2-c2ccccc2)cc1]

Examples of Compound B-1 to Compound B-150 listed in Group 2 in which at least one hydrogen is replaced with deuterium are shown in Compound B-151 to Compound B-195, exemplified below.

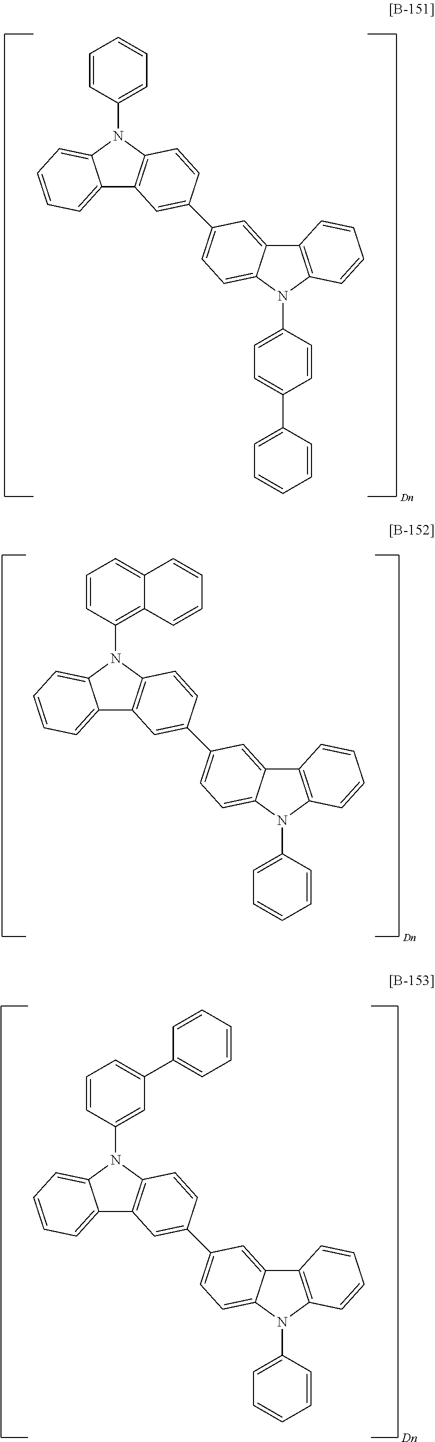

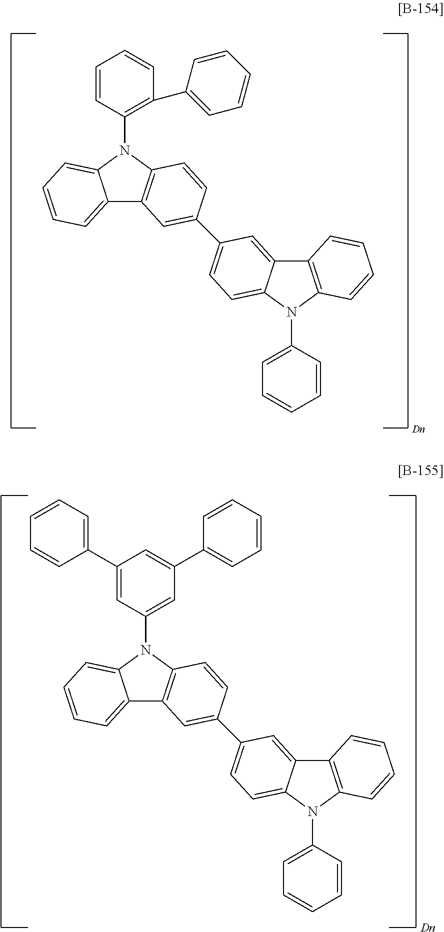

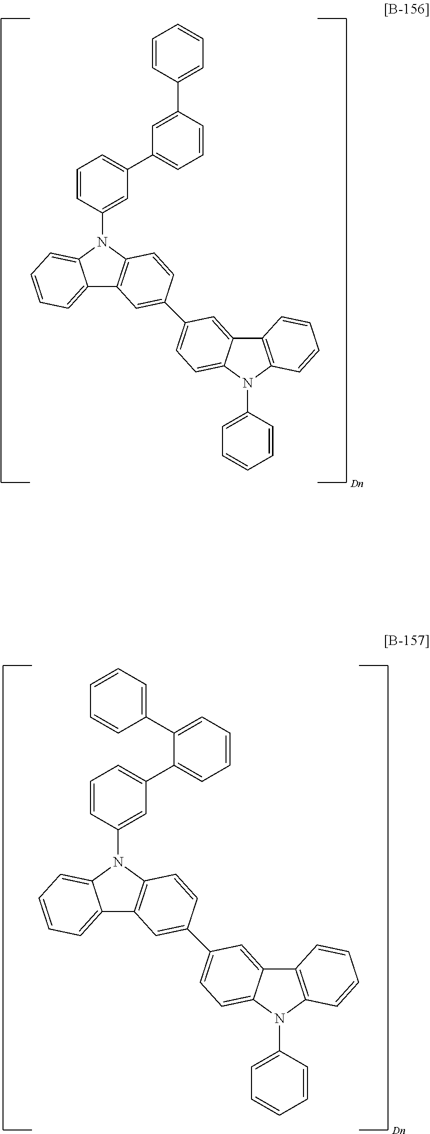

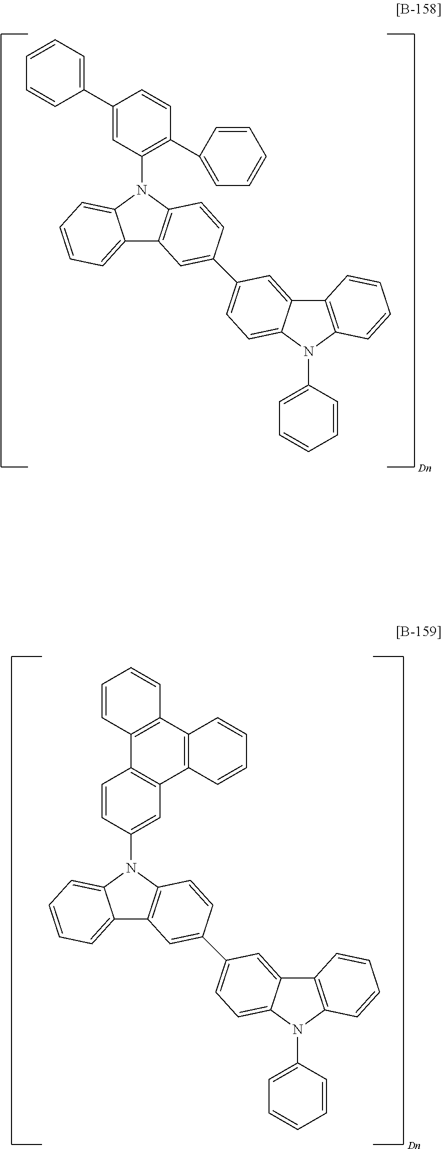

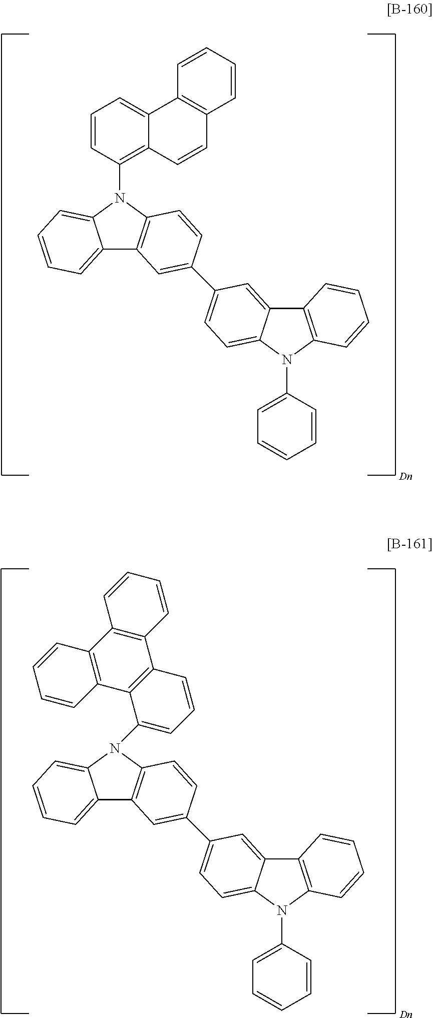

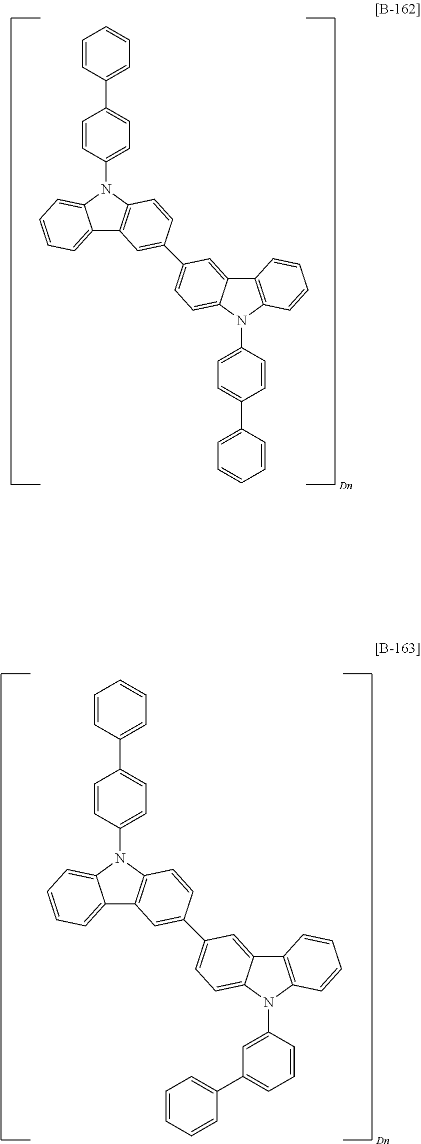

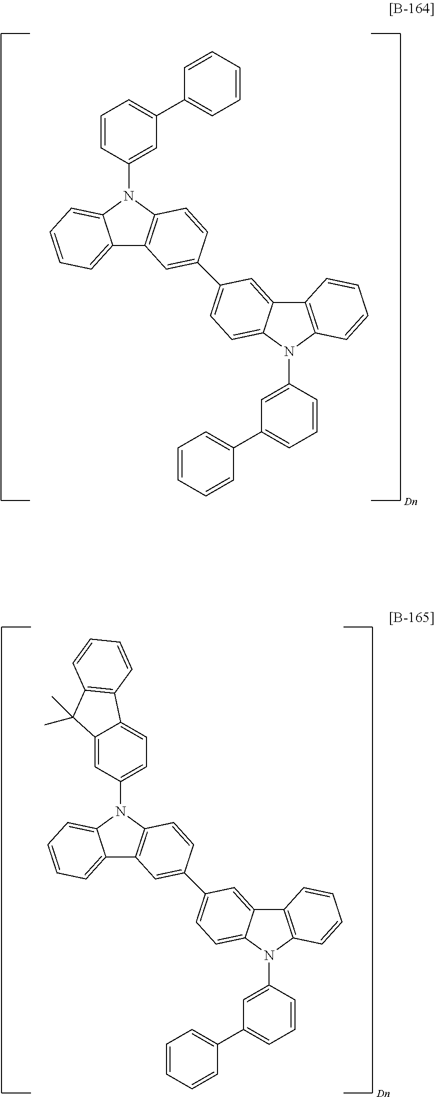

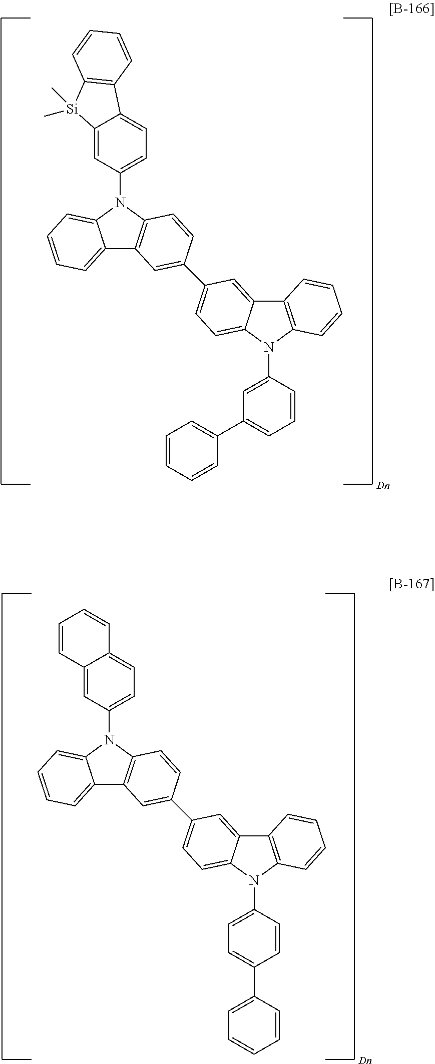

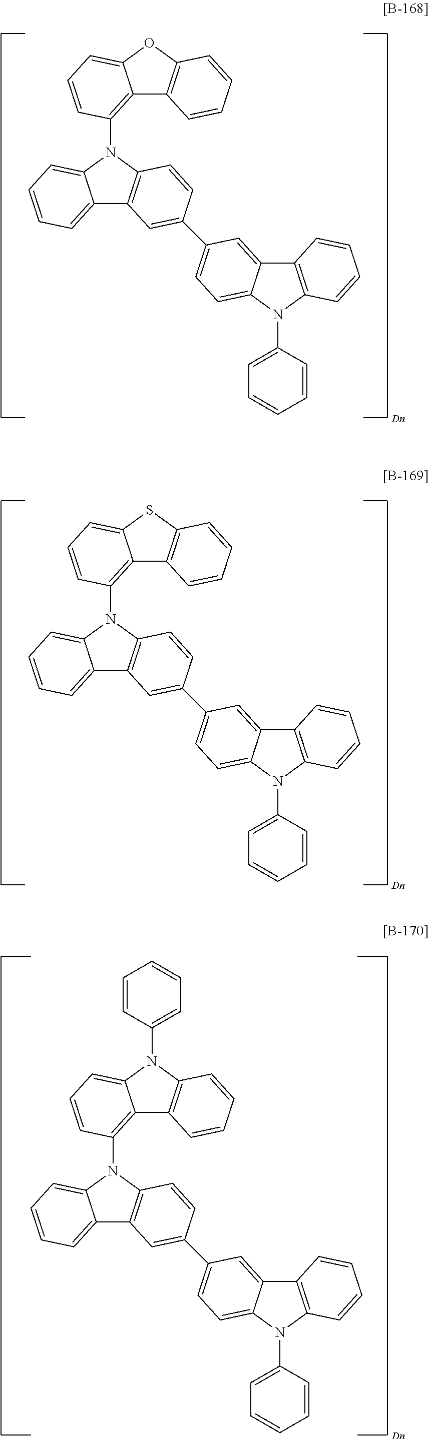

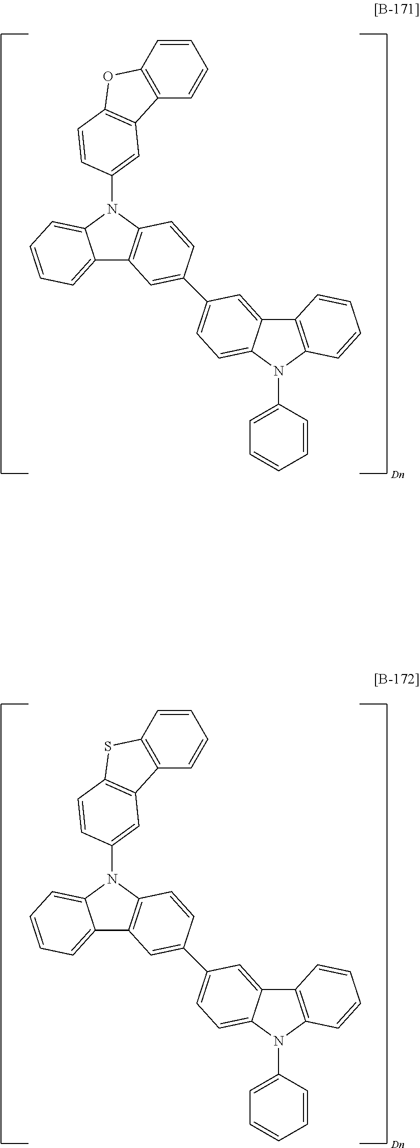

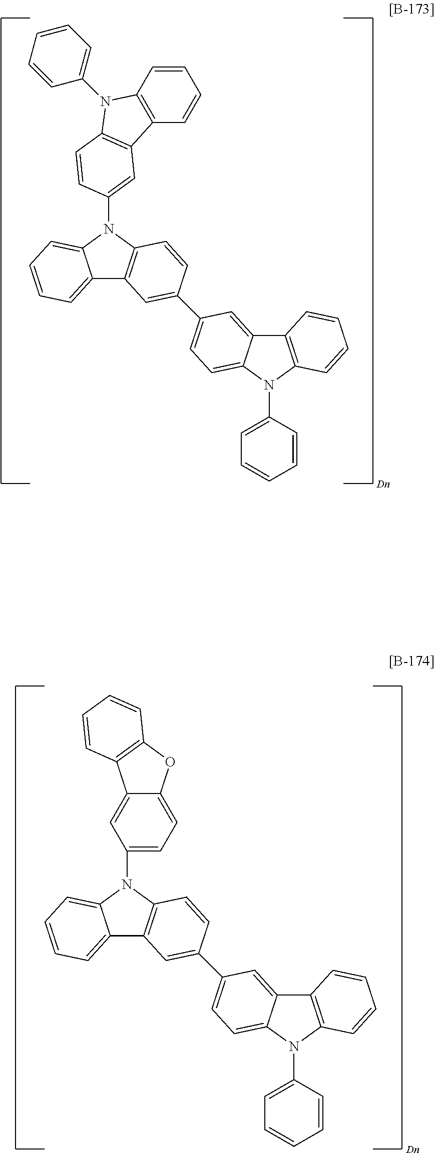

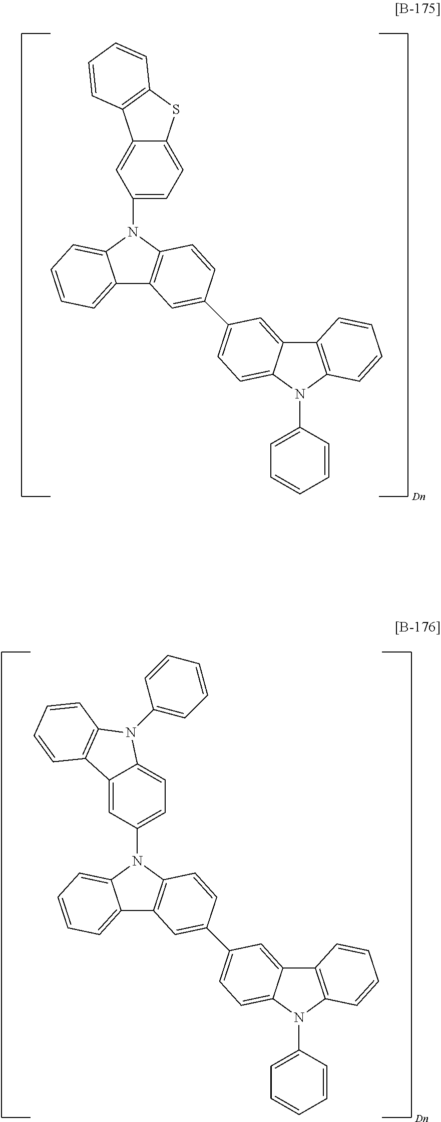

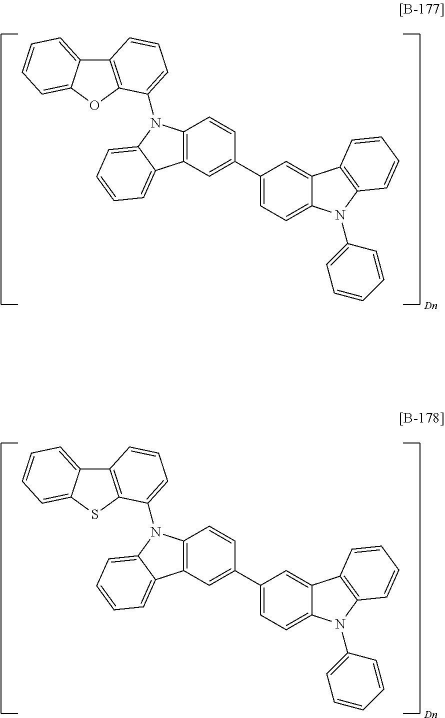

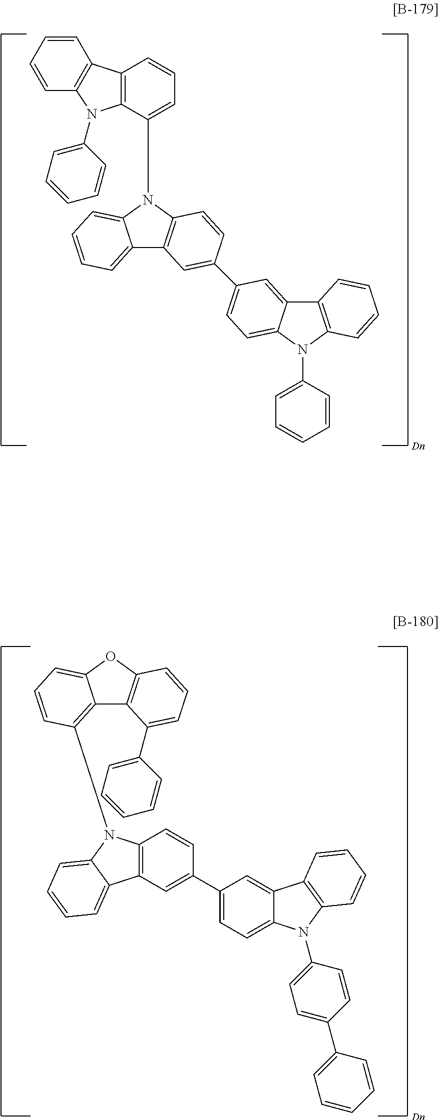

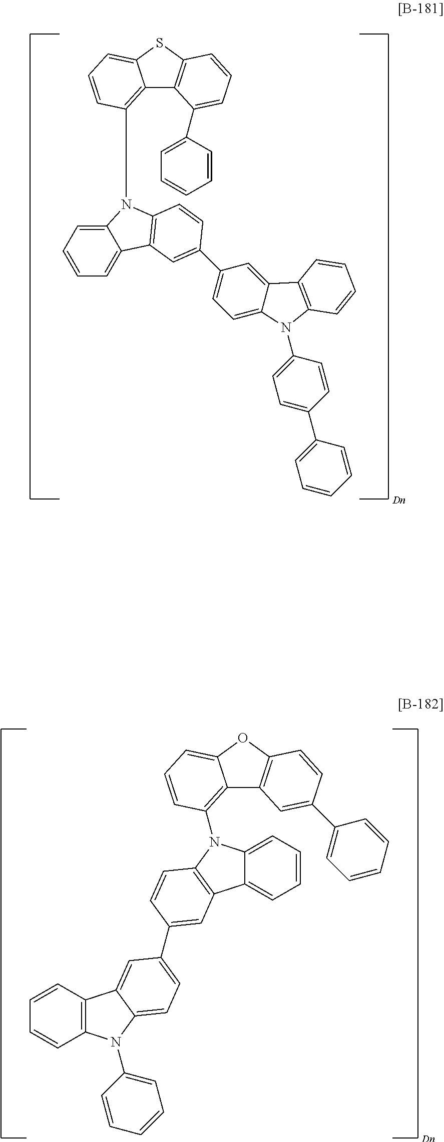

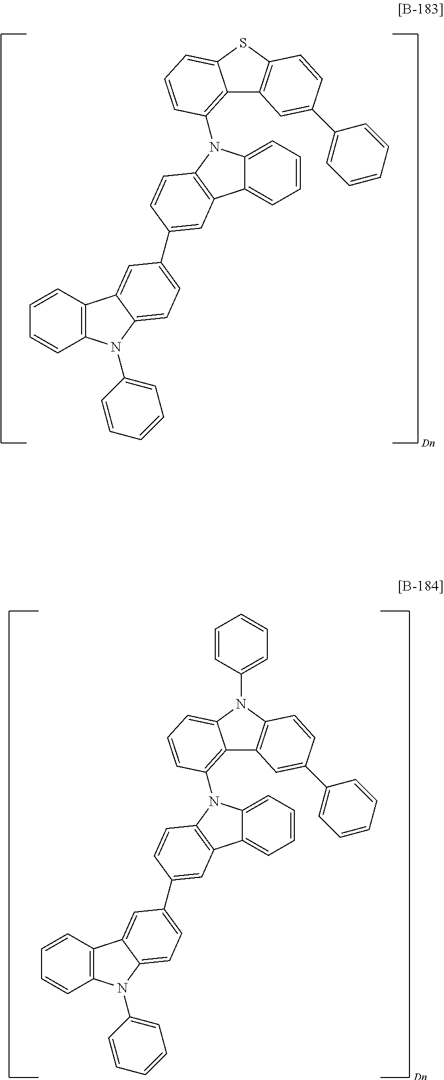

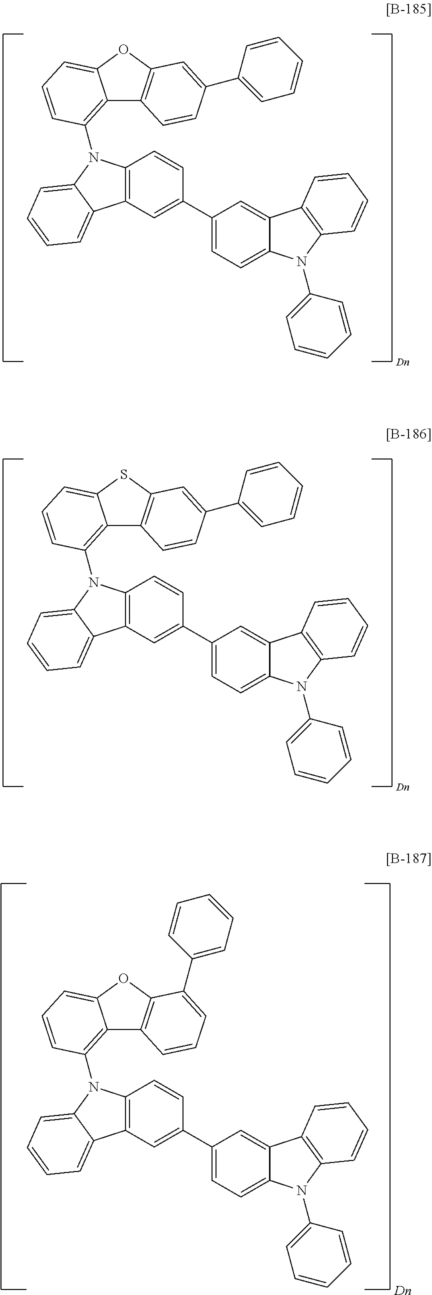

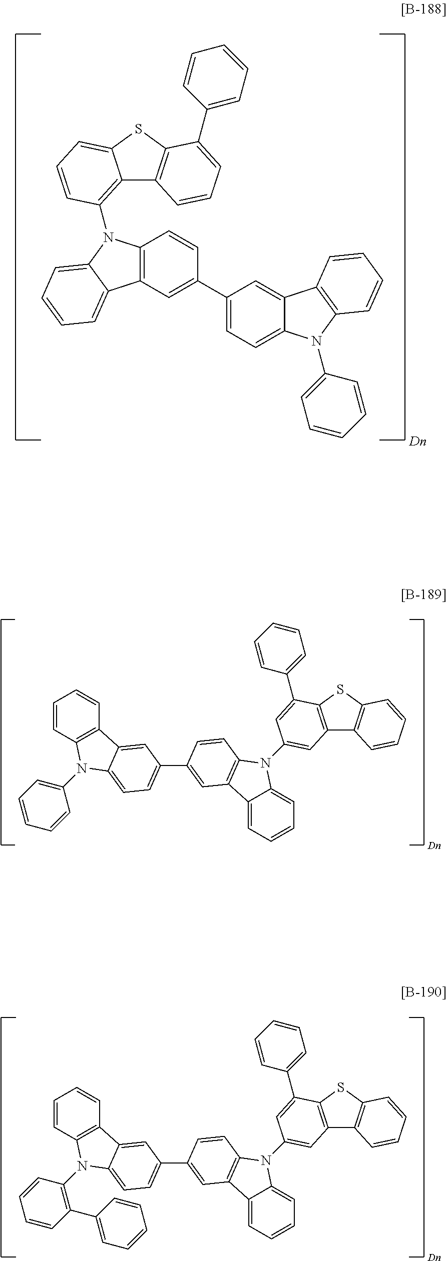

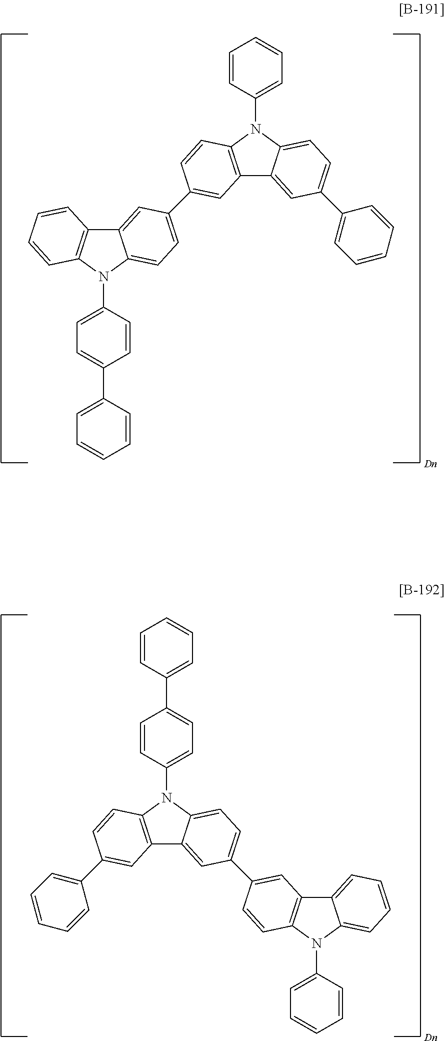

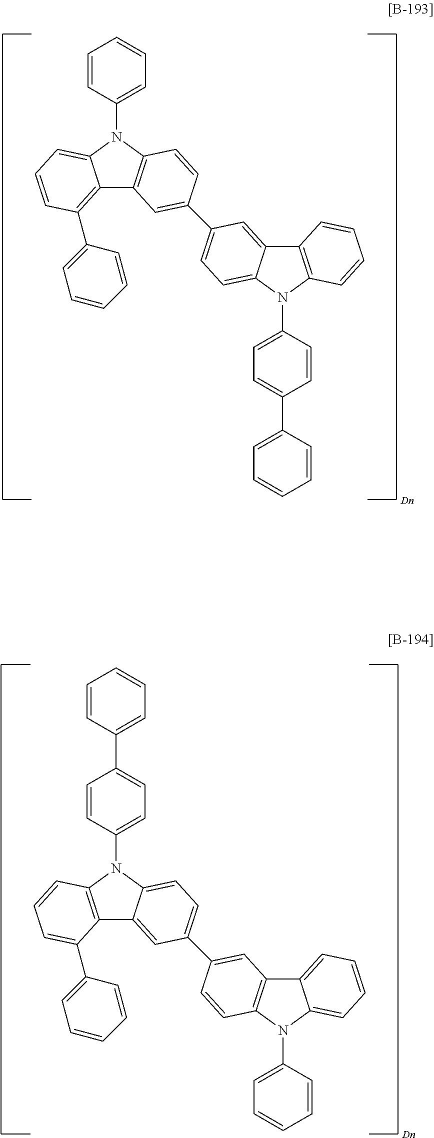

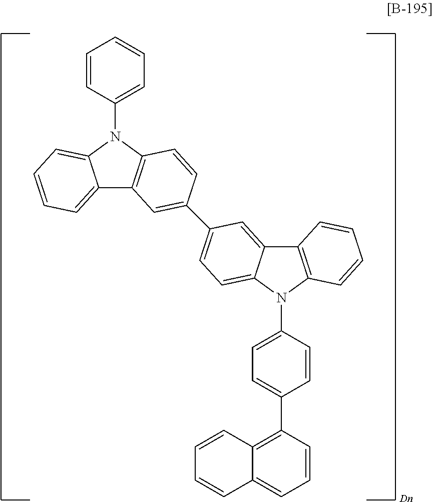

(Dn refers to the number of deuterium atoms substituted, and n is an integer between 1 and the maximum number of substitutions possible)

The position of deuterium substitution and the substitution degree of deuterium substitution may include all changeable ranges within Compound B-1 to Compound B-195.

Specific structures of Compound B-151 to Compound B-195 are presented in Compound B-196 to Compound B-234 below.

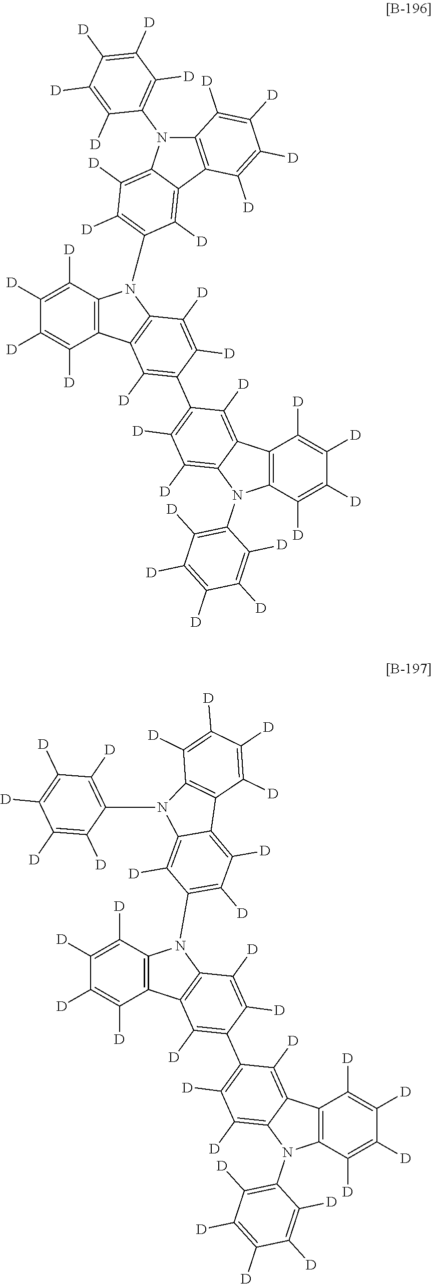

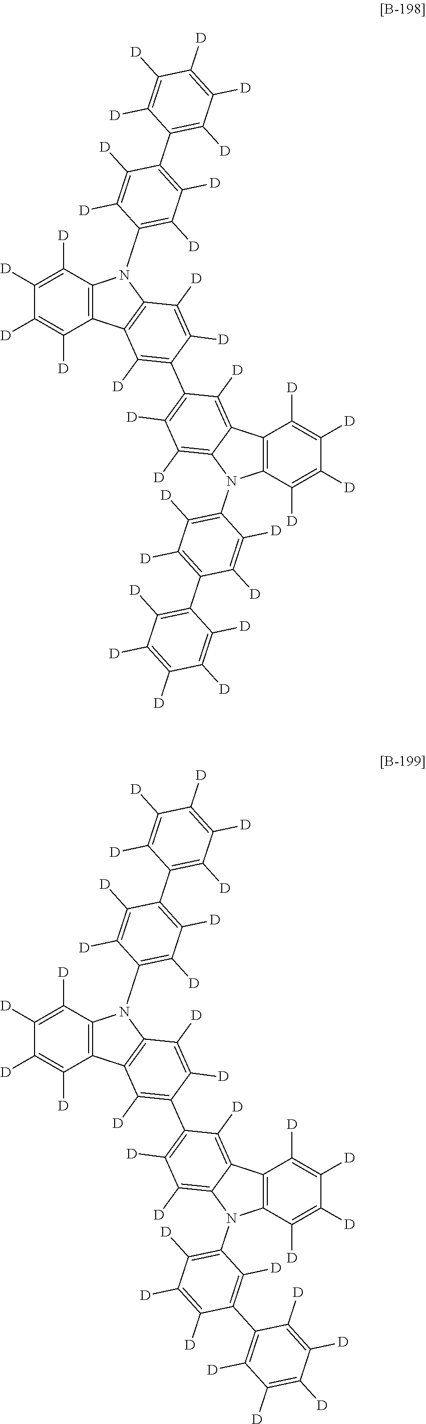

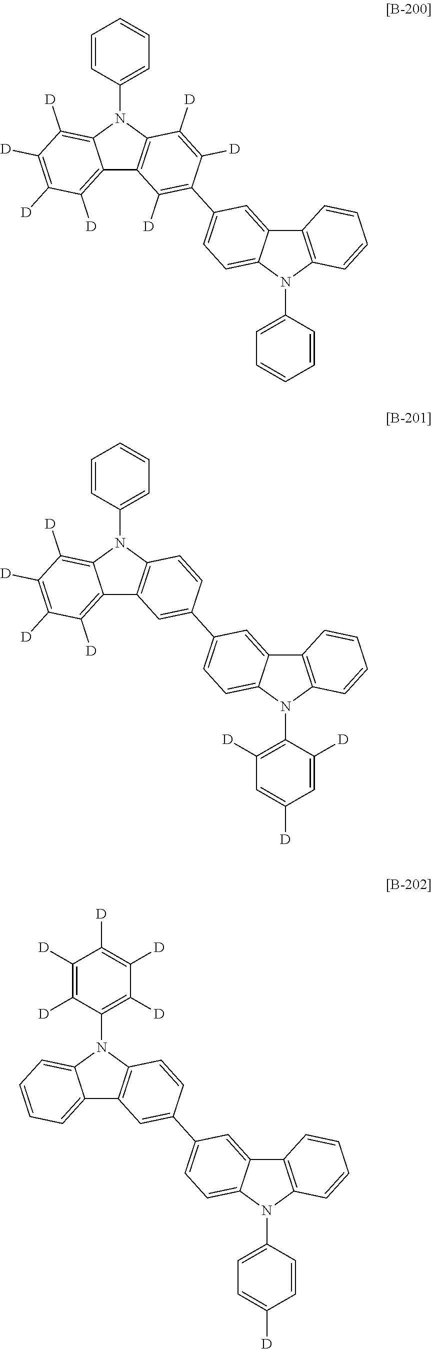 [SMILES: [2H]c1c([2H])c([2H])c2c(c1[2H])c1c([2H])c(-c3ccc4c(c3)c3ccccc3n4-c3ccccc3)c([2H])c([2H])c1n2-c1ccccc1 ; [2H]c1cc([2H])c(-n2c3ccccc3c3cc(-c4ccc5c(c4)c4c([2H])c([2H])c([2H])c([2H])c4n5-c4ccccc4)ccc32)c([2H])c1 ; [2H]c1ccc(-n2c3ccccc3c3cc(-c4ccc5c(c4)c4ccccc4n5-c4c([2H])c([2H])c([2H])c([2H])c4[2H])ccc32)cc1]

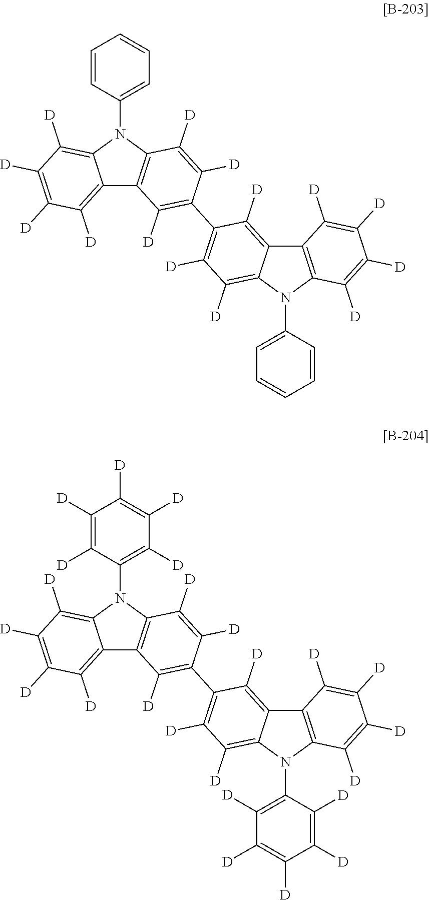 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1ccccc1 ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H]]

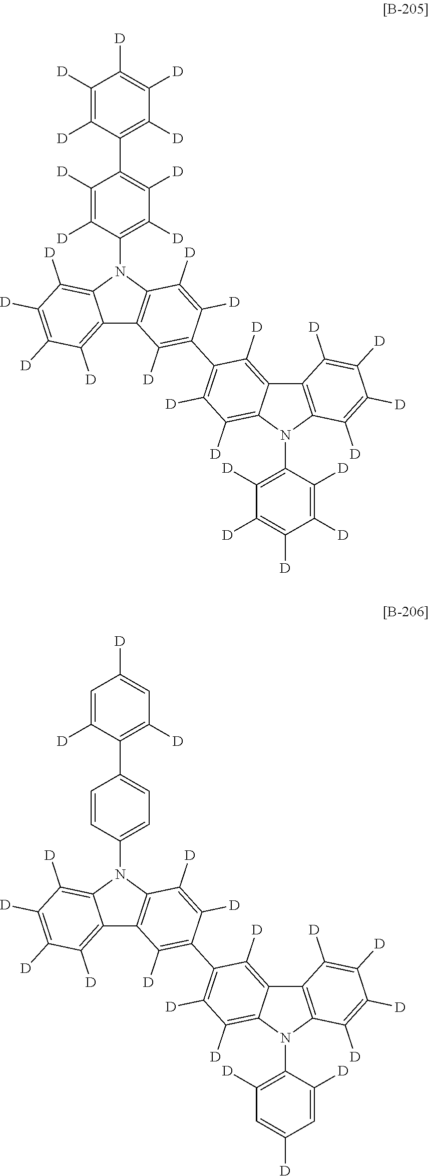 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c([2H])c2[2H])c([2H])c1[2H] ; [2H]c1cc([2H])c(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)c([2H])c1]

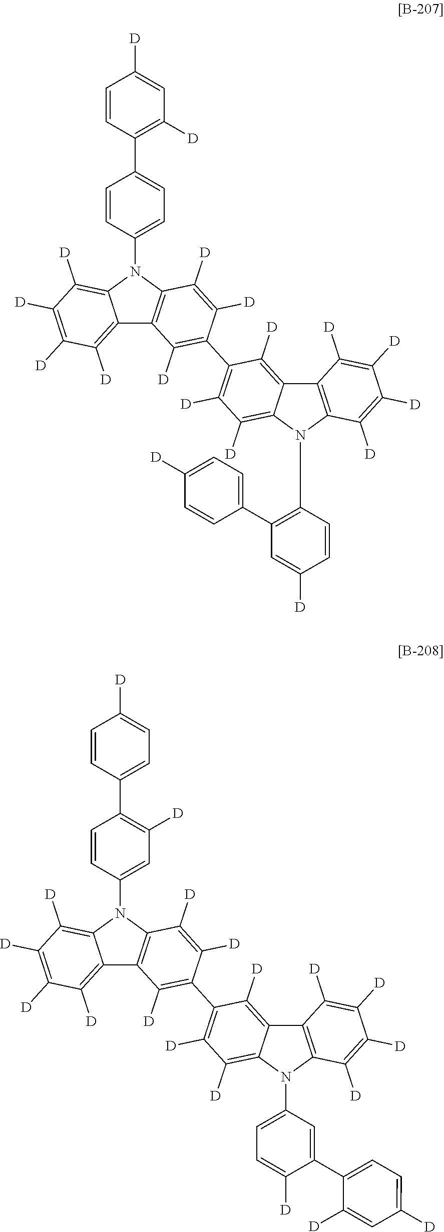 [SMILES: [2H]c1ccc(-c2cc([2H])ccc2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc(-c5ccc([2H])cc5[2H])cc4)c([2H])c([2H])c32)cc1 ; [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc([2H])c(-c6ccc([2H])cc6[2H])c5)c([2H])c([2H])c43)cc2[2H])cc1]

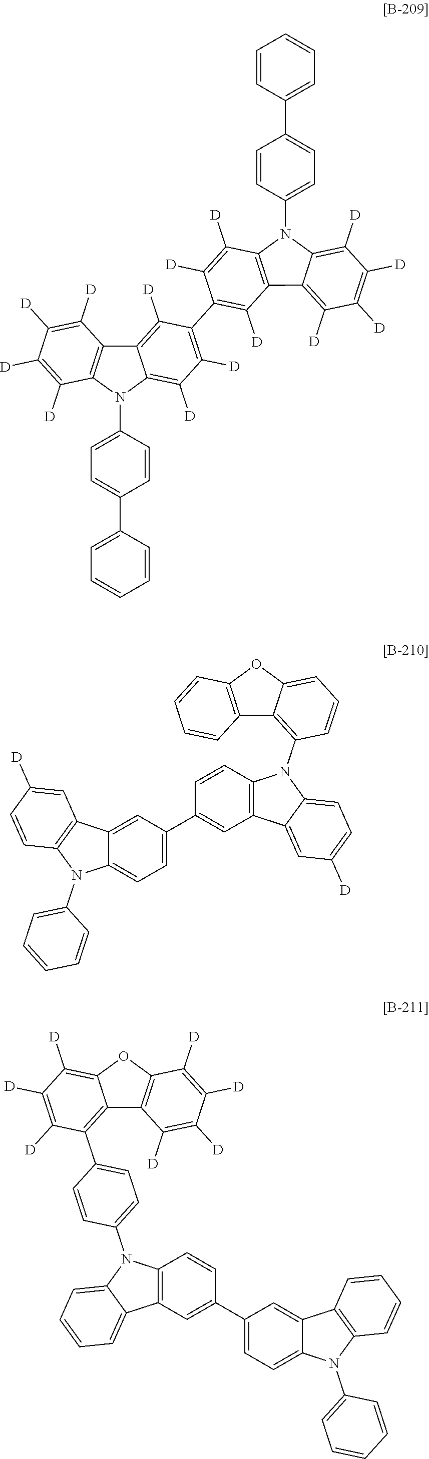 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1 ; [2H]c1ccc2c(c1)c1cc(-c3ccc4c(c3)c3cc([2H])ccc3n4-c3cccc4oc5ccccc5c34)ccc1n2-c1ccccc1 ; [2H]c1c([2H])c([2H])c2c(oc3c([2H])c([2H])c([2H])c(-c4ccc(-n5c6ccccc6c6cc(-c7ccc8c(c7)c7ccccc7n8-c7ccccc7)ccc65)cc4)c32)c1[2H]]

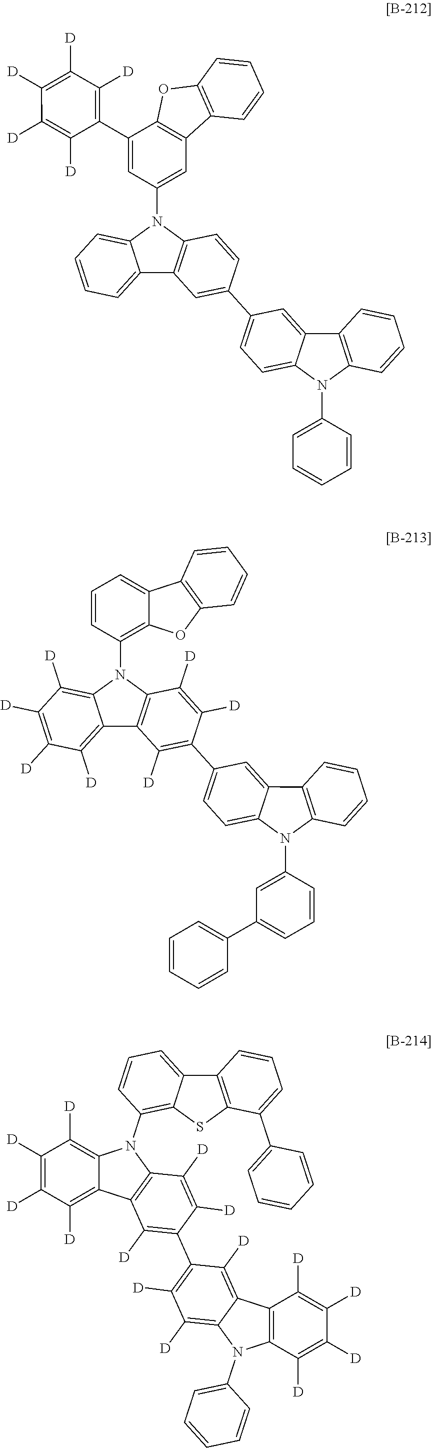 [SMILES: [2H]c1c([2H])c([2H])c(-c2cc(-n3c4ccccc4c4cc(-c5ccc6c(c5)c5ccccc5n6-c5ccccc5)ccc43)cc3c2oc2ccccc23)c([2H])c1[2H] ; [2H]c1c([2H])c([2H])c2c(c1[2H])c1c([2H])c(-c3ccc4c(c3)c3ccccc3n4-c3cccc(-c4ccccc4)c3)c([2H])c([2H])c1n2-c1cccc2c1oc1ccccc12 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3sc3c(-c5ccccc5)cccc34)c([2H])c([2H])c1n2-c1ccccc1]

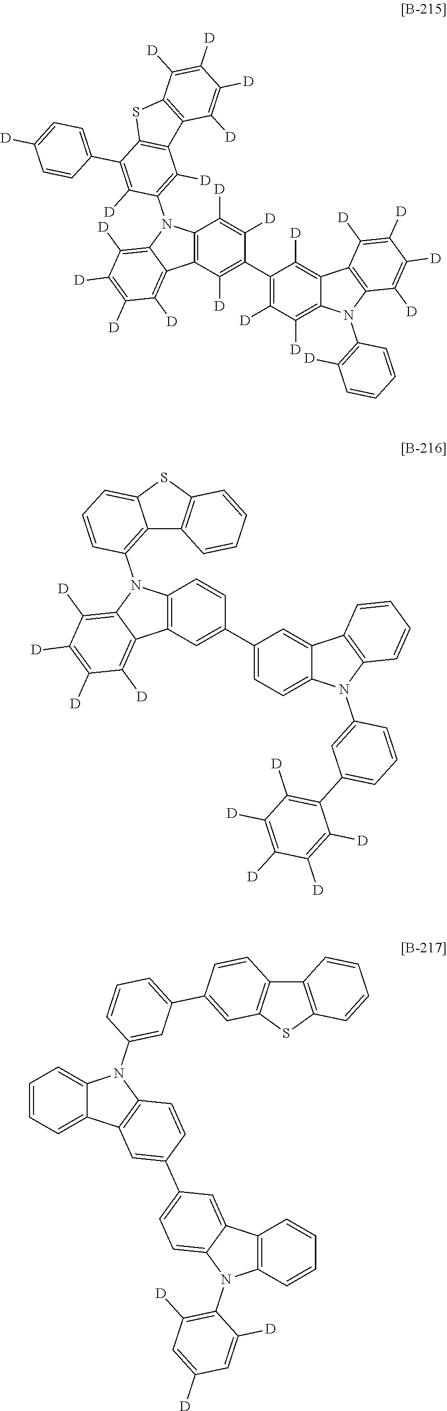 [SMILES: [2H]c1ccc(-c2c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccccc5[2H])c([2H])c([2H])c43)c([2H])c3c2sc2c([2H])c([2H])c([2H])c([2H])c23)cc1 ; [2H]c1c([2H])c([2H])c(-c2cccc(-n3c4ccccc4c4cc(-c5ccc6c(c5)c5c([2H])c([2H])c([2H])c([2H])c5n6-c5cccc6sc7ccccc7c56)ccc43)c2)c([2H])c1[2H] ; [2H]c1cc([2H])c(-n2c3ccccc3c3cc(-c4ccc5c(c4)c4ccccc4n5-c4cccc(-c5ccc6c(c5)sc5ccccc56)c4)ccc32)c([2H])c1]

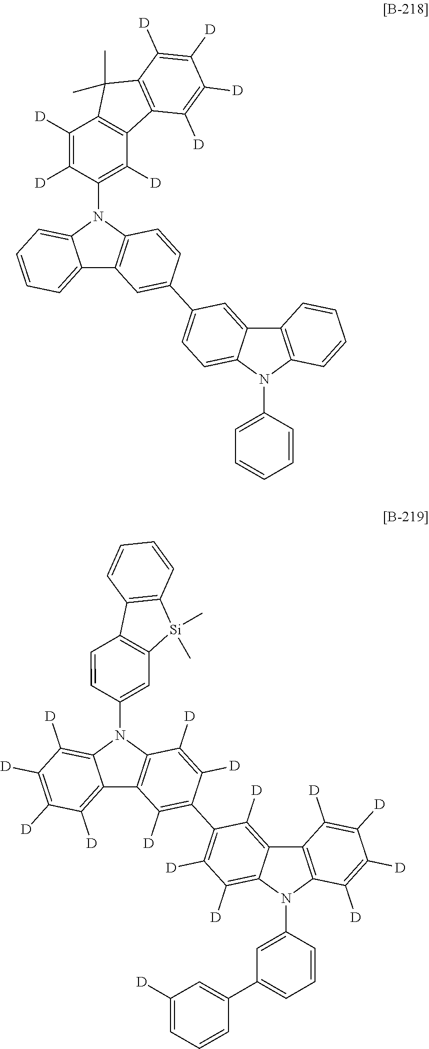 [SMILES: [2H]c1c([2H])c([2H])c2c(c1[2H])-c1c([2H])c(-n3c4ccccc4c4cc(-c5ccc6c(c5)c5ccccc5n6-c5ccccc5)ccc43)c([2H])c([2H])c1C2(C)C ; [2H]c1cccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)[Si](C)(C)c5ccccc5-6)c([2H])c([2H])c43)c2)c1]

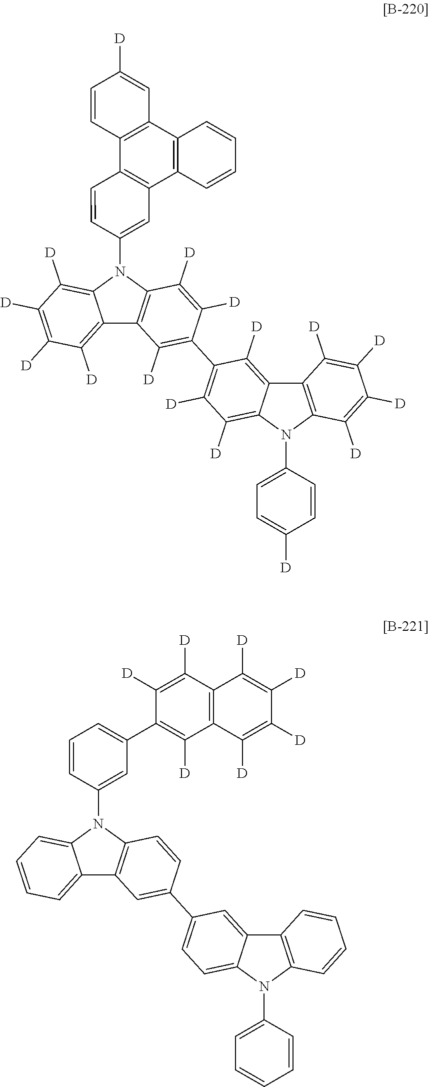 [SMILES: [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c6ccc([2H])cc6c6ccccc6c5c4)c([2H])c([2H])c32)cc1 ; [2H]c1c([2H])c([2H])c2c([2H])c(-c3cccc(-n4c5ccccc5c5cc(-c6ccc7c(c6)c6ccccc6n7-c6ccccc6)ccc54)c3)c([2H])c([2H])c2c1[2H]]

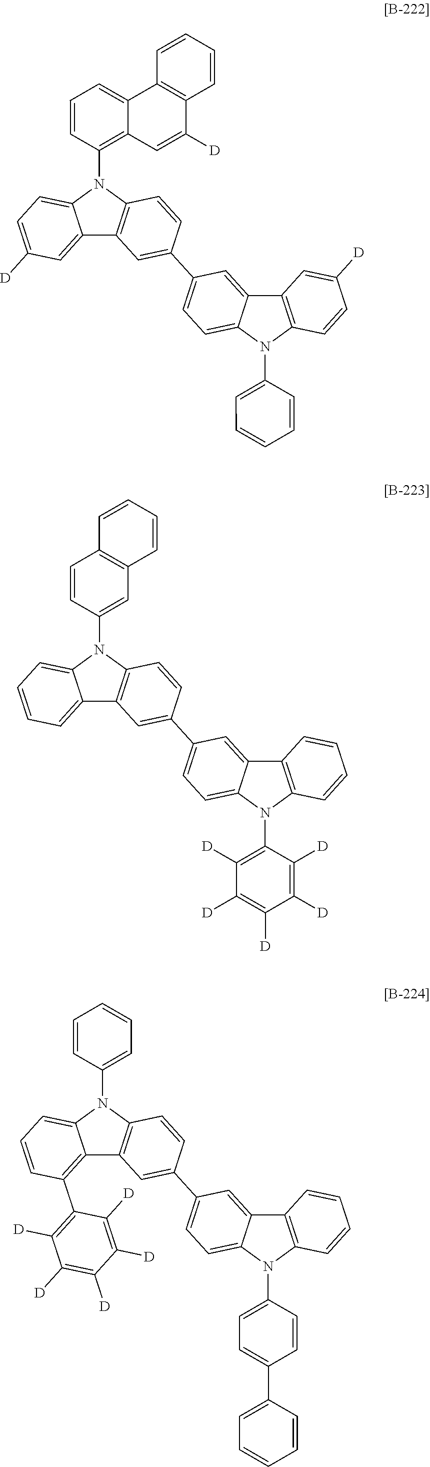 [SMILES: [2H]c1ccc2c(c1)c1cc(-c3ccc4c(c3)c3cc([2H])ccc3n4-c3cccc4c3cc([2H])c3ccccc34)ccc1n2-c1ccccc1 ; [2H]c1c([2H])c([2H])c(-n2c3ccccc3c3cc(-c4ccc5c(c4)c4ccccc4n5-c4ccc5ccccc5c4)ccc32)c([2H])c1[2H] ; [2H]c1c([2H])c([2H])c(-c2cccc3c2c2cc(-c4ccc5c(c4)c4ccccc4n5-c4ccc(-c5ccccc5)cc4)ccc2n3-c2ccccc2)c([2H])c1[2H]]

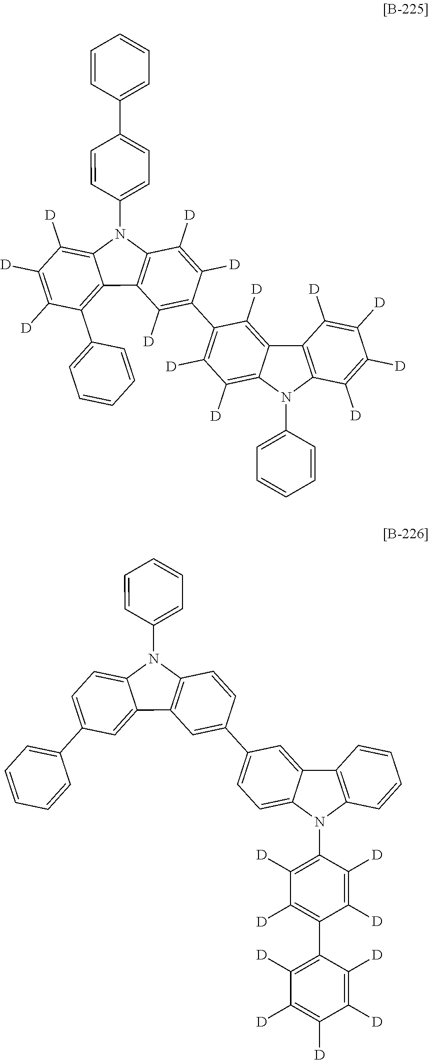 [SMILES: [2H]c1c([2H])c([2H])c2c(c1[2H])c1c([2H])c(-c3c([2H])c([2H])c4c(c3[2H])c3c(-c5ccccc5)c([2H])c([2H])c([2H])c3n4-c3ccc(-c4ccccc4)cc3)c([2H])c([2H])c1n2-c1ccccc1 ; [2H]c1c([2H])c([2H])c(-c2c([2H])c([2H])c(-n3c4ccccc4c4cc(-c5ccc6c(c5)c5cc(-c7ccccc7)ccc5n6-c5ccccc5)ccc43)c([2H])c2[2H])c([2H])c1[2H]]

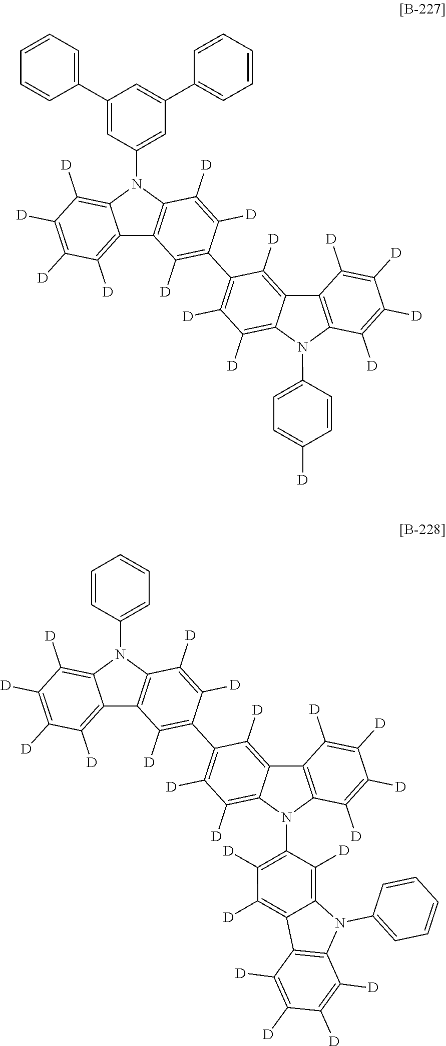 [SMILES: [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cc(-c5ccccc5)cc(-c5ccccc5)c4)c([2H])c([2H])c32)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c([2H])c5n(-c5ccccc5)c4c3[2H])c([2H])c([2H])c1n2-c1ccccc1]

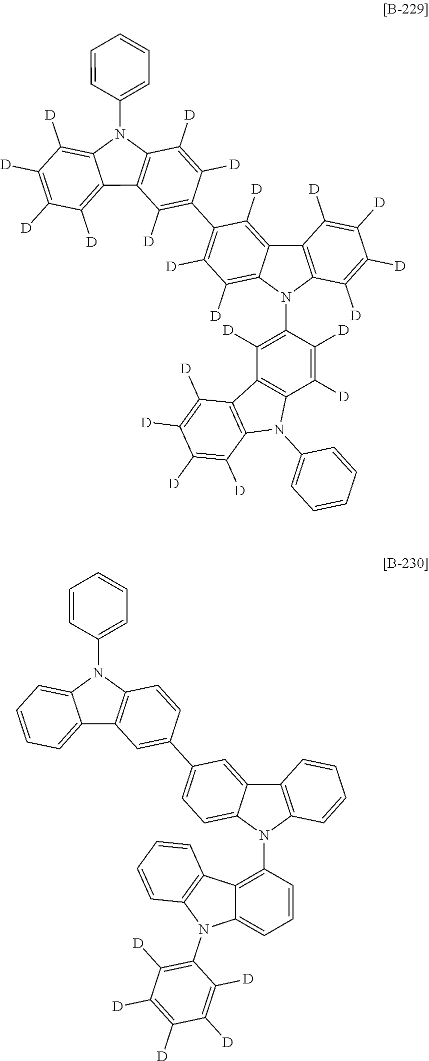 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3)c([2H])c([2H])c1n2-c1ccccc1 ; [2H]c1c([2H])c([2H])c(-n2c3ccccc3c3c(-n4c5ccccc5c5cc(-c6ccc7c(c6)c6ccccc6n7-c6ccccc6)ccc54)cccc32)c([2H])c1[2H]]

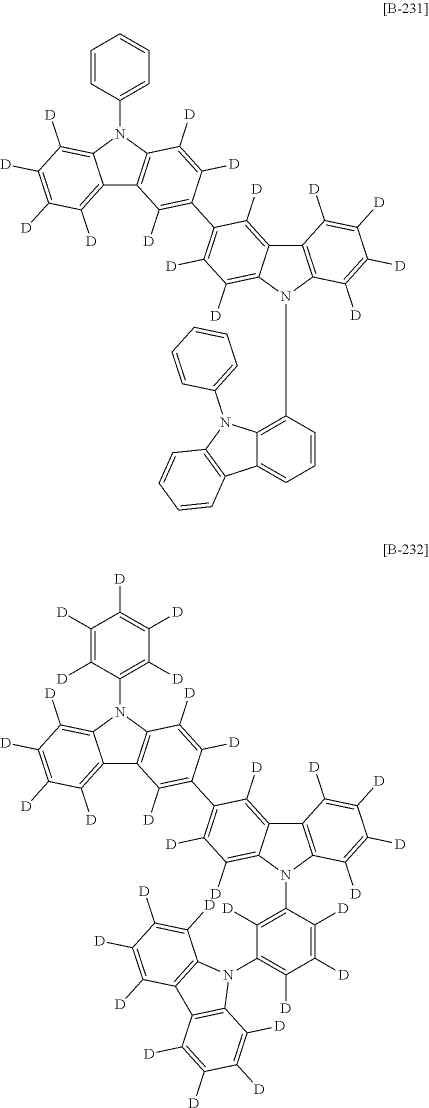 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c5ccccc5n(-c5ccccc5)c34)c([2H])c([2H])c1n2-c1ccccc1 ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c(-n5c6c([2H])c([2H])c([2H])c([2H])c6c6c([2H])c([2H])c([2H])c([2H])c65)c4[2H])c([2H])c([2H])c32)c([2H])c1[2H]]

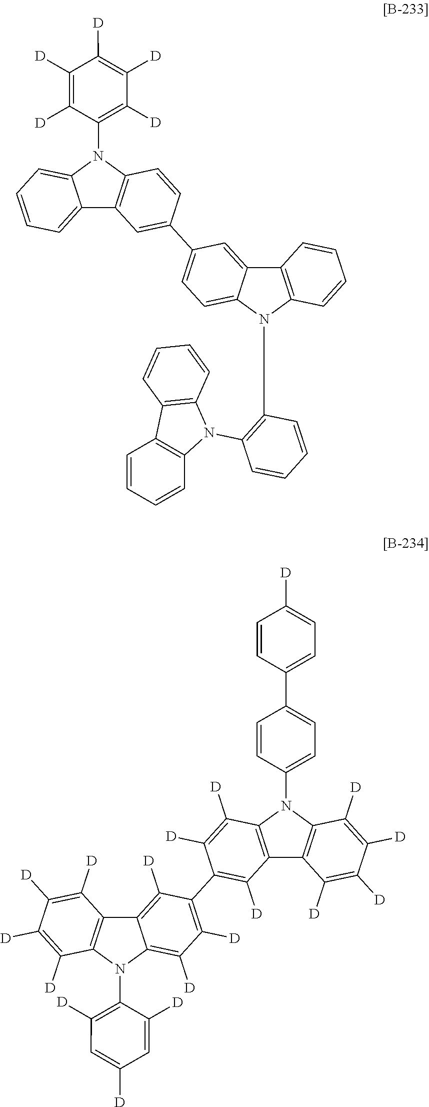 [SMILES: [2H]c1c([2H])c([2H])c(-n2c3ccccc3c3cc(-c4ccc5c(c4)c4ccccc4n5-c4ccccc4-n4c5ccccc5c5ccccc54)ccc32)c([2H])c1[2H] ; [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)cc1]

In an implementation, the second compound for the organic optoelectronic device may be, e.g., a compound of Group 3.

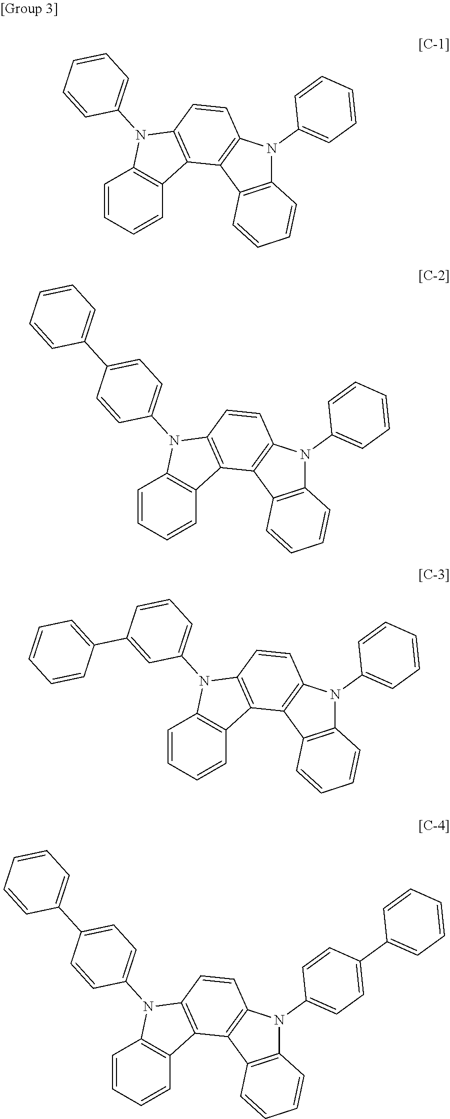 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccc(-c7ccccc7)cc6)c5ccc43)cc2)cc1]

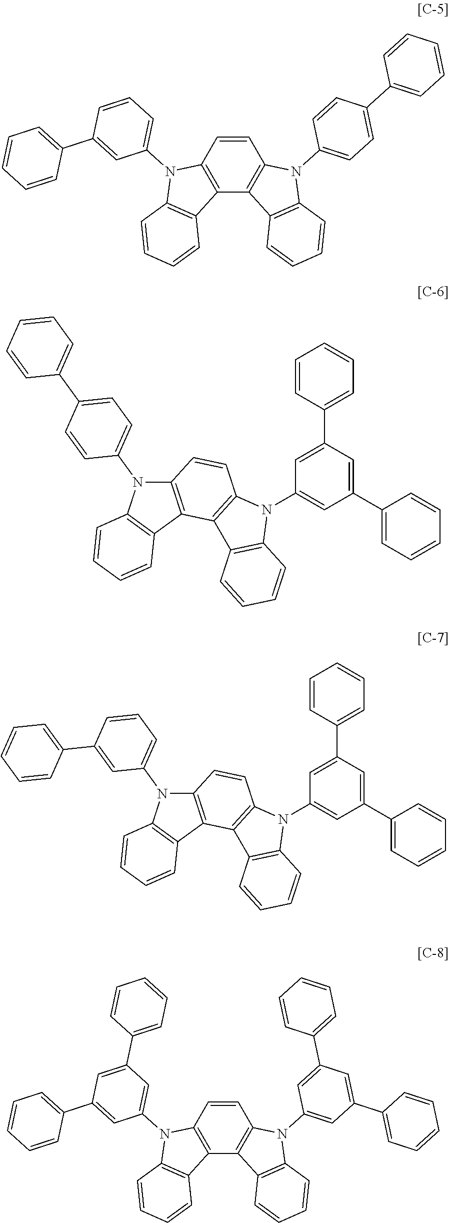 [SMILES: c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6cc(-c7ccccc7)cc(-c7ccccc7)c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1 ; c1ccc(-c2cc(-c3ccccc3)cc(-n3c4ccccc4c4c5c6ccccc6n(-c6cc(-c7ccccc7)cc(-c7ccccc7)c6)c5ccc43)c2)cc1]

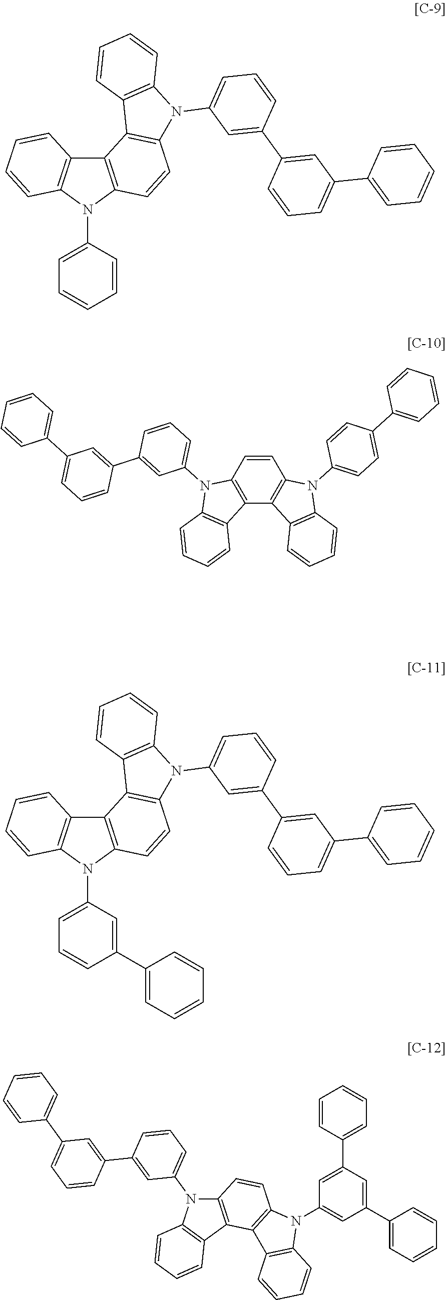 [SMILES: c1ccc(-c2cccc(-c3cccc(-n4c5ccccc5c5c6c7ccccc7n(-c7ccccc7)c6ccc54)c3)c2)cc1 ; c1ccc(-c2ccc(-n3c4ccccc4c4c5c6ccccc6n(-c6cccc(-c7cccc(-c8ccccc8)c7)c6)c5ccc43)cc2)cc1 ; c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2cccc(-c3cccc(-n4c5ccccc5c5c6c7ccccc7n(-c7cc(-c8ccccc8)cc(-c8ccccc8)c7)c6ccc54)c3)c2)cc1]

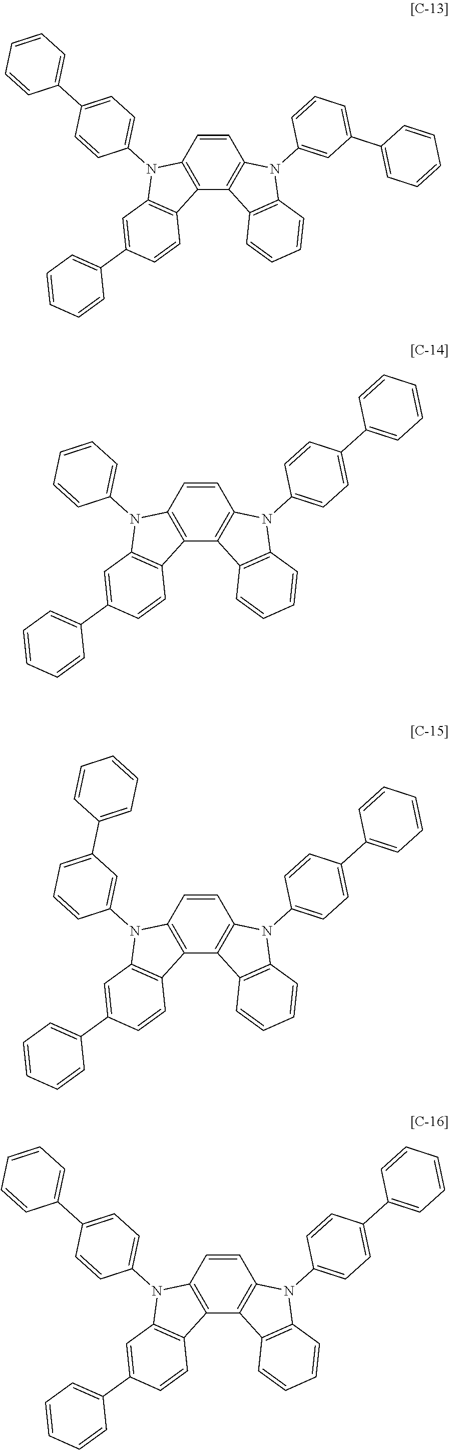 [SMILES: c1ccc(-c2ccc(-n3c4cc(-c5ccccc5)ccc4c4c5c6ccccc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccccc6)c5ccc43)cc2)cc1 ; c1ccc(-c2ccc(-n3c4ccccc4c4c5c6ccc(-c7ccccc7)cc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccc(-c7ccccc7)cc6)c5ccc43)cc2)cc1]

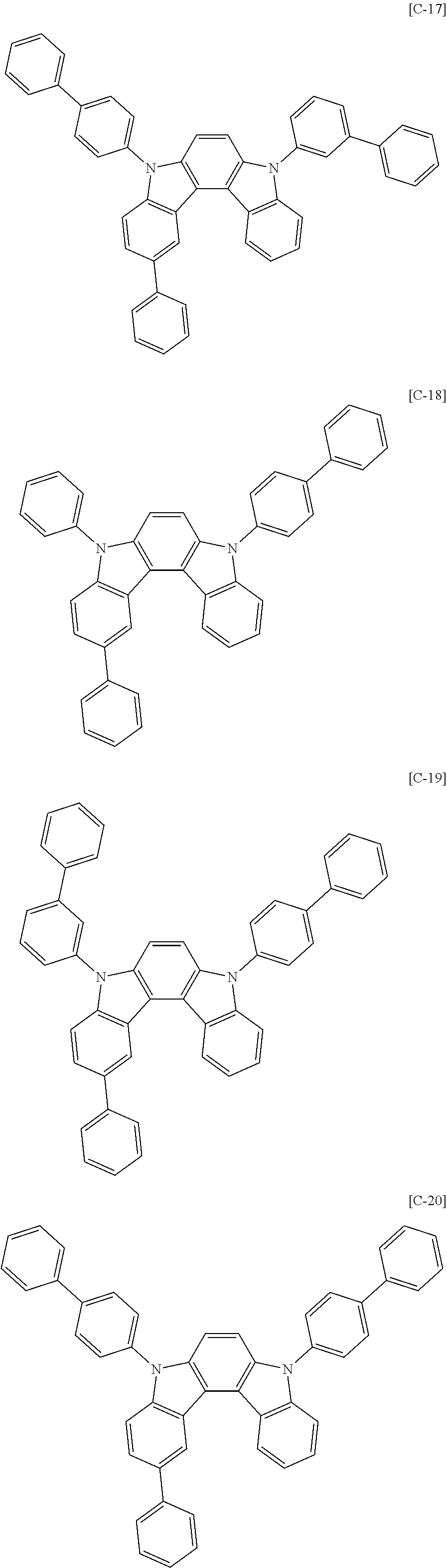 [SMILES: c1ccc(-c2ccc(-n3c4ccc(-c5ccccc5)cc4c4c5c6ccccc6n(-c6cccc(-c7ccccc7)c6)c5ccc43)cc2)cc1 ; c1ccc(-c2ccc(-n3c4ccccc4c4c5c6cc(-c7ccccc7)ccc6n(-c6ccccc6)c5ccc43)cc2)cc1 ; c1ccc(-c2ccc(-n3c4ccccc4c4c5c6cc(-c7ccccc7)ccc6n(-c6cccc(-c7ccccc7)c6)c5ccc43)cc2)cc1 ; c1ccc(-c2ccc(-n3c4ccccc4c4c5c6cc(-c7ccccc7)ccc6n(-c6ccc(-c7ccccc7)cc6)c5ccc43)cc2)cc1]

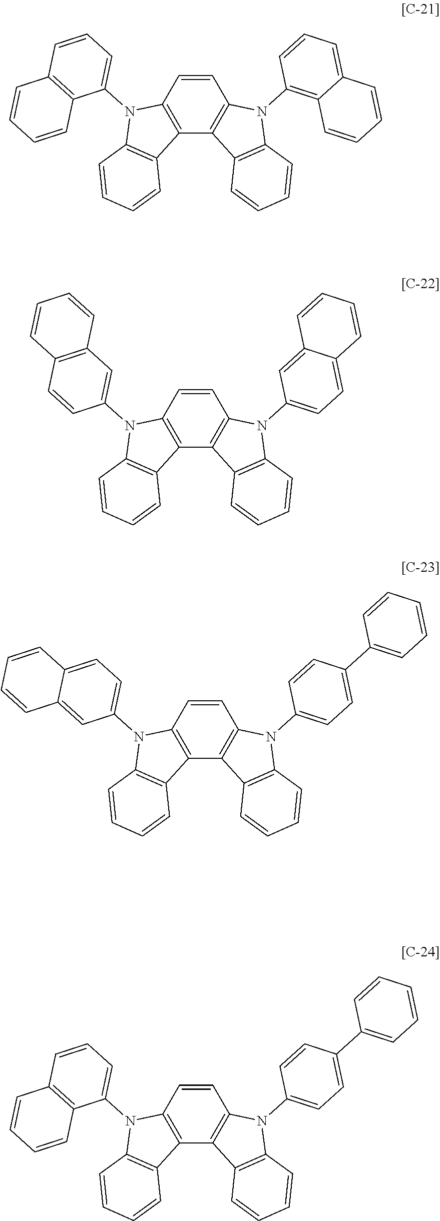 [SMILES: c1ccc2c(-n3c4ccccc4c4c5c6ccccc6n(-c6cccc7ccccc67)c5ccc43)cccc2c1 ; c1ccc2cc(-n3c4ccccc4c4c5c6ccccc6n(-c6ccc7ccccc7c6)c5ccc43)ccc2c1 ; c1ccc(-c2ccc(-n3c4ccccc4c4c5c6ccccc6n(-c6ccc7ccccc7c6)c5ccc43)cc2)cc1 ; c1ccc(-c2ccc(-n3c4ccccc4c4c5c6ccccc6n(-c6cccc7ccccc67)c5ccc43)cc2)cc1]

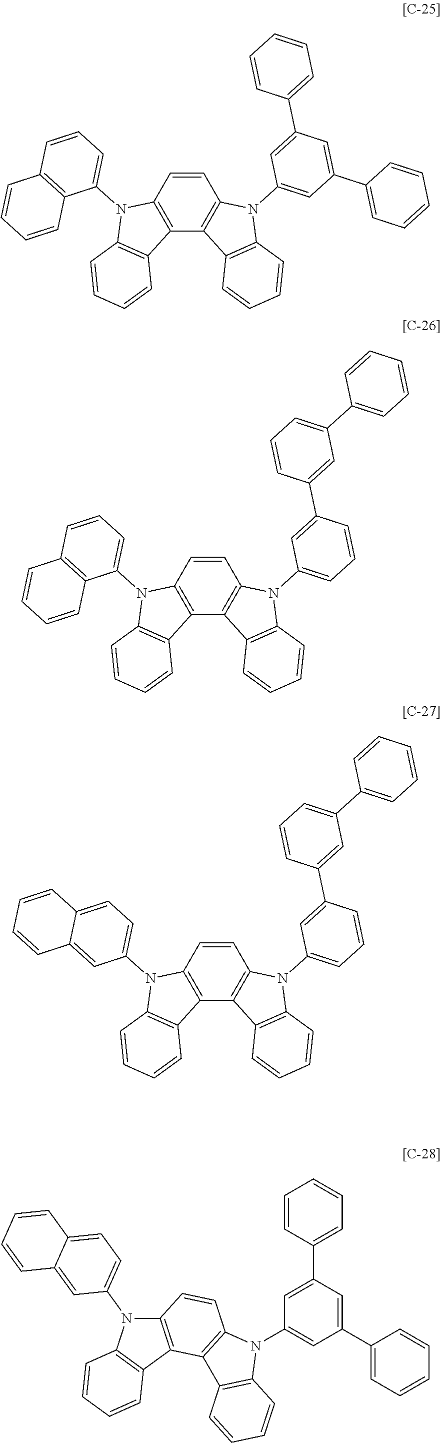 [SMILES: c1ccc(-c2cc(-c3ccccc3)cc(-n3c4ccccc4c4c5c6ccccc6n(-c6cccc7ccccc67)c5ccc43)c2)cc1 ; c1ccc(-c2cccc(-c3cccc(-n4c5ccccc5c5c6c7ccccc7n(-c7cccc8ccccc78)c6ccc54)c3)c2)cc1 ; c1ccc(-c2cccc(-c3cccc(-n4c5ccccc5c5c6c7ccccc7n(-c7ccc8ccccc8c7)c6ccc54)c3)c2)cc1 ; c1ccc(-c2cc(-c3ccccc3)cc(-n3c4ccccc4c4c5c6ccccc6n(-c6ccc7ccccc7c6)c5ccc43)c2)cc1]

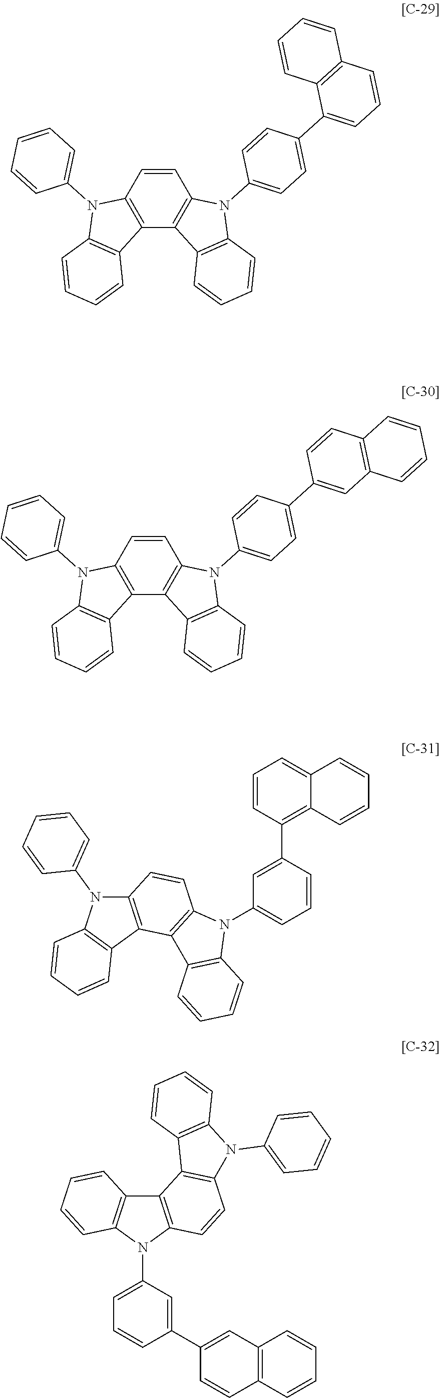 [SMILES: c1ccc(-n2c3ccccc3c3c4c5ccccc5n(-c5ccc(-c6cccc7ccccc67)cc5)c4ccc32)cc1 ; c1ccc(-n2c3ccccc3c3c4c5ccccc5n(-c5ccc(-c6ccc7ccccc7c6)cc5)c4ccc32)cc1 ; c1ccc(-n2c3ccccc3c3c4c5ccccc5n(-c5cccc(-c6cccc7ccccc67)c5)c4ccc32)cc1 ; c1ccc(-n2c3ccccc3c3c4c5ccccc5n(-c5cccc(-c6ccc7ccccc7c6)c5)c4ccc32)cc1]

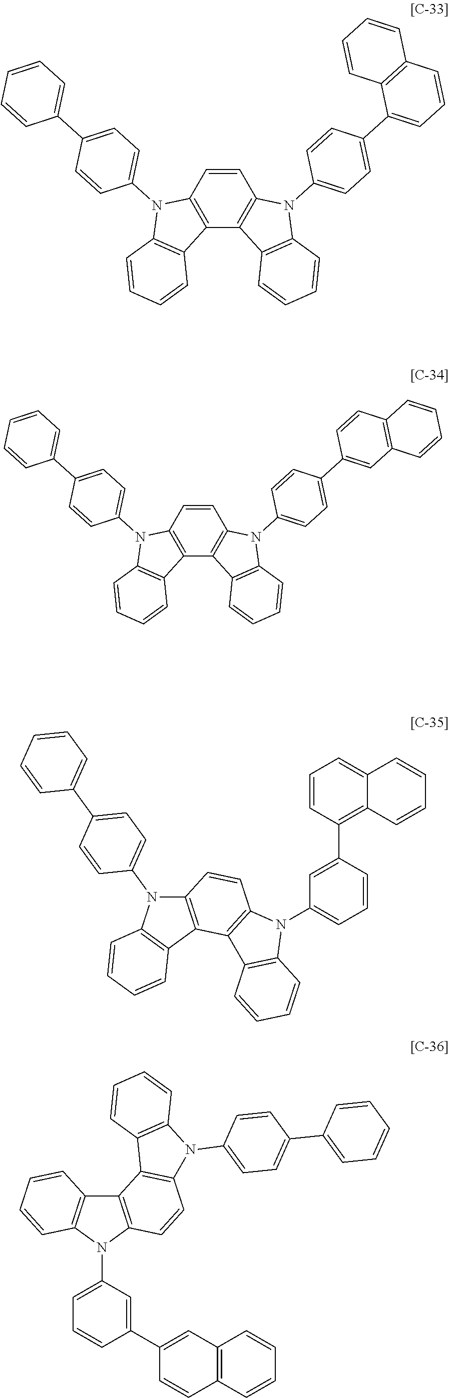 [SMILES: c1ccc(-c2ccc(-n3c4ccccc4c4c5c6ccccc6n(-c6ccc(-c7cccc8ccccc78)cc6)c5ccc43)cc2)cc1 ; c1ccc(-c2ccc(-n3c4ccccc4c4c5c6ccccc6n(-c6ccc(-c7ccc8ccccc8c7)cc6)c5ccc43)cc2)cc1 ; c1ccc(-c2ccc(-n3c4ccccc4c4c5c6ccccc6n(-c6cccc(-c7cccc8ccccc78)c6)c5ccc43)cc2)cc1 ; c1ccc(-c2ccc(-n3c4ccccc4c4c5c6ccccc6n(-c6cccc(-c7ccc8ccccc8c7)c6)c5ccc43)cc2)cc1]

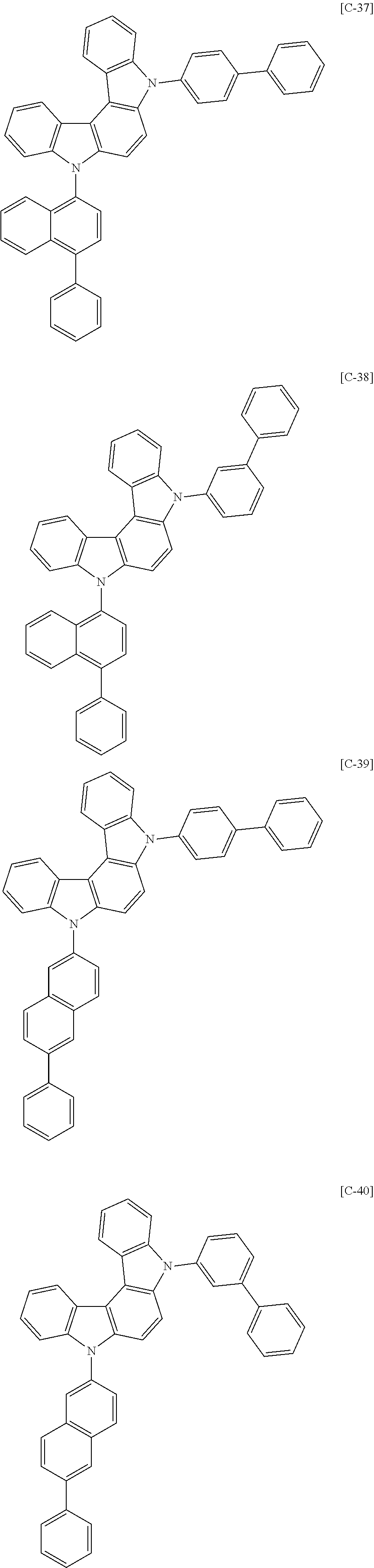

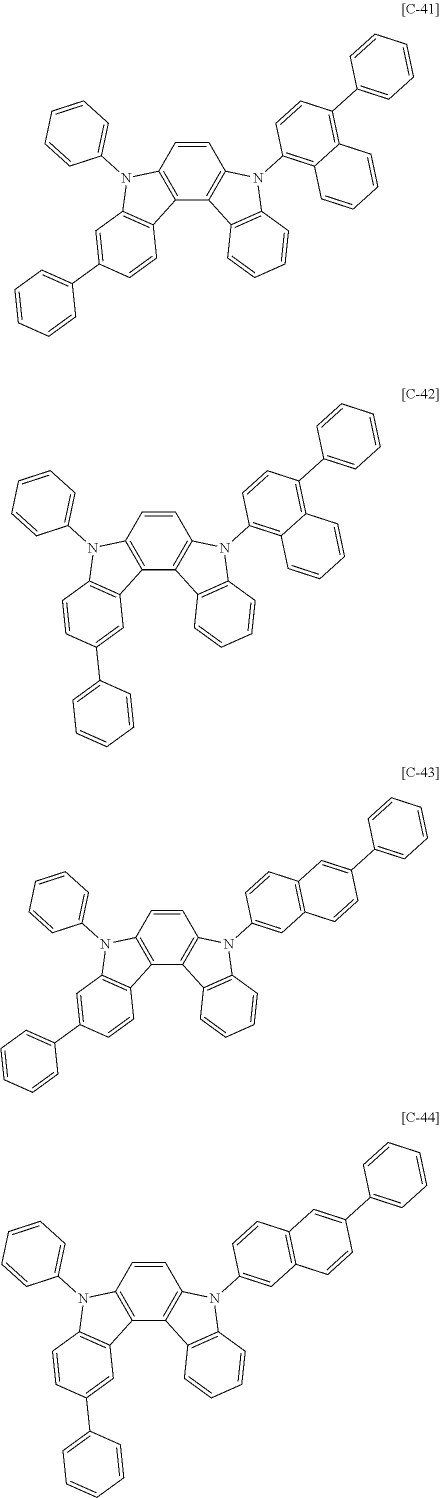 [SMILES: c1ccc(-c2ccc3c4c5c6ccccc6n(-c6ccc(-c7ccccc7)c7ccccc67)c5ccc4n(-c4ccccc4)c3c2)cc1 ; c1ccc(-c2ccc3c(c2)c2c4c5ccccc5n(-c5ccc(-c6ccccc6)c6ccccc56)c4ccc2n3-c2ccccc2)cc1 ; c1ccc(-c2ccc3cc(-n4c5ccccc5c5c6c7ccc(-c8ccccc8)cc7n(-c7ccccc7)c6ccc54)ccc3c2)cc1 ; c1ccc(-c2ccc3cc(-n4c5ccccc5c5c6c7cc(-c8ccccc8)ccc7n(-c7ccccc7)c6ccc54)ccc3c2)cc1]

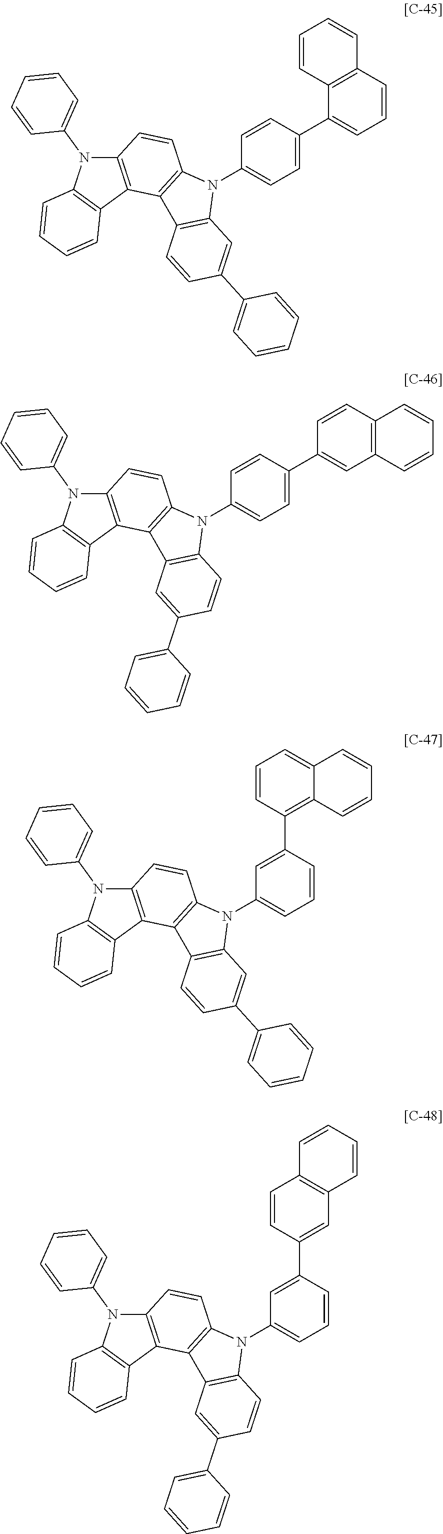 [SMILES: c1ccc(-c2ccc3c4c5c6ccccc6n(-c6ccccc6)c5ccc4n(-c4ccc(-c5cccc6ccccc56)cc4)c3c2)cc1 ; c1ccc(-c2ccc3c(c2)c2c4c5ccccc5n(-c5ccccc5)c4ccc2n3-c2ccc(-c3ccc4ccccc4c3)cc2)cc1 ; c1ccc(-c2ccc3c4c5c6ccccc6n(-c6ccccc6)c5ccc4n(-c4cccc(-c5cccc6ccccc56)c4)c3c2)cc1 ; c1ccc(-c2ccc3c(c2)c2c4c5ccccc5n(-c5ccccc5)c4ccc2n3-c2cccc(-c3ccc4ccccc4c3)c2)cc1]

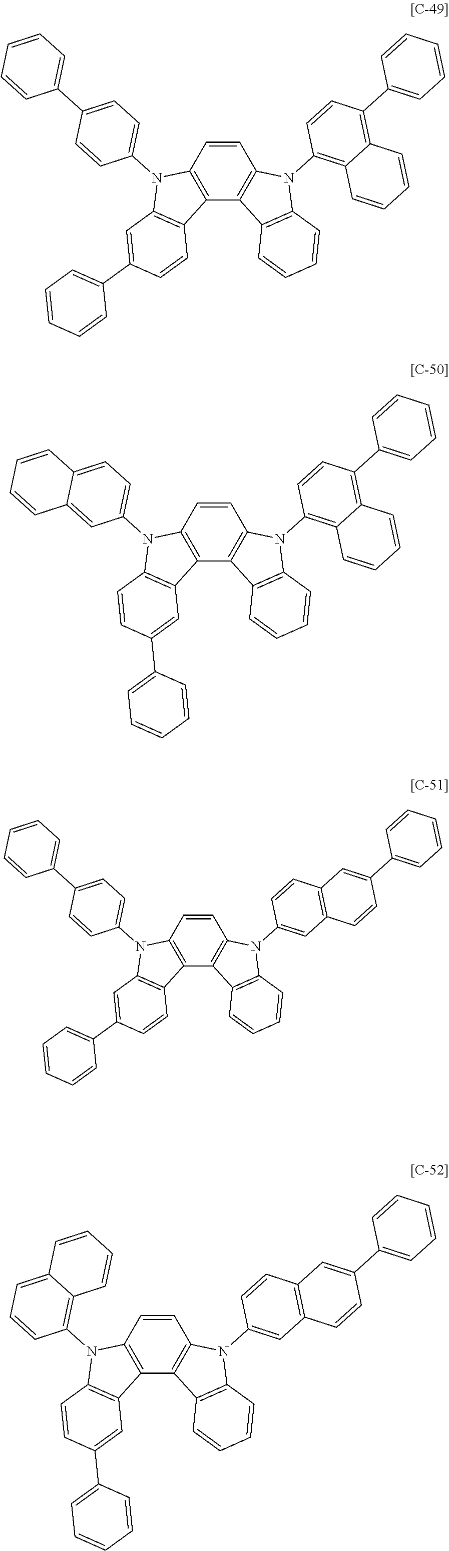 [SMILES: c1ccc(-c2ccc(-n3c4cc(-c5ccccc5)ccc4c4c5c6ccccc6n(-c6ccc(-c7ccccc7)c7ccccc67)c5ccc43)cc2)cc1 ; c1ccc(-c2ccc3c(c2)c2c4c5ccccc5n(-c5ccc(-c6ccccc6)c6ccccc56)c4ccc2n3-c2ccc3ccccc3c2)cc1 ; c1ccc(-c2ccc(-n3c4cc(-c5ccccc5)ccc4c4c5c6ccccc6n(-c6ccc7cc(-c8ccccc8)ccc7c6)c5ccc43)cc2)cc1 ; c1ccc(-c2ccc3cc(-n4c5ccccc5c5c6c7cc(-c8ccccc8)ccc7n(-c7cccc8ccccc78)c6ccc54)ccc3c2)cc1]

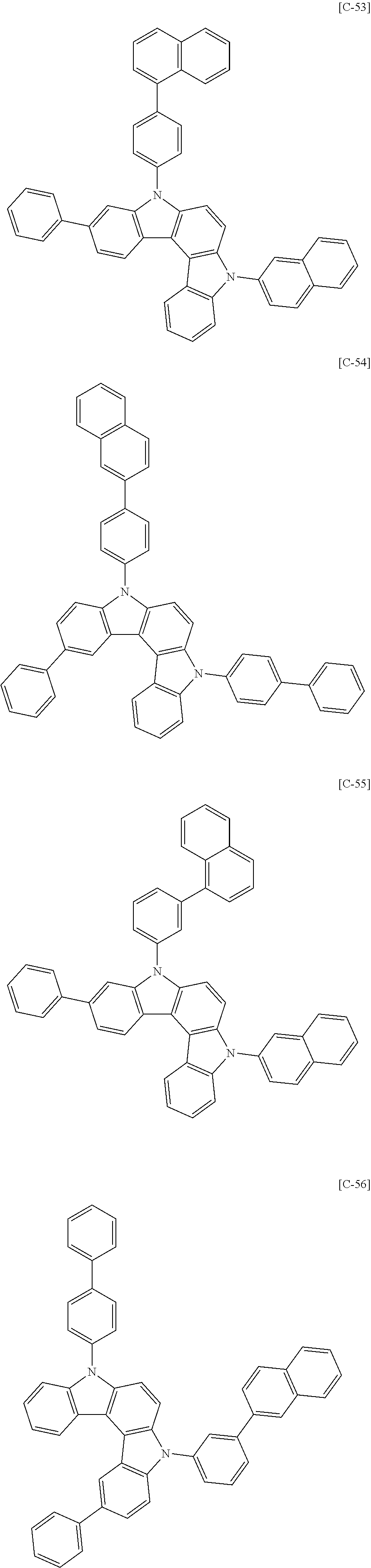 [SMILES: c1ccc(-c2ccc3c4c5c6ccccc6n(-c6ccc7ccccc7c6)c5ccc4n(-c4ccc(-c5cccc6ccccc56)cc4)c3c2)cc1 ; c1ccc(-c2ccc(-n3c4ccccc4c4c5c6cc(-c7ccccc7)ccc6n(-c6ccc(-c7ccc8ccccc8c7)cc6)c5ccc43)cc2)cc1 ; c1ccc(-c2ccc3c4c5c6ccccc6n(-c6ccc7ccccc7c6)c5ccc4n(-c4cccc(-c5cccc6ccccc56)c4)c3c2)cc1 ; c1ccc(-c2ccc(-n3c4ccccc4c4c5c6cc(-c7ccccc7)ccc6n(-c6cccc(-c7ccc8ccccc8c7)c6)c5ccc43)cc2)cc1]

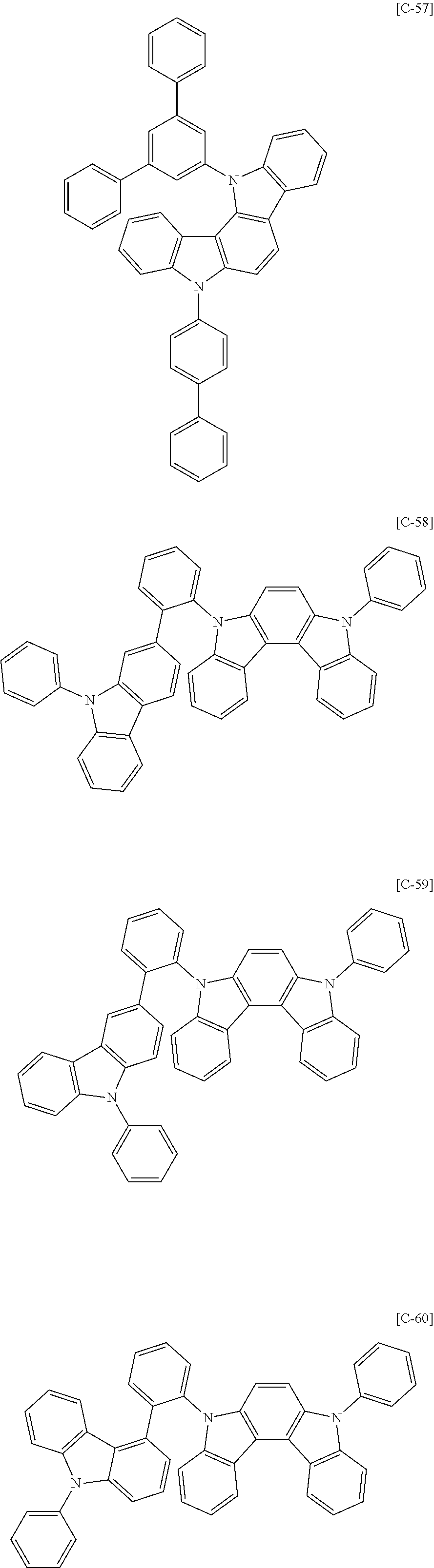 [SMILES: c1ccc(-c2ccc(-n3c4ccccc4c4c3ccc3c5ccccc5n(-c5cc(-c6ccccc6)cc(-c6ccccc6)c5)c34)cc2)cc1 ; c1ccc(-n2c3ccccc3c3ccc(-c4ccccc4-n4c5ccccc5c5c6c7ccccc7n(-c7ccccc7)c6ccc54)cc32)cc1 ; c1ccc(-n2c3ccccc3c3cc(-c4ccccc4-n4c5ccccc5c5c6c7ccccc7n(-c7ccccc7)c6ccc54)ccc32)cc1 ; c1ccc(-n2c3ccccc3c3c(-c4ccccc4-n4c5ccccc5c5c6c7ccccc7n(-c7ccccc7)c6ccc54)cccc32)cc1]

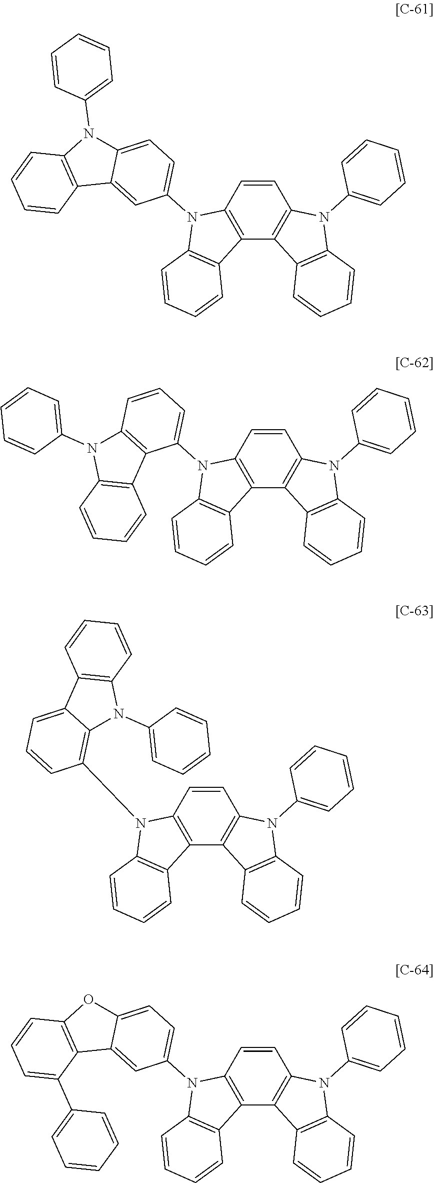 [SMILES: c1ccc(-n2c3ccccc3c3cc(-n4c5ccccc5c5c6c7ccccc7n(-c7ccccc7)c6ccc54)ccc32)cc1 ; c1ccc(-n2c3ccccc3c3c(-n4c5ccccc5c5c6c7ccccc7n(-c7ccccc7)c6ccc54)cccc32)cc1 ; c1ccc(-n2c3ccccc3c3c4c5ccccc5n(-c5cccc6c7ccccc7n(-c7ccccc7)c56)c4ccc32)cc1 ; c1ccc(-c2cccc3oc4ccc(-n5c6ccccc6c6c7c8ccccc8n(-c8ccccc8)c7ccc65)cc4c23)cc1]

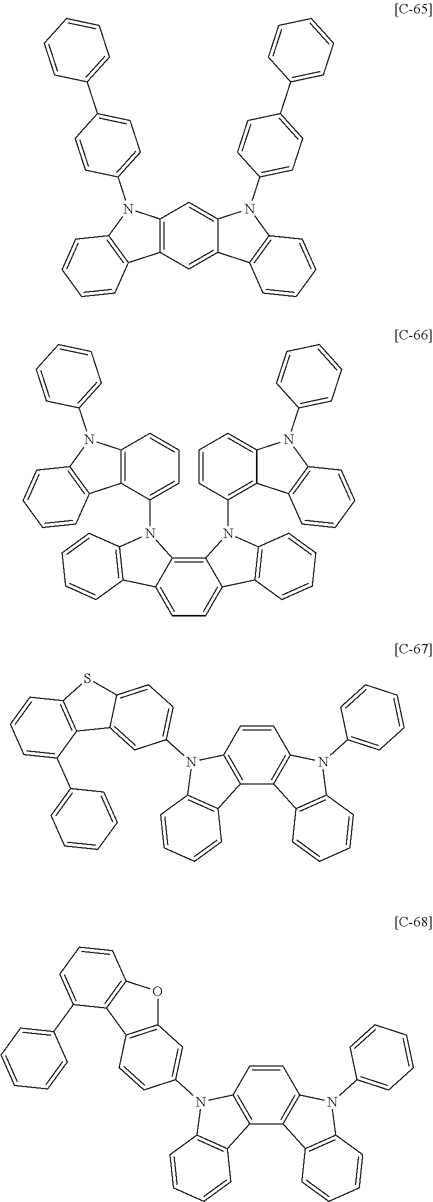 [SMILES: c1ccc(-c2ccc(-n3c4ccccc4c4cc5c6ccccc6n(-c6ccc(-c7ccccc7)cc6)c5cc43)cc2)cc1 ; c1ccc(-n2c3ccccc3c3c(-n4c5ccccc5c5ccc6c7ccccc7n(-c7cccc8c7c7ccccc7n8-c7ccccc7)c6c54)cccc32)cc1 ; c1ccc(-c2cccc3sc4ccc(-n5c6ccccc6c6c7c8ccccc8n(-c8ccccc8)c7ccc65)cc4c23)cc1 ; c1ccc(-c2cccc3oc4cc(-n5c6ccccc6c6c7c8ccccc8n(-c8ccccc8)c7ccc65)ccc4c23)cc1]

Examples of Compound C-1 to Compound C-68 in which at least one hydrogen is replaced with deuterium are shown in Compound C-59 to Compound C-83, exemplified below

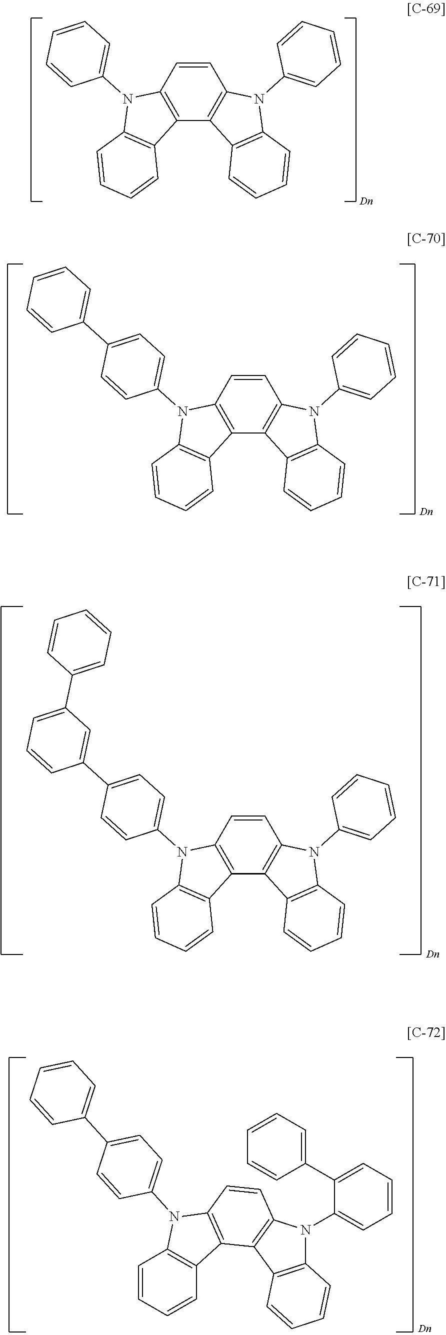

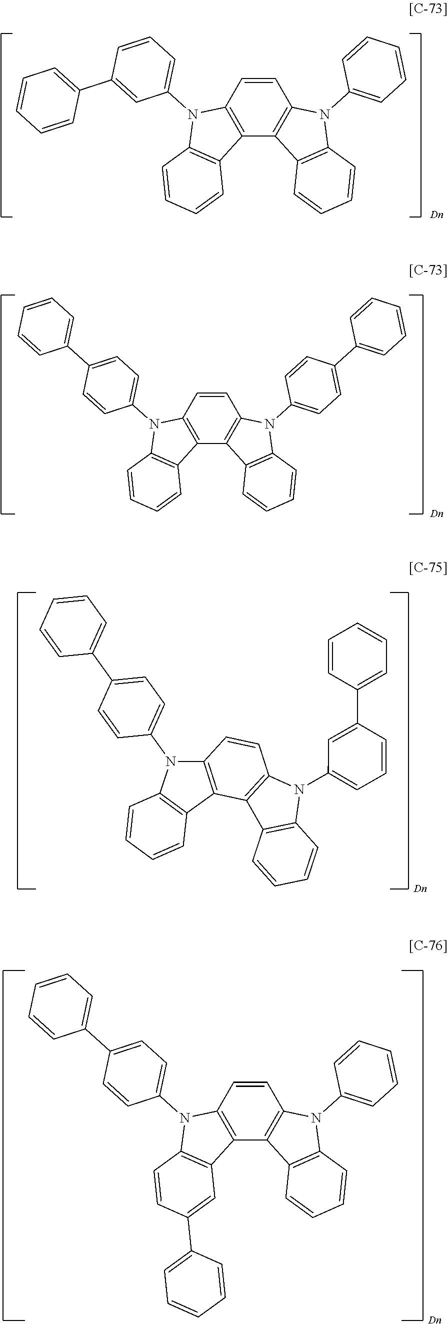

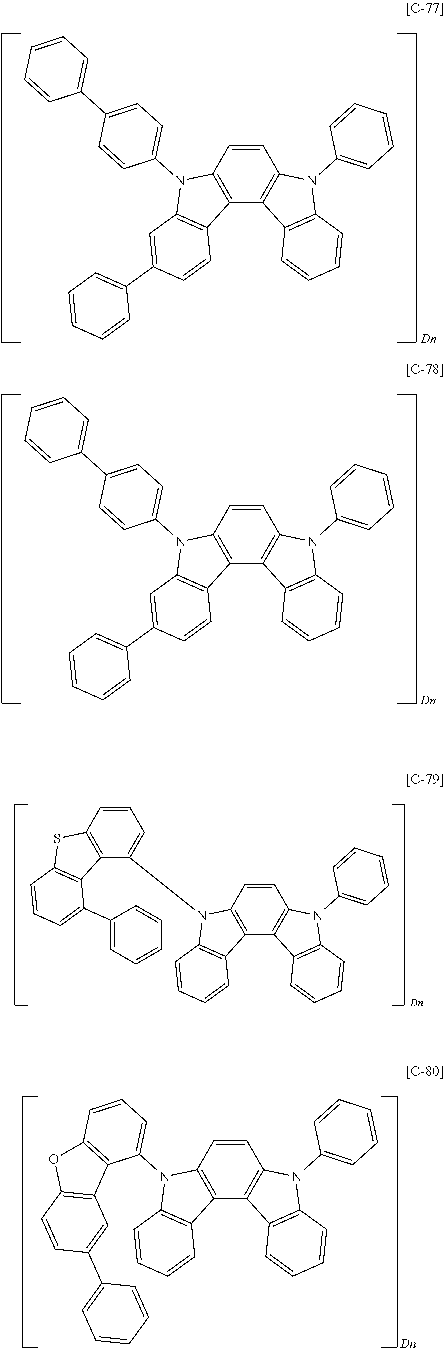

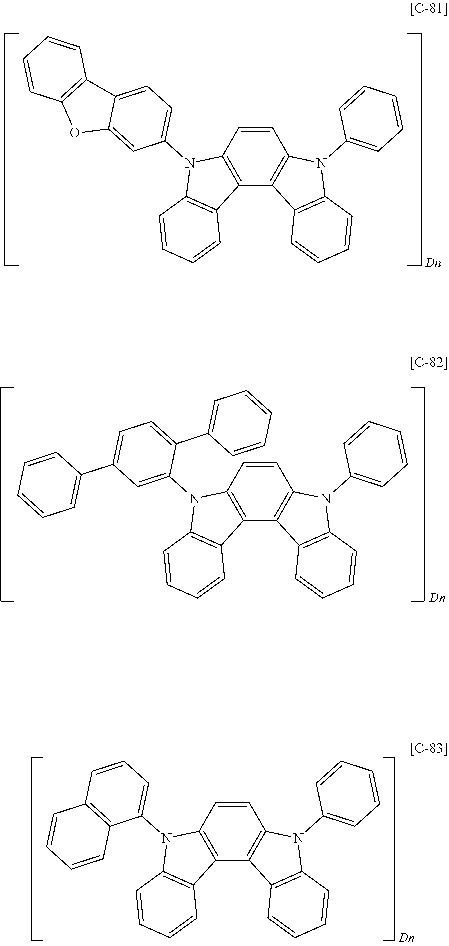

(Dn refers to the number of deuterium atoms substituted, and n is an integer between 1 and the maximum number of substitutions possible)

The position of deuterium substitution and the substitution degree of deuterium substitution may include all changeable ranges within the range of Compound C-1 to Compound C-83.

Specific structures of Compounds C-69 to C-83 are presented in Compound C-84 to Compound C-113 below.

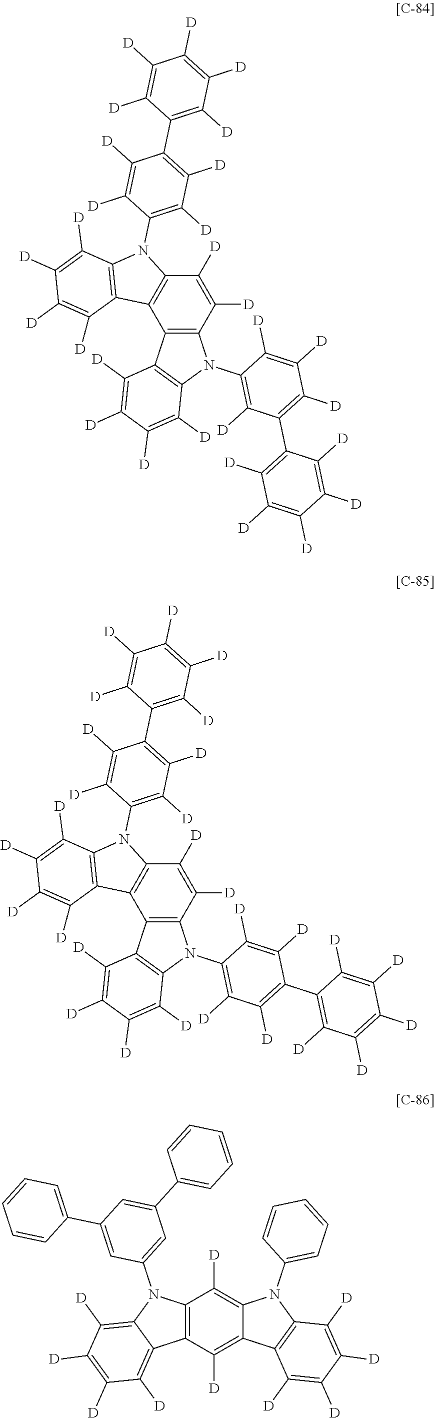

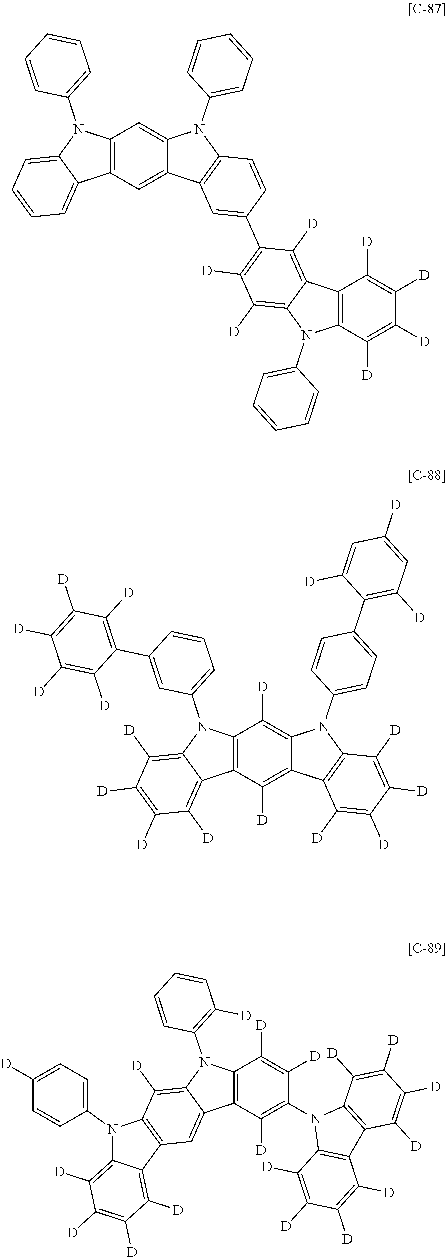 [SMILES: [2H]c1c([2H])c([2H])c2c(c1[2H])c1c([2H])c(-c3ccc4c(c3)c3cc5c6ccccc6n(-c6ccccc6)c5cc3n4-c3ccccc3)c([2H])c([2H])c1n2-c1ccccc1 ; [2H]c1cc([2H])c(-c2ccc(-n3c4c([2H])c([2H])c([2H])c([2H])c4c4c([2H])c5c6c([2H])c([2H])c([2H])c([2H])c6n(-c6cccc(-c7c([2H])c([2H])c([2H])c([2H])c7[2H])c6)c5c([2H])c43)cc2)c([2H])c1 ; [2H]c1ccc(-n2c3c([2H])c4c(cc3c3c([2H])c([2H])c([2H])c([2H])c32)c2c([2H])c(-n3c5c([2H])c([2H])c([2H])c([2H])c5c5c([2H])c([2H])c([2H])c([2H])c53)c([2H])c([2H])c2n4-c2ccccc2[2H])cc1]

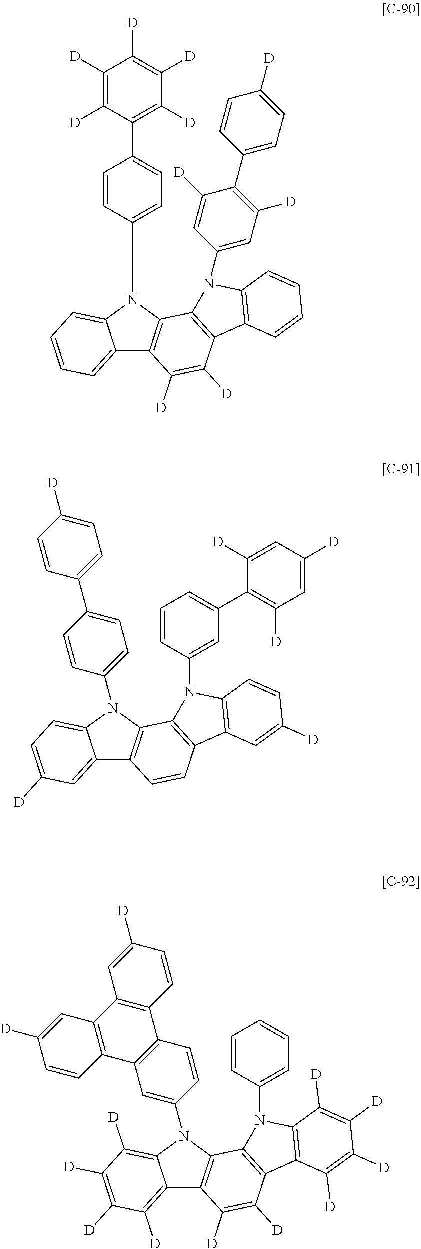 [SMILES: [2H]c1ccc(-c2c([2H])cc(-n3c4ccccc4c4c([2H])c([2H])c5c6ccccc6n(-c6ccc(-c7c([2H])c([2H])c([2H])c([2H])c7[2H])cc6)c5c43)cc2[2H])cc1 ; [2H]c1ccc(-c2ccc(-n3c4ccc([2H])cc4c4ccc5c6cc([2H])ccc6n(-c6cccc(-c7c([2H])cc([2H])cc7[2H])c6)c5c43)cc2)cc1 ; [2H]c1ccc2c3ccc(-n4c5c([2H])c([2H])c([2H])c([2H])c5c5c([2H])c([2H])c6c7c([2H])c([2H])c([2H])c([2H])c7n(-c7ccccc7)c6c54)cc3c3ccc([2H])cc3c2c1]

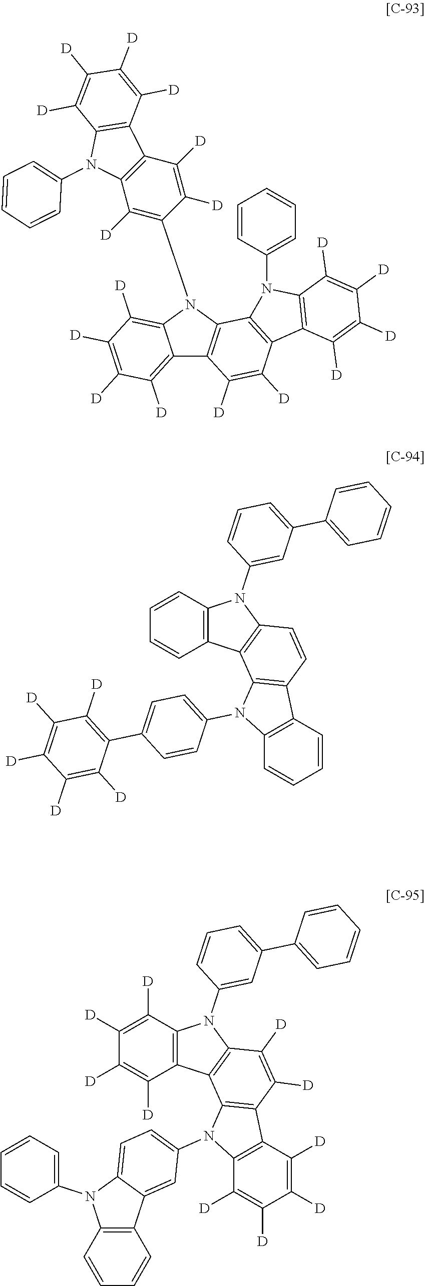 [SMILES: [2H]c1c([2H])c([2H])c2c(c1[2H])c1c([2H])c([2H])c(-n3c4c([2H])c([2H])c([2H])c([2H])c4c4c([2H])c([2H])c5c6c([2H])c([2H])c([2H])c([2H])c6n(-c6ccccc6)c5c43)c([2H])c1n2-c1ccccc1 ; [2H]c1c([2H])c([2H])c(-c2ccc(-n3c4ccccc4c4ccc5c(c6ccccc6n5-c5cccc(-c6ccccc6)c5)c43)cc2)c([2H])c1[2H] ; [2H]c1c([2H])c([2H])c2c(c1[2H])c1c(c([2H])c([2H])c3c4c([2H])c([2H])c([2H])c([2H])c4n(-c4ccc5c(c4)c4ccccc4n5-c4ccccc4)c31)n2-c1cccc(-c2ccccc2)c1]

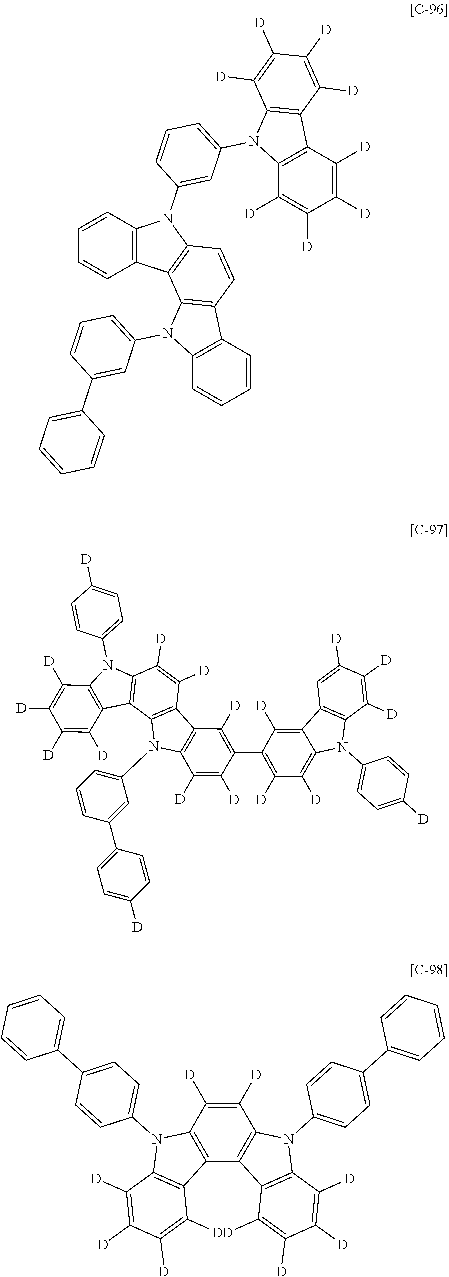 [SMILES: [2H]c1c([2H])c([2H])c2c(c1[2H])c1c([2H])c([2H])c([2H])c([2H])c1n2-c1cccc(-n2c3ccccc3c3c2ccc2c4ccccc4n(-c4cccc(-c5ccccc5)c4)c23)c1 ; [2H]c1ccc(-c2cccc(-n3c4c([2H])c([2H])c(-c5c([2H])c([2H])c6c(c5[2H])c5cc([2H])c([2H])c([2H])c5n6-c5ccc([2H])cc5)c([2H])c4c4c([2H])c([2H])c5c(c6c([2H])c([2H])c([2H])c([2H])c6n5-c5ccc([2H])cc5)c43)c2)cc1 ; [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1]

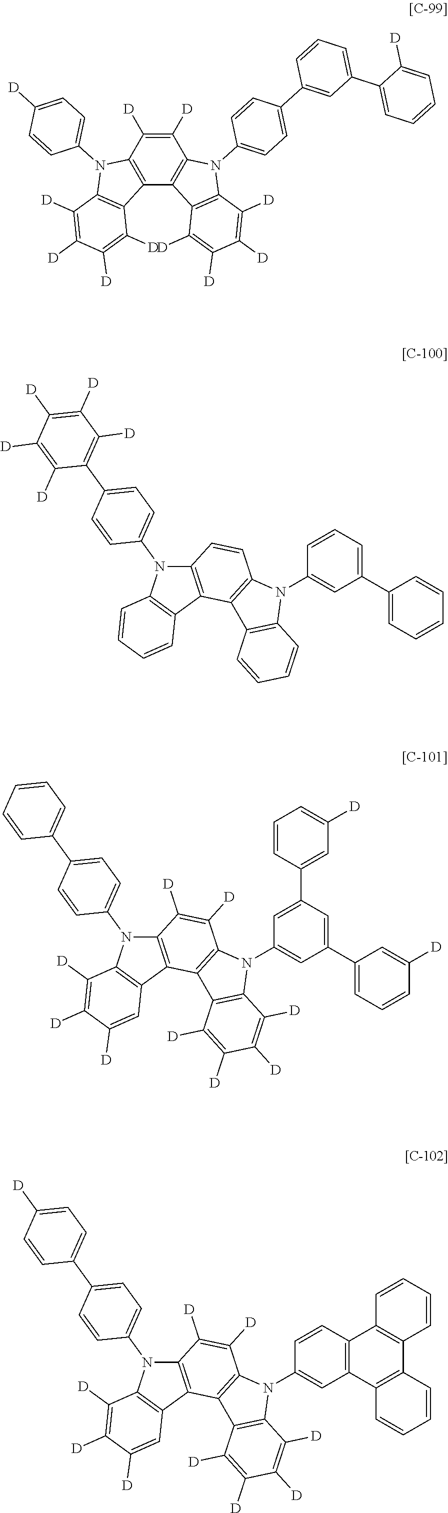

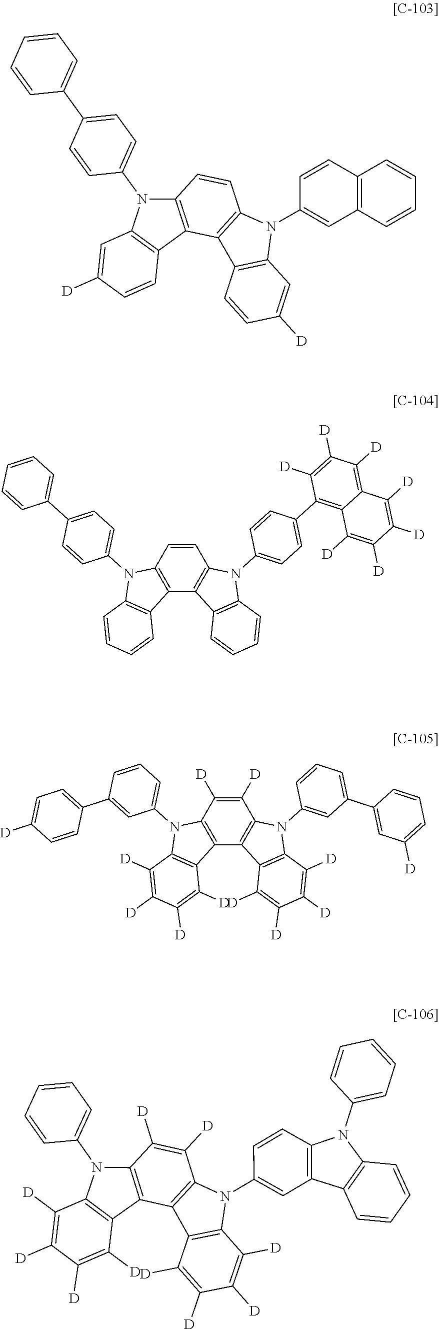 [SMILES: [2H]c1ccc2c3c4c5ccc([2H])cc5n(-c5ccc6ccccc6c5)c4ccc3n(-c3ccc(-c4ccccc4)cc3)c2c1 ; [2H]c1c([2H])c([2H])c2c(-c3ccc(-n4c5ccccc5c5c6c7ccccc7n(-c7ccc(-c8ccccc8)cc7)c6ccc54)cc3)c([2H])c([2H])c([2H])c2c1[2H] ; [2H]c1ccc(-c2cccc(-n3c4c([2H])c([2H])c([2H])c([2H])c4c4c5c6c([2H])c([2H])c([2H])c([2H])c6n(-c6cccc(-c7cccc([2H])c7)c6)c5c([2H])c([2H])c43)c2)cc1 ; [2H]c1c([2H])c([2H])c2c(c1[2H])c1c3c4c([2H])c([2H])c([2H])c([2H])c4n(-c4ccc5c(c4)c4ccccc4n5-c4ccccc4)c3c([2H])c([2H])c1n2-c1ccccc1]

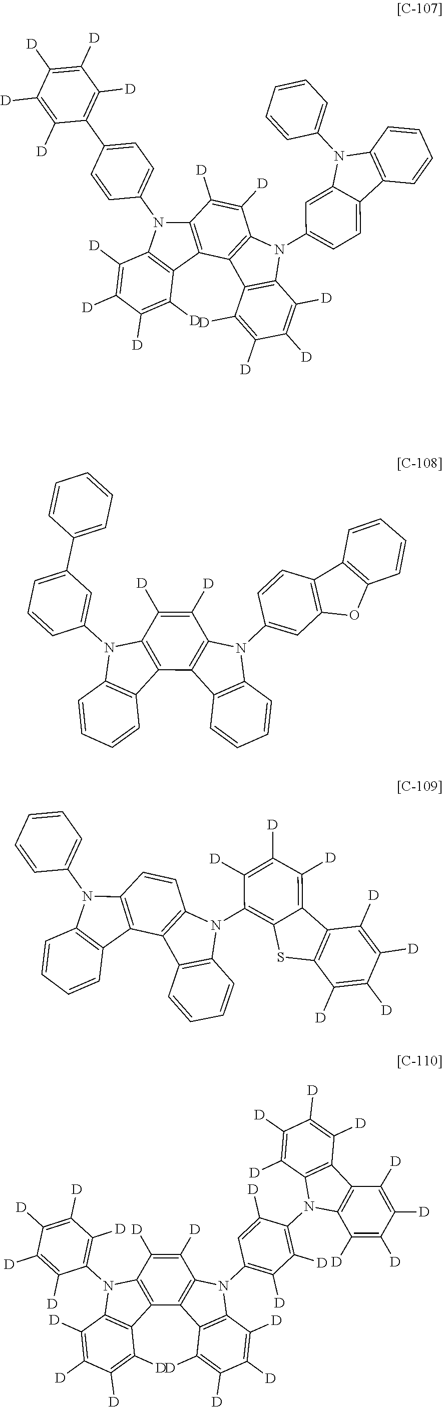

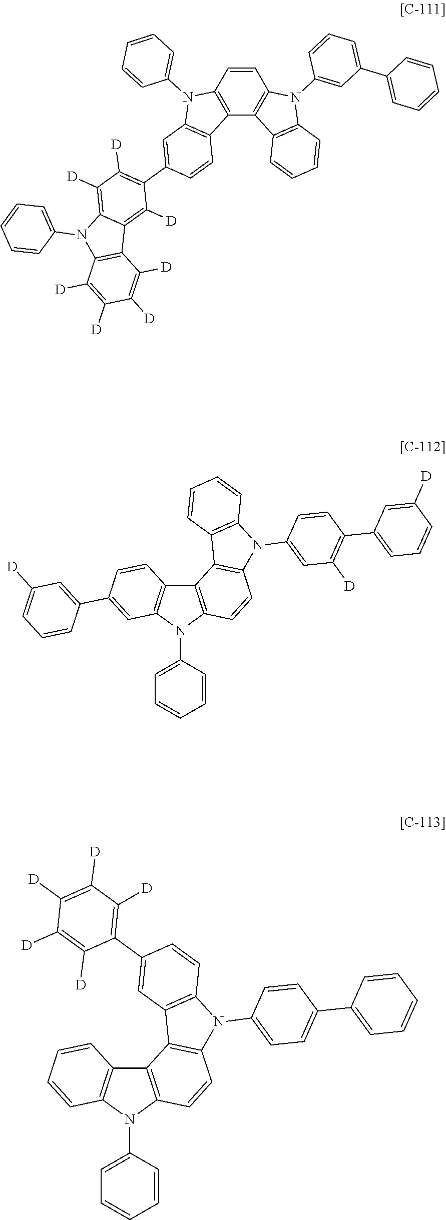 [SMILES: [2H]c1c([2H])c([2H])c2c(c1[2H])c1c([2H])c(-c3ccc4c5c6c7ccccc7n(-c7cccc(-c8ccccc8)c7)c6ccc5n(-c5ccccc5)c4c3)c([2H])c([2H])c1n2-c1ccccc1 ; [2H]c1cccc(-c2ccc3c4c5c6ccccc6n(-c6ccc(-c7cccc([2H])c7)c([2H])c6)c5ccc4n(-c4ccccc4)c3c2)c1 ; [2H]c1c([2H])c([2H])c(-c2ccc3c(c2)c2c4c5ccccc5n(-c5ccccc5)c4ccc2n3-c2ccc(-c3ccccc3)cc2)c([2H])c1[2H]]

In an implementation, the second compound for the organic optoelectronic device may be, e.g., a compound of Group 4.

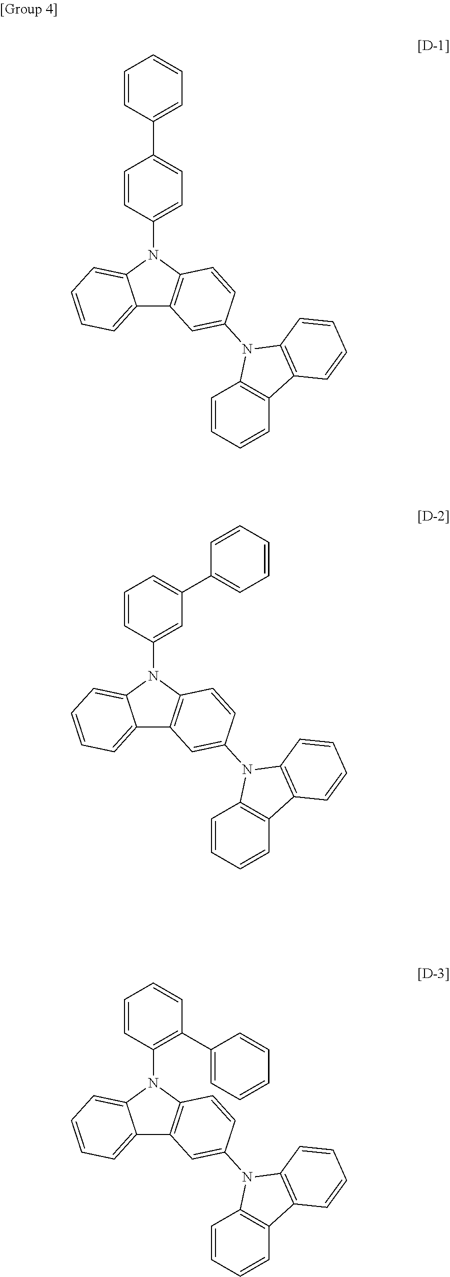 [SMILES: c1ccc(-c2ccc(-n3c4ccccc4c4cc(-n5c6ccccc6c6ccccc65)ccc43)cc2)cc1 ; c1ccc(-c2cccc(-n3c4ccccc4c4cc(-n5c6ccccc6c6ccccc65)ccc43)c2)cc1 ; c1ccc(-c2ccccc2-n2c3ccccc3c3cc(-n4c5ccccc5c5ccccc54)ccc32)cc1]

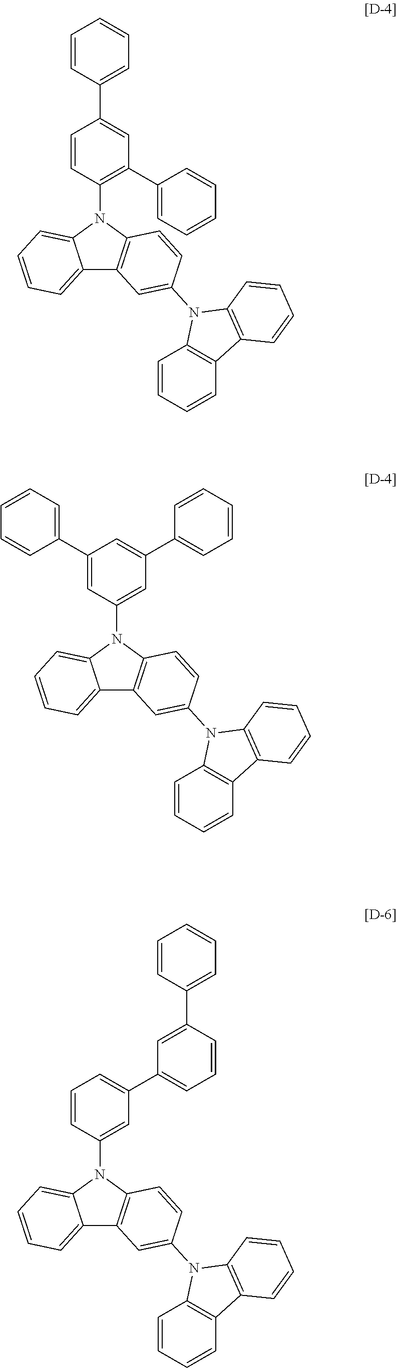 [SMILES: c1ccc(-c2ccc(-n3c4ccccc4c4cc(-n5c6ccccc6c6ccccc65)ccc43)c(-c3ccccc3)c2)cc1 ; c1ccc(-c2cc(-c3ccccc3)cc(-n3c4ccccc4c4cc(-n5c6ccccc6c6ccccc65)ccc43)c2)cc1 ; c1ccc(-c2cccc(-c3cccc(-n4c5ccccc5c5cc(-n6c7ccccc7c7ccccc76)ccc54)c3)c2)cc1]

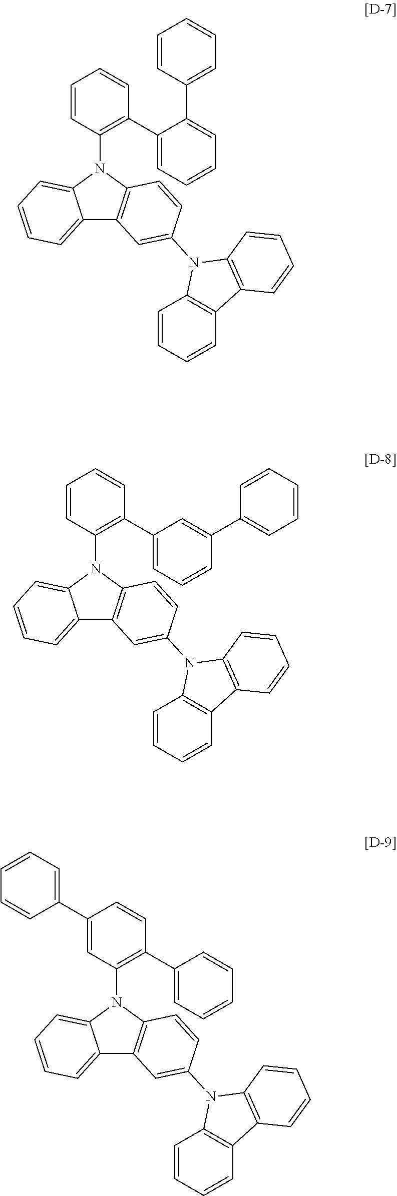 [SMILES: c1ccc(-c2ccccc2-c2ccccc2-n2c3ccccc3c3cc(-n4c5ccccc5c5ccccc54)ccc32)cc1 ; c1ccc(-c2cccc(-c3ccccc3-n3c4ccccc4c4cc(-n5c6ccccc6c6ccccc65)ccc43)c2)cc1 ; c1ccc(-c2ccc(-c3ccccc3)c(-n3c4ccccc4c4cc(-n5c6ccccc6c6ccccc65)ccc43)c2)cc1]

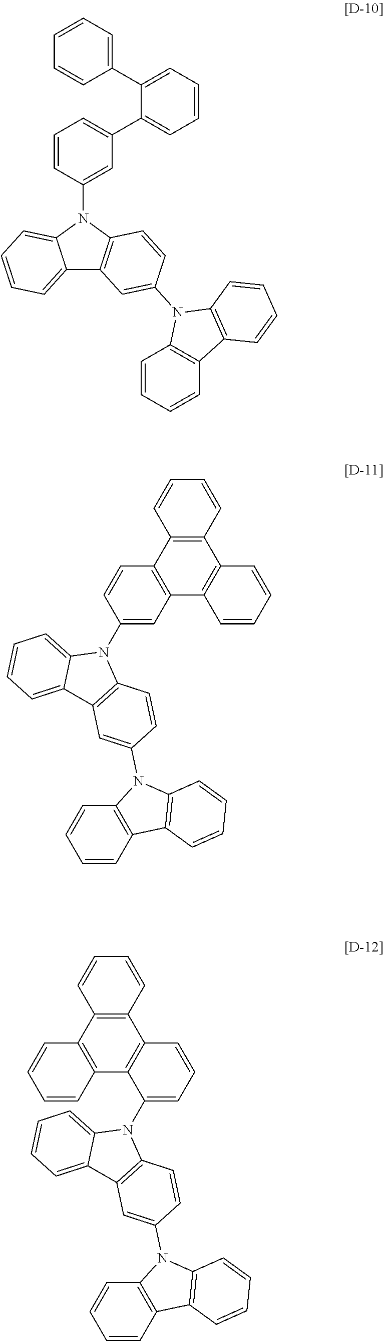 [SMILES: c1ccc(-c2ccccc2-c2cccc(-n3c4ccccc4c4cc(-n5c6ccccc6c6ccccc65)ccc43)c2)cc1 ; c1ccc2c(c1)c1ccccc1c1cc(-n3c4ccccc4c4cc(-n5c6ccccc6c6ccccc65)ccc43)ccc21 ; c1ccc2c(c1)c1ccccc1c1c(-n3c4ccccc4c4cc(-n5c6ccccc6c6ccccc65)ccc43)cccc21]

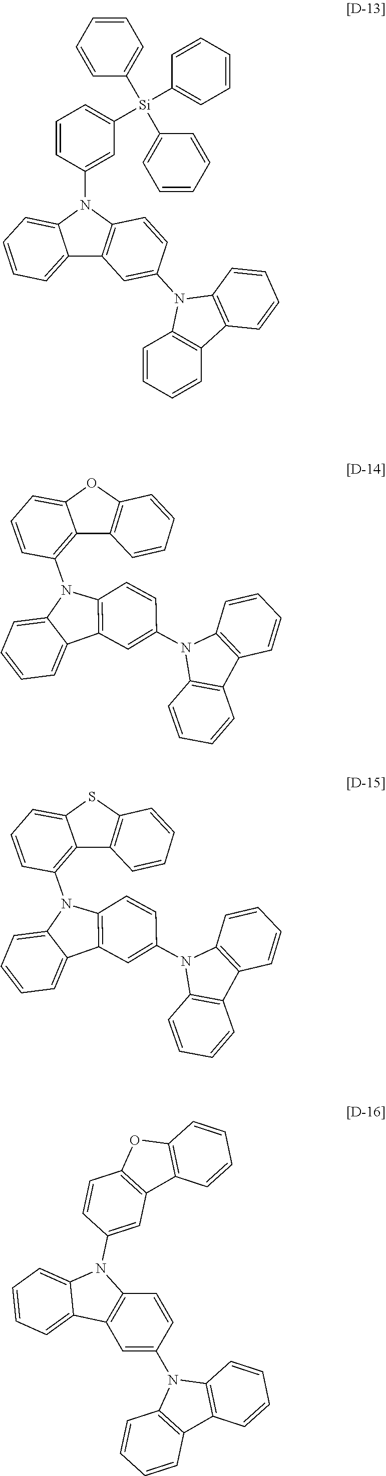 [SMILES: c1ccc([Si](c2ccccc2)(c2ccccc2)c2cccc(-n3c4ccccc4c4cc(-n5c6ccccc6c6ccccc65)ccc43)c2)cc1 ; c1ccc2c(c1)oc1cccc(-n3c4ccccc4c4cc(-n5c6ccccc6c6ccccc65)ccc43)c12 ; c1ccc2c(c1)sc1cccc(-n3c4ccccc4c4cc(-n5c6ccccc6c6ccccc65)ccc43)c12 ; c1ccc2c(c1)oc1ccc(-n3c4ccccc4c4cc(-n5c6ccccc6c6ccccc65)ccc43)cc12]

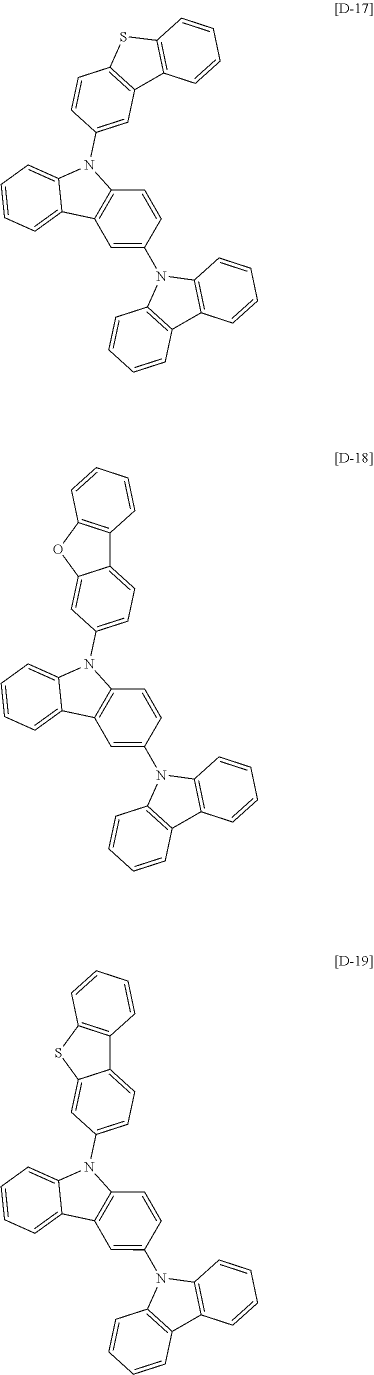 [SMILES: c1ccc2c(c1)sc1ccc(-n3c4ccccc4c4cc(-n5c6ccccc6c6ccccc65)ccc43)cc12 ; c1ccc2c(c1)oc1cc(-n3c4ccccc4c4cc(-n5c6ccccc6c6ccccc65)ccc43)ccc12 ; c1ccc2c(c1)sc1cc(-n3c4ccccc4c4cc(-n5c6ccccc6c6ccccc65)ccc43)ccc12]

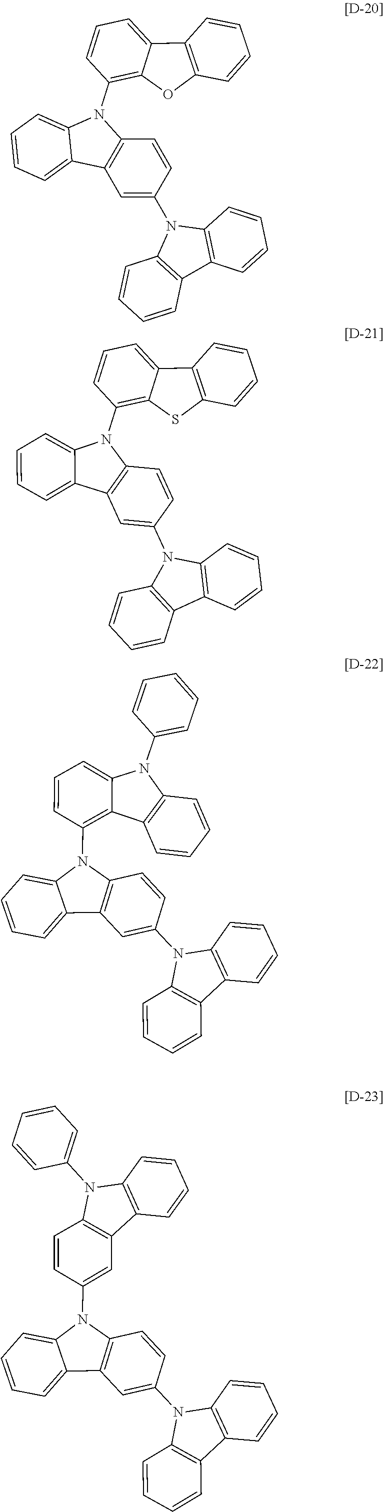

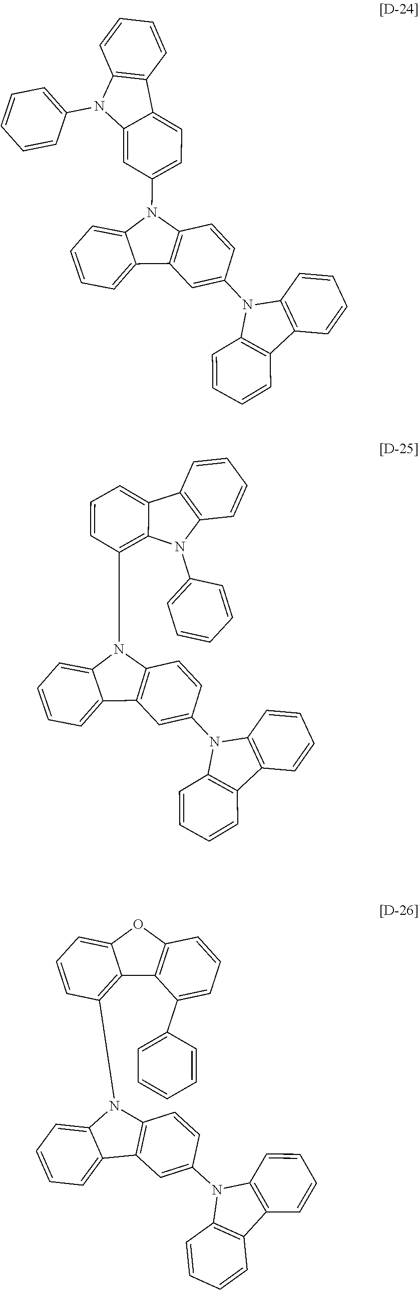

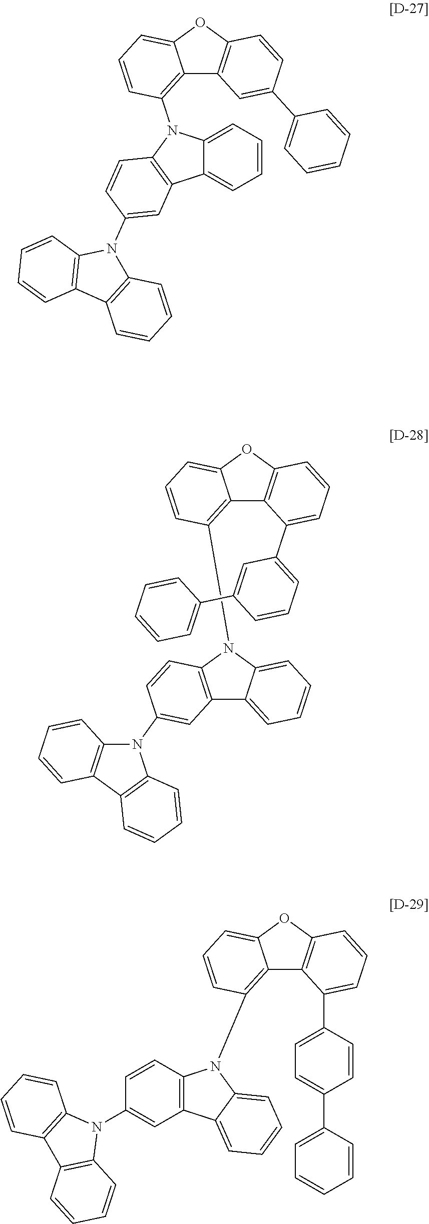 [SMILES: c1ccc(-c2ccc3oc4cccc(-n5c6ccccc6c6cc(-n7c8ccccc8c8ccccc87)ccc65)c4c3c2)cc1 ; c1ccc(-c2cccc(-c3cccc4oc5cccc(-n6c7ccccc7c7cc(-n8c9ccccc9c9ccccc98)ccc76)c5c34)c2)cc1 ; c1ccc(-c2ccc(-c3cccc4oc5cccc(-n6c7ccccc7c7cc(-n8c9ccccc9c9ccccc98)ccc76)c5c34)cc2)cc1]

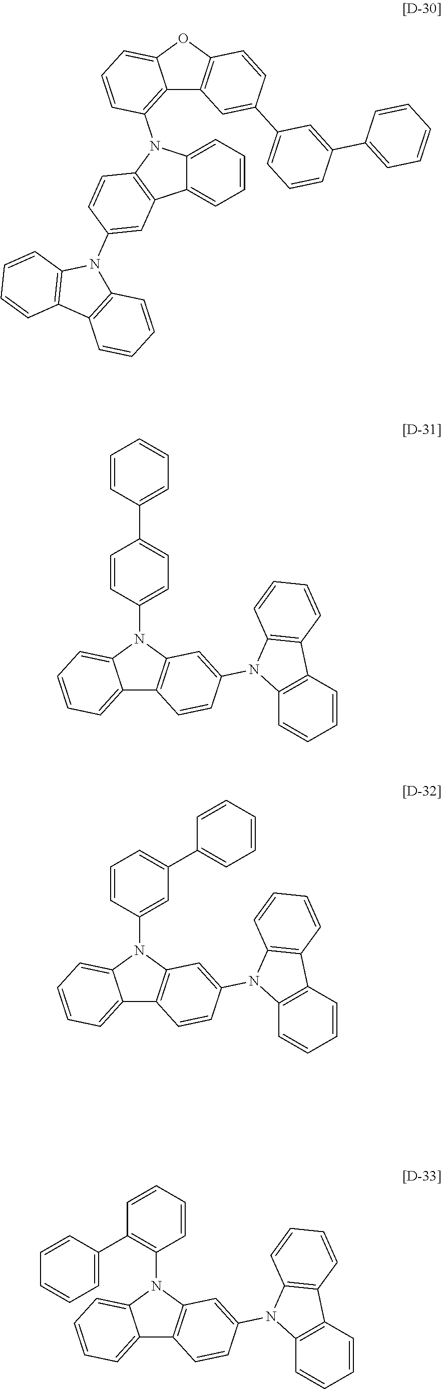 [SMILES: c1ccc(-c2cccc(-c3ccc4oc5cccc(-n6c7ccccc7c7cc(-n8c9ccccc9c9ccccc98)ccc76)c5c4c3)c2)cc1 ; c1ccc(-c2ccc(-n3c4ccccc4c4ccc(-n5c6ccccc6c6ccccc65)cc43)cc2)cc1 ; c1ccc(-c2cccc(-n3c4ccccc4c4ccc(-n5c6ccccc6c6ccccc65)cc43)c2)cc1 ; c1ccc(-c2ccccc2-n2c3ccccc3c3ccc(-n4c5ccccc5c5ccccc54)cc32)cc1]

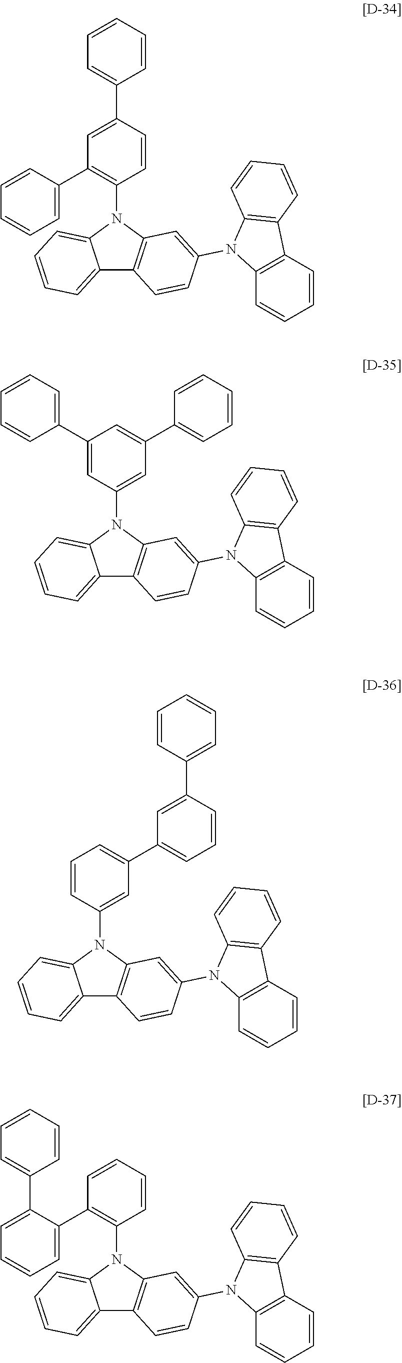 [SMILES: c1ccc(-c2ccc(-n3c4ccccc4c4ccc(-n5c6ccccc6c6ccccc65)cc43)c(-c3ccccc3)c2)cc1 ; c1ccc(-c2cc(-c3ccccc3)cc(-n3c4ccccc4c4ccc(-n5c6ccccc6c6ccccc65)cc43)c2)cc1 ; c1ccc(-c2cccc(-c3cccc(-n4c5ccccc5c5ccc(-n6c7ccccc7c7ccccc76)cc54)c3)c2)cc1 ; c1ccc(-c2ccccc2-c2ccccc2-n2c3ccccc3c3ccc(-n4c5ccccc5c5ccccc54)cc32)cc1]

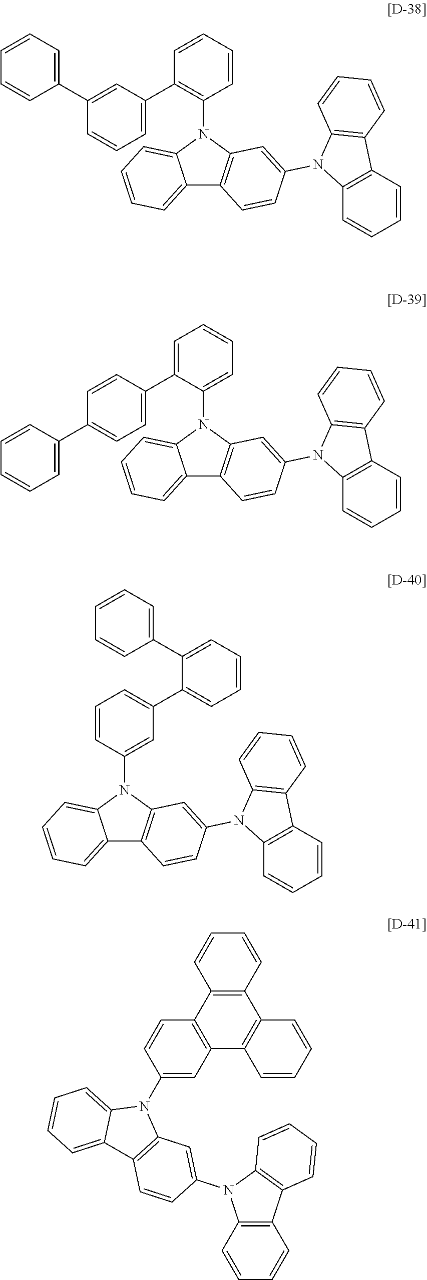 [SMILES: c1ccc(-c2cccc(-c3ccccc3-n3c4ccccc4c4ccc(-n5c6ccccc6c6ccccc65)cc43)c2)cc1 ; c1ccc(-c2ccc(-c3ccccc3-n3c4ccccc4c4ccc(-n5c6ccccc6c6ccccc65)cc43)cc2)cc1 ; c1ccc(-c2ccccc2-c2cccc(-n3c4ccccc4c4ccc(-n5c6ccccc6c6ccccc65)cc43)c2)cc1 ; c1ccc2c(c1)c1ccccc1c1cc(-n3c4ccccc4c4ccc(-n5c6ccccc6c6ccccc65)cc43)ccc21]

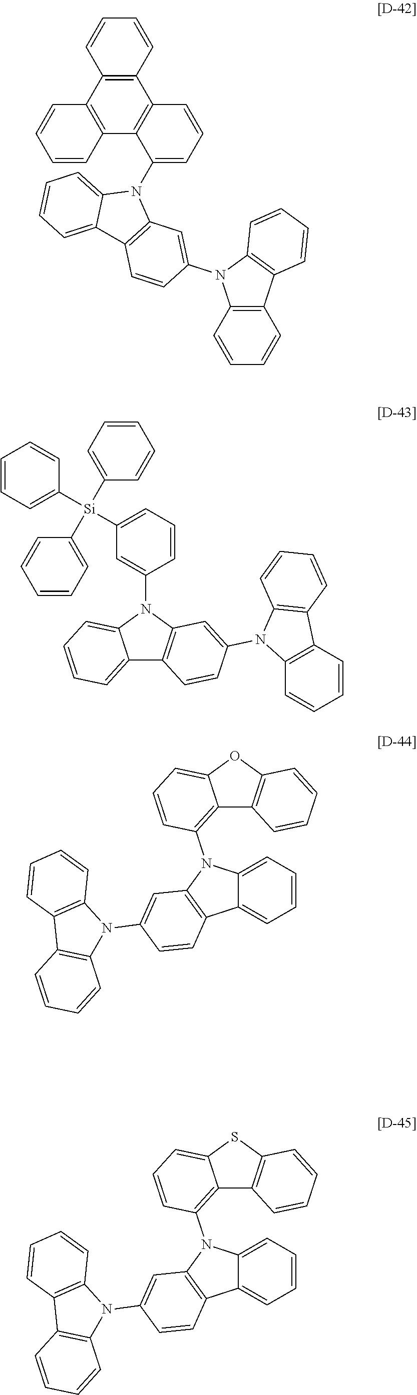 [SMILES: c1ccc2c(c1)c1ccccc1c1c(-n3c4ccccc4c4ccc(-n5c6ccccc6c6ccccc65)cc43)cccc21 ; c1ccc([Si](c2ccccc2)(c2ccccc2)c2cccc(-n3c4ccccc4c4ccc(-n5c6ccccc6c6ccccc65)cc43)c2)cc1 ; c1ccc2c(c1)oc1cccc(-n3c4ccccc4c4ccc(-n5c6ccccc6c6ccccc65)cc43)c12 ; c1ccc2c(c1)sc1cccc(-n3c4ccccc4c4ccc(-n5c6ccccc6c6ccccc65)cc43)c12]

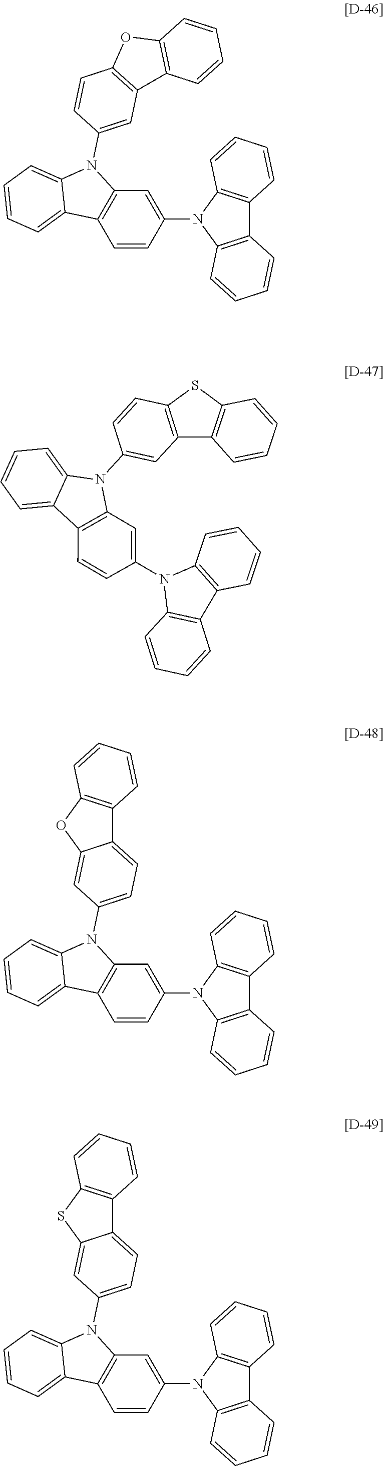 [SMILES: c1ccc2c(c1)oc1ccc(-n3c4ccccc4c4ccc(-n5c6ccccc6c6ccccc65)cc43)cc12 ; c1ccc2c(c1)sc1ccc(-n3c4ccccc4c4ccc(-n5c6ccccc6c6ccccc65)cc43)cc12 ; c1ccc2c(c1)oc1cc(-n3c4ccccc4c4ccc(-n5c6ccccc6c6ccccc65)cc43)ccc12 ; c1ccc2c(c1)sc1cc(-n3c4ccccc4c4ccc(-n5c6ccccc6c6ccccc65)cc43)ccc12]

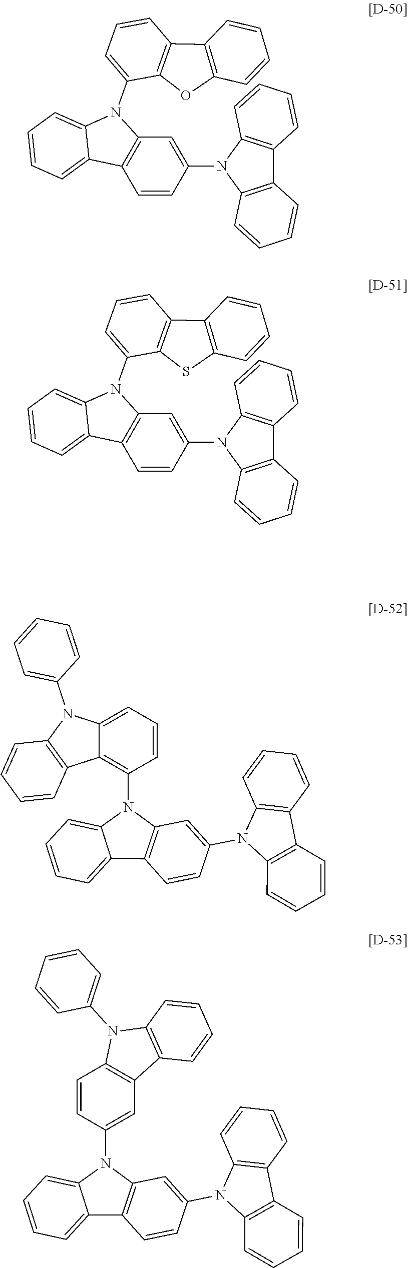 [SMILES: c1ccc2c(c1)oc1c(-n3c4ccccc4c4ccc(-n5c6ccccc6c6ccccc65)cc43)cccc12 ; c1ccc2c(c1)sc1c(-n3c4ccccc4c4ccc(-n5c6ccccc6c6ccccc65)cc43)cccc12 ; c1ccc(-n2c3ccccc3c3c(-n4c5ccccc5c5ccc(-n6c7ccccc7c7ccccc76)cc54)cccc32)cc1 ; c1ccc(-n2c3ccccc3c3cc(-n4c5ccccc5c5ccc(-n6c7ccccc7c7ccccc76)cc54)ccc32)cc1]

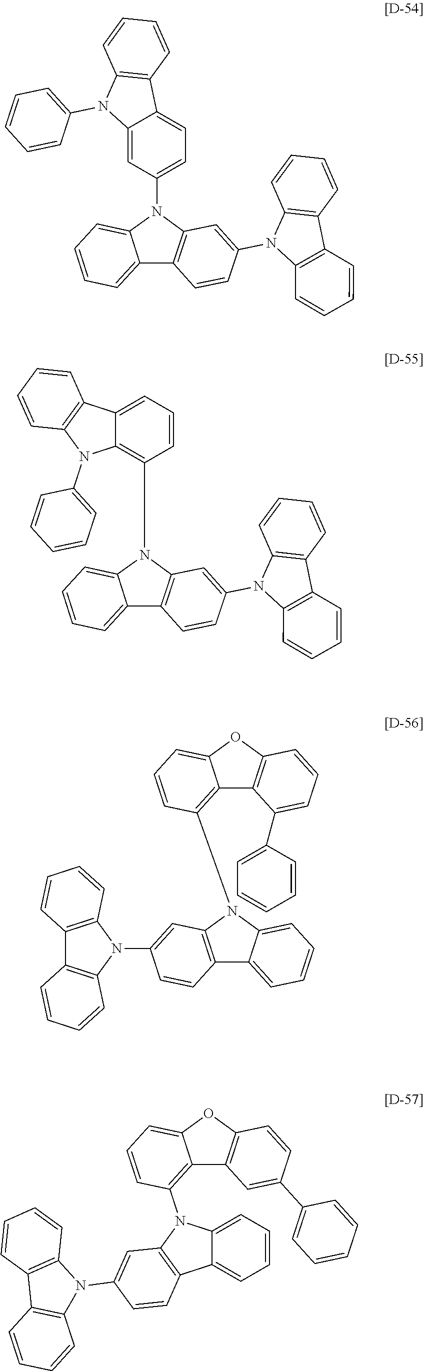 [SMILES: c1ccc(-n2c3ccccc3c3ccc(-n4c5ccccc5c5ccc(-n6c7ccccc7c7ccccc76)cc54)cc32)cc1 ; c1ccc(-n2c3ccccc3c3cccc(-n4c5ccccc5c5ccc(-n6c7ccccc7c7ccccc76)cc54)c32)cc1 ; c1ccc(-c2cccc3oc4cccc(-n5c6ccccc6c6ccc(-n7c8ccccc8c8ccccc87)cc65)c4c23)cc1 ; c1ccc(-c2ccc3oc4cccc(-n5c6ccccc6c6ccc(-n7c8ccccc8c8ccccc87)cc65)c4c3c2)cc1]

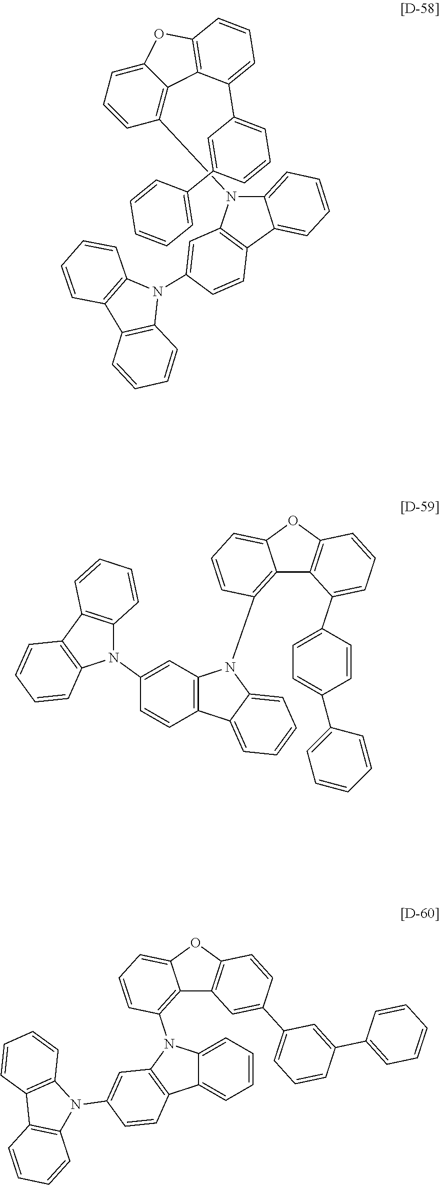 [SMILES: c1ccc(-c2cccc(-c3cccc4oc5cccc(-n6c7ccccc7c7ccc(-n8c9ccccc9c9ccccc98)cc76)c5c34)c2)cc1 ; c1ccc(-c2ccc(-c3cccc4oc5cccc(-n6c7ccccc7c7ccc(-n8c9ccccc9c9ccccc98)cc76)c5c34)cc2)cc1 ; c1ccc(-c2cccc(-c3ccc4oc5cccc(-n6c7ccccc7c7ccc(-n8c9ccccc9c9ccccc98)cc76)c5c4c3)c2)cc1]

Examples of Compound D-1 to Compound D-60 listed in Group 2 in which at least one hydrogen is replaced with deuterium are shown below.

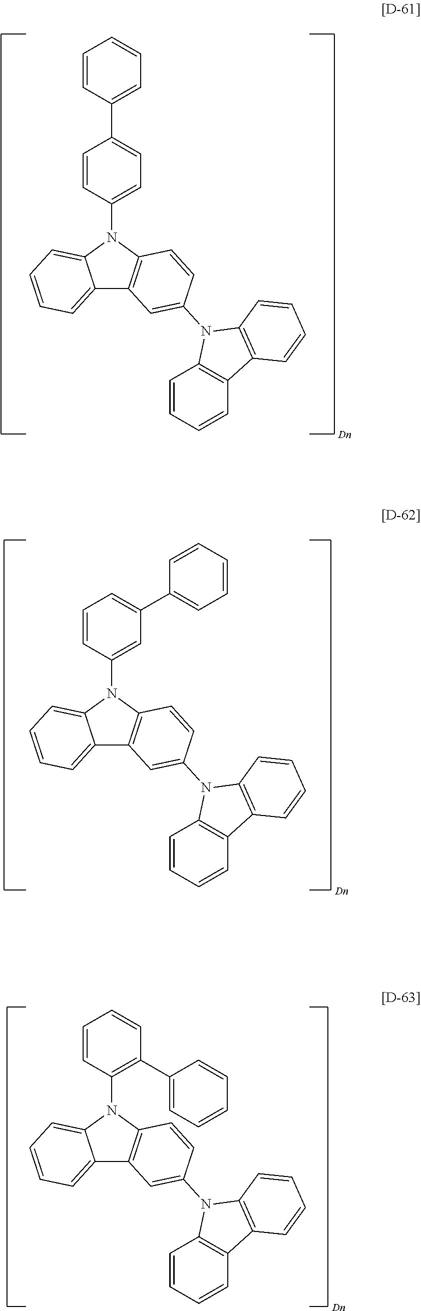

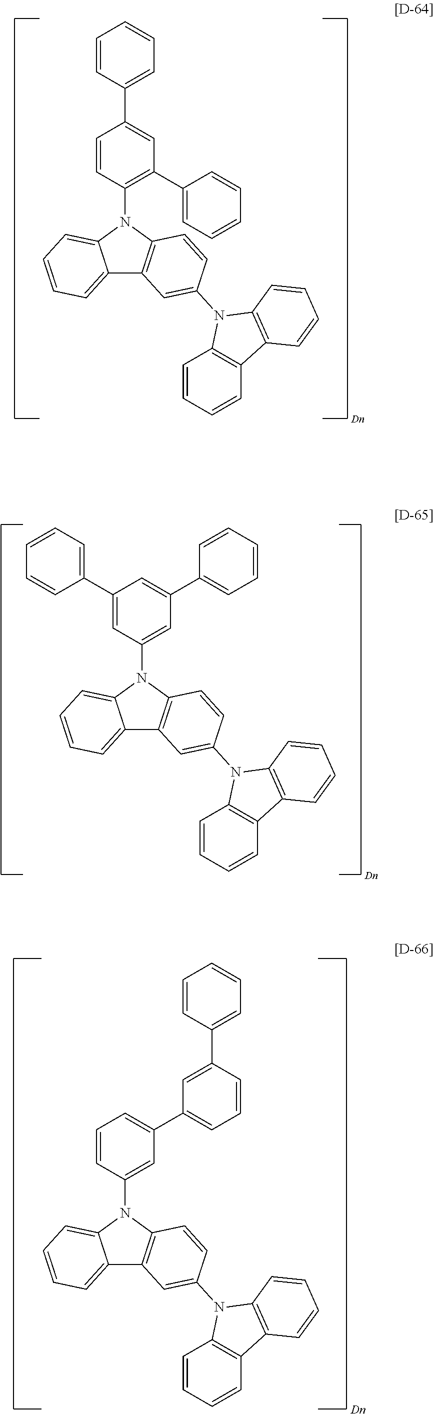

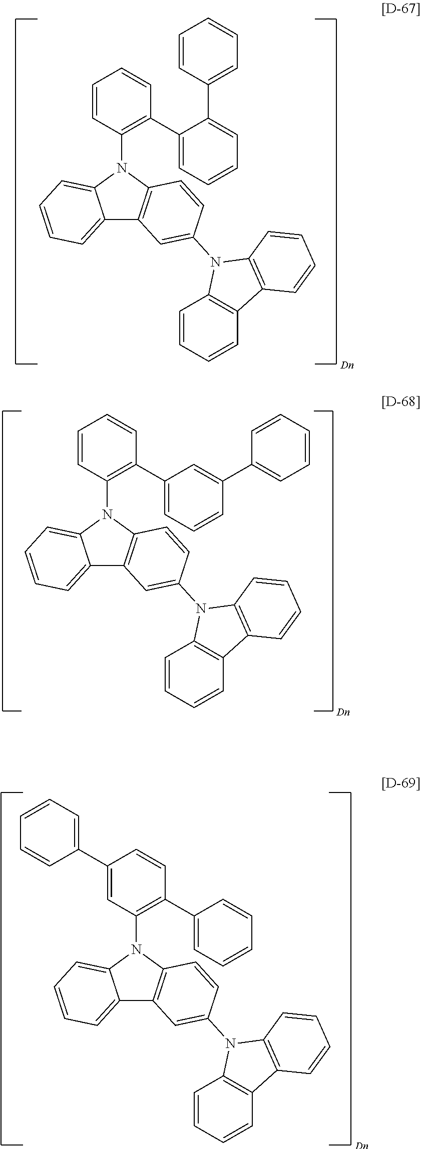

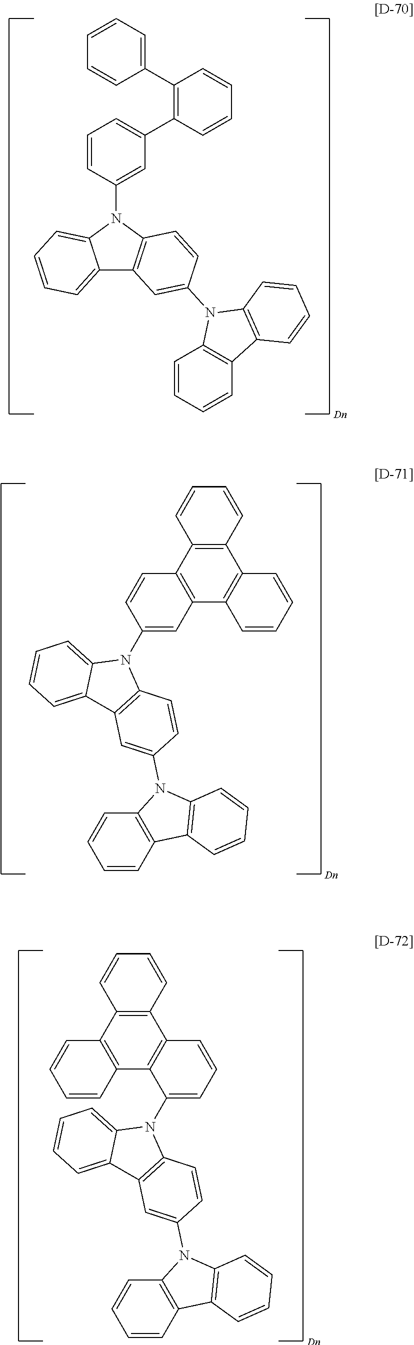

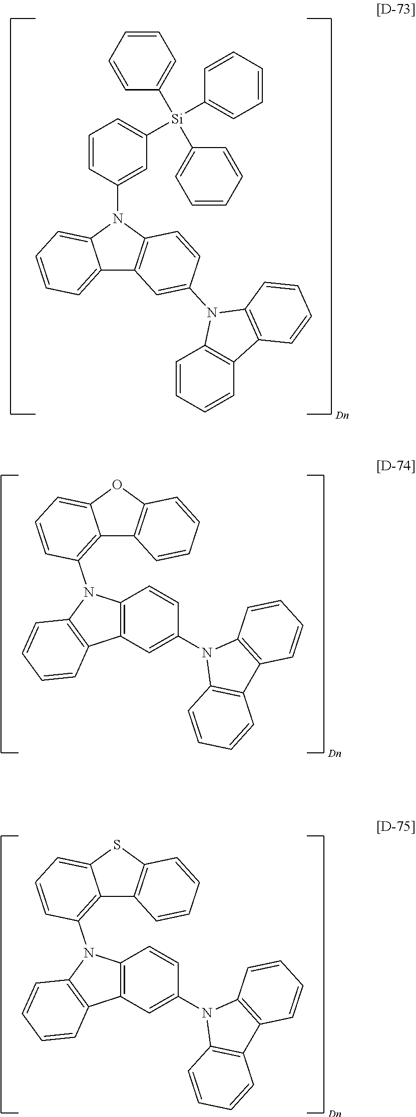

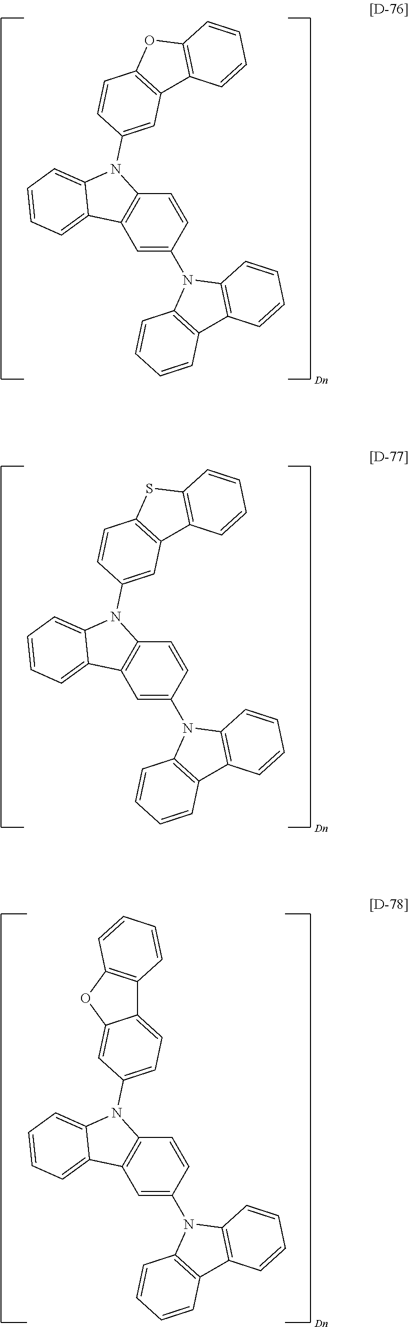

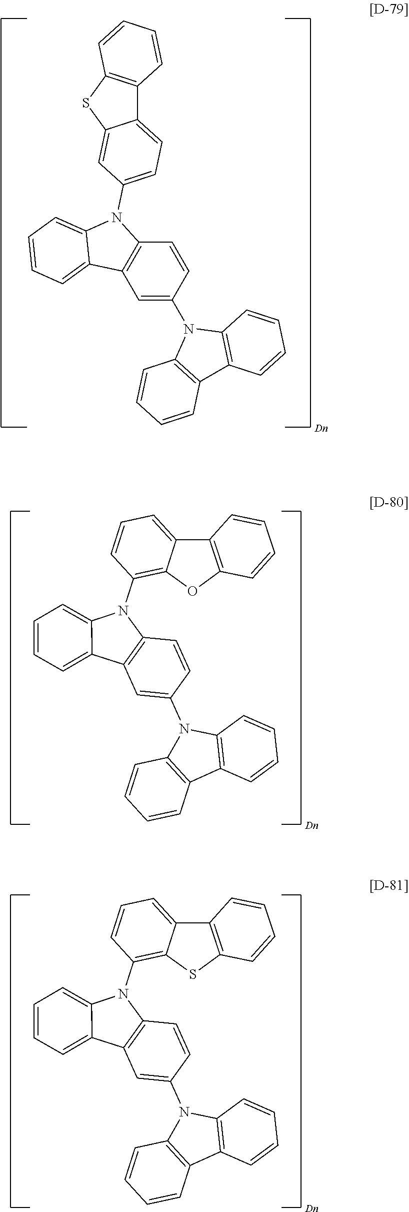

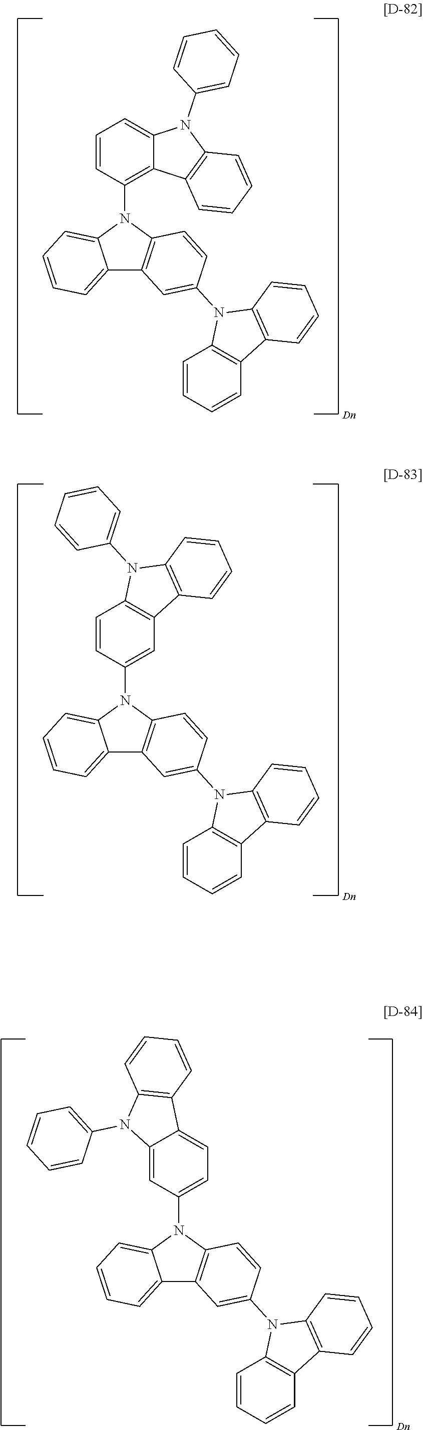

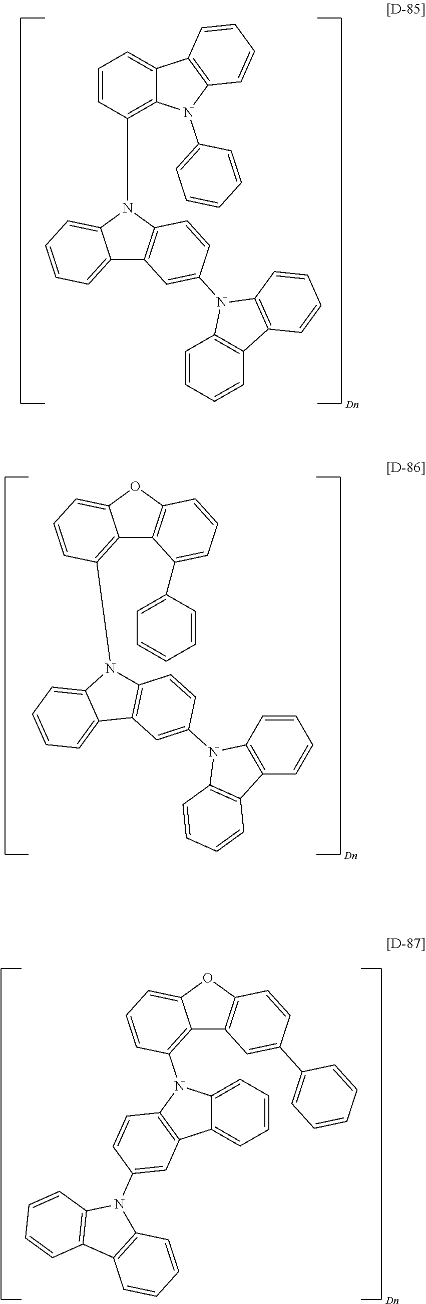

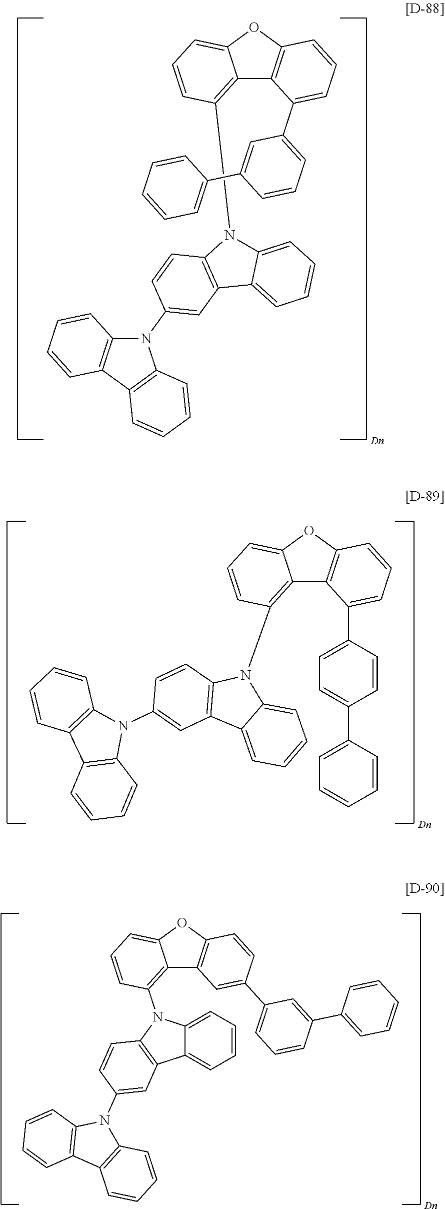

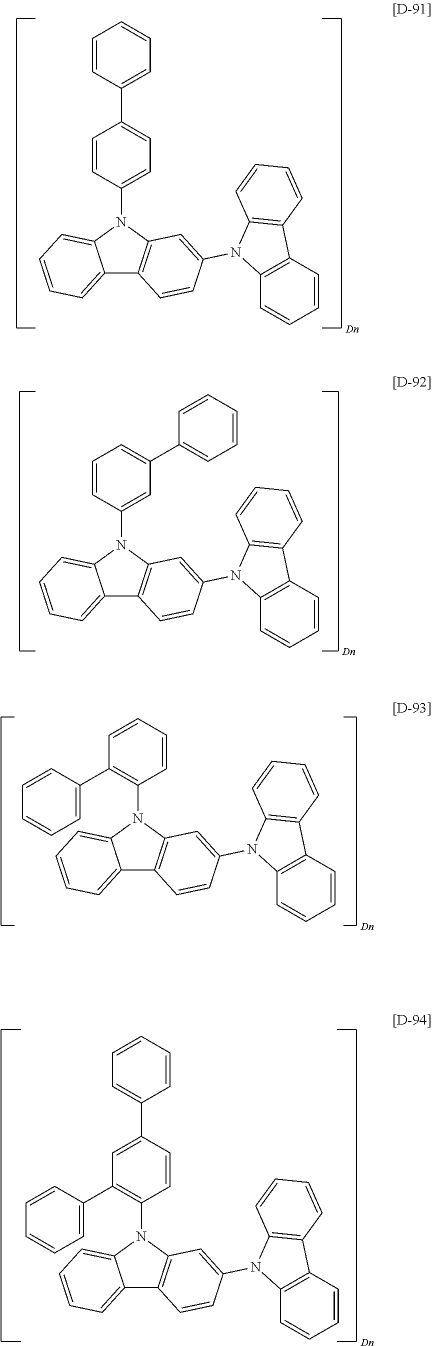

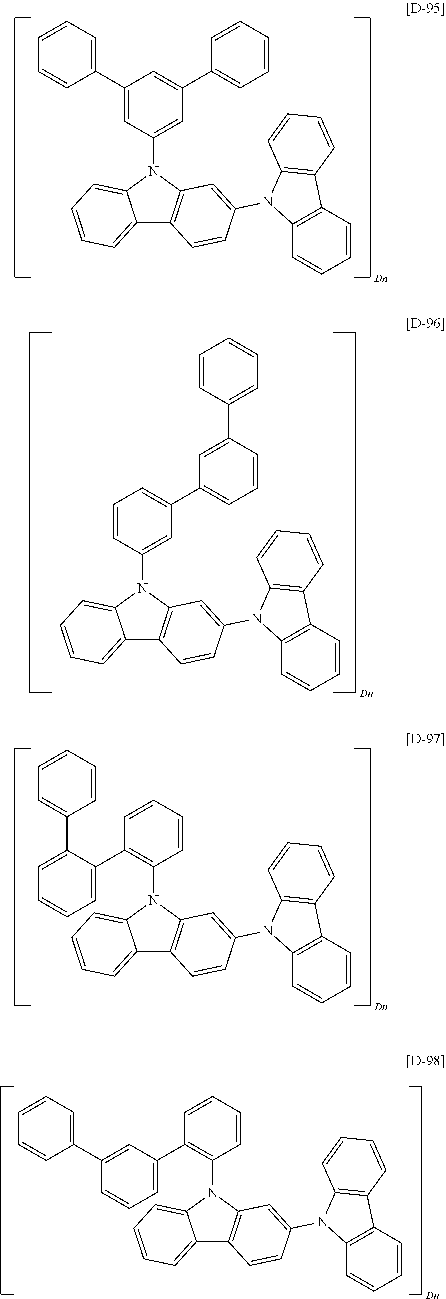

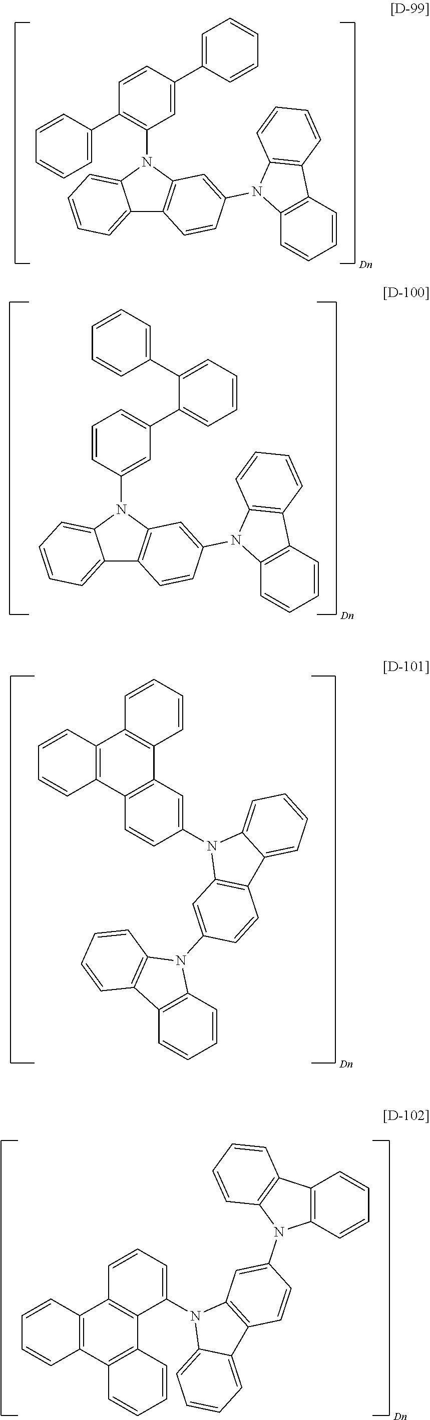

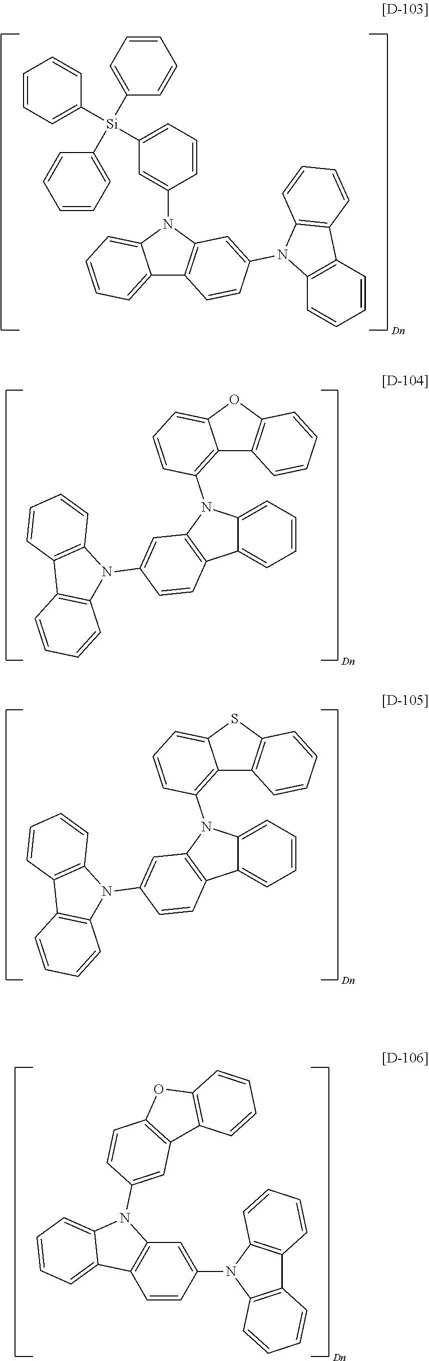

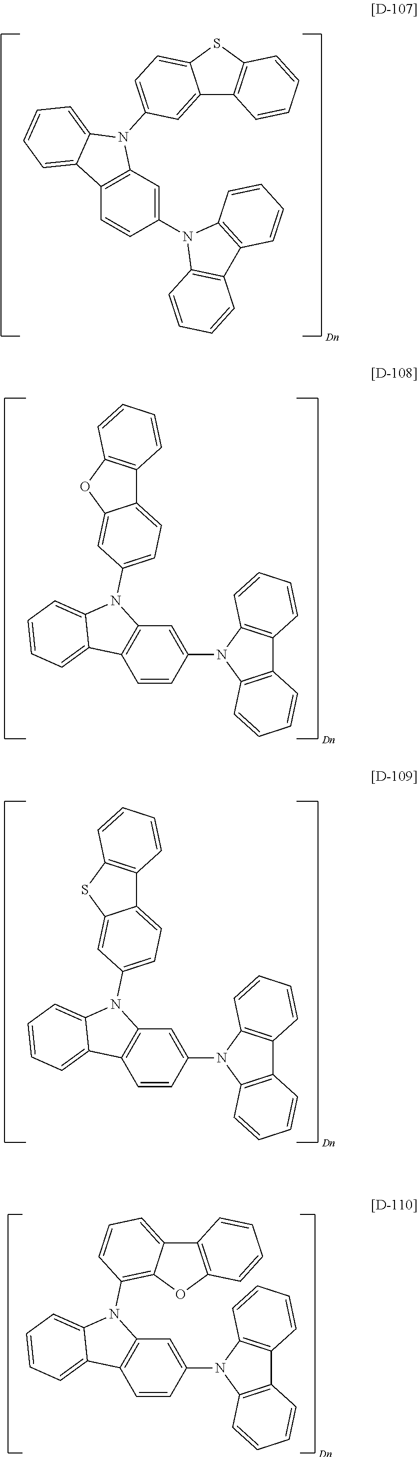

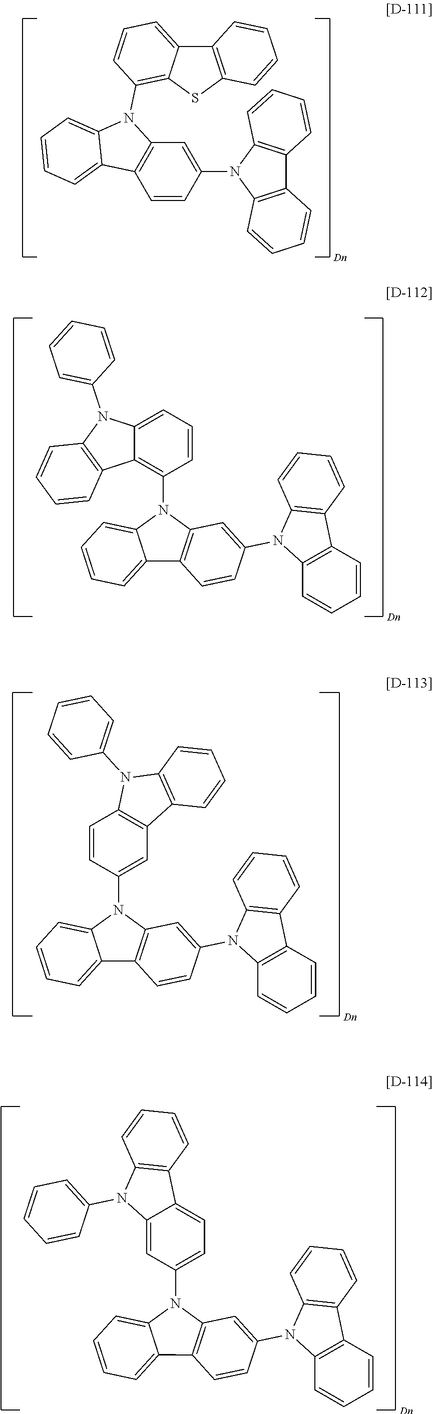

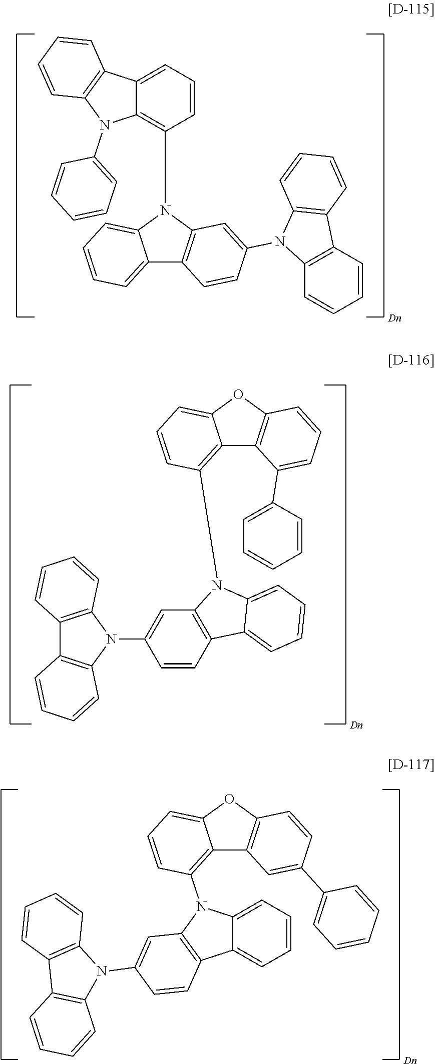

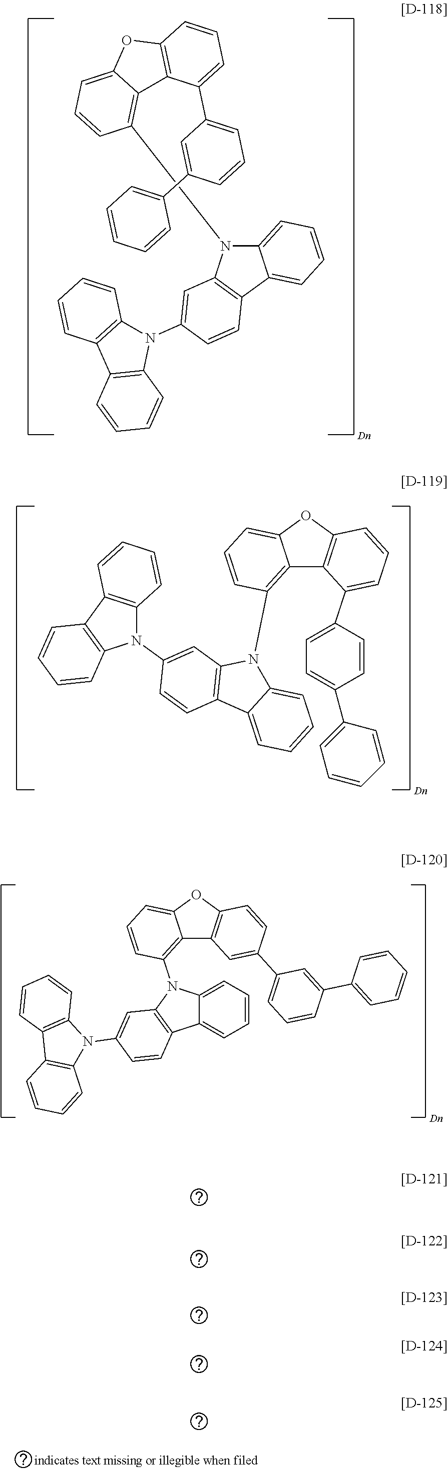

(Dn refers to the number of deuterium atoms substituted, and n is an integer between 1 and the maximum number of substitutions possible)

In an implementation, the second compound may be represented by, e.g., Chemical Formula 5.

In an implementation, the first compound may be represented by, e.g., one of Chemical Formula 1-2 to Chemical Formula 1-4, and the second compound may be represented by, e.g., Chemical Formula 5-3.

In an implementation, the first compound may be represented by one of, e.g., Chemical Formula 1-2a to Chemical Formula 1-4a.

In an implementation, the first compound may be represented by, e.g., Chemical Formula 1-3a or Chemical Formula 1-4a.

In an implementation, R¹ to R¹⁰ may each independently be, e.g., hydrogen, deuterium, a substituted or unsubstituted C1 to C10 alkyl group, a substituted or unsubstituted C3 to C10 cycloalkyl group, a substituted or unsubstituted phenyl group, or a substituted or unsubstituted biphenyl group.

In an implementation, R¹ to R¹⁰ may each independently be, e.g., hydrogen, deuterium, a substituted or unsubstituted methyl group, a substituted or unsubstituted ethyl group, a substituted or unsubstituted propyl group, a substituted or unsubstituted butyl group, a substituted or unsubstituted tert-butyl group, a substituted or unsubstituted cyclohexyl group, or a substituted or unsubstituted phenyl group.

In an implementation, in Chemical Formula 5-3, L⁵ may be, e.g., a substituted or unsubstituted C6 to C12 arylene group, R¹⁸ to R²¹ may each independently be, e.g., hydrogen, deuterium, or a substituted or unsubstituted phenyl group, Ar⁵ may be, e.g., a substituted or unsubstituted C6 to C12 aryl group, m18, m20, and m21 may each independently be, e.g., an integer of 1 to 4, and m19 may be, e.g., an integer of 1 to 3.

By including the first compound and the second compound together, good interface characteristics may be exhibited, and by finely controlling the mobility of holes and electrons, the balance of electrons and holes in the active layer (e.g., the light emitting layer, 130) of the organic optoelectronic device may be increased, thereby helping increase luminescence efficiency, while at the same time helping improve life-span by reducing the unbound charge due to the imbalance in the mobility of electrons and holes.

In an implementation, the first compound and the second compound may be included as a host, and an organic light emitting diode 100 including a light emitting layer 130 to which the first compound and the second compound are applied may be finely controlled so that holes and electrons injected from the anode 110 and the cathode 120, respectively, have appropriate mobility within the light emitting layer 130, thereby strongly inducing exciton generation within the light emitting layer and helping increase luminous efficiency within the light emitting layer.

The composition for an organic optoelectronic device may include the first compound and the second compound in various ratios. In an implementation, the composition for an organic optoelectronic device may include the first compound and the second compound in a weight ratio of, e.g., 10:90 to 90:10, and within the above range, may include the first compound and the second compound in a weight ratio of, e.g., about 20:80 to about 80:20, about 30:70 to about 70:30, about 40:60 to about 60:40, or about 50:50.

In an implementation, the first compound may be included in an amount greater than or equal to that of the second compound, e.g., the first compound may be included in an amount of about 50 wt % to about 90 wt %, based on the total weight of the first compound and the second compound.

In an implementation, the first compound may be included in an amount less than or equal to that of the second compound, e.g., the first compound may be included in an amount of about 10 wt % to about 50 wt %, based on the total weight of the first compound and the second compound.

The composition for an organic optoelectronic device may further include a luminescent dopant in addition to the first compound and the second compound. The luminescent dopant may be a material that causes luminescence when mixed in a small amount in the composition for an organic optoelectronic device and generally a material such as a metal complex that emits luminescence by multiple excitation that excites to a triplet state or higher may be used. The luminescent dopant may be, e.g., an inorganic, organic, or organic/inorganic compound, and may be included in one or more types.

The luminescent dopant may be, e.g., a phosphorescent dopant, a delayed fluorescent dopant, a fluorescent dopant, or a combination thereof. In an implementation, using a phosphorescent dopant or a delayed fluorescent dopant as a phosphorescent sensitizer or an assist dopant may help effectively transfer energy received from the host to the fluorescent dopant.

The phosphorescent dopant may be an organometallic compound, and the energy transfer to the fluorescent dopant may be increased, so that the excitons formed in the light emitting layer rapidly emit light within the light emitting layer, thereby helping reduce the deterioration of the light emitting diode.

The phosphorescent dopant may be an organometallic compound, and may be e.g., an organometallic compound including iridium (Ir), platinum (Pt), osmium (Os), titanium (Ti), zirconium (Zr), hafnium (Hf), europium (Eu), terbium (Tb), thulium (Tm), rhodium (Rh), or a combination thereof, and may be an organometallic compound including an organic ligand including a nitrogen-containing ring. The nitrogen-containing ring may be, e.g., a substituted or unsubstituted pyridine, a substituted or unsubstituted pyrimidine, a substituted or unsubstituted triazine, a substituted or unsubstituted carbazole, a substituted or unsubstituted imidazole, a substituted or unsubstituted benzimidazole, or a combination thereof.

The phosphorescent dopant may be, e.g., one of Compounds P1 to P32, and P41 to P52.

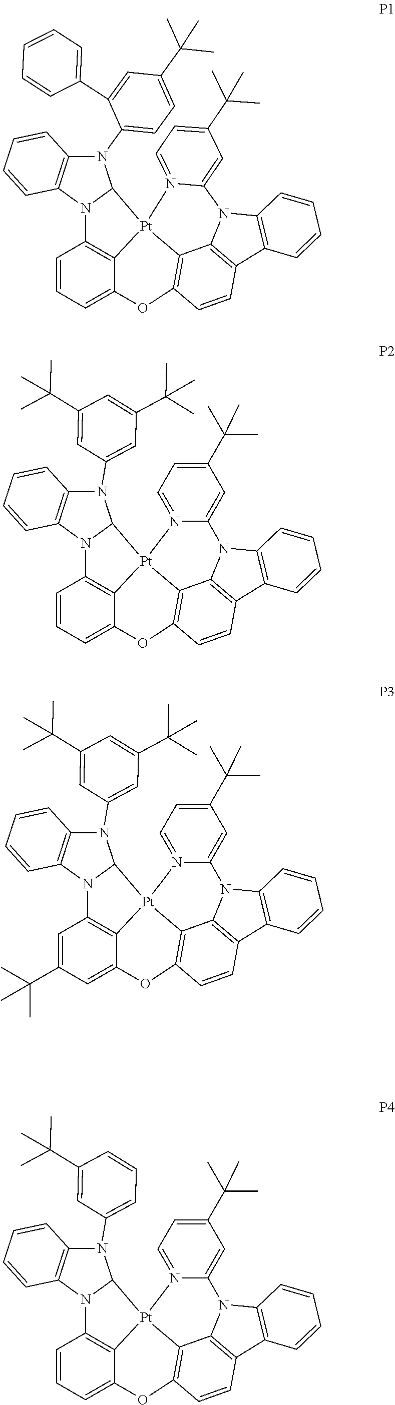

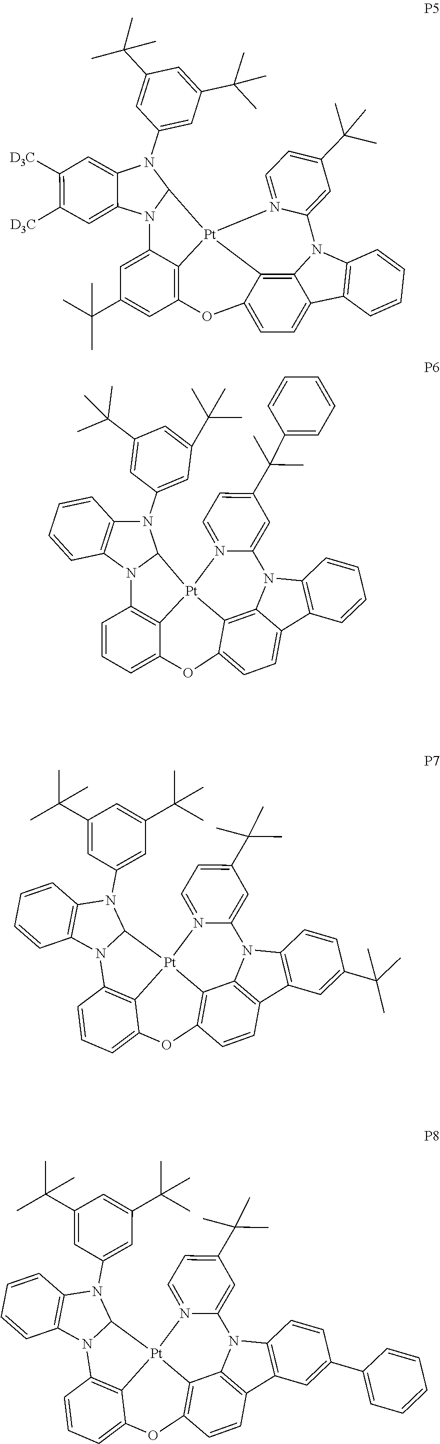

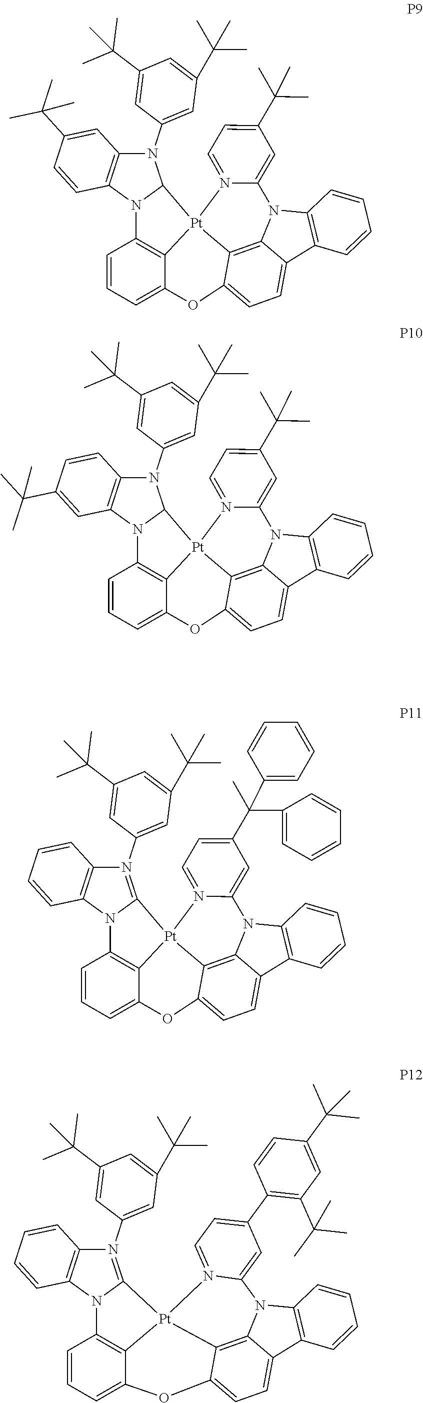

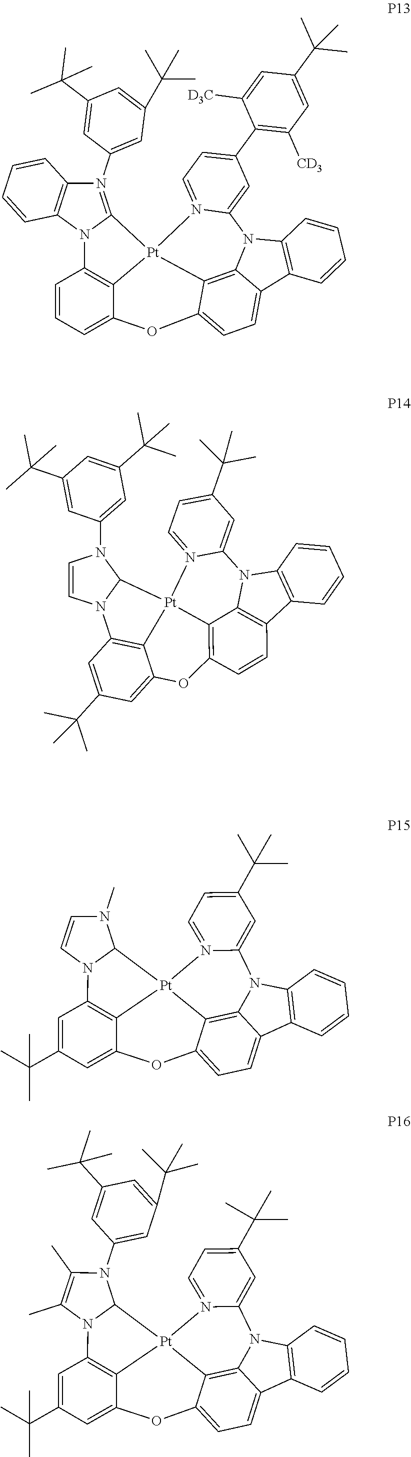

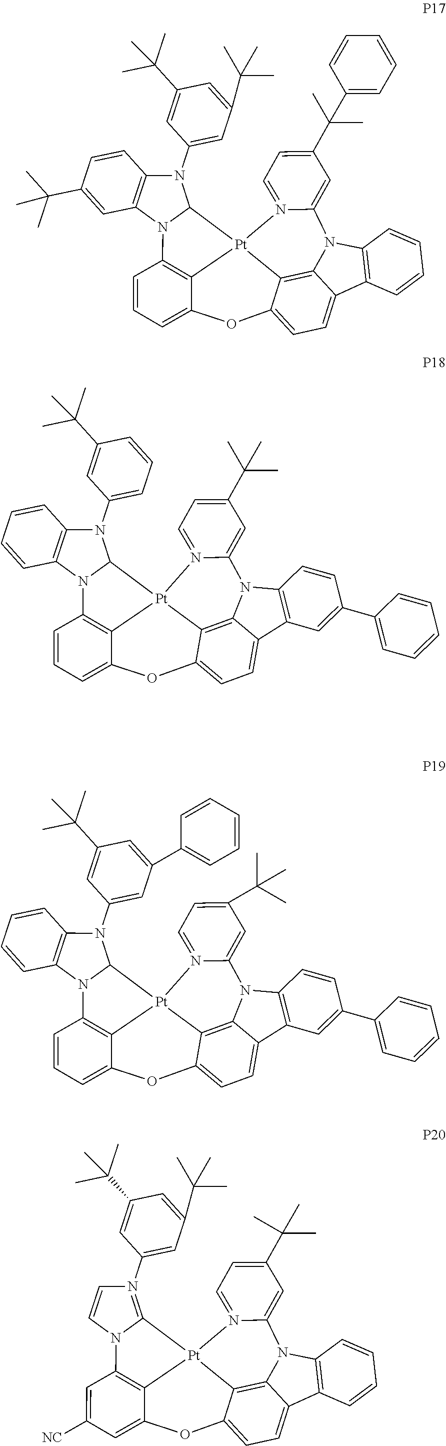

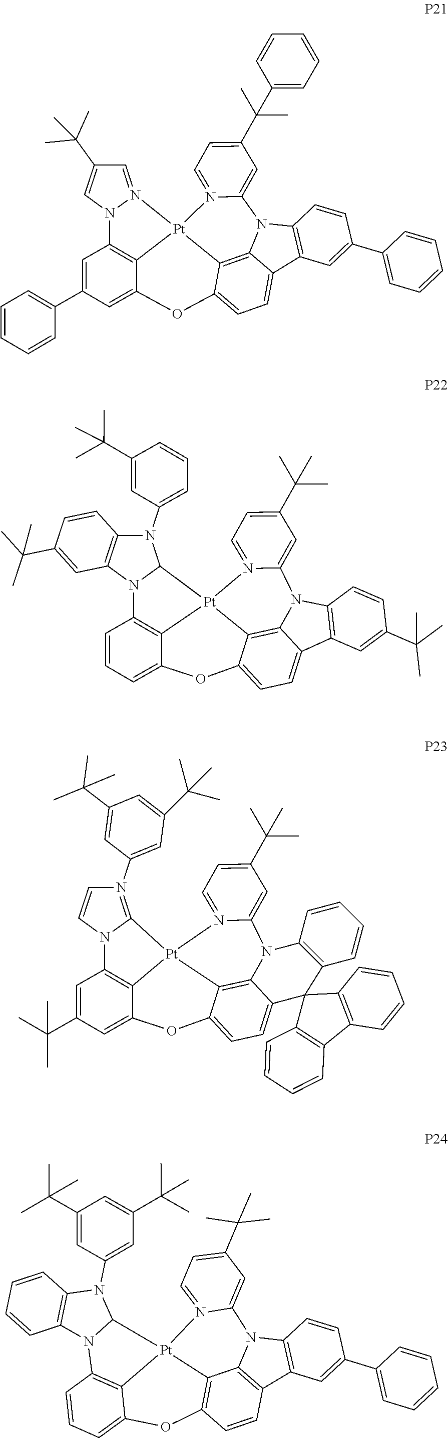

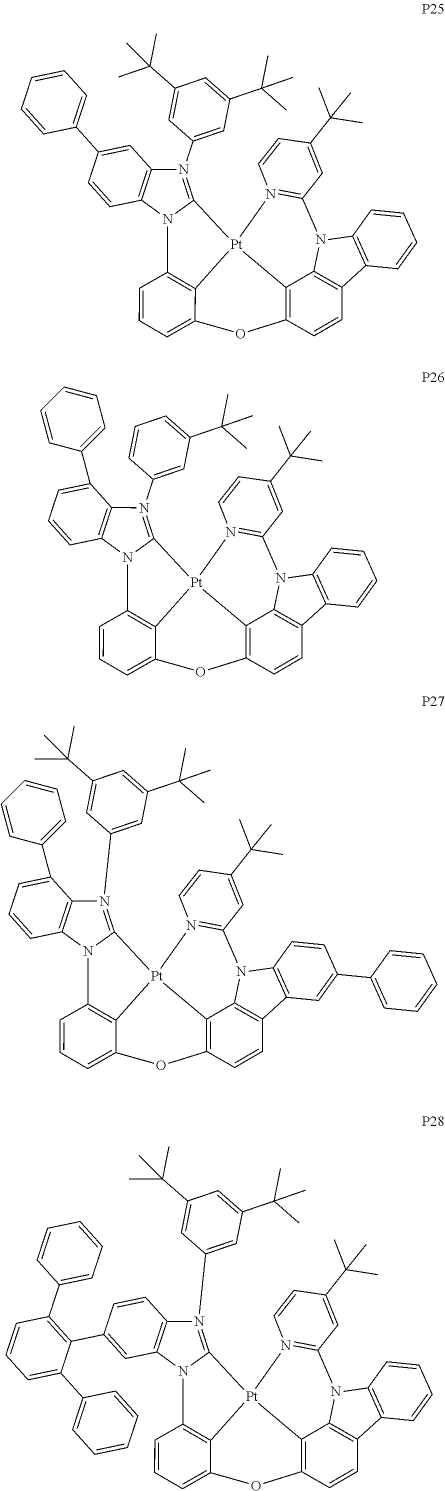

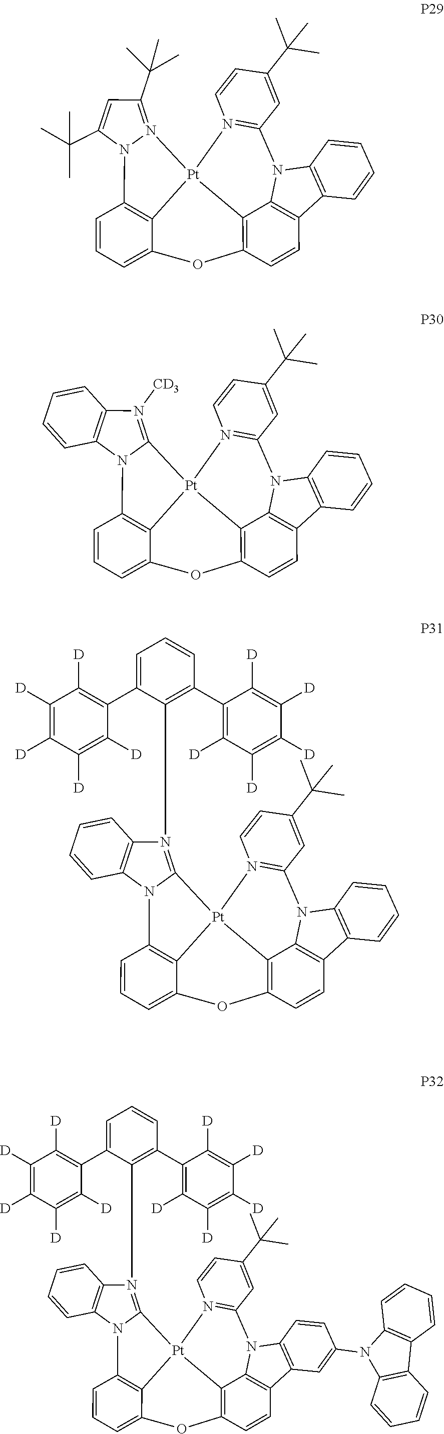

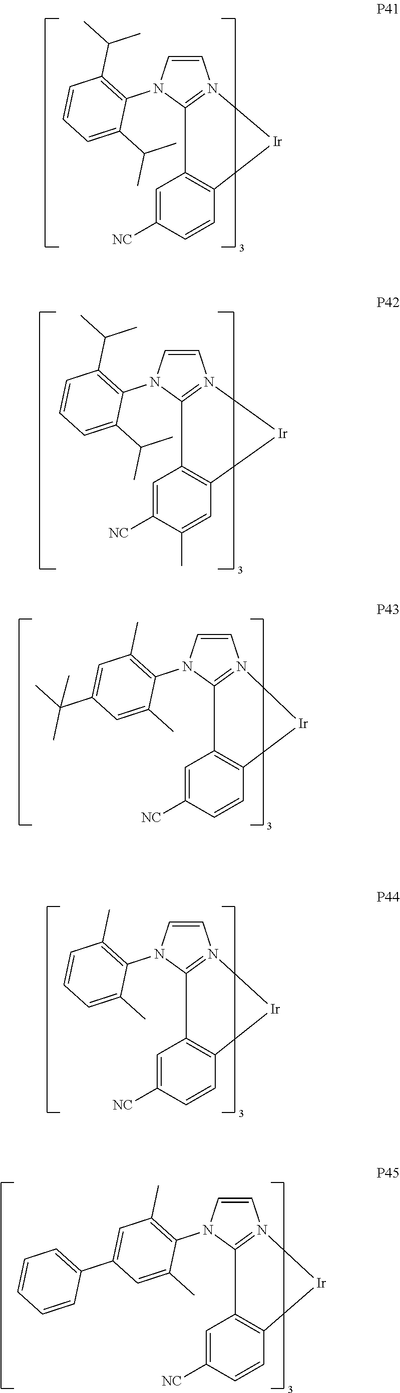

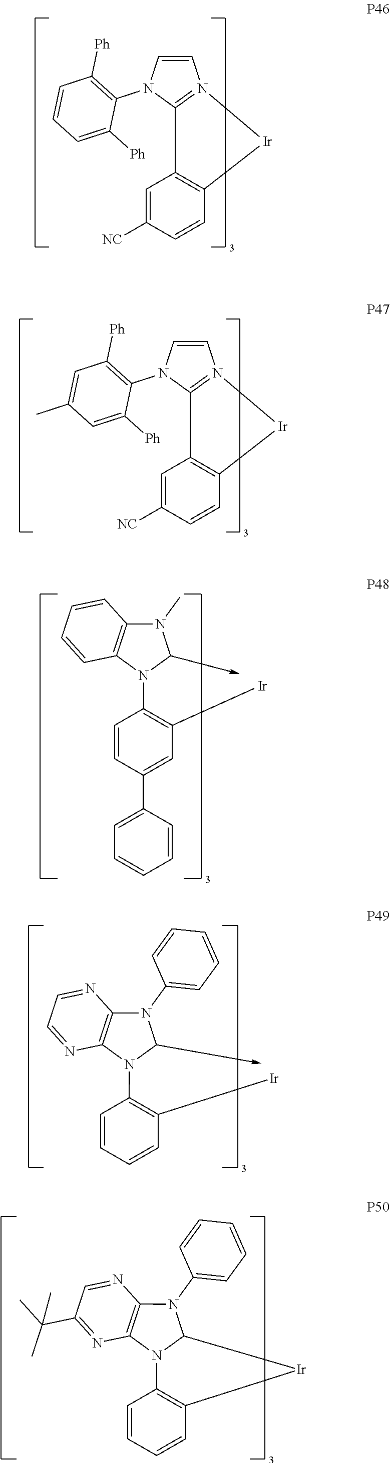

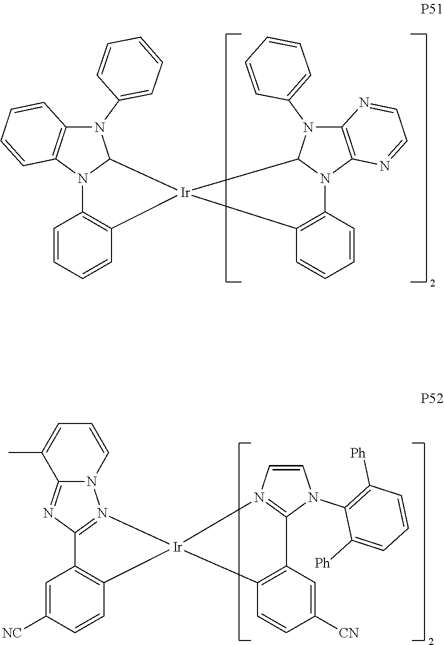

The delayed fluorescent dopant may be a metal atom-free compound with a ΔEST of less than or equal to 0.2 eV, and because the up-conversion process by reverse intersystem crossing (RISC) may be advantageous, the efficiency of a light emitting diode including it may be improved.

The delayed fluorescent dopant may be, e.g., one of Compounds T1 to T12.

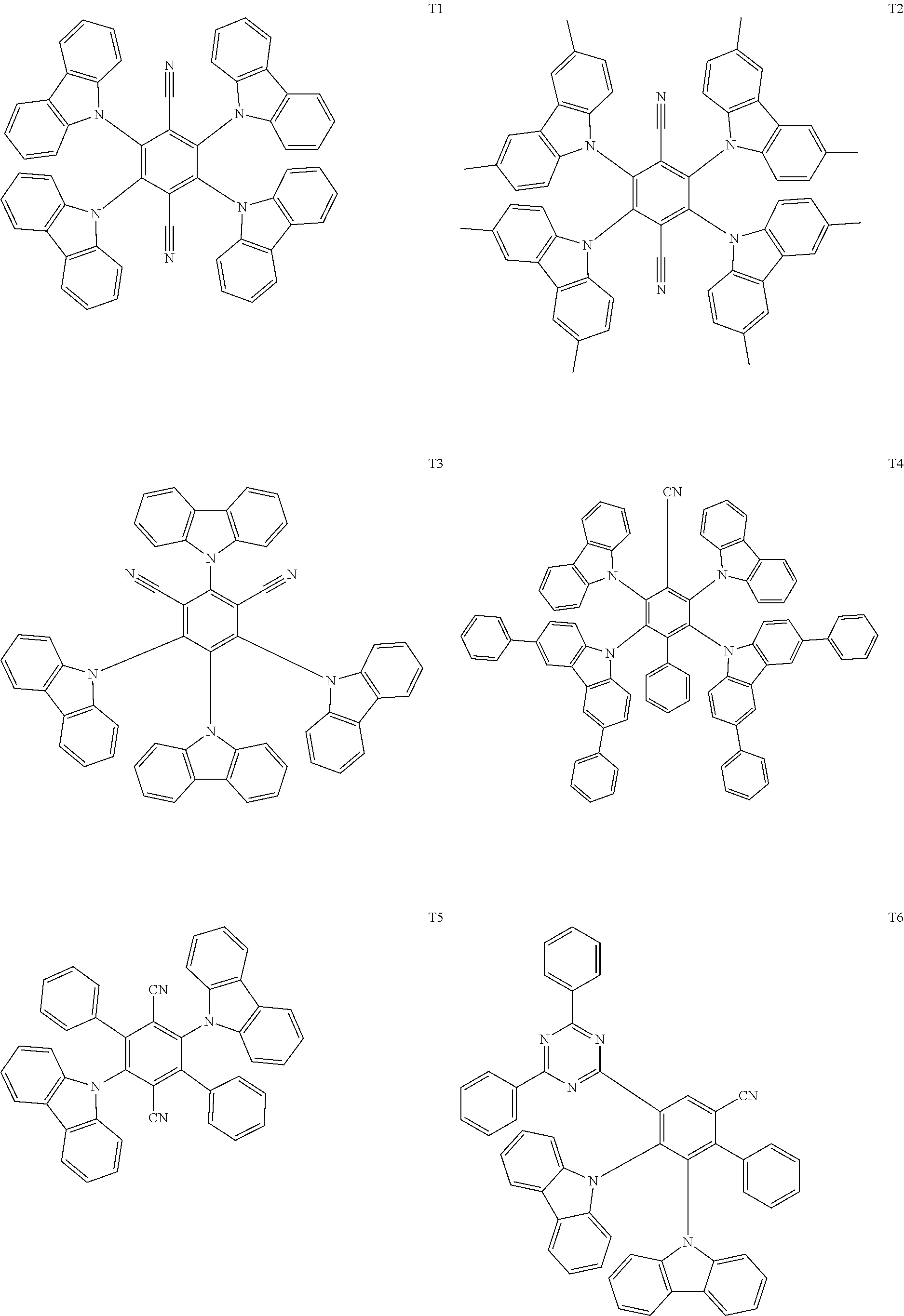

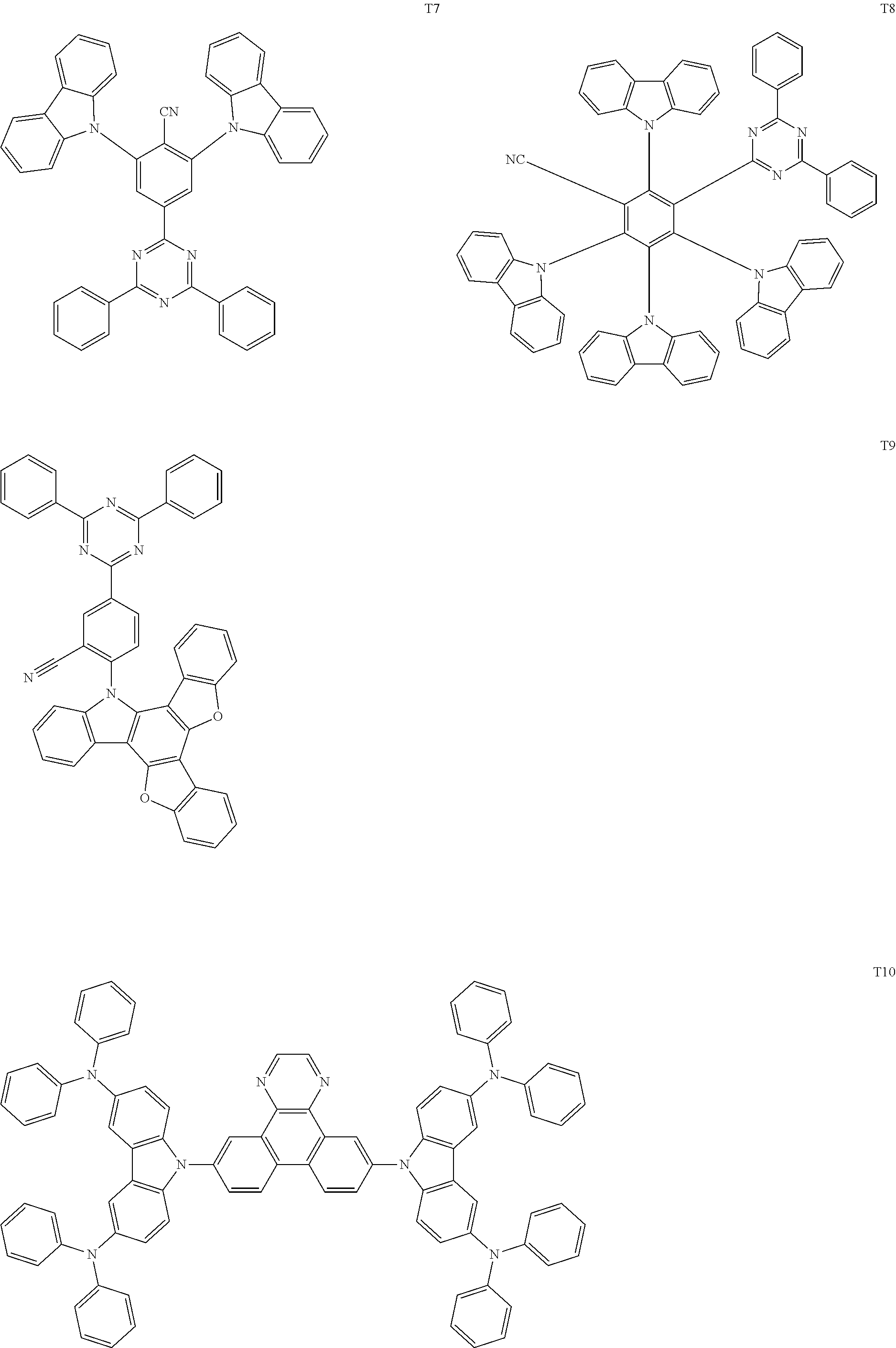 [SMILES: N#Cc1c(-n2c3ccccc3c3ccccc32)cc(-c2nc(-c3ccccc3)nc(-c3ccccc3)n2)cc1-n1c2ccccc2c2ccccc21 ; N#Cc1c(-n2c3ccccc3c3ccccc32)c(-c2nc(-c3ccccc3)nc(-c3ccccc3)n2)c(-n2c3ccccc3c3ccccc32)c(-n2c3ccccc3c3ccccc32)c1-n1c2ccccc2c2ccccc21 ; N#Cc1cc(-c2nc(-c3ccccc3)nc(-c3ccccc3)n2)ccc1-n1c2ccccc2c2c3oc4ccccc4c3c3oc4ccccc4c3c21 ; c1ccc(N(c2ccccc2)c2ccc3c(c2)c2cc(N(c4ccccc4)c4ccccc4)ccc2n3-c2ccc3c4ccc(-n5c6ccc(N(c7ccccc7)c7ccccc7)cc6c6cc(N(c7ccccc7)c7ccccc7)ccc65)cc4c4nccnc4c3c2)cc1]

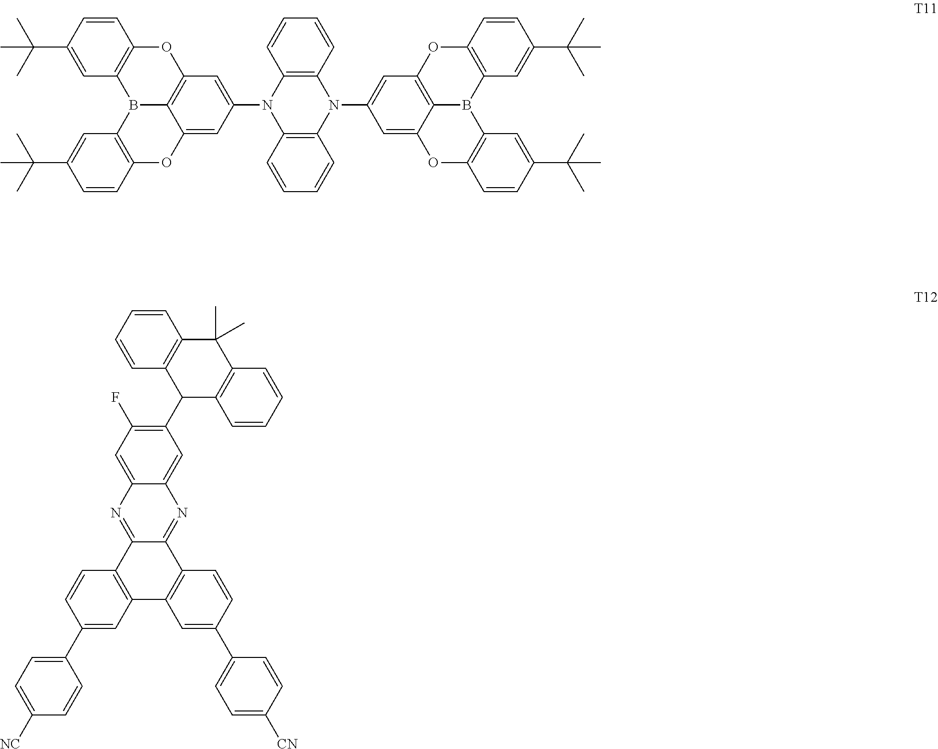 [SMILES: CC(C)(C)c1ccc2c(c1)B1c3cc(C(C)(C)C)ccc3Oc3cc(N4c5ccccc5N(c5cc6c7c(c5)Oc5ccc(C(C)(C)C)cc5B7c5cc(C(C)(C)C)ccc5O6)c5ccccc54)cc(c31)O2 ; CC1(C)c2ccccc2C(c2cc3nc4c5ccc(-c6ccc(C#N)cc6)cc5c5cc(-c6ccc(C#N)cc6)ccc5c4nc3cc2F)c2ccccc21]

The fluorescent dopant may be, e.g., a polycyclic compound, and may improve luminescence efficiency and life-span characteristics of the light emitting diode by receiving energy transfer within the light emitting layer due to high absorbance.

The fluorescent dopant may be a condensed polycyclic compound containing, e.g., boron (B), nitrogen (N) or a combination thereof, and may be, e.g., one of Compounds D1 to D38.

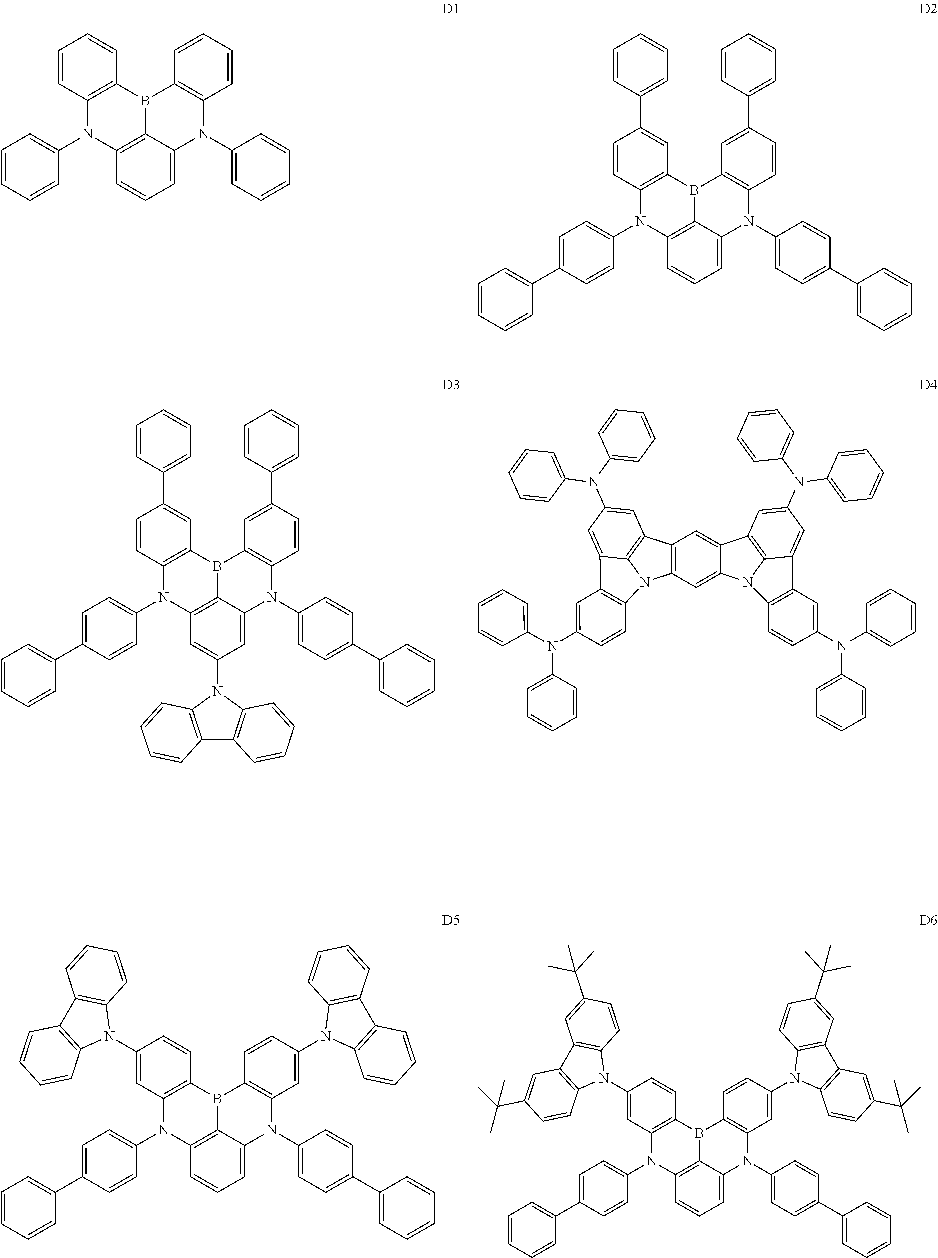

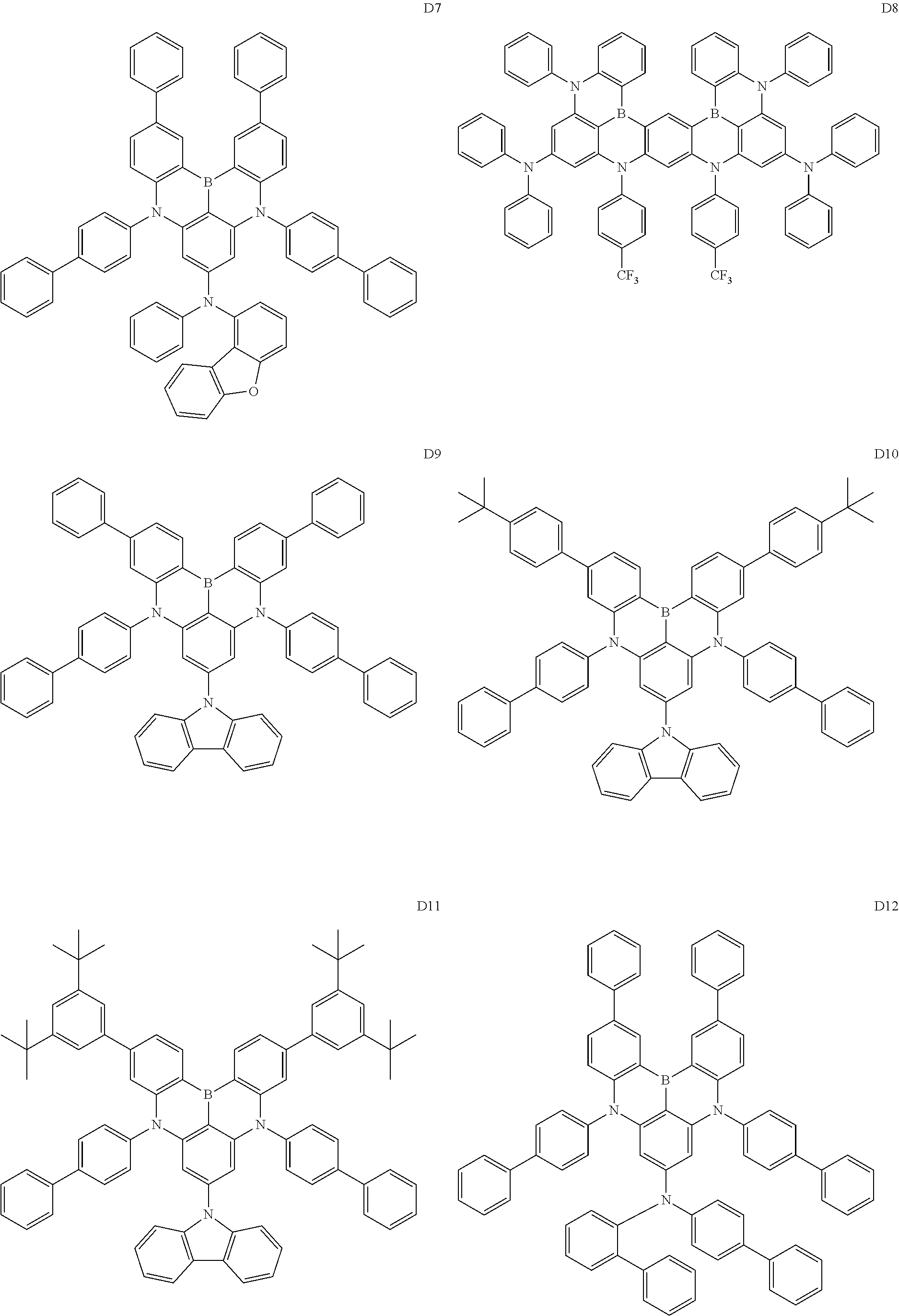

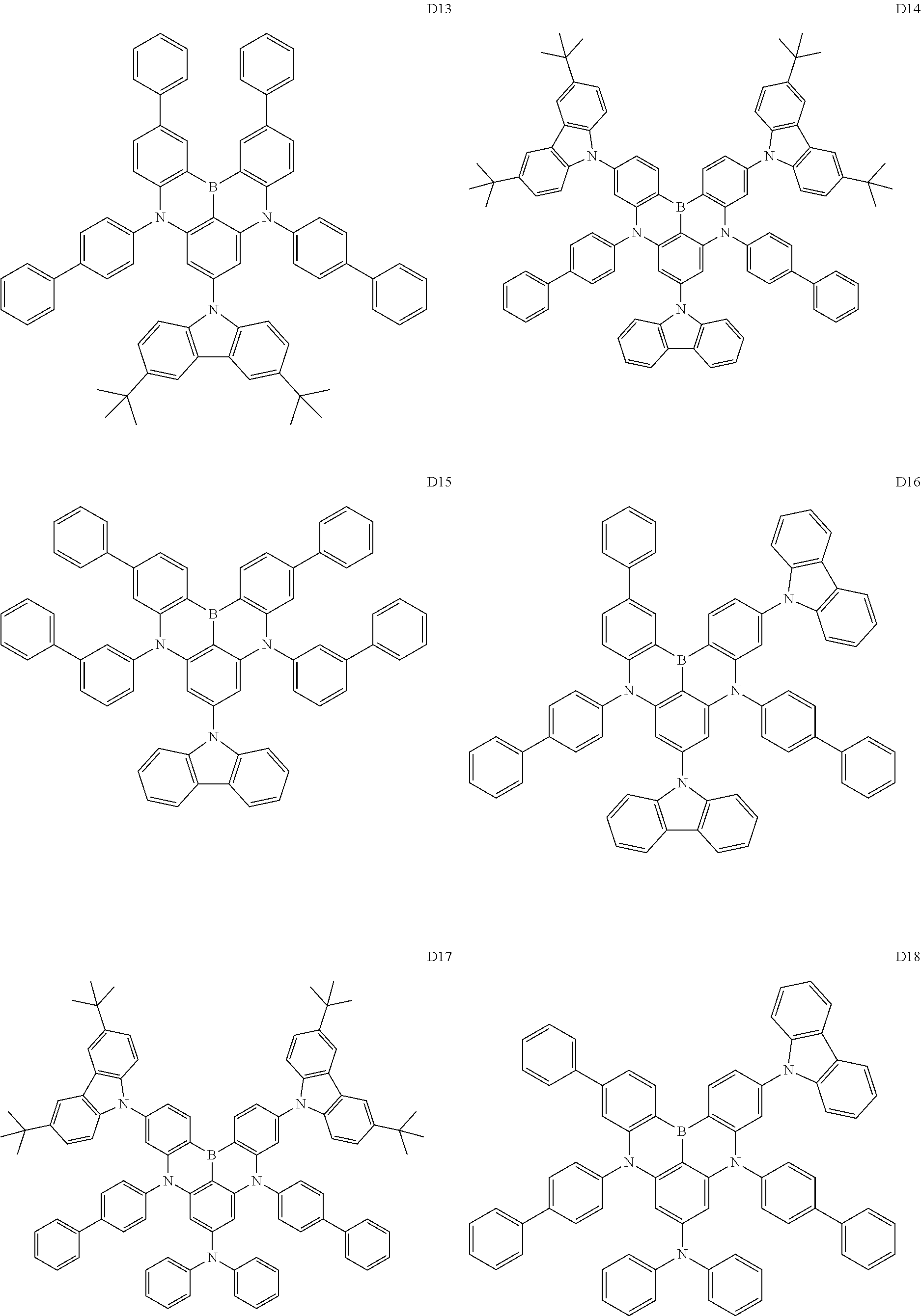

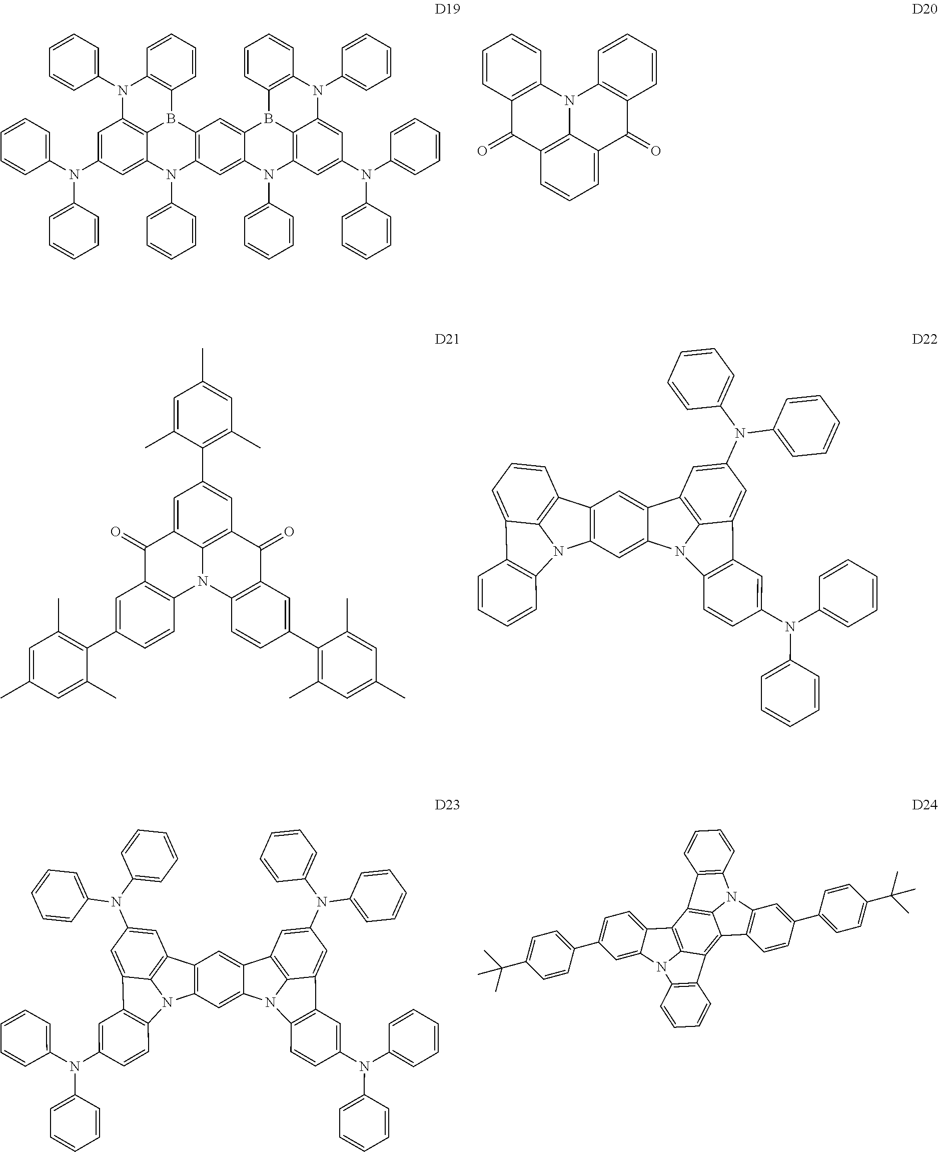

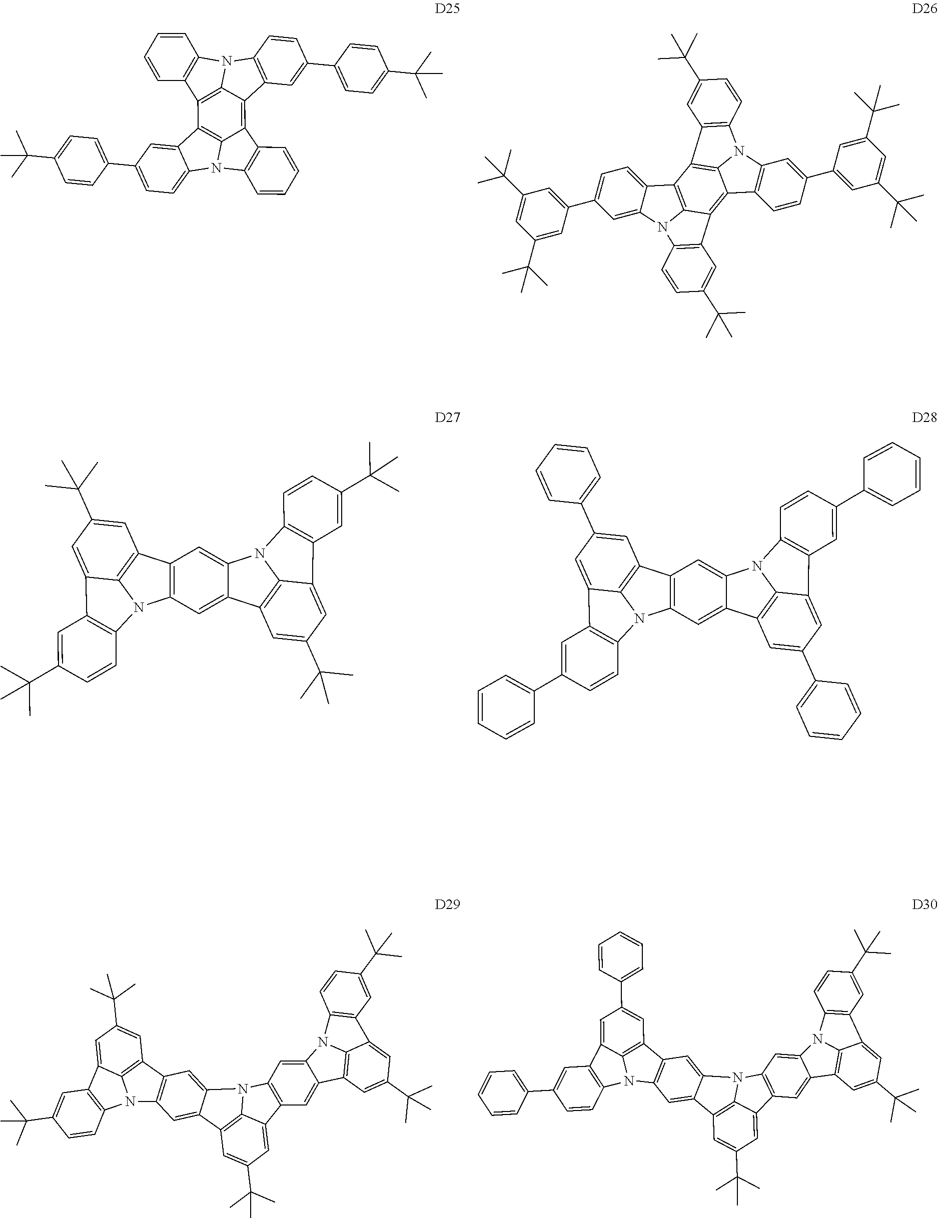

The phosphorescent dopant, the delayed fluorescent dopant, or the fluorescent dopant may be included in an amount of, e.g., less than or equal to about 20 wt %, respectively, and within the above range, may be included in an amount of, e.g., about 0.1 to about 20 wt %, about 0.1 to about 15 wt %, about 0.1 to about 10 wt %, about 0.1 to about 7 wt %, about 0.1 to about 5 wt %, about 0.1 to about 4 wt %, about 1 to about 20 wt %, about 1 to about 15 wt %, about 1 to about 10 wt %, about 1 to about 7 wt %, about 1 to about 5 wt % or about 1 to about 4 wt %, respectively, based on the total amount of the composition for an organic optoelectronic device.

The composition for an organic optoelectronic device may additionally include an additive, and the additive may be, e.g., an organic material, an inorganic material, an organic/inorganic material, or a combination thereof

Hereinafter, an organic optoelectronic device using the aforementioned compound for the organic optoelectronic device or composition for the organic optoelectronic device will be described.

The organic optoelectronic device is not particularly limited as long as it is a device that may convert electrical energy and optical energy into each other, and examples thereof may include an organic light emitting diode, an organic photoelectric device, an organic solar cell, or an organic photo-conductor drum.

The organic optoelectronic device may include, e.g., an anode and a cathode facing each other, and an organic layer between the anode and the cathode, and the organic layer may include the aforementioned compound or composition. The organic layer may include, e.g., an active layer, such as a light emitting layer or a light absorbing layer, and the aforementioned compound or composition may be included in the active layer. The organic layer may include, e.g., an auxiliary layer between the anode and the active layer or between the cathode and the active layer, and the aforementioned compound or composition may be included in the auxiliary layer.

Here, an organic light emitting diode, which is an example of an organic optoelectronic device, is described as an example, but it may be equally applied to other organic optoelectronic devices.

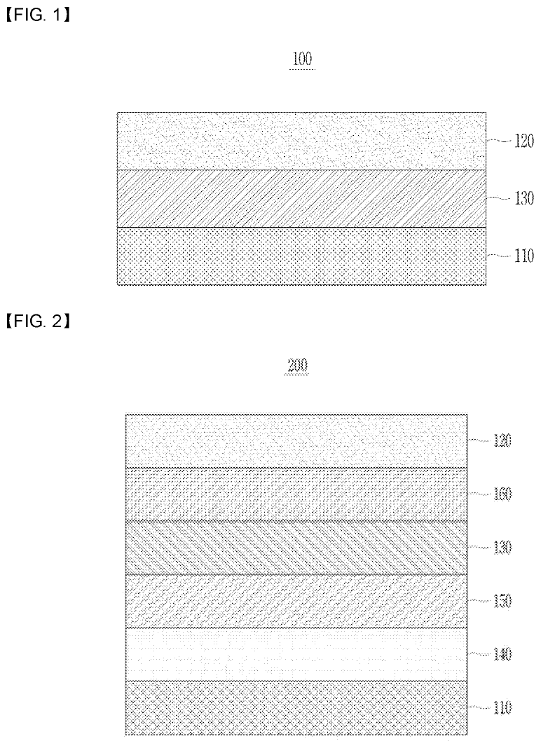

FIG. 1 is a cross-sectional view showing an example of an organic light emitting diode, which is an example of an organic optoelectronic device according to some example embodiments.

Referring to FIG. 1, an organic light emitting diode 100 according to some example embodiments may include, e.g., an anode 110, a cathode 120 facing each other, and a light emitting layer 130 between the anode 110 and the cathode 120.

The anode 110 may be made of a conductor with a high work function to facilitate hole injection and may be, e.g., made of a metal, a metal oxide, or a conductive polymer. The anode 110 may be, e.g., made of a metal such as nickel, platinum, vanadium, chromium, copper, zinc, gold, or an alloy thereof, a metal oxide such as zinc oxide, indium oxide, indium tin oxide (ITO), or indium zinc oxide (IZO); a combination of a metal and an oxide such as ZnO and Al or SnO₂ and Sb; or a conductive polymer such as poly(3-methylthiophene), poly(3,4-(ethylene-1,2-dioxy)thiophene) (PEDOT), polypyrrole, or polyaniline.

The cathode 120 may be, e.g., made of a conductor with a low work function to help facilitate electron injection, e.g., and may be made of a metal, a metal oxide, or a conductive polymer. The cathode 120 may be made of a metal such as magnesium, calcium, sodium, potassium, titanium, indium, yttrium, lithium, gadolinium, aluminum, silver, tin, lead, cesium, barium, or an alloy thereof, or a multilayer structure material such as LiF/Al, LiO₂/Al, LiF/Ca, or BaF₂/Ca.

The light emitting layer 130 may include the aforementioned compound for the organic optoelectronic device or composition for the organic optoelectronic device. The light emitting layer 130 may further include another organic compound as a mixed host. The light emitting layer 130 may further include the aforementioned luminescent dopant and may include, e.g., a fluorescent dopant, a phosphorescent photosensitizer, or a combination thereof as described above.

In an implementation, the light emitting layer 130 may emit light in the blue emission spectrum by combining the aforementioned compound for the organic optoelectronic device or composition for the organic optoelectronic device and a luminescent dopant. The peak wavelength of the blue emission spectrum may be, e.g., between about 410 nm and 480 nm, and within that range may be, e.g., between about 420 nm and 470 nm or between about 430 nm and 470 nm. In an implementation, at least one of the first compound and the aforementioned second compound of the composition for an organic optoelectronic device may have a higher triplet energy level, thereby helping facilitate exciton transfer to a blue luminescent dopant, thereby helping enable implementation of a high-efficiency, long life-span organic optoelectronic device.

The aforementioned compound for the organic optoelectronic device may increase the stability of the material during device operation and improve the life-span and driving voltage by helping transfer the LUMO phore (LUMO part of the molecule) to an additionally connected phenyl group.

In an implementation, the aforementioned composition for the organic optoelectronic device may help further improve life-span by helping further increase the balance of electrons and holes within the light emitting layer 130 and help further increase luminous efficiency by including the first compound, which may be a bipolar compound having relatively strong hole transport characteristics, and the second compound, which may be a bipolar compound having relatively strong electron transport characteristics, while reducing the unbound charge due to the imbalance in the mobility of electrons and holes.

In an implementation, an organic light emitting diode 100 including a light emitting layer 130 in which an organic optoelectronic device composition may be applied as a mixed host may help enhance luminous efficiency in the light emitting layer 130 by helping finely control holes and electrons injected from the anode 110 and cathode 120 respectively to have appropriate mobility within the light emitting layer 130, thereby strongly inducing exciton generation within the light emitting layer 130.

In an implementation, a difference in mobility of holes and electrons injected from the anode 110 and cathode 120 respectively within the light emitting layer 130 may help reduce or prevent the generation of excitons at inappropriate locations, such as the interface between the light emitting layer 130 and adjacent layers, or the accumulation of unbound charges at the interface between the light emitting layer 130 and adjacent layers. Accordingly, the roll-off phenomenon in which the luminous efficiency of the organic light emitting diode 100 rapidly decreases due to non-luminescent excitons or non-bound charges may be reduced or prevented, and thus the life-span of the organic light emitting diode 100 may ultimately be improved.

The organic light emitting diode 100 may be manufactured by forming an anode 110 or a cathode 120 on a substrate, forming a light emitting layer 130 using dry film forming methods such as vacuum evaporation, sputtering, plasma plating, and ion plating, and forming a cathode 120 or an anode 110 thereon.

FIG. 2 is a cross-sectional view showing another example of an organic light emitting diode, which is an example of an organic optoelectronic device according to some example embodiments.

Referring to FIG. 2, the organic optoelectronic device 200 according to the present embodiments may include, e.g., an anode 110, a cathode 120, and a light emitting layer 130, similar to the aforementioned embodiments. However, unlike the aforementioned embodiments, the organic optoelectronic device 200 according to the embodiment illustrated in FIG. 2 further includes, e.g., a hole transport layer 140, a hole transport auxiliary layer 150, and an electron transport layer 160.

The hole transport layer 140 may be, e.g., between the anode 110 and the light emitting layer 130, and the hole transport auxiliary layer 150 may be, e.g., between the light emitting layer 130 and the hole transport layer 140. The electron transport layer 160 may be, e.g., located between the cathode 120 and the light emitting layer 130.

The hole transport layer 140 may help facilitate hole transfer from the anode 110 to the light emitting layer 130, and may include, e.g., an amine compound. In an implementation, the amine compound may have at least one aryl group or heteroaryl group with hole characteristics. In an implementation, the amine compound may be represented by Chemical Formula 6A or 6B.

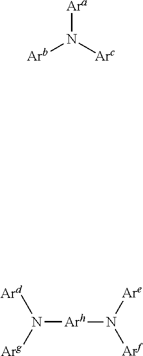

In Chemical Formula 6A or 6B, Ar^{a} to Ar^{g} may each independently be, e.g., hydrogen, deuterium, a substituted or unsubstituted C1 to C20 alkyl group, a substituted or unsubstituted C6 to C30 aryl group, a substituted or unsubstituted C2 to C30 heteroaryl group, or a combination thereof.

At least one of Ar^{a} to Ar^{c} and at least one of Ar^{d} to Ar^{g} may be, e.g., a substituted or unsubstituted C6 to C30 aryl group, a substituted or unsubstituted C2 to C30 heteroaryl group, or a combination thereof.

Ar^{h} may be, e.g., a single bond, a substituted or unsubstituted C1 to C20 alkylene group, a substituted or unsubstituted C6 to C30 arylene group, a substituted or unsubstituted C2 to C30 heteroarylene group, or a combination thereof.

The hole transport auxiliary layer 150 may form an interface with the light emitting layer 130 by being between the hole transport layer 140 and the light emitting layer 130 and in contact with the light emitting layer 130. The hole transport auxiliary layer 150 may help further reduce or prevent exciton generation at inappropriate locations, such as the interface between the aforementioned light emitting layer 130 and adjacent layers, or accumulation of unbound charges at the interface between the light emitting layer 130 and adjacent layers. Accordingly, the roll-off phenomenon in which the luminous efficiency of the organic light emitting diode 100 rapidly decreases due to non-luminescent excitons or unbound charges may be further reduced or prevented, and thus the life-span of the organic light emitting diode 100 may be further improved.

The electron transport layer 160 may help further increase electron injection or electron mobility and block holes between the cathode 120 and the light emitting layer 130.

The electron transport layer 160 may include, e.g., one or more compounds of Group 3.

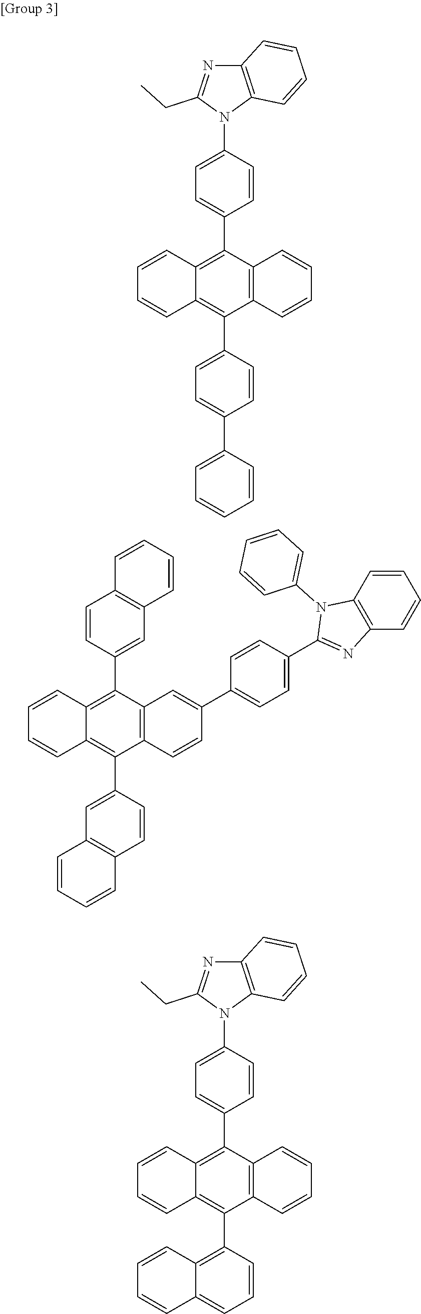

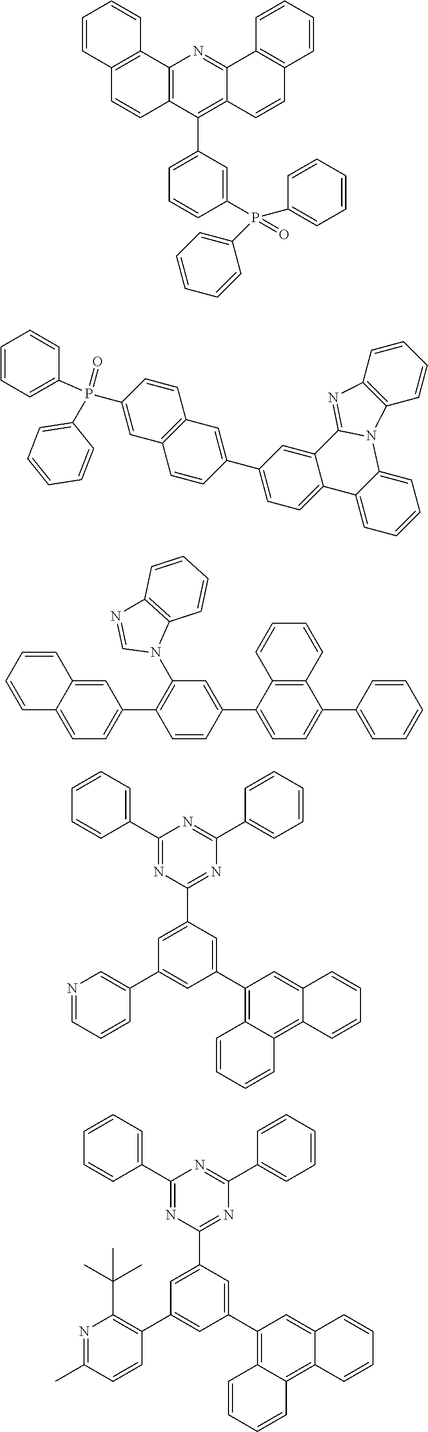

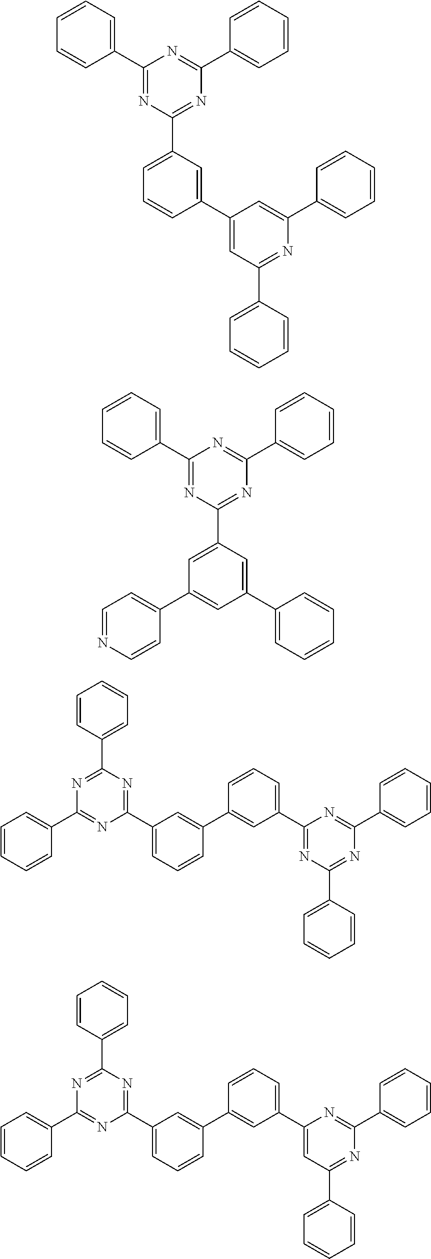

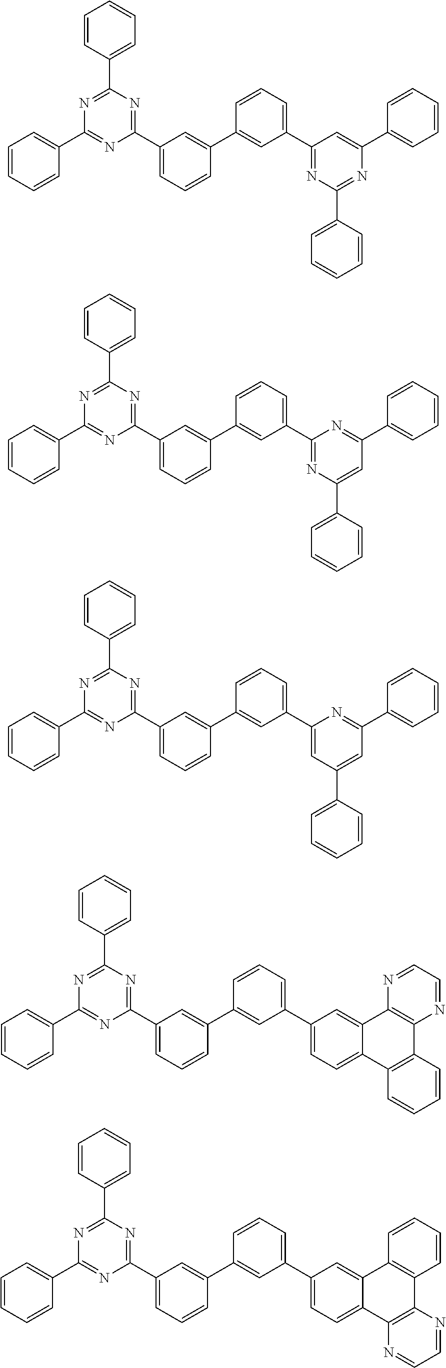 [SMILES: c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

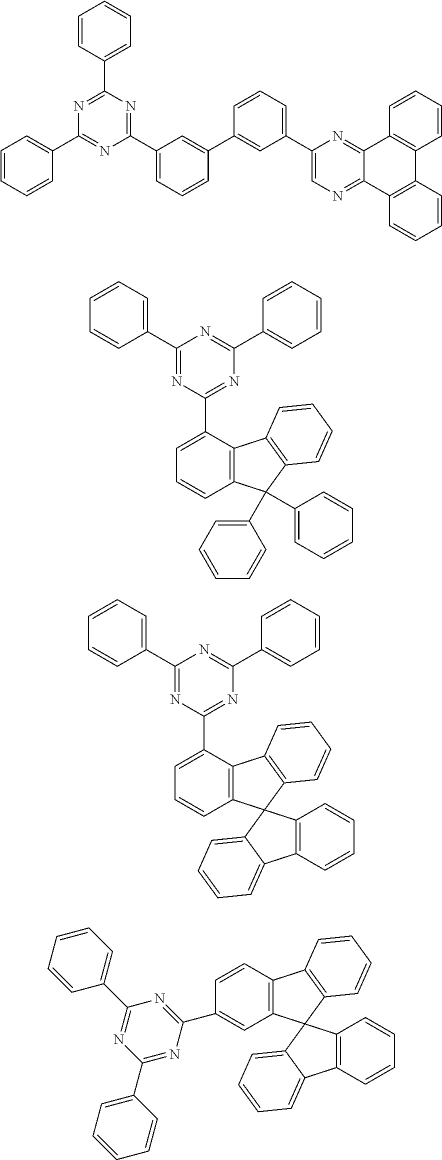

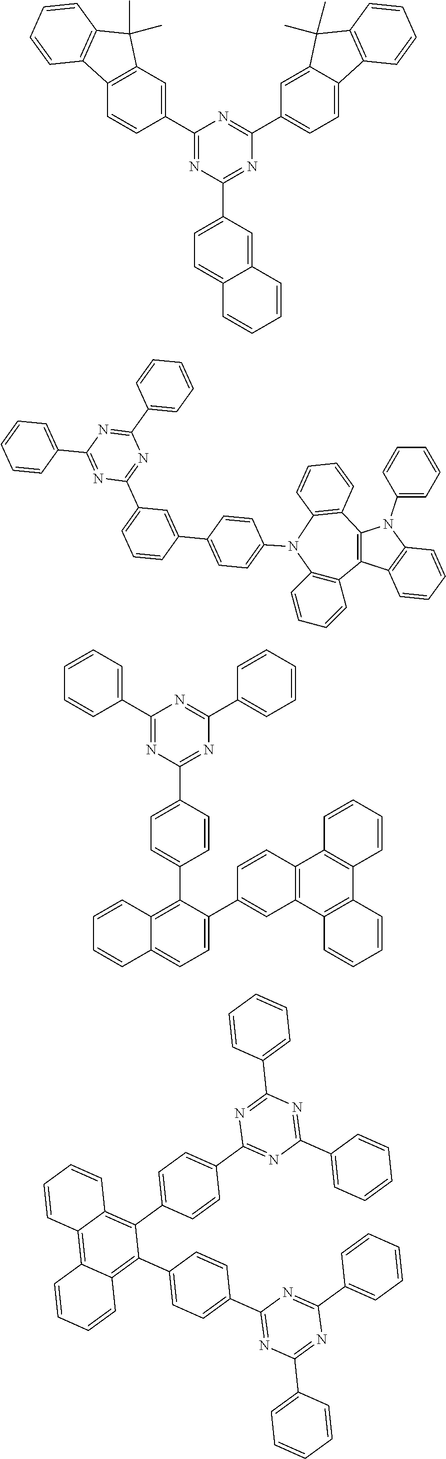

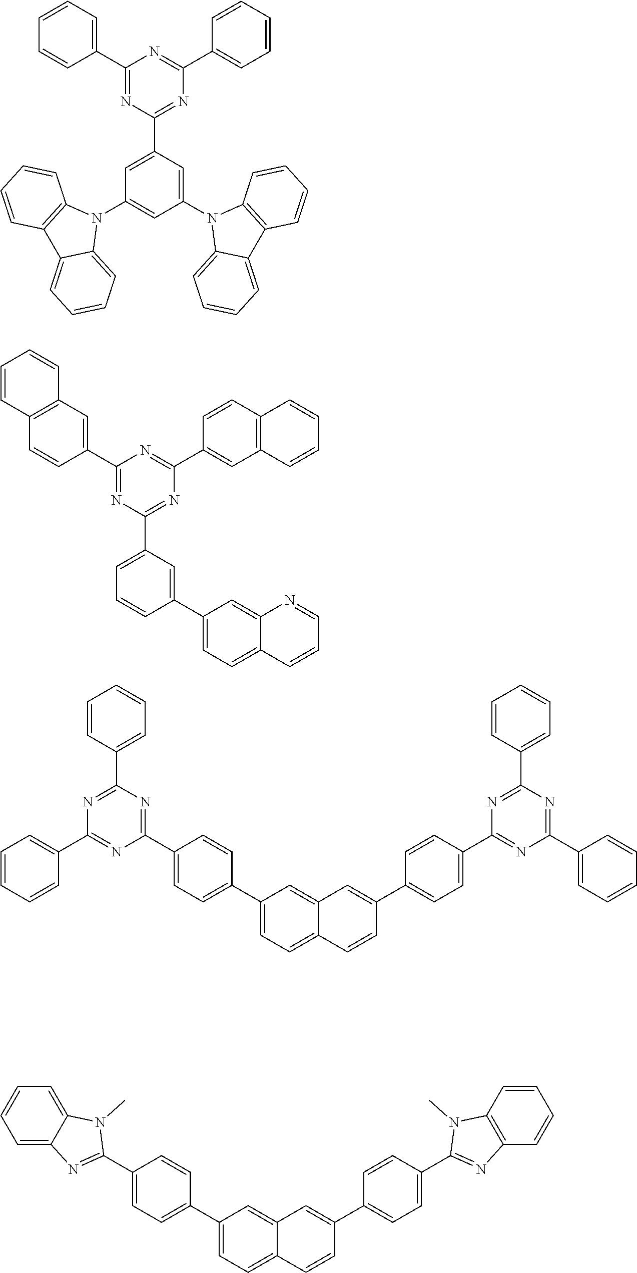 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2ccc3cccnc3c2)cc(-c2nc(-c3ccc4ccccc4c3)nc(-c3ccc4ccccc4c3)n2)c1 ; c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1 ; Cn1c(-c2ccc(-c3ccc4ccc(-c5ccc(-c6nc7ccccc7n6C)cc5)cc4c3)cc2)nc2ccccc21]

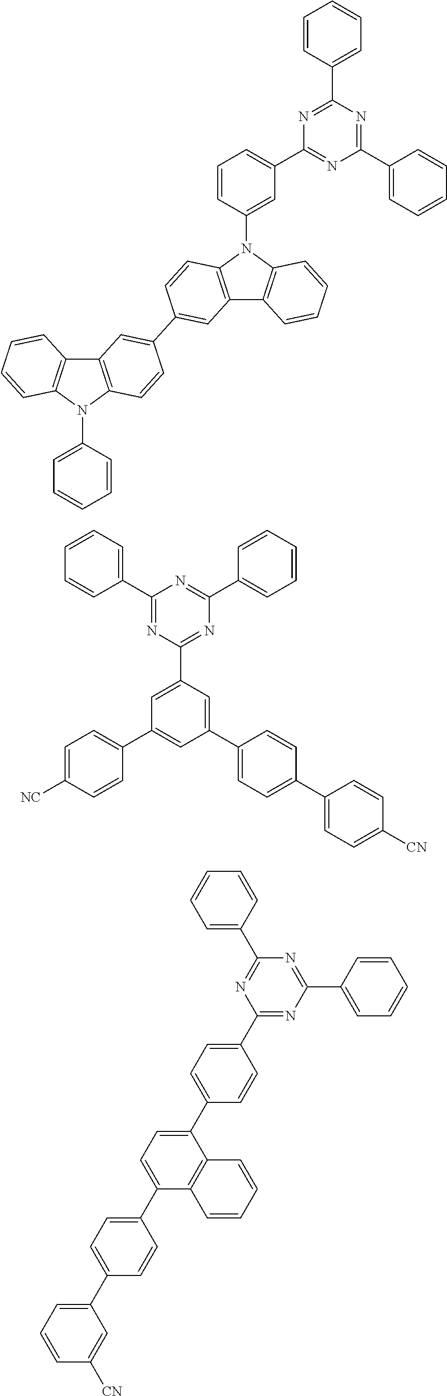

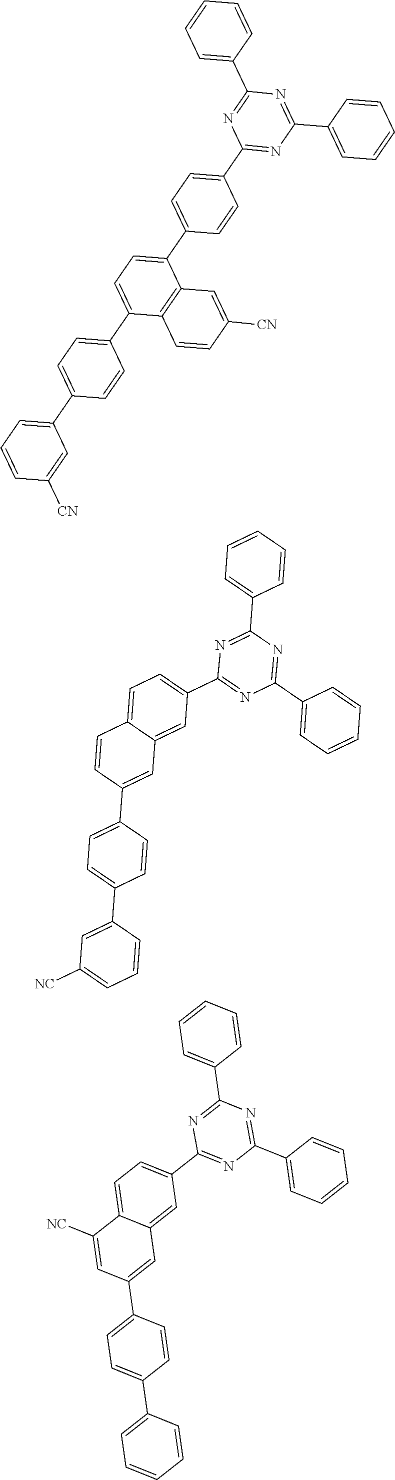 [SMILES: N#Cc1cccc(-c2ccc(-c3ccc(-c4ccc(-c5nc(-c6ccccc6)nc(-c6ccccc6)n5)cc4)c4cc(C#N)ccc34)cc2)c1 ; N#Cc1cccc(-c2ccc(-c3ccc4ccc(-c5nc(-c6ccccc6)nc(-c6ccccc6)n5)cc4c3)cc2)c1 ; N#Cc1cc(-c2ccc(-c3ccccc3)cc2)cc2cc(-c3nc(-c4ccccc4)nc(-c4ccccc4)n3)ccc12]

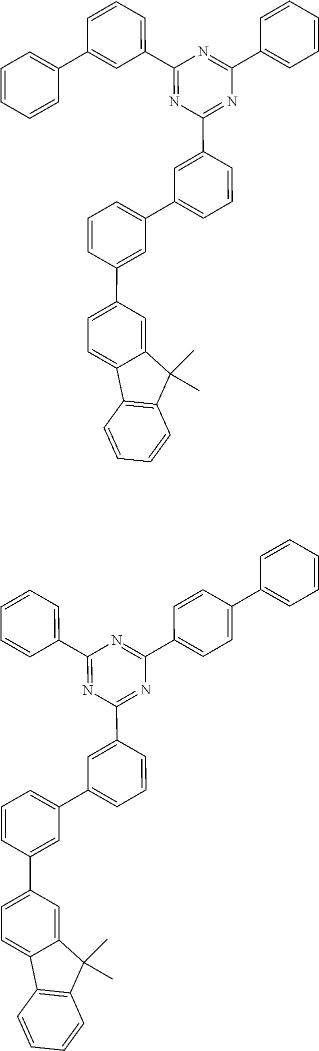 [SMILES: CC1(C)c2ccccc2-c2ccc(-c3cccc(-c4cccc(-c5nc(-c6ccccc6)nc(-c6cccc(-c7ccccc7)c6)n5)c4)c3)cc21 ; CC1(C)c2ccccc2-c2ccc(-c3cccc(-c4cccc(-c5nc(-c6ccccc6)nc(-c6ccc(-c7ccccc7)cc6)n5)c4)c3)cc21]

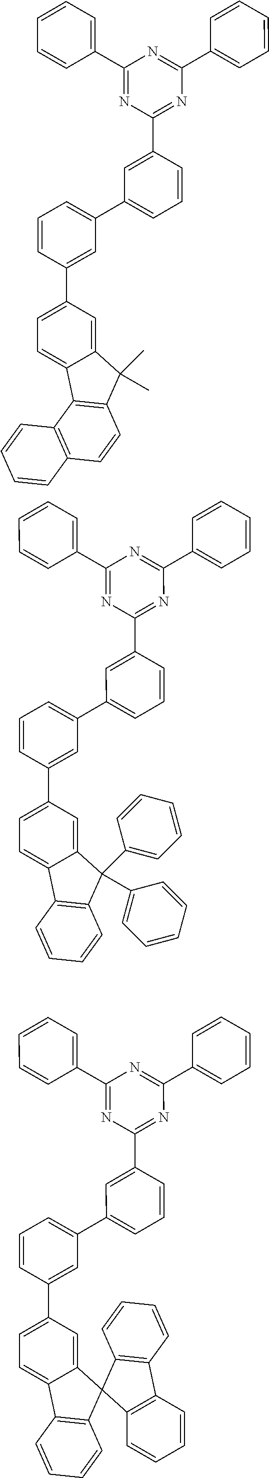

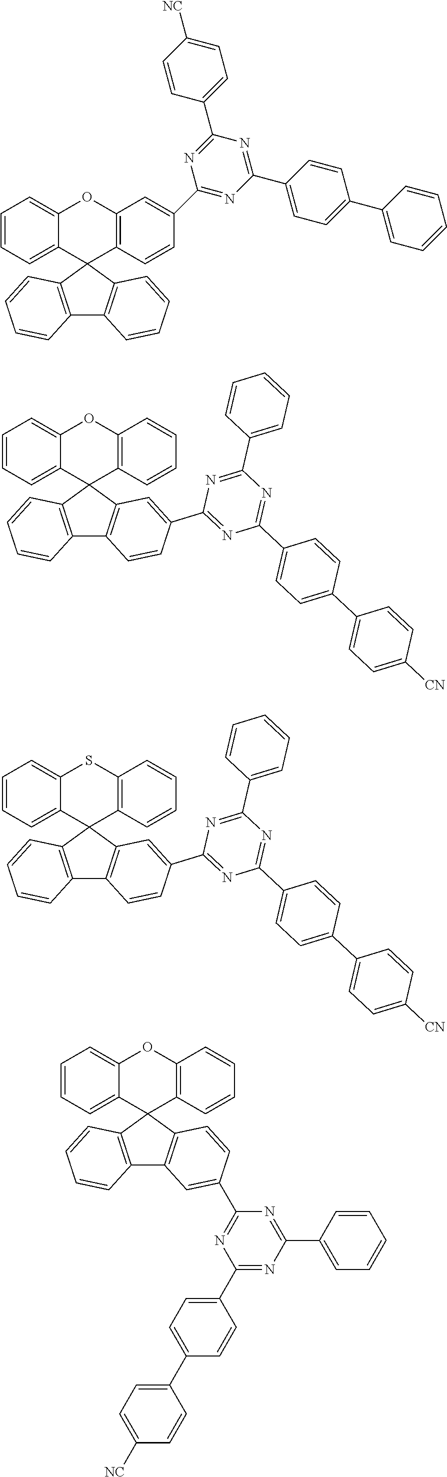 [SMILES: N#Cc1ccc(-c2nc(-c3ccc(-c4ccccc4)cc3)nc(-c3ccc4c(c3)Oc3ccccc3C43c4ccccc4-c4ccccc43)n2)cc1 ; N#Cc1ccc(-c2ccc(-c3nc(-c4ccccc4)nc(-c4ccc5c(c4)C4(c6ccccc6Oc6ccccc64)c4ccccc4-5)n3)cc2)cc1 ; N#Cc1ccc(-c2ccc(-c3nc(-c4ccccc4)nc(-c4ccc5c(c4)C4(c6ccccc6Sc6ccccc64)c4ccccc4-5)n3)cc2)cc1 ; N#Cc1ccc(-c2ccc(-c3nc(-c4ccccc4)nc(-c4ccc5c(c4)-c4ccccc4C54c5ccccc5Oc5ccccc54)n3)cc2)cc1]

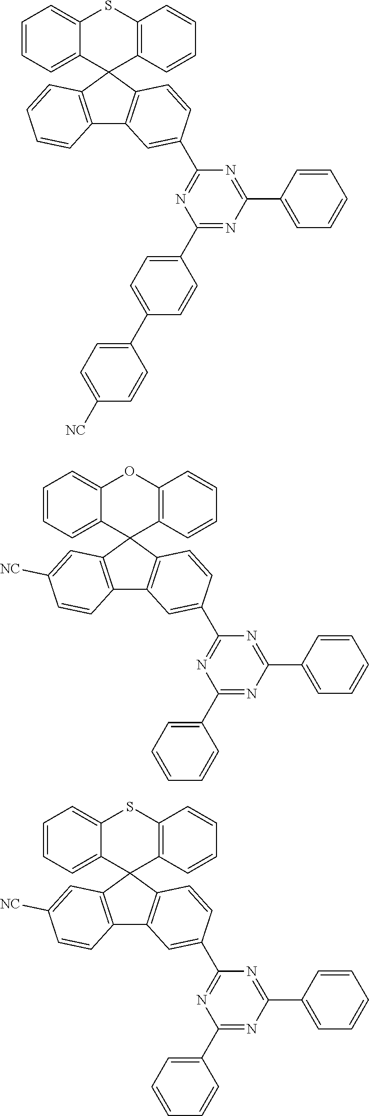

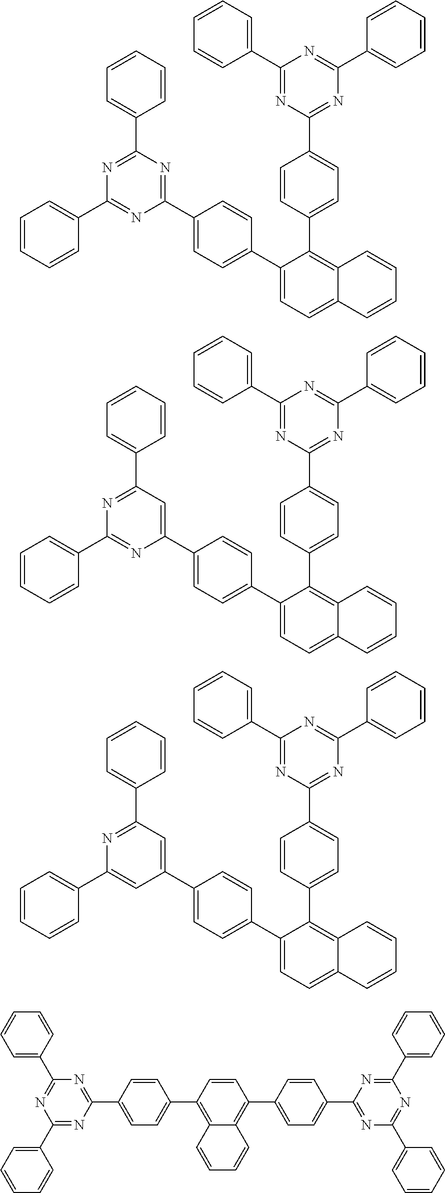

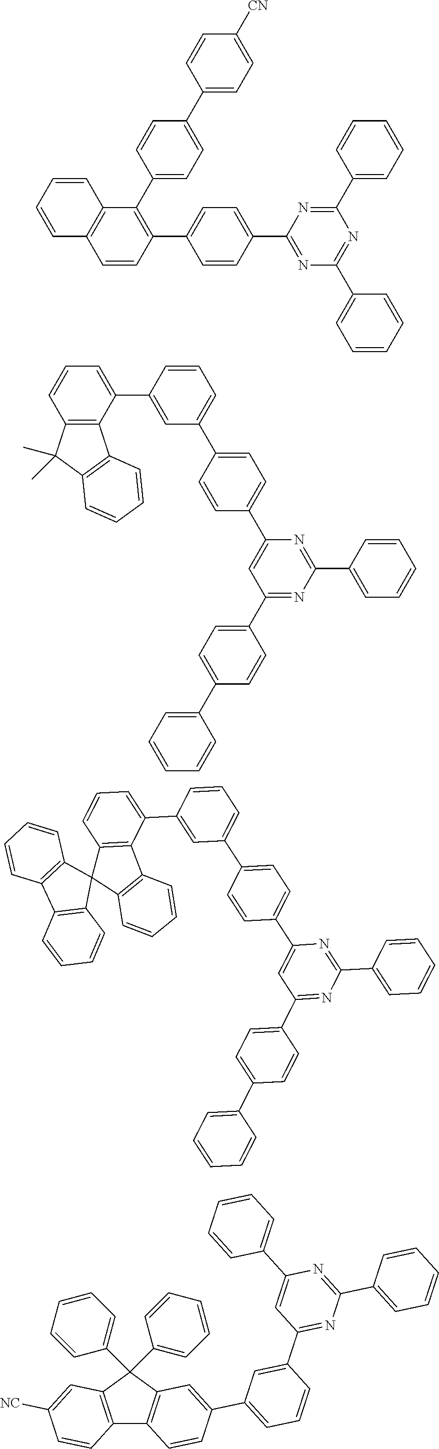

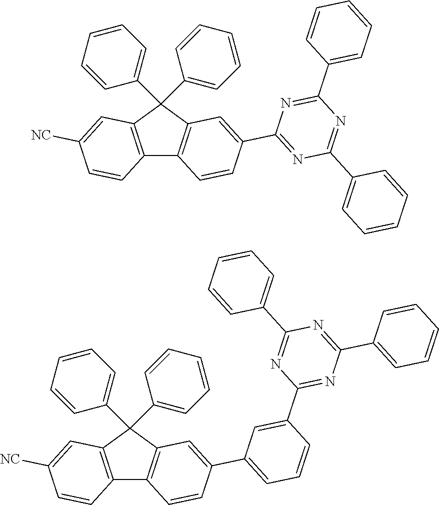 [SMILES: N#Cc1ccc2c(c1)C(c1ccccc1)(c1ccccc1)c1cc(-c3nc(-c4ccccc4)nc(-c4ccccc4)n3)ccc1-2 ; N#Cc1ccc2c(c1)C(c1ccccc1)(c1ccccc1)c1cc(-c3cccc(-c4nc(-c5ccccc5)nc(-c5ccccc5)n4)c3)ccc1-2]

The electron transport layer 160 may include, e.g., one or more compounds of Group 3.

The organic optoelectronic device, including the aforementioned organic light emitting diodes, can be applied to display devices.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in Examples and Synthesis Examples were purchased from Sigma-Aldrich Co. Ltd., TCI Inc., Tokyo chemical industry, or P&H tech as far as there is no particular comment or were synthesized by suitable methods.

### Synthesis of Compound for Organic Optoelectronic Device

### Synthesis Example 1: Synthesis of Compound 85

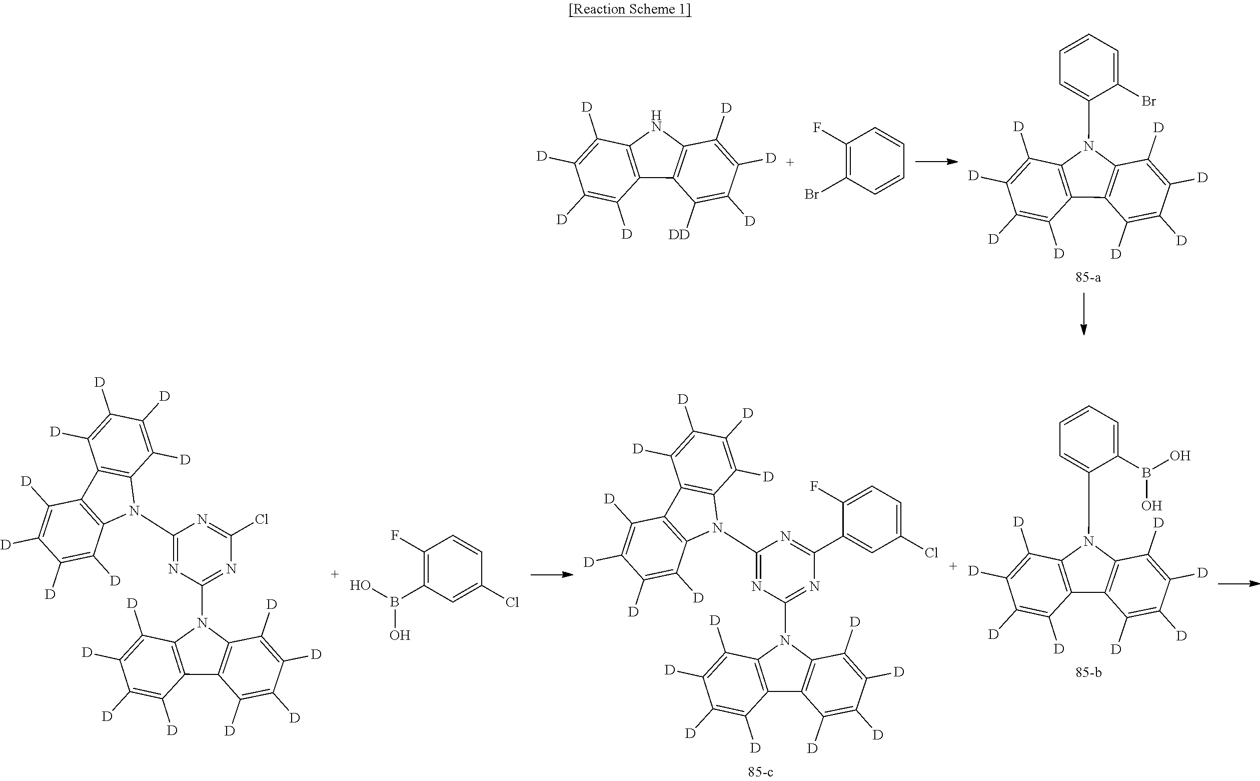

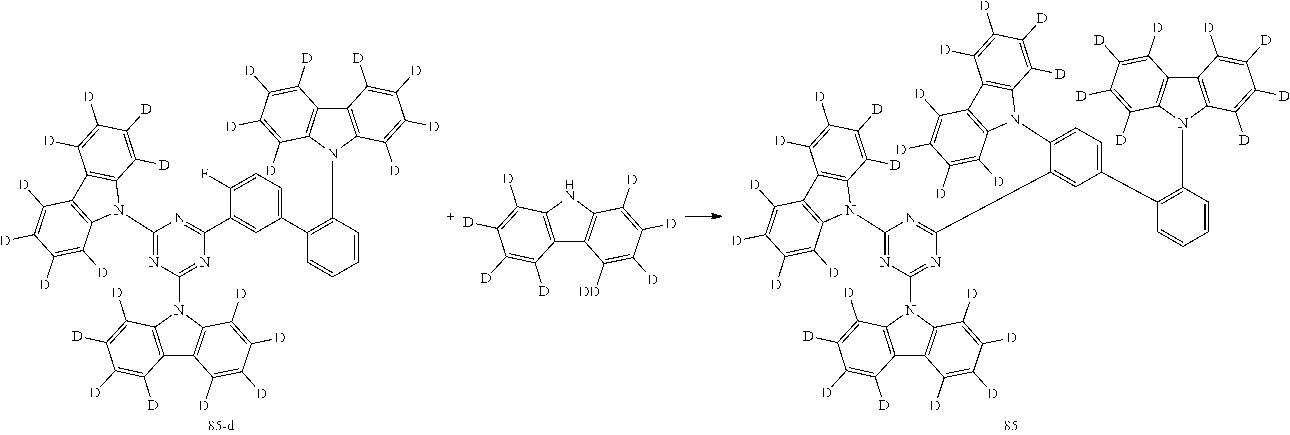

### First Step: Synthesis of Intermediate 85-a

9H-carbazole-d8 (26.7 g 152 mmol), 1-bromo-2-fluorobenzene (40 g, 229 mmol), potassium phosphate tribasic (64.7 g, 305 mmol), and N-methylpyrrolidone (760 ml) were added to a 2-neck 2,000 ml round-bottomed flask and heated and stirred at 160 °C for 18 hours in a nitrogen atmosphere. The obtained mixture was purified by column chromatography to obtain Intermediate 85-a (41.5 g, 126 mmol)

### Second Step: Synthesis of Intermediate 85-b

Intermediate 85-a (41.5 g, 126 mmol) and anhydrous tetrahydrofuran (420 ml) were added to a 1 L round-bottomed flask and cooled to −78 °C under nitrogen. 60 ml of 2.5 M N-butyl lithium solution in a dropwise fashion using a dropping funnel, stirred for 1 hour, then an additional 58 ml of triisopropyl borate was added in a dropwise fashion and stirred overnight to allow the solution to naturally reach ambient temperature. The resulting mixture was neutralized with a 2M hydrochloric acid solution to obtain a solid, and the solid was filtered, and washed with water to obtain Intermediate 85-b (33.2 g, 108 mmol).

### Third Step: Synthesis of Intermediate 85-c

In a 2-neck 1,000 mL round-bottomed flask, 9,9′-(6-chloro-1,3,5-triazine-2,4-diyl)bis(9H-carbazole-1,2,3,4,5,6,7,8-d8) (15 g, 32.5 mmol), (5-chloro-2-fluorophenyl)boronic acid (6.2 g, 35.7 mmol), Pd(PPh₃)₄(1.1 g, 1 mmol), potassium carbonate (9 g, 65 mmol), 1,4-dioxane (160 mL), and D. I. water (54 mL) were added and then, heated under reflux for 14 hours. The obtained mixture was recrystallized with xylene to obtain Intermediate 85-c (16.5 g, 29.7 mmol).

### Fourth Step: Synthesis of Intermediate 85-d

In a 2-neck 500 ml round-bottomed flask, Intermediate 85-c (15.3 g, 27.5 mmol), Intermediate 85-b (9.7 g, 33 mmol), Pd(Amphos)C1₂ (1 g, 1.4 mmol), potassium carbonate (15 g, 55 mmol), 1,4-dioxane (140 ml), and D. I. water (50 ml) were added and then, heated under reflux for 16 hours under nitrogen. The obtained mixture was recrystallized with xylene to obtain Intermediate 85-d (18.1 g, 23.5 mmol)

### Fifth Step: Synthesis of Compound 85

In a 2-neck 500 ml round-bottomed flask, 9H-carbazole-d8 (3.8 g 21.5 mmol), NaH (1.0 g, 26 mmol), and dimethylformamide (90 ml) were added and then, stirred at ambient temperature for 30 minutes under nitrogen, and Intermediate 85-d (18 g, 21 mmol) was added thereto and then, stirred by heating at 120 °C for 3 hours. The obtained mixture was recrystallized with xylene to obtain Compound 85 (12.5 g, 13.5 mmol).

### Synthesis Example 2: Synthesis of Compound 84

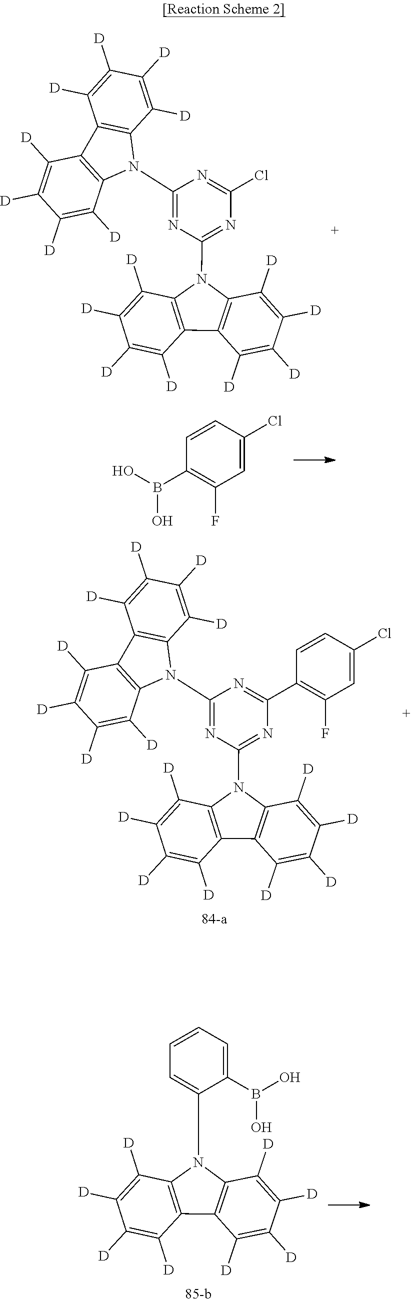

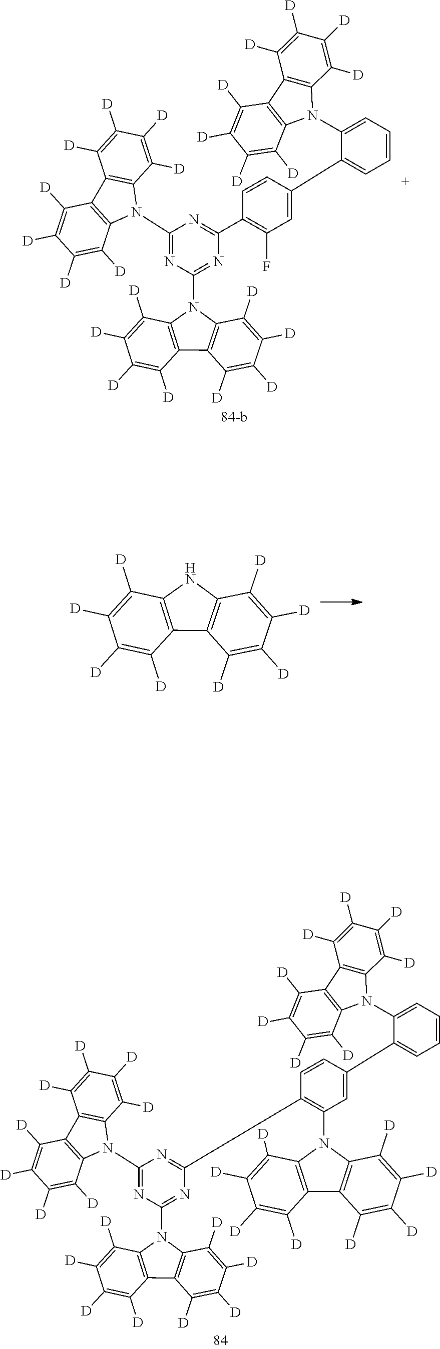

### First Step: Synthesis of Intermediate 84-a

Intermediate 84-a (17.1 g, 31 mol) was synthesized in the same manner as in the synthesis of Intermediate 85-c except that (4-chloro-2-fluorophenyl)boronic acid (8.3 g, 47.6 mmol) was used instead of the (5-chloro-2-fluorophenyl)boronic acid (6.2 g, 35.7 mmol).

### Second Step: Synthesis of Intermediate 84-b

Intermediate 84-b (15.9 g, 31 mmol) was synthesized in the same manner as in the synthesis of Intermediate 85-d except that Intermediate 84-a (17.1 g, 30.8 mmol) was used instead of Intermediate 85-c (15.3 g, 27.5 mmol).

### Third Step: Synthesis of Compound 84

Compound 84 (8 g, 8.6 mmol) was synthesized in the same manner as in the synthesis of Compound 85 except that Intermediate 84-b (15.9 g, 31 mmol) was used instead of Intermediate 85-d (18 g, 21 mmol).

### Synthesis Example 3: Synthesis of Compound 81

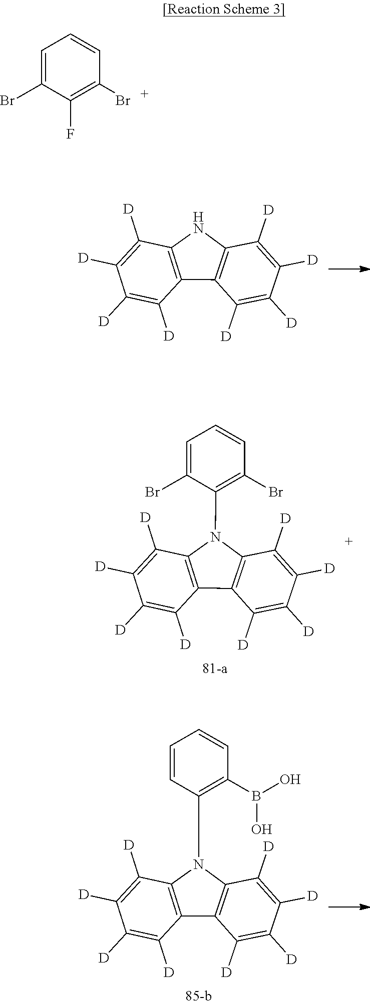 [SMILES: Fc1c(Br)cccc1Br ; [2H]c1c([2H])c([2H])c2c([nH]c3c([2H])c([2H])c([2H])c([2H])c32)c1[2H] ; [2H]c1c([2H])c([2H])c2c(c1[2H])c1c([2H])c([2H])c([2H])c([2H])c1n2-c1c(Br)cccc1Br ; [2H]c1c([2H])c([2H])c2c(c1[2H])c1c([2H])c([2H])c([2H])c([2H])c1n2-c1ccccc1B(O)O]

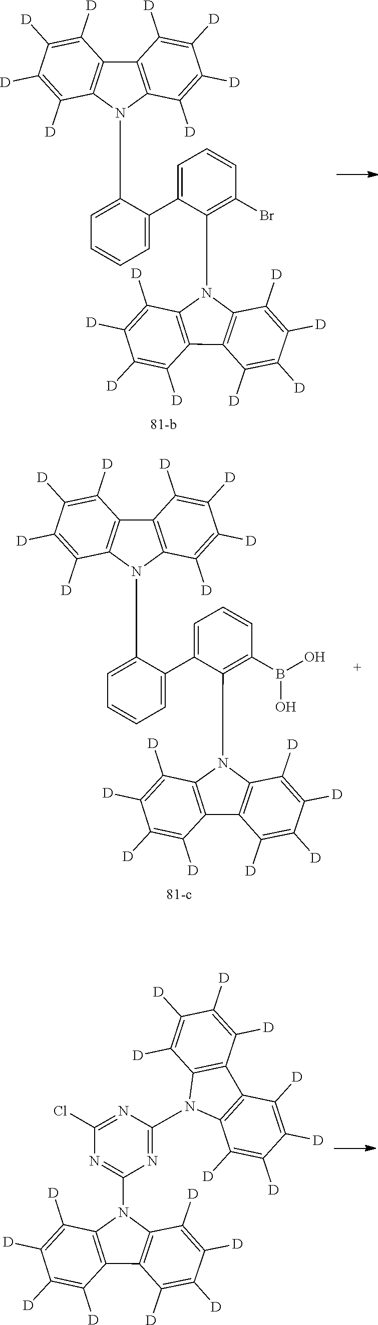 [SMILES: [2H]c1c([2H])c([2H])c2c(c1[2H])c1c([2H])c([2H])c([2H])c([2H])c1n2-c1ccccc1-c1cccc(Br)c1-n1c2c([2H])c([2H])c([2H])c([2H])c2c2c([2H])c([2H])c([2H])c([2H])c21 ; [2H]c1c([2H])c([2H])c2c(c1[2H])c1c([2H])c([2H])c([2H])c([2H])c1n2-c1ccccc1-c1cccc(B(O)O)c1-n1c2c([2H])c([2H])c([2H])c([2H])c2c2c([2H])c([2H])c([2H])c([2H])c21 ; [2H]c1c([2H])c([2H])c2c(c1[2H])c1c([2H])c([2H])c([2H])c([2H])c1n2-c1nc(Cl)nc(-n2c3c([2H])c([2H])c([2H])c([2H])c3c3c([2H])c([2H])c([2H])c([2H])c32)n1]

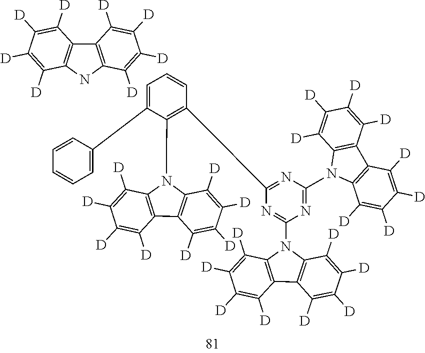

### First Step: Synthesis of Intermediate 81-a

Intermediate 81-a (22.3 g, 54 mmol) was synthesized in the same manner as in the synthesis of Intermediate 85-a except that 1,3-dibromo-2-fluorobenzene (15 g, 59 mmol) was used instead of the 1-bromo-2-fluorobenzene (40 g, 229 mmol).

### Second Step: Synthesis of Intermediate 81-b

In a 2-neck 1,000 mL round-bottomed flask, Intermediate 81-a (15 g, 36.7 mmol), Intermediate 85-b (10.8 g, 36.7 mmol), Pd(PPh₃)₄(1.3 g, 1.1 mmol), potassium carbonate (10 g, 73 mmol), tetrahydrofuran (180 ml), and D. I. water (60 ml) were added and then, heated under reflux for 14 hours. The obtained mixture was purified by column chromatography to obtain Intermediate 81-b (11.6 g, 20 mmol).

### Third Step: Synthesis of Intermediate 81-c

Intermediate 81-c (14.2 g, 26 mmol) was synthesized in the same manner as in the synthesis of Intermediate 85-b except that Intermediate 81-b (16.9 g, 29.2 mmol) was used instead of Intermediate 85-a (41.5 g, 126 mmol).

### Fourth Step: Synthesis of Compound 81

In a 2-neck 500 mL round-bottomed flask, 9,9′-(6-chloro-1,3,5-triazine-2,4-diyl)bis(9H-carbazole-1,2,3,4,5,6,7,8-d8) (12 g, 26.2 mmol), Intermediate 81-c (14.2 g, 26 mmol), Pd(PPh₃)₄(0.9 g, 0.8 mmol), potassium carbonate (7.2 g, 52 mmol), tetrahydrofuran (90 mL), and D. I. water (45 mL) were added and then, heated under reflux for 16 hours. The obtained mixture was recrystallized with xylene to obtain Compound 81 (16.9 g, 18.2 mmol).

### Synthesis Example 4: Synthesis of Compound D-62 (Dn=20)

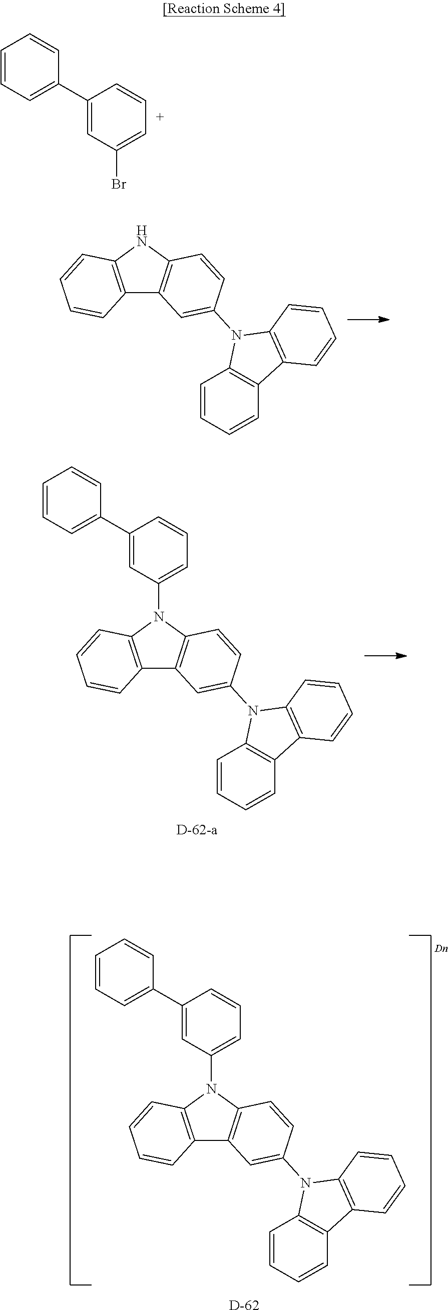

### First Step: Synthesis of Intermediate D-62-a

In a 2 neck 500 ml round-bottomed flask, 3-bromo-1,1′-biphenyl (33.7 g, 144 mmol), 9H-3,9′-bicarbazole (40 g, 120 mmol), Pd₂(dba)₃ (5.5 g, 6 mmol), sodium tert-butoxide (13.9 g, 144 mmol), a tri-tert-butylphosphine solution (8.5 mL, 18 mmol), and xylene (400 ml) were added and then, stirred by heating at 120 °C for 6 hours under nitrogen. The obtained mixture was purified by column chromatography to obtain Intermediate D-62-a (38.85 g, 80 mmol)

### Second Step: Synthesis of Compound D-62 (Dn=20)

Intermediate D-62-a (20 g, 41.3 mmol), triflic acid (18 ml), benzene-d6 (110 ml), and dichloromethane (100 ml) were mixed and then, stirred at 10 °C for 18 hours. The mixture was cooled to ambient temperature, quenched with heavy water (D₂O), and neutralized with a 20 wt % tripotassium phosphate solution to separate an organic layer, which was column-purified to synthesize Compound D-62 (n=20 or so) (19.1 g, 38 mmol).

### Comparison Synthesis Example 1: Synthesis of Compound R-2

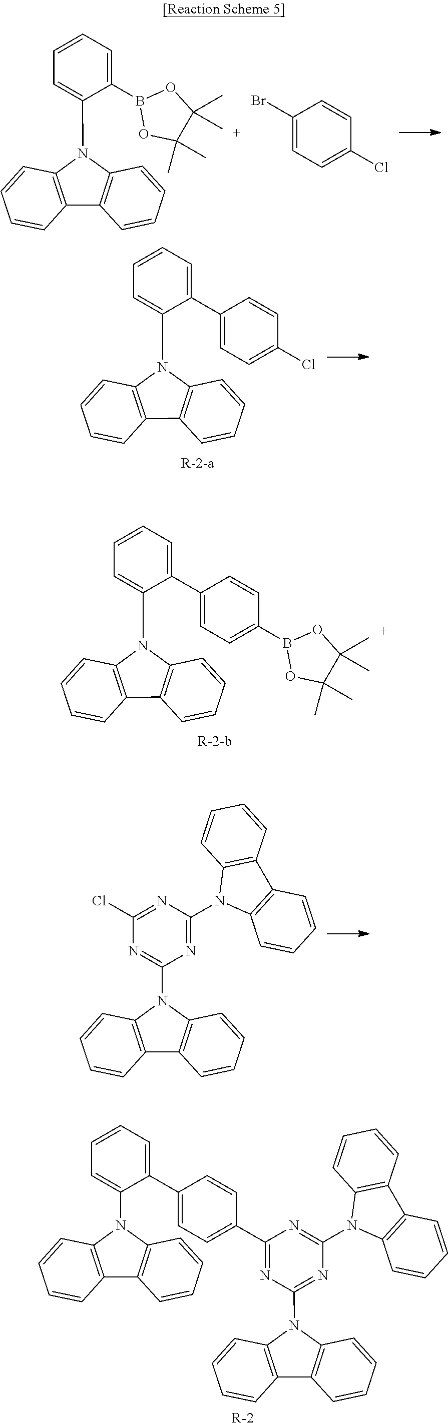

### First Step: Synthesis of Intermediate R-2-a

In a 2-neck 500 mL round-bottomed flask, 9-(2-(4,4,5,5-tetramethyl-1,3,2-dioxaborolan-2-yl)phenyl)-9H-carbazole (30 g, 81.2 mmol), 1-bromo-4-chlorobenzene (15.6 g, 81.2 mmol), Pd(PPh₃)₄(2.8 g, 2.4 mmol), potassium carbonate (22.5 g, 162 mmol), tetrahydrofuran (270 mL), and D. I. water (135 mL) were added and then, stirred under reflux for 8 hours. The obtained mixture was purified by column chromatography to obtain Intermediate R-2-a (24.5 g, 69.2 mmol).

### Second Step: Synthesis of Intermediate R-2-b

In a 2-neck 500 mL round-bottomed flask, Intermediate R-2-a (24.5 g, 69.2 mmol), 4,4,4′,4′,5,5,5′,5′-octamethyl-2,2′-bi(1,3,2-dioxaborolane) (22.9 g, 90 mmol), Pd₂(dba)₃ (3.2 g, 3.5 mmol), potassium acetate (17 g, 173 mmol), and toluene (160 ml) were added and then, stirred under reflux for 10 hours. The obtained mixture was extracted with water and methylene chloride and then, concentrated to obtain Intermediate R-2-b (24 g, 46.6 mmol).

### Third Step: Synthesis of Compound R-2

In a 2-neck 500 mL round-bottomed flask, Intermediate R-2-b (24 g, 46.6 mmol), 9,9′-(6-chloro-1,3,5-triazine-2,4-diyl)bis(9H-carbazole) (21.6 g, 48.5 mmol), Pd(PPh₃)₄(1.7 g, 1.5 mmol), potassium carbonate (13.4 g, 97 mmol), tetrahydrofuran (160 mL), and D. I. water (80 mL) were added and then, stirred under reflux for 16 hours. The obtained mixture was recrystallized with chlorobenzene to obtain Compound R-2 (28.7 g, 39.4 mmol).

### Comparison Synthesis Example 2: Synthesis of Compound R-3

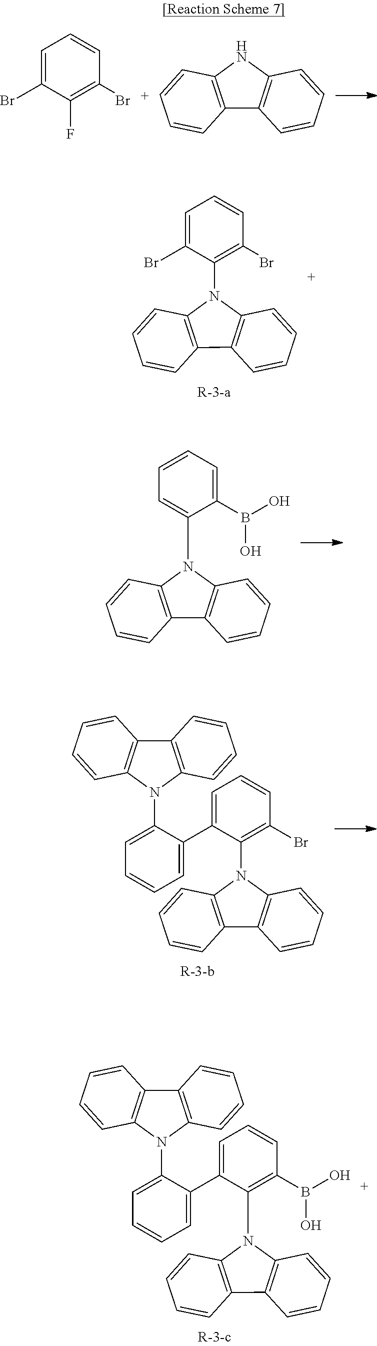

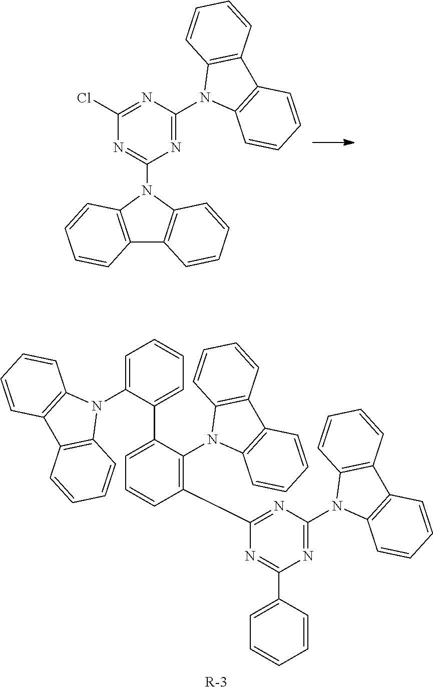 [SMILES: Clc1nc(-n2c3ccccc3c3ccccc32)nc(-n2c3ccccc3c3ccccc32)n1 ; c1ccc(-c2nc(-c3cccc(-c4ccccc4-n4c5ccccc5c5ccccc54)c3-n3c4ccccc4c4ccccc43)nc(-n3c4ccccc4c4ccccc43)n2)cc1 ; "R-3"]

### First Step: Synthesis of Intermediate R-3-a

Intermediate R-3-a (11.8 g, 29.4 mmol) was synthesized in the same manner as in the synthesis of Intermediate 81-a except that 9H-carbazole (6.6 g, 39.4 mmol) was used instead of the 9H-carbazole-d8 (10.4 mmol).

### Second Step: Synthesis of Intermediate R-3-b

Intermediate R-3-b (7.7 g, 13.7 mmol) was synthesized in the same manner as in the synthesis of Intermediate 81-b except that Intermediate R-3-a (11.8 g, 29.4 mmol) was used instead of Intermediate 81-a (15 g, 36.7 mmol).

### Third Step: Synthesis of Intermediate R-3-c

Intermediate R-3-c (6 g, 11.3 mmol) was synthesized in the same manner as in the synthesis of Intermediate 81-c except that Intermediate R-3-b was used instead of Intermediate 81-b.

### Fourth Step: Synthesis of Compound R-3

In a 2-neck 250 mL round-bottomed flask, Intermediate R-3-c (6 g, 11.3 mmol), 9-(4-chloro-6-phenyl-1,3,5-triazin-2-yl)-9H-carbazole (4 g, 11.3 mmol), Pd(PPh₃)₄(0.4 g, 0.3 mmol), potassium carbonate (3.1 g, 22.7 mmol), tetrahydrofuran (40 mL), and D. I. water (20 mL) were added and then, heated under reflux for 14 hours. The obtained mixture was recrystallized with chlorobenzene to obtain Compound R-3 (7.8 g, 9.3 mmol).

### Manufacturing of Organic Light Emitting Diode

### Example 1

A glass substrate coated with a thin film of ITO (indium tin oxide) was ultrasonically cleaned with distilled water. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. The prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to a thickness of 600 Å to form a hole transport layer. mCP(1,3-bis(carbazol-9-yl)benzene) was deposited to a thickness of 100 Å on the hole transport layer to form a hole transport auxiliary layer. Compound 85 (host) obtained in Synthesis Example 1 and P31 (dopant) were vacuum-deposited on the hole transport auxiliary layer to form a 400 Å-thick light emitting layer. Herein, Compound 85 and P31 were used in a weight ratio of 87:13. Subsequently, BCP (2,9-dimethyl-4,7-diphenyl-1,10-phenanthroline) was deposited on the light emitting layer to a thickness of 50 Å to form an electron transport auxiliary layer, and Compound B and LiQ were simultaneously vacuum deposited in a weight ratio of 1:1 to form a 300 Å-thick electron transport layer. An organic light emitting diode was manufactured by sequentially vacuum depositing 10 Å of LiQ and 1,200 Å of Al on the electron transport layer to form a cathode.

The organic light emitting diode was manufactured with the structure of ITO/Compound A (3% NDP-9 doping, 100 Å)/Compound A (600 Å)/mCP (100 Å)/EML [87 wt % of Compound 1:13 wt % of P31](400 Å)/BCP (50 Å)/Compound B:LiQ (300 Å)/LiQ (10 Å)/Al (1,200 Å).

### Compound A: N-(9,9-diphenyl-9H-fluoren-2-yl)-N,9-diphenyl-9H-carbazol-2-amine

### Compound B: 8-{4-[bis(naphthalene-2-yl)-1,3,5-triazin-2-yl]phenyl}quinoline

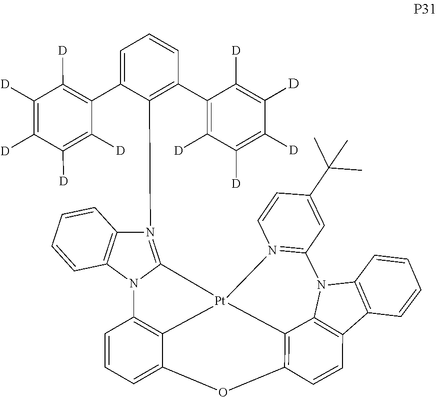

### Examples 2 to 3 and Comparative Example 1

Each organic light emitting diode was manufactured in the same manner as in Example 1, except that the light emitting layers were formed using the compounds described in Table 1 instead of Compound 85 according to Example 1 as the host of the light emitting layer.

### Example 4

A glass substrate coated with a thin film of ITO (indium tin oxide) was ultrasonically cleaned with distilled water. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. The prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to a thickness of 600 Å to form a hole transport layer. mCP was deposited to a thickness of 100 Å on the hole transport layer to form a hole transport auxiliary layer. On the hole transport auxiliary layer, Compound 85 (first host) obtained in Synthesis Example 1 and Compound D-62 (Dn=20) (second host) obtained in Synthesis Example 4 were used as hosts, and 13 wt % of P31 was doped as a phosphorescent sensitizer and 1.5 wt % of D3 was doped as a fluorescent dopant to form a 400 Å-thick light emitting layer by vacuum deposition. Herein, Compound 85 and Compound D-62 were used in a weight ratio of 5:5. Subsequently, BCP was deposited on the light emitting layer to a thickness of 50 Å to form an electron transport auxiliary layer, and Compound B and LiQ were simultaneously vacuum deposited in a weight ratio of 1:1 to form a 300 Å-thick electron transport layer. An organic light emitting diode was manufactured by sequentially vacuum depositing 10 Å of LiQ and 1,200 Å of Al on the electron transport layer to form a cathode.

The organic light emitting diode was manufactured with the structure of ITO/Compound A (3% NDP-9 doping, 100 Å)/Compound A (600 Å)/mCP (100 Å)/EML [host (85.5 wt % of Compound 85: Compound D-62=5:5 (w/w): 13 wt % of P31:1.5 wt % of D3](400 Å)/BCP (50 Å)/Compound B:LiQ (300 Å)/LiQ (10 Å)/Al (1,200 Å).

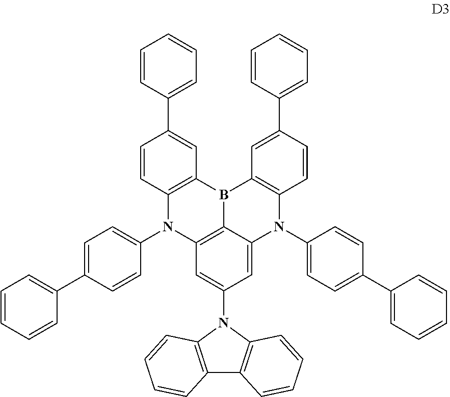 [SMILES: c1ccc(-c2ccc(N3c4ccc(-c5ccccc5)cc4B4c5cc(-c6ccccc6)ccc5N(c5ccc(-c6ccccc6)cc5)c5cc(-n6c7ccccc7c7ccccc76)cc3c54)cc2)cc1]

### Examples 5 to 6 Comparative Example 2 to 3

Each organic light emitting diode was manufactured in the same manner as in Example 4, except that the light emitting layers were formed using the compositions described in Table 2 instead of the composition according to Example 4 as the host for the light emitting layer.

### Evaluation

The efficiency and life-span characteristics of the organic light emitting diodes according to each of Examples and Comparative Examples were evaluated.

The specific measurement methods are as follows, and the results are in Tables 1 and 2.

### (1) Measurement of Current Density Change Depending on Voltage Change

The obtained organic light emitting diodes were measured regarding a current value flowing in the unit diode, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000 Å), while the voltage of the organic light emitting diodes was increased from 0 V to 10 V

### (3) Measurement of Luminous Efficiency

The luminance and the current density measured from (1) and (2) above and a voltage were used to calculate the luminous efficiency (cd/A) at the same current density (10 mA/cm²).

The relative values calculated based on the luminous efficiency according to Comparative Example 1 are listed in Table 1.

### (4) Measurement of Life-Span

Time that the luminous efficiency (cd/A) of the organic light emitting diodes decreased to 95%, while maintaining the luminance (cd/m²) at 2000 cd/m², was measured.

The relative values calculated based on the life-span measurement values according to Comparative Example 2 are listed in Table 2.

TABLE 1

| No. | Host | Efficiency (%) |
|---|---|---|
| Example 1 | Compound 85 | 113 |
| Example 2 | Compound 84 | 104 |
| Example 3 | Compound 81 | 115 |
| Comparative Example 1 | Compound R-2 | 100 |

TABLE 2

| | | | Life-span |
|---|---|---|---|
| No. | First host | Second host | (%) |
| Example 4 | Compound 85 | Compound D-62 | 695 |
| Example 5 | Compound 84 | (Dn = 20) | 1727 |
| Example 6 | Compound 81 | | 1551 |
| Comparative Example 2 | Compound R-2 | | 100 |
| Comparative Example 3 | Compound R-3 | | 409 |

Referring to Tables 1 and 2, the organic light emitting diodes according to Example has improved efficiency and life-span characteristics compared to the organic light emitting diodes according to Comparative Examples.

By way of summation and review, some example embodiments may provide a compound for an organic optoelectronic device that may help lower the driving voltage and help realize a high-efficiency and long life-span organic optoelectronic device. Some example embodiments may provide a composition for an organic optoelectronic device including the compound for the organic optoelectronic device. Some example embodiments may provide an organic optoelectronic device including the compound for the organic optoelectronic device or the composition for an organic optoelectronic device. Some example embodiments may provide a display device including the organic optoelectronic device.

Through some embodiments, high-efficiency, long life-span organic optoelectronic devices may be realized while lowering the operating voltage.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device represented by Chemical Formula 1: 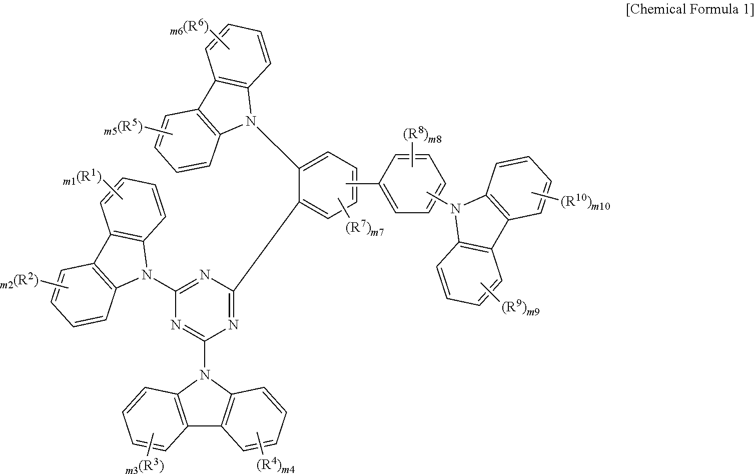
wherein, in Chemical Formula 1,
R¹ to R¹⁰ are each independently hydrogen, deuterium, a substituted or unsubstituted C1 to C20 alkyl group, a substituted or unsubstituted C3 to C20 cycloalkyl group, a substituted or unsubstituted phenyl group, a substituted or unsubstituted biphenyl group, or a substituted or unsubstituted terphenyl group,
m1 to m6, and m8 to m10 are each independently an integer of 1 to 4, and
m7 is an integer of 1 to 3, and
when m1 is 2, 3, or 4, each R¹ is the same or different from each other,
when m2 is 2, 3, or 4, each R² is the same or different from each other,
when m3 is 2, 3, or 4, each R³ is the same or different from each other,
when m4 is 2, 3, or 4, each R⁴ is the same or different from each other,
when m5 is 2, 3, or 4, each R⁵ is the same or different from each other,
when m6 is 2, 3, or 4, each R⁶ is the same or different from each other,
when m7 is 2 or 3, each R⁷ is the same or different from each other,
when m8 is 2, 3, or 4, each R⁸ is the same or different from each other,
when m9 is 2, 3, or 4, each R⁹ is the same or different from each other, and
when m10 is 2, 3, or 4, each R¹⁰ is the same or different from each other.

2. The compound for the organic optoelectronic device as claimed in claim 1, wherein:
Chemical Formula 1 is represented by one of Chemical Formula 1-1 to Chemical Formula 1-4: 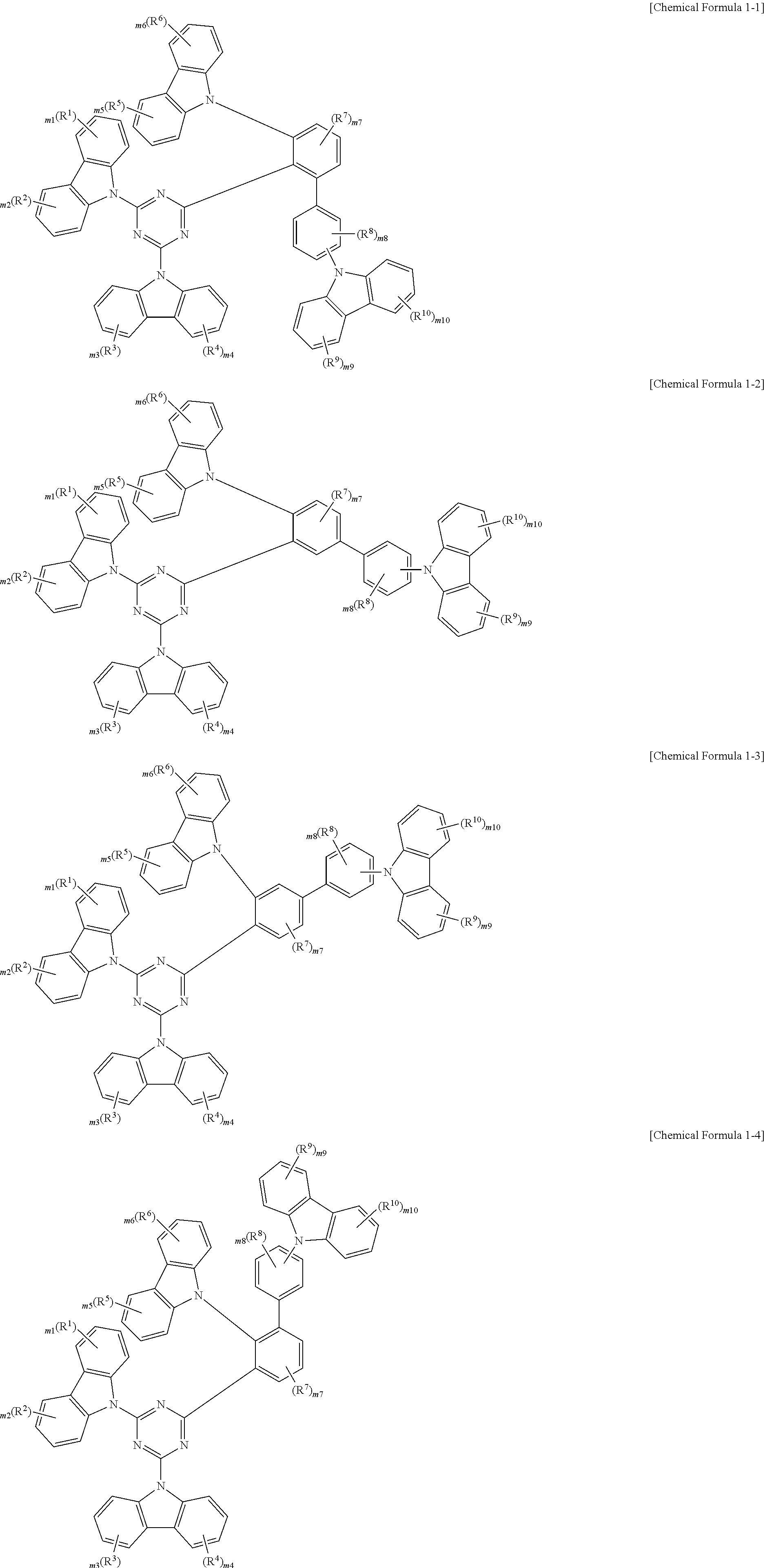
in Chemical Formula 1-1 to Chemical Formula 1-4, R¹ to R¹⁰, and m1 to m10 are defined the same as those of Chemical Formula 1.

3. The compound for the organic optoelectronic device as claimed in claim 1, wherein:
Chemical Formula 1 is represented by one of Chemical Formula 1-1a, Chemical Formula 1-2a, Chemical Formula 1-3a, and Chemical Formula 1-4a: 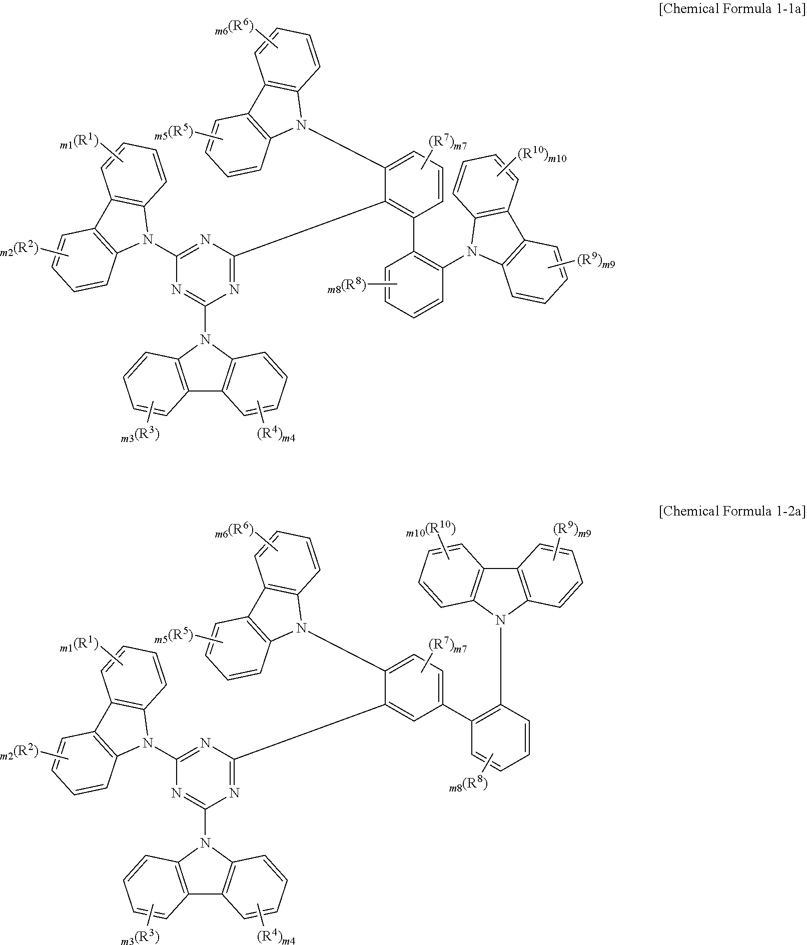 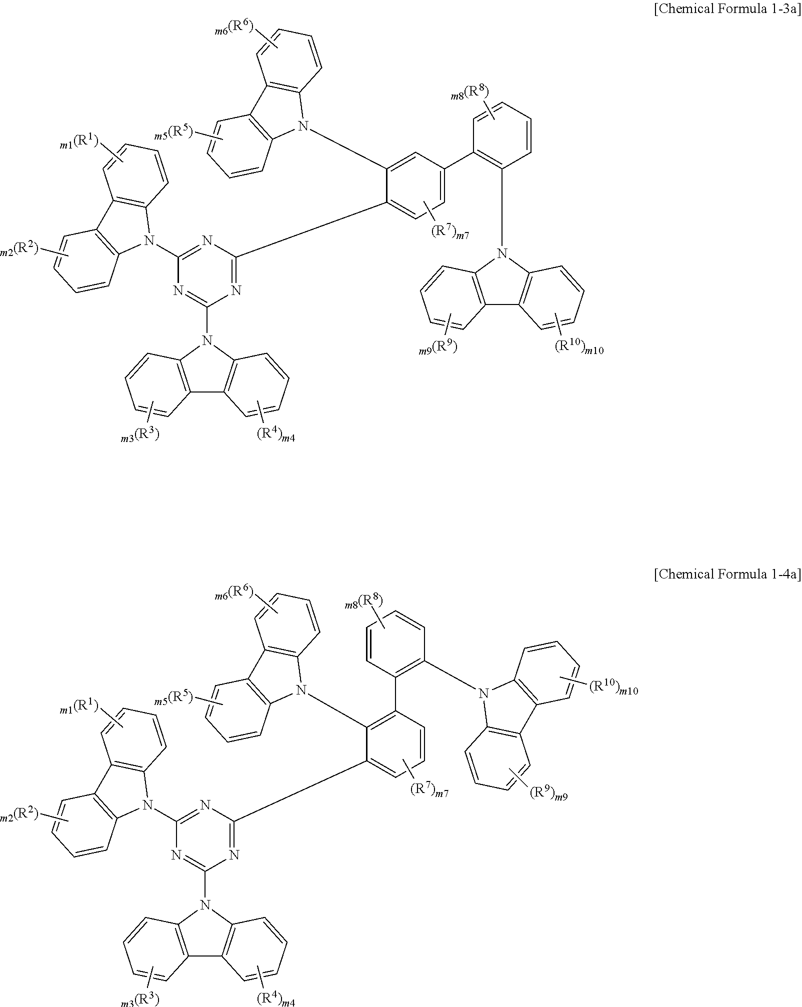
in Chemical Formula 1-1a, Chemical Formula 1-2a, Chemical Formula 1-3a, and Chemical Formula 1-4a, R¹ to R¹⁰, and m1 to m10 are defined the same as those of Chemical Formula 1.

4. The compound for the organic optoelectronic device as claimed in claim 1, wherein R¹ to R¹⁰ are each independently hydrogen, deuterium, a substituted or unsubstituted C1 to C10 alkyl group, a substituted or unsubstituted C3 to C10 cycloalkyl group, a substituted or unsubstituted phenyl group, or a substituted or unsubstituted biphenyl group.

5. The compound for the organic optoelectronic device as claimed in claim 1, wherein:
the compound is a compound of Group 1: 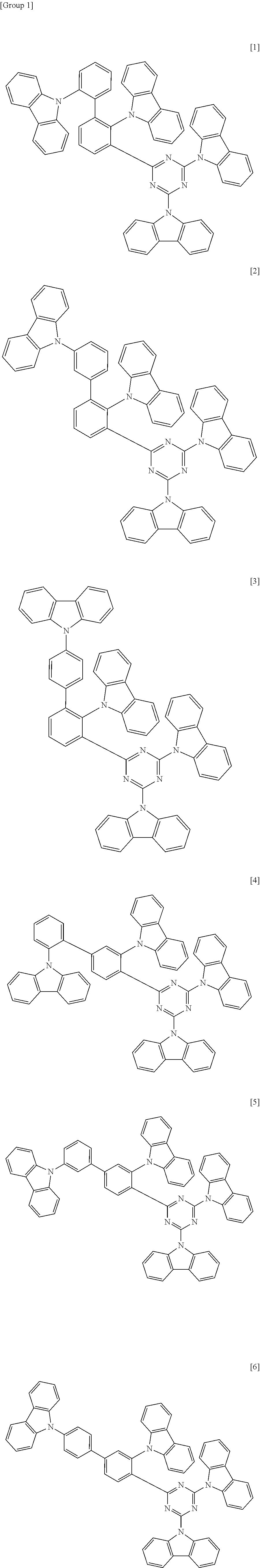 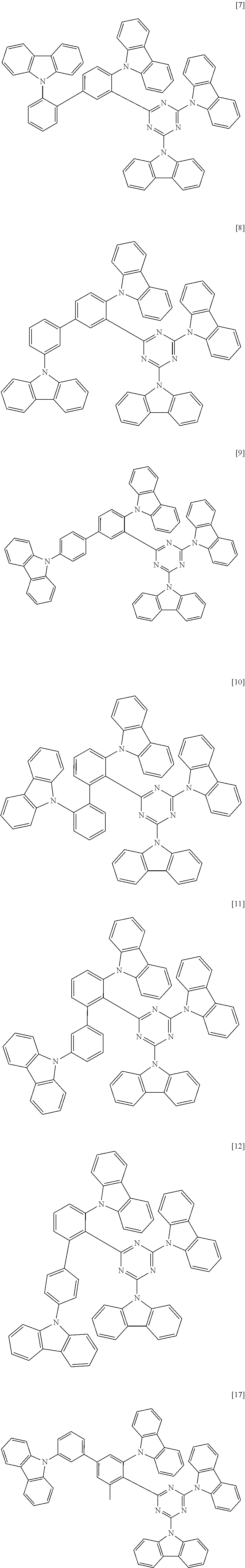 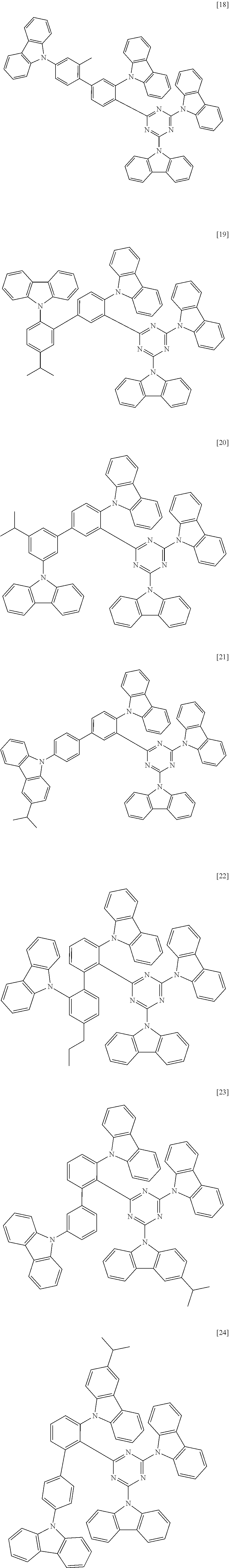 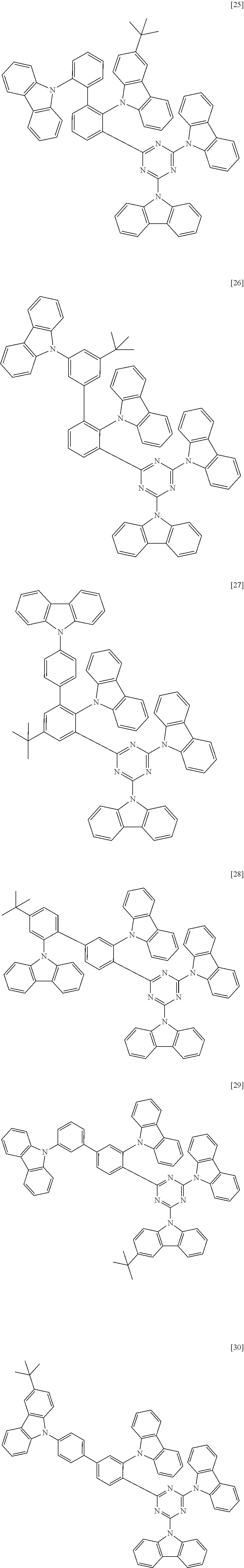 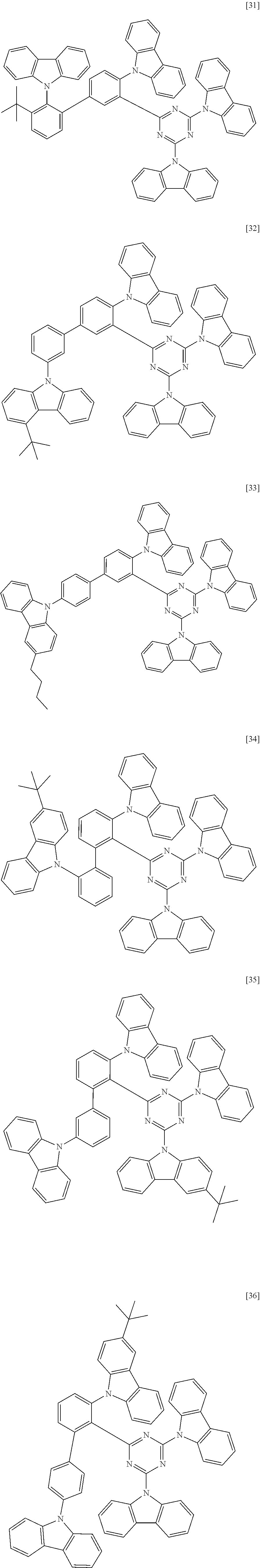 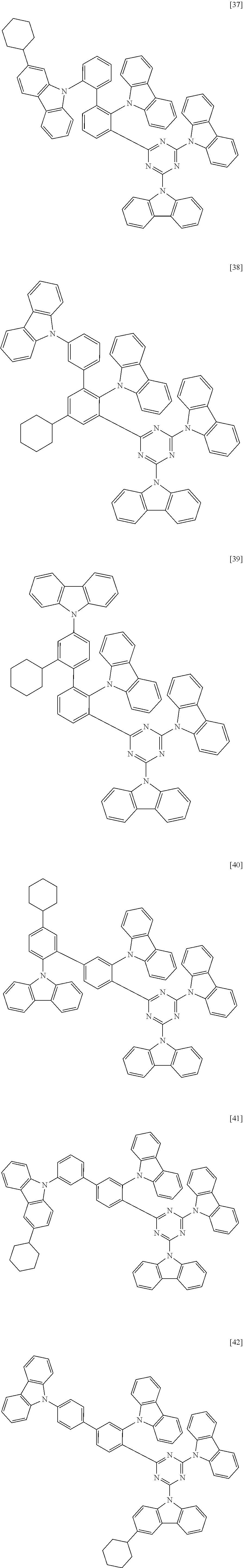 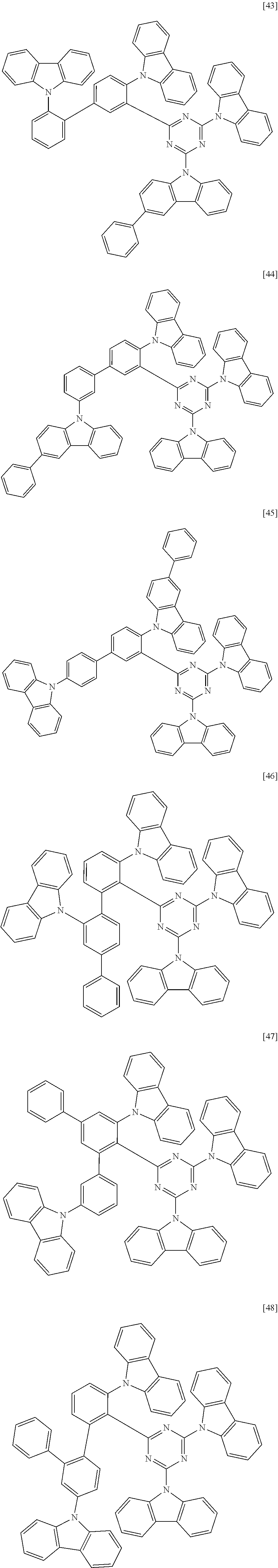 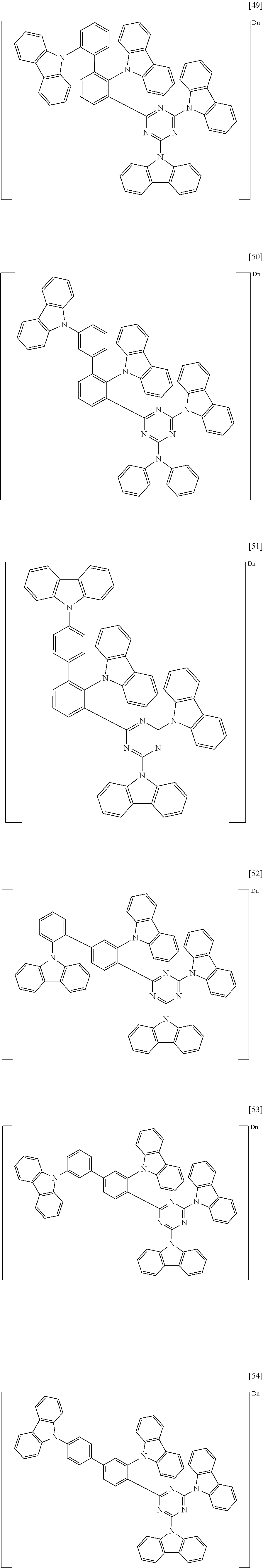 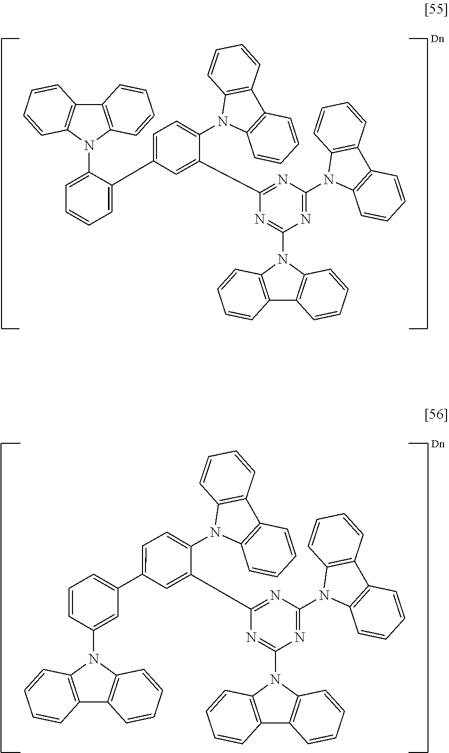
and, Dn refers to the number of deuterium atoms substituted, and n is an integer between 1 and the maximum number of substitutions possible.

6. The compound for the organic optoelectronic device as claimed in claim 5, wherein the compound is a compound of Group 1-1:

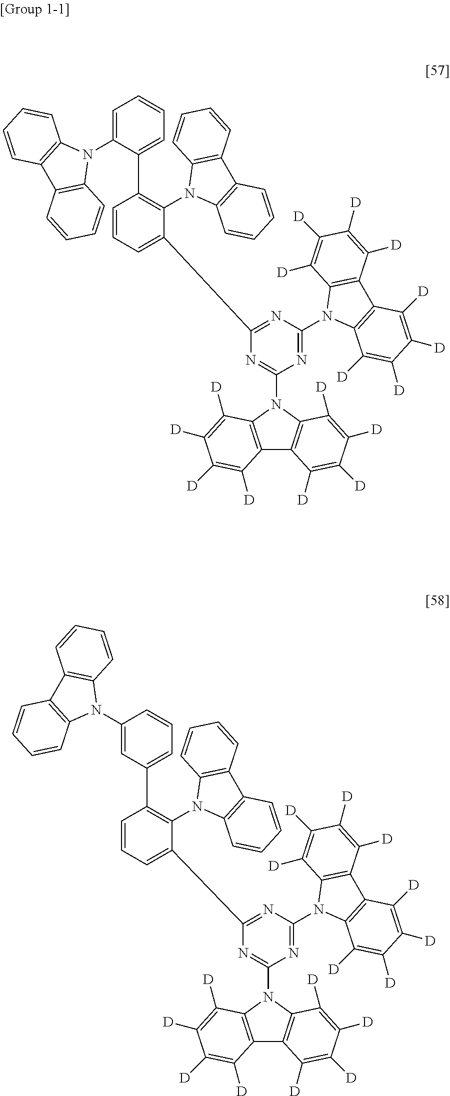 [SMILES: [2H]c1c([2H])c([2H])c2c(c1[2H])c1c([2H])c([2H])c([2H])c([2H])c1n2-c1nc(-c2cccc(-c3ccccc3-n3c4ccccc4c4ccccc43)c2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2cccc(-c3cccc(-n4c5ccccc5c5ccccc54)c3)c2-n2c3ccccc3c3ccccc32)nc(-n2c3c([2H])c([2H])c([2H])c([2H])c3c3c([2H])c([2H])c([2H])c([2H])c32)n1]

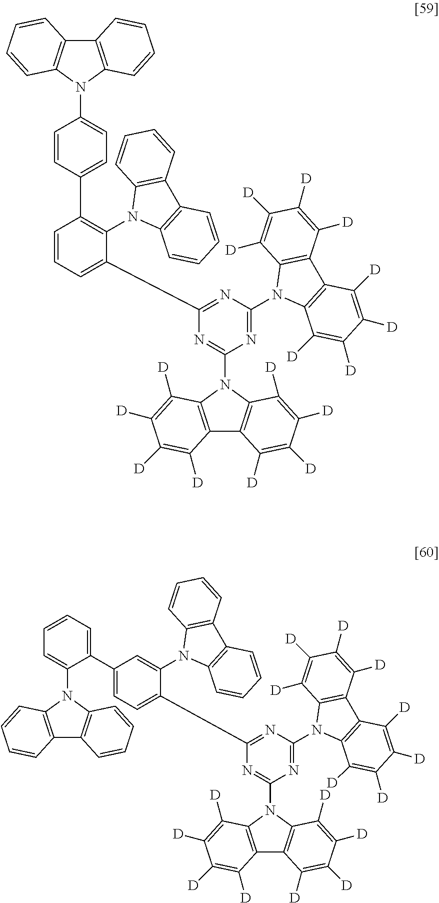 [SMILES: [2H]c1c([2H])c([2H])c2c(c1[2H])c1c([2H])c([2H])c([2H])c([2H])c1n2-c1nc(-c2cccc(-c3ccc(-n4c5ccccc5c5ccccc54)cc3)c2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2ccc(-c3ccccc3-n3c4ccccc4c4ccccc43)cc2-n2c3ccccc3c3ccccc32)nc(-n2c3c([2H])c([2H])c([2H])c([2H])c3c3c([2H])c([2H])c([2H])c([2H])c32)n1]

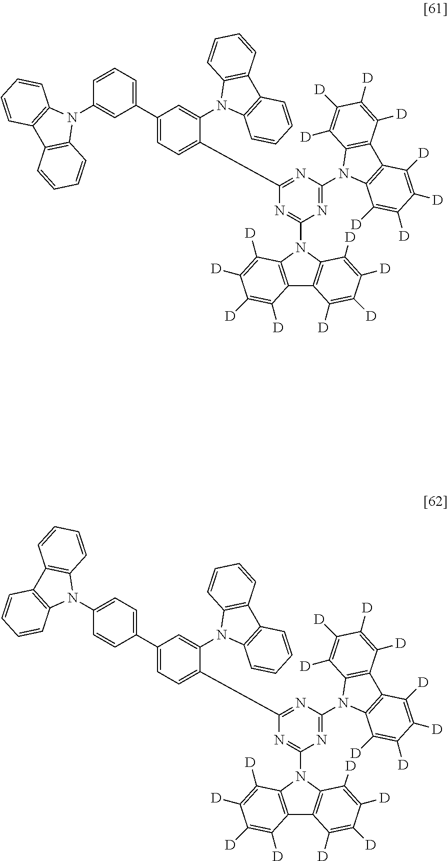 [SMILES: [2H]c1c([2H])c([2H])c2c(c1[2H])c1c([2H])c([2H])c([2H])c([2H])c1n2-c1nc(-c2ccc(-c3cccc(-n4c5ccccc5c5ccccc54)c3)cc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2ccc(-c3ccc(-n4c5ccccc5c5ccccc54)cc3)cc2-n2c3ccccc3c3ccccc32)nc(-n2c3c([2H])c([2H])c([2H])c([2H])c3c3c([2H])c([2H])c([2H])c([2H])c32)n1]

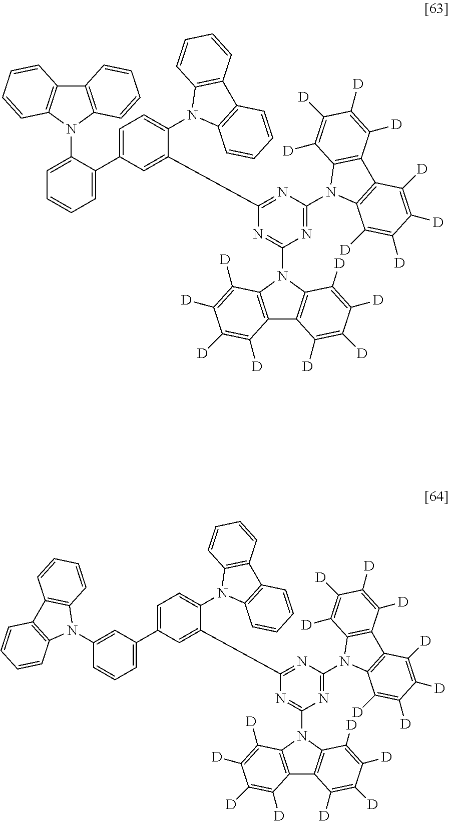 [SMILES: [2H]c1c([2H])c([2H])c2c(c1[2H])c1c([2H])c([2H])c([2H])c([2H])c1n2-c1nc(-c2cc(-c3ccccc3-n3c4ccccc4c4ccccc43)ccc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2cc(-c3cccc(-n4c5ccccc5c5ccccc54)c3)ccc2-n2c3ccccc3c3ccccc32)nc(-n2c3c([2H])c([2H])c([2H])c([2H])c3c3c([2H])c([2H])c([2H])c([2H])c32)n1]

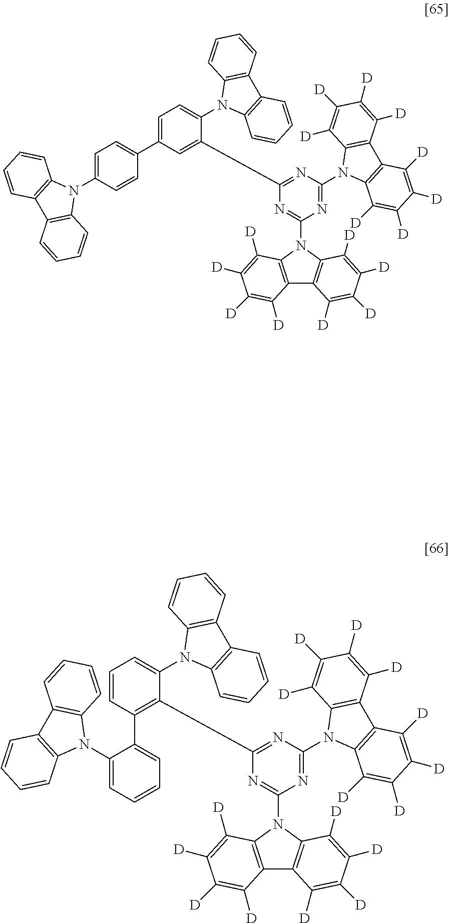 [SMILES: [2H]c1c([2H])c([2H])c2c(c1[2H])c1c([2H])c([2H])c([2H])c([2H])c1n2-c1nc(-c2cc(-c3ccc(-n4c5ccccc5c5ccccc54)cc3)ccc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2c(-c3ccccc3-n3c4ccccc4c4ccccc43)cccc2-n2c3ccccc3c3ccccc32)nc(-n2c3c([2H])c([2H])c([2H])c([2H])c3c3c([2H])c([2H])c([2H])c([2H])c32)n1]

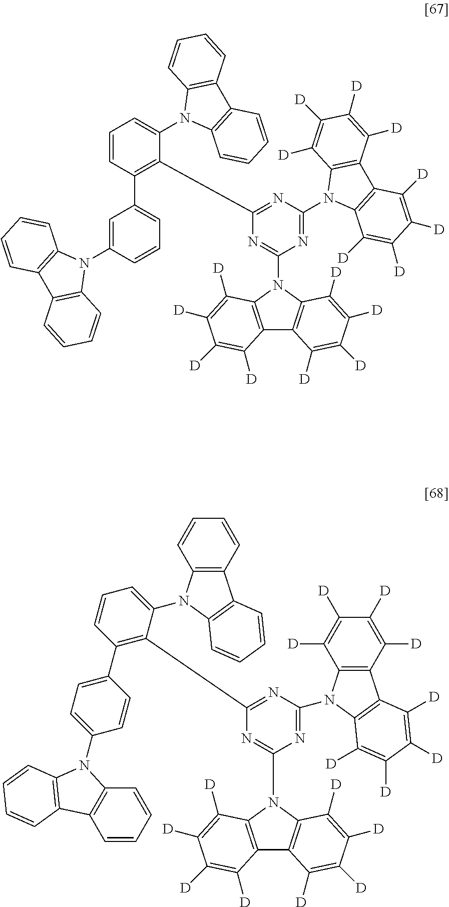 [SMILES: [2H]c1c([2H])c([2H])c2c(c1[2H])c1c([2H])c([2H])c([2H])c([2H])c1n2-c1nc(-c2c(-c3cccc(-n4c5ccccc5c5ccccc54)c3)cccc2-n2c3ccccc3c3ccccc32)nc(-n2c3c([2H])c([2H])c([2H])c([2H])c3c3c([2H])c([2H])c([2H])c([2H])c32)n1 ; [2H]c1c([2H])c([2H])c2c(c1[2H])c1c([2H])c([2H])c([2H])c([2H])c1n2-c1nc(-c2c(-c3ccc(-n4c5ccccc5c5ccccc54)cc3)cccc2-n2c3ccccc3c3ccccc32)nc(-n2c3c([2H])c([2H])c([2H])c([2H])c3c3c([2H])c([2H])c([2H])c([2H])c32)n1]

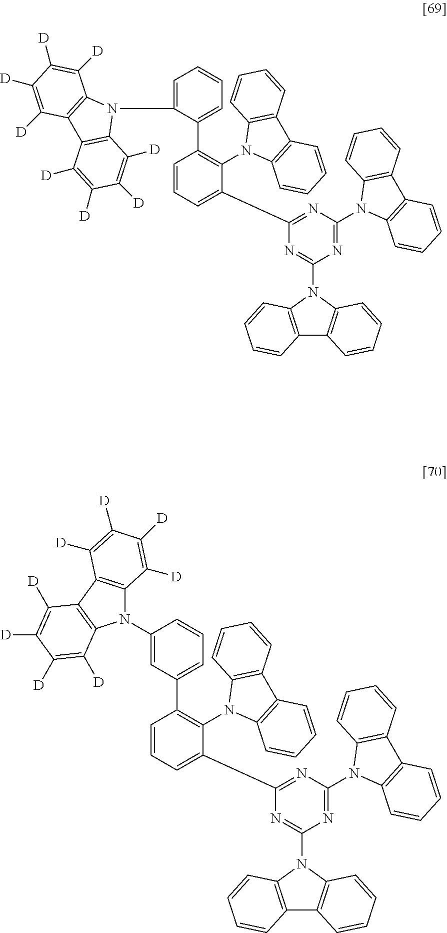 [SMILES: [2H]c1c([2H])c([2H])c2c(c1[2H])c1c([2H])c([2H])c([2H])c([2H])c1n2-c1ccccc1-c1cccc(-c2nc(-n3c4ccccc4c4ccccc43)nc(-n3c4ccccc4c4ccccc43)n2)c1-n1c2ccccc2c2ccccc21 ; [2H]c1c([2H])c([2H])c2c(c1[2H])c1c([2H])c([2H])c([2H])c([2H])c1n2-c1cccc(-c2cccc(-c3nc(-n4c5ccccc5c5ccccc54)nc(-n4c5ccccc5c5ccccc54)n3)c2-n2c3ccccc3c3ccccc32)c1]

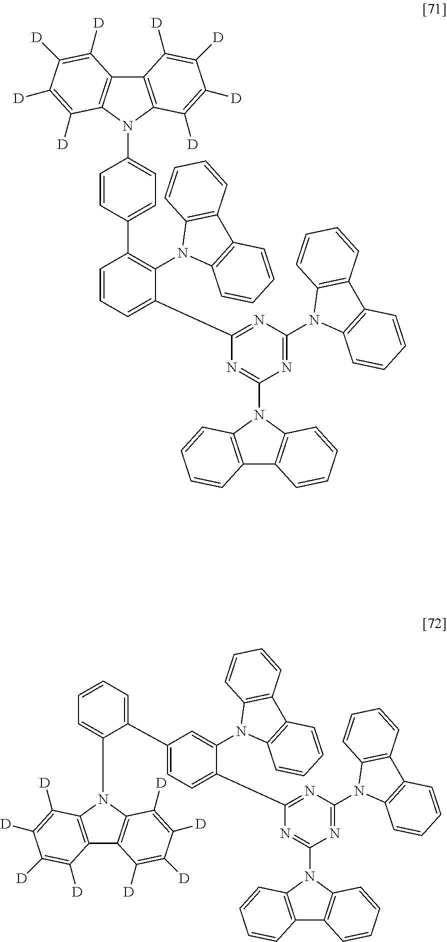 [SMILES: [2H]c1c([2H])c([2H])c2c(c1[2H])c1c([2H])c([2H])c([2H])c([2H])c1n2-c1ccc(-c2cccc(-c3nc(-n4c5ccccc5c5ccccc54)nc(-n4c5ccccc5c5ccccc54)n3)c2-n2c3ccccc3c3ccccc32)cc1 ; [2H]c1c([2H])c([2H])c2c(c1[2H])c1c([2H])c([2H])c([2H])c([2H])c1n2-c1ccccc1-c1ccc(-c2nc(-n3c4ccccc4c4ccccc43)nc(-n3c4ccccc4c4ccccc43)n2)c(-n2c3ccccc3c3ccccc32)c1]

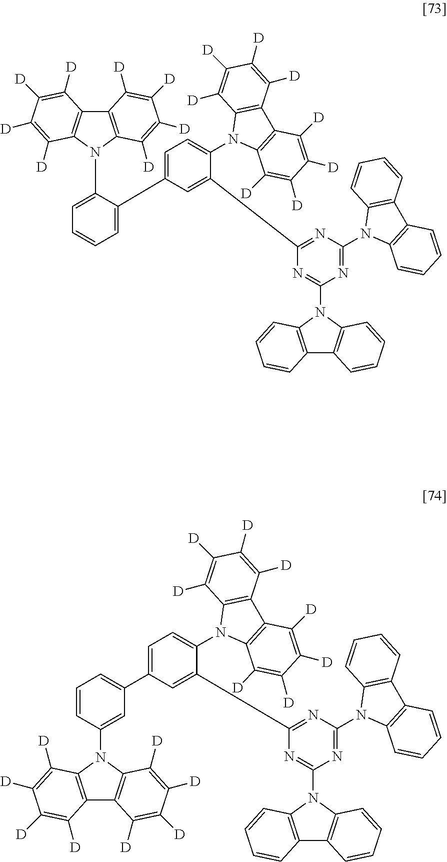 [SMILES: [2H]c1c([2H])c([2H])c2c(c1[2H])c1c([2H])c([2H])c([2H])c([2H])c1n2-c1ccccc1-c1ccc(-n2c3c([2H])c([2H])c([2H])c([2H])c3c3c([2H])c([2H])c([2H])c([2H])c32)c(-c2nc(-n3c4ccccc4c4ccccc43)nc(-n3c4ccccc4c4ccccc43)n2)c1 ; [2H]c1c([2H])c([2H])c2c(c1[2H])c1c([2H])c([2H])c([2H])c([2H])c1n2-c1cccc(-c2ccc(-n3c4c([2H])c([2H])c([2H])c([2H])c4c4c([2H])c([2H])c([2H])c([2H])c43)c(-c3nc(-n4c5ccccc5c5ccccc54)nc(-n4c5ccccc5c5ccccc54)n3)c2)c1]

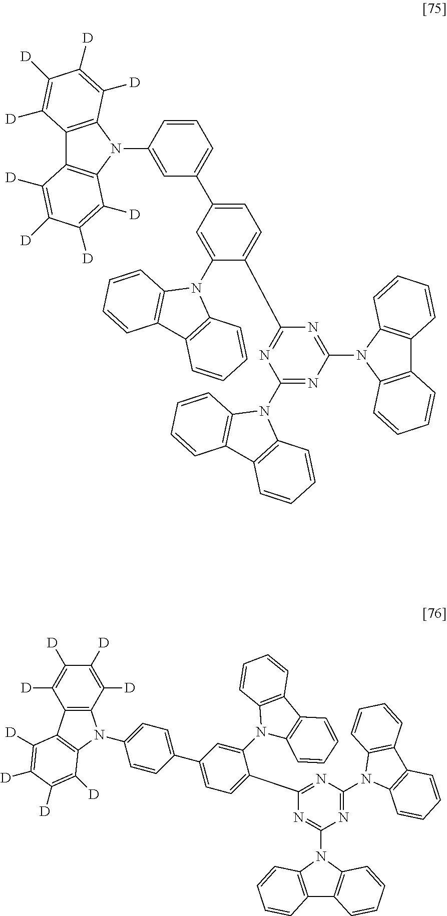 [SMILES: [2H]c1c([2H])c([2H])c2c(c1[2H])c1c([2H])c([2H])c([2H])c([2H])c1n2-c1cccc(-c2ccc(-c3nc(-n4c5ccccc5c5ccccc54)nc(-n4c5ccccc5c5ccccc54)n3)c(-n3c4ccccc4c4ccccc43)c2)c1 ; [2H]c1c([2H])c([2H])c2c(c1[2H])c1c([2H])c([2H])c([2H])c([2H])c1n2-c1ccc(-c2ccc(-c3nc(-n4c5ccccc5c5ccccc54)nc(-n4c5ccccc5c5ccccc54)n3)c(-n3c4ccccc4c4ccccc43)c2)cc1]

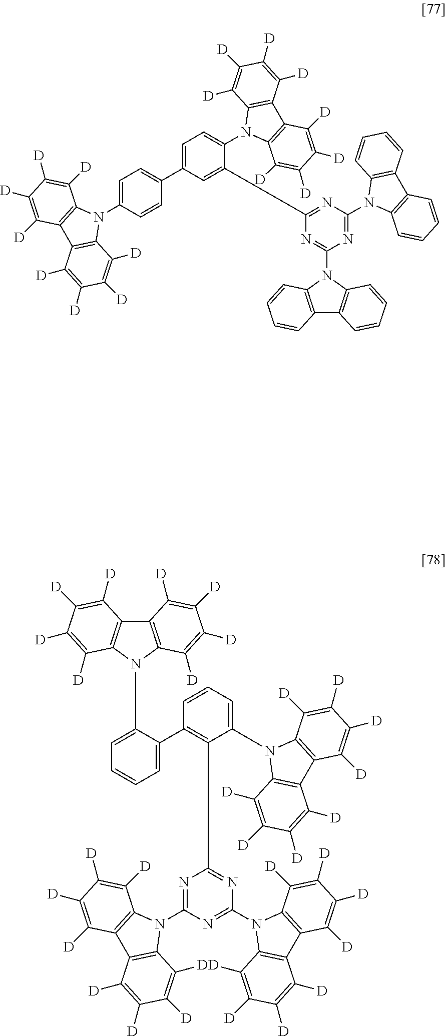

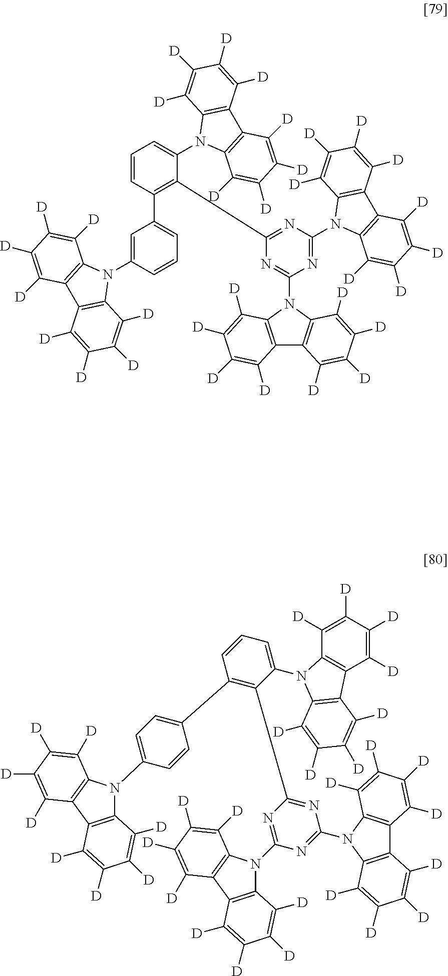

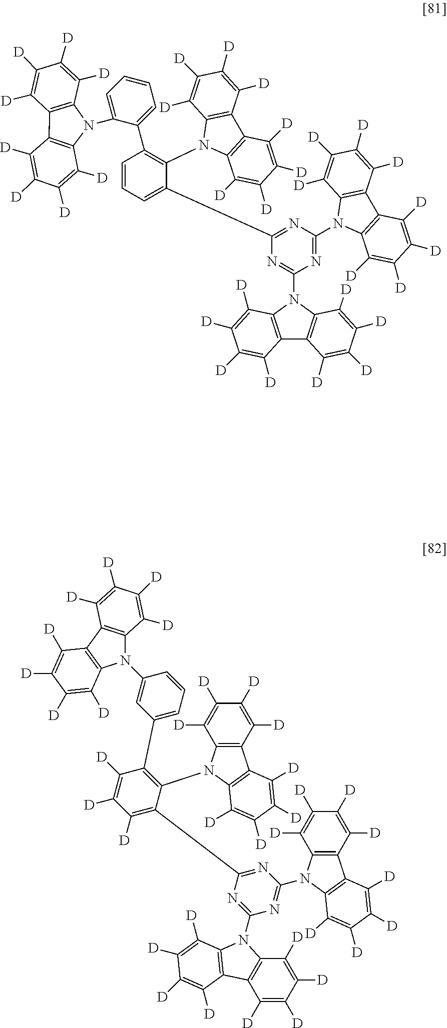

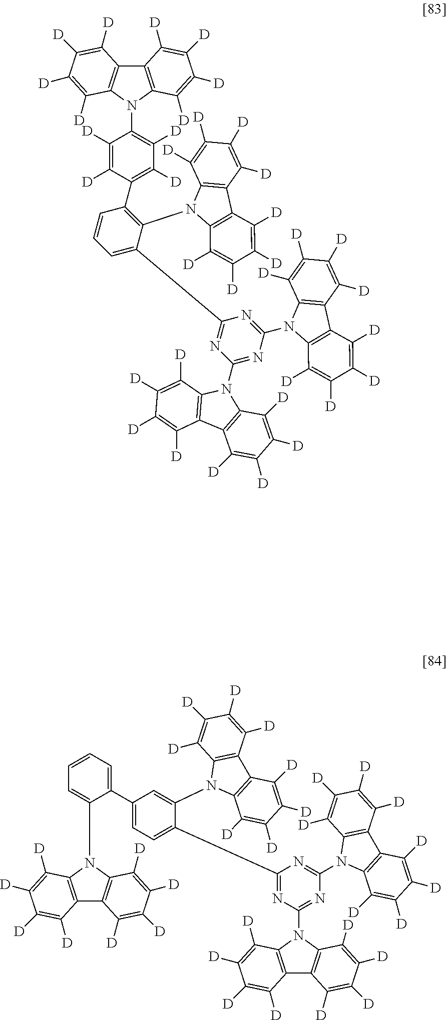

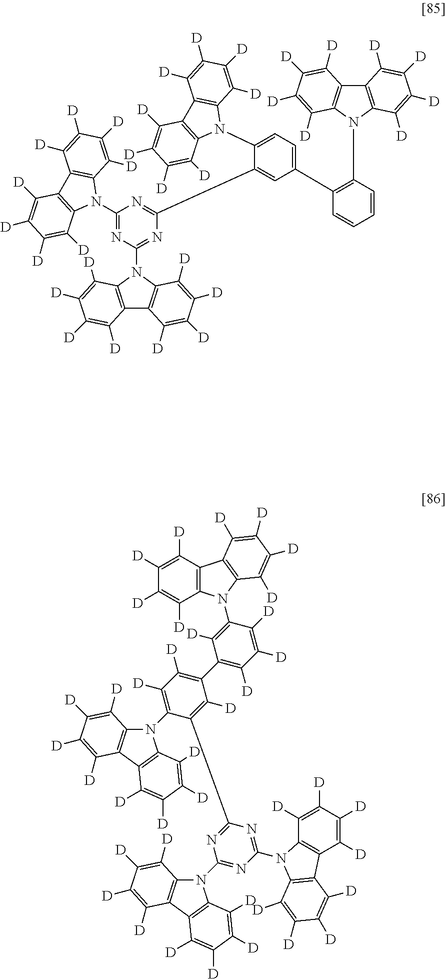

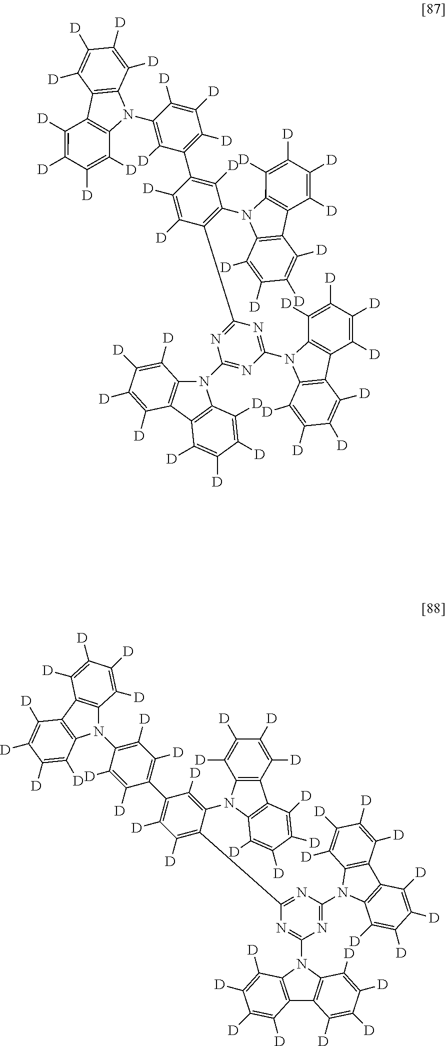

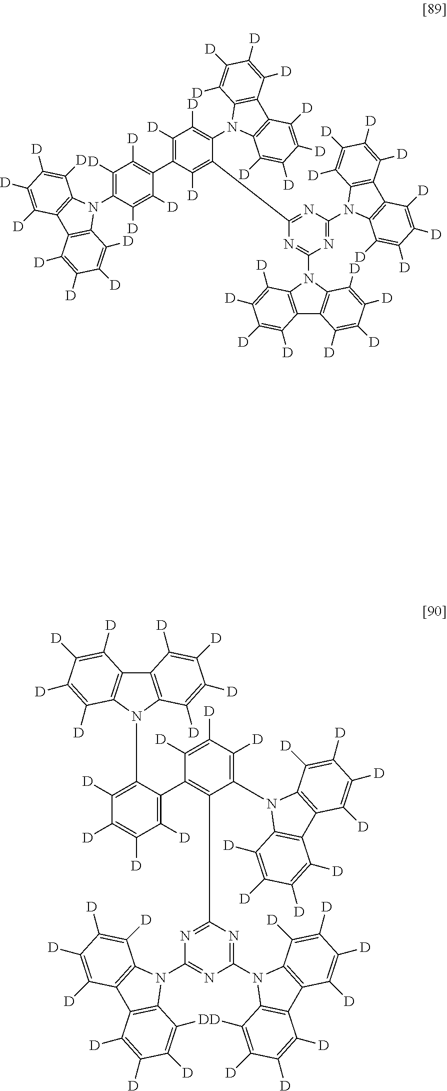

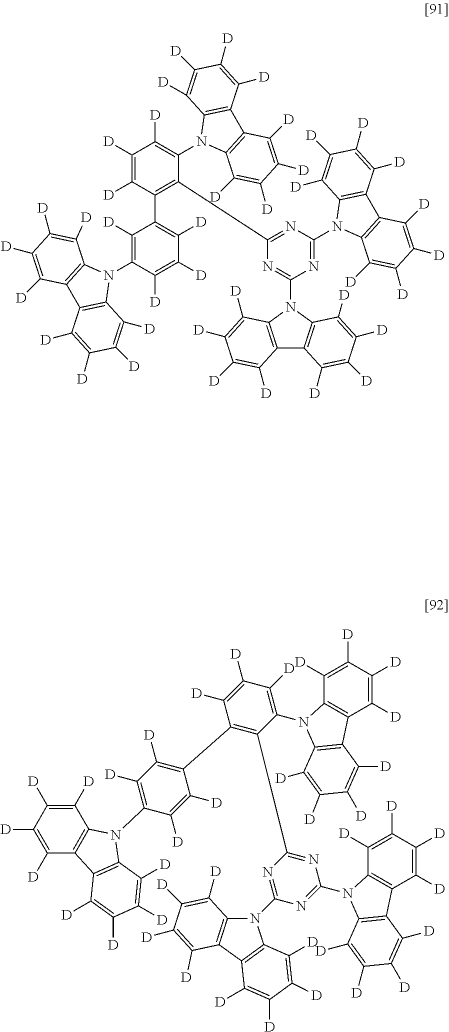

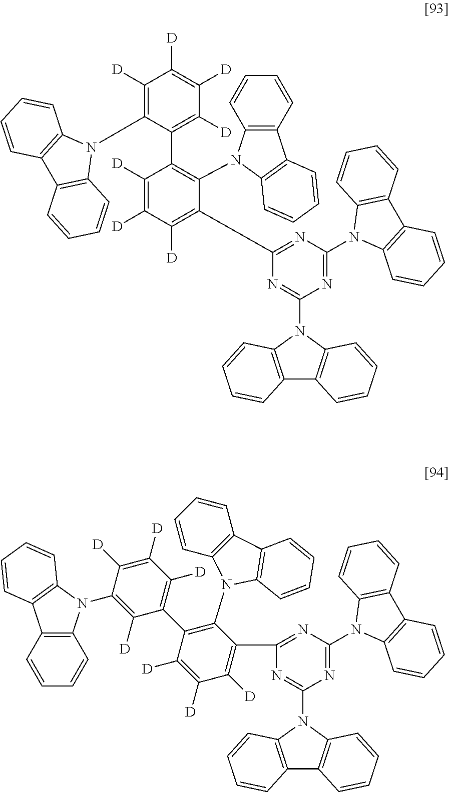 [SMILES: [2H]c1c([2H])c([2H])c(-n2c3ccccc3c3ccccc32)c(-c2c([2H])c([2H])c([2H])c(-c3nc(-n4c5ccccc5c5ccccc54)nc(-n4c5ccccc5c5ccccc54)n3)c2-n2c3ccccc3c3ccccc32)c1[2H] ; [2H]c1c([2H])c(-c2nc(-n3c4ccccc4c4ccccc43)nc(-n3c4ccccc4c4ccccc43)n2)c(-n2c3ccccc3c3ccccc32)c(-c2c([2H])c([2H])c([2H])c(-n3c4ccccc4c4ccccc43)c2[2H])c1[2H]]

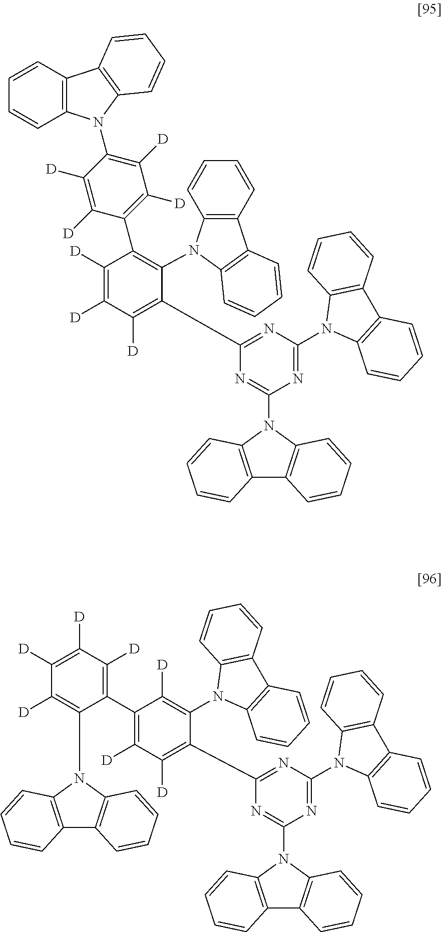 [SMILES: [2H]c1c([2H])c(-c2nc(-n3c4ccccc4c4ccccc43)nc(-n3c4ccccc4c4ccccc43)n2)c(-n2c3ccccc3c3ccccc32)c(-c2c([2H])c([2H])c(-n3c4ccccc4c4ccccc43)c([2H])c2[2H])c1[2H] ; [2H]c1c([2H])c([2H])c(-n2c3ccccc3c3ccccc32)c(-c2c([2H])c([2H])c(-c3nc(-n4c5ccccc5c5ccccc54)nc(-n4c5ccccc5c5ccccc54)n3)c(-n3c4ccccc4c4ccccc43)c2[2H])c1[2H]]

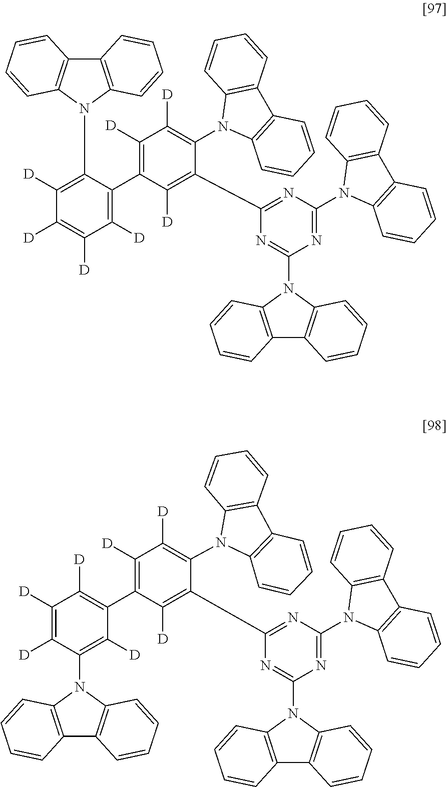 [SMILES: [2H]c1c([2H])c([2H])c(-n2c3ccccc3c3ccccc32)c(-c2c([2H])c([2H])c(-n3c4ccccc4c4ccccc43)c(-c3nc(-n4c5ccccc5c5ccccc54)nc(-n4c5ccccc5c5ccccc54)n3)c2[2H])c1[2H] ; [2H]c1c([2H])c(-c2c([2H])c([2H])c(-n3c4ccccc4c4ccccc43)c(-c3nc(-n4c5ccccc5c5ccccc54)nc(-n4c5ccccc5c5ccccc54)n3)c2[2H])c([2H])c(-n2c3ccccc3c3ccccc32)c1[2H]]

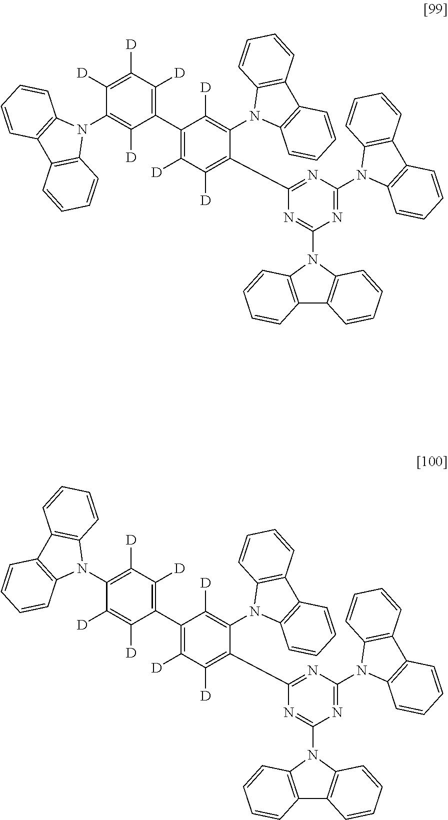 [SMILES: [2H]c1c([2H])c(-c2c([2H])c([2H])c(-c3nc(-n4c5ccccc5c5ccccc54)nc(-n4c5ccccc5c5ccccc54)n3)c(-n3c4ccccc4c4ccccc43)c2[2H])c([2H])c(-n2c3ccccc3c3ccccc32)c1[2H] ; [2H]c1c([2H])c(-n2c3ccccc3c3ccccc32)c([2H])c([2H])c1-c1c([2H])c([2H])c(-c2nc(-n3c4ccccc4c4ccccc43)nc(-n3c4ccccc4c4ccccc43)n2)c(-n2c3ccccc3c3ccccc32)c1[2H]]

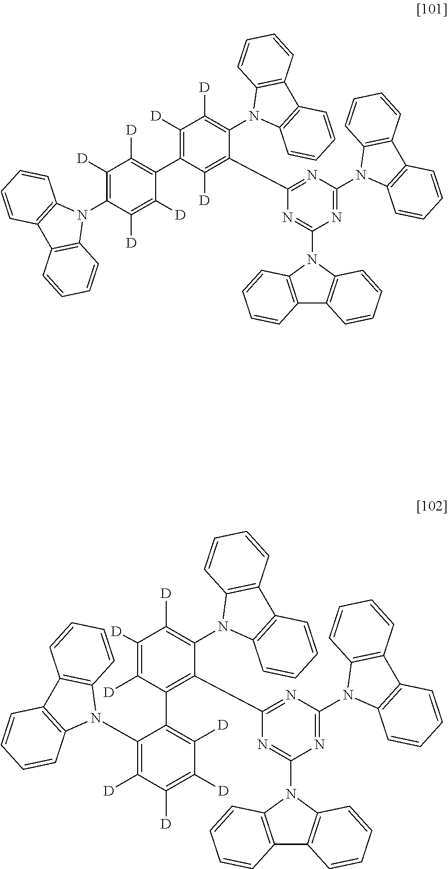 [SMILES: [2H]c1c([2H])c(-n2c3ccccc3c3ccccc32)c([2H])c([2H])c1-c1c([2H])c([2H])c(-n2c3ccccc3c3ccccc32)c(-c2nc(-n3c4ccccc4c4ccccc43)nc(-n3c4ccccc4c4ccccc43)n2)c1[2H] ; [2H]c1c([2H])c([2H])c(-n2c3ccccc3c3ccccc32)c(-c2c([2H])c([2H])c([2H])c(-n3c4ccccc4c4ccccc43)c2-c2nc(-n3c4ccccc4c4ccccc43)nc(-n3c4ccccc4c4ccccc43)n2)c1[2H]]

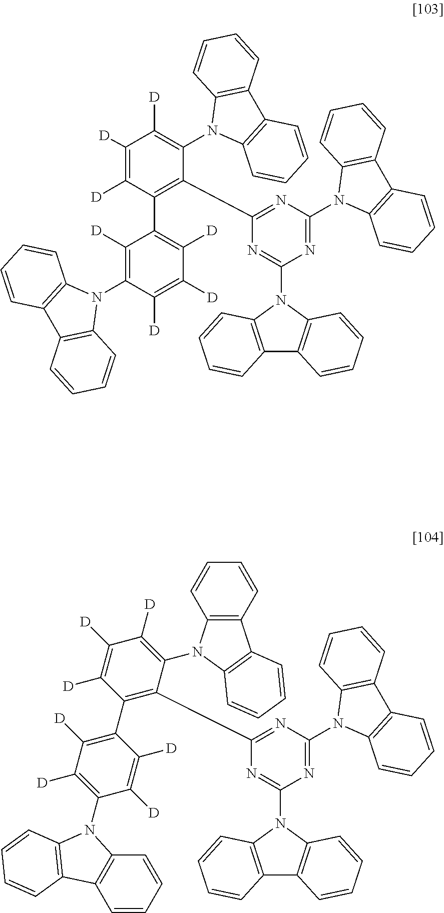 [SMILES: [2H]c1c([2H])c(-c2c([2H])c([2H])c([2H])c(-n3c4ccccc4c4ccccc43)c2-c2nc(-n3c4ccccc4c4ccccc43)nc(-n3c4ccccc4c4ccccc43)n2)c([2H])c(-n2c3ccccc3c3ccccc32)c1[2H] ; [2H]c1c([2H])c(-c2c([2H])c([2H])c(-n3c4ccccc4c4ccccc43)c([2H])c2[2H])c(-c2nc(-n3c4ccccc4c4ccccc43)nc(-n3c4ccccc4c4ccccc43)n2)c(-n2c3ccccc3c3ccccc32)c1[2H]]

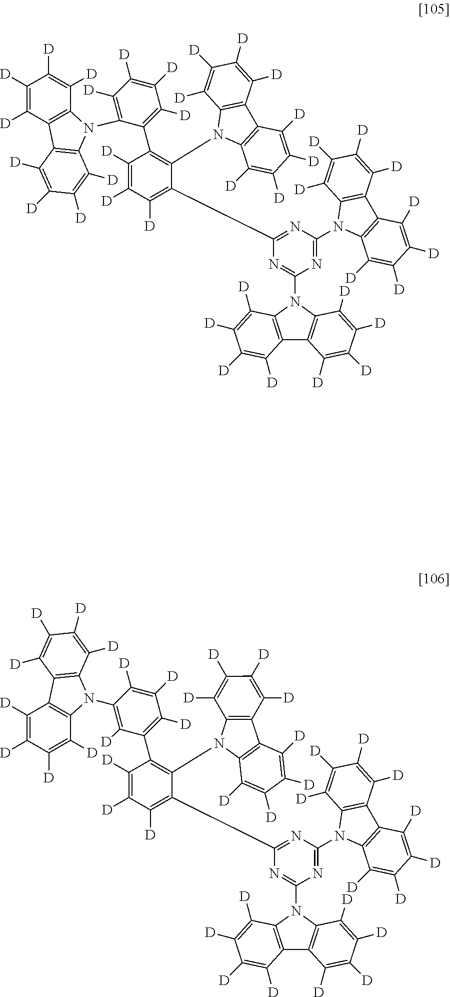

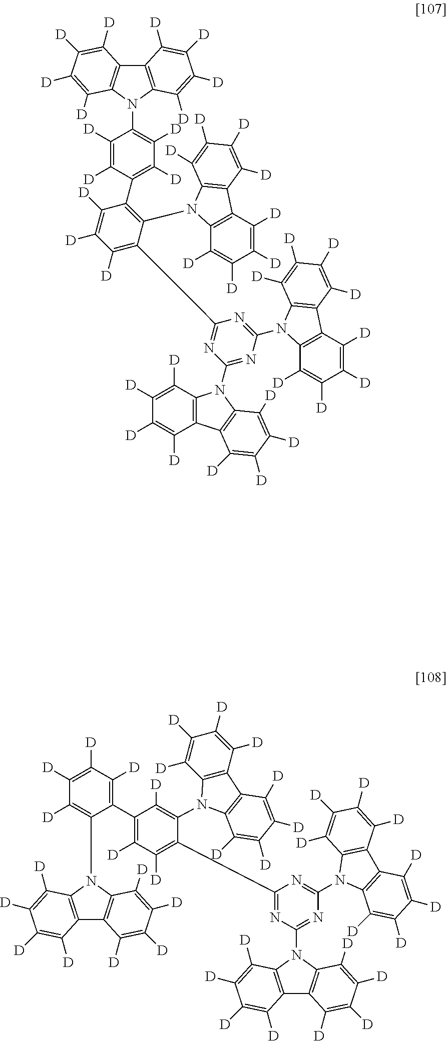

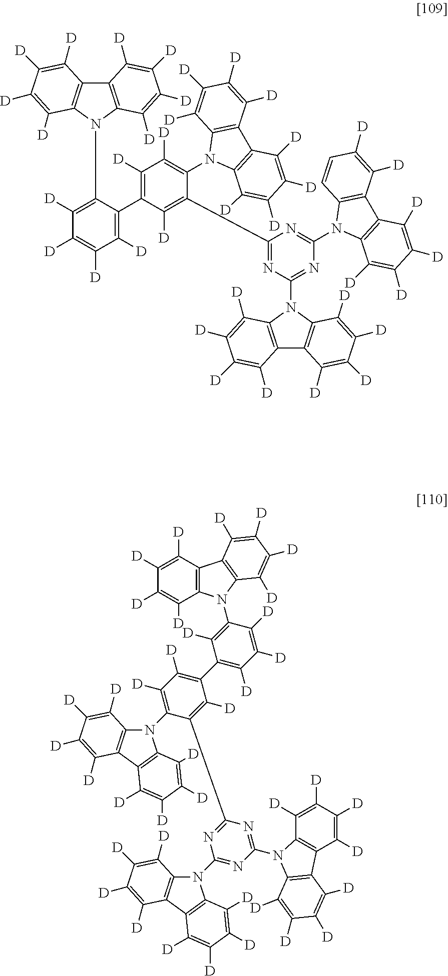

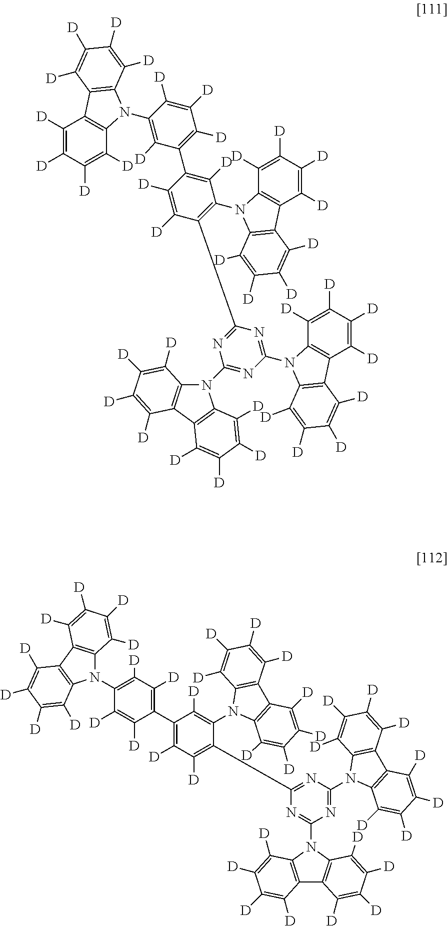

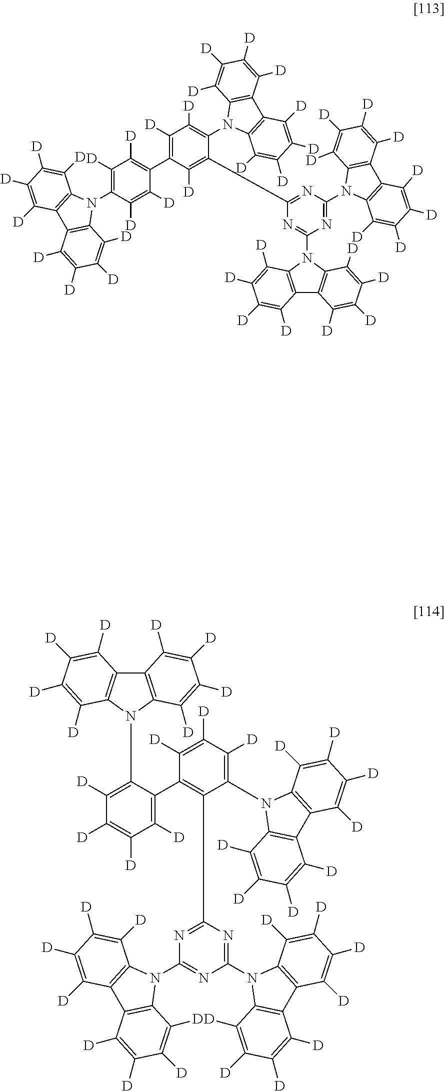

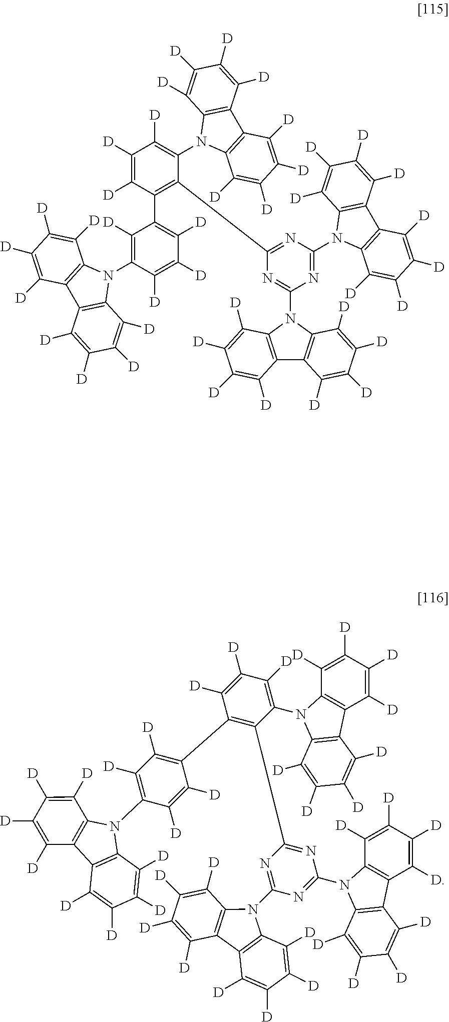

7. A composition for an organic optoelectronic device, the composition comprising:
a first compound; and
a second compound,
wherein:
the first compound is the compound for the organic optoelectronic device as claimed in claim 1, and
the second compound is represented by Chemical Formula 2, a combination of Chemical Formula 3 and Chemical Formula 4, or Chemical Formula 5: 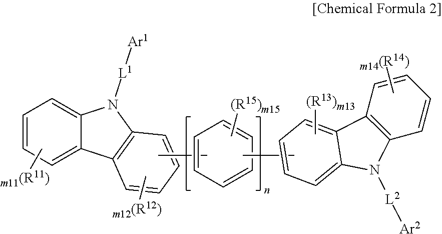
in Chemical Formula 2,
R¹¹ to R¹⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar¹ and Ar² are each independently a substituted or unsubstituted C6 to C20 aryl group or a substituted or unsubstituted C2 to C30 heterocyclic group,
L¹ and L² are each independently a single bond or a substituted or unsubstituted C6 to C20 arylene group,
m11, m14, and m15 are each independently an integer of 1 to 4, and
m12 and m13 are each independently an integer of 1 to 3,
n is an integer of 0 to 2, and
when m11 is 2, 3, or 4, each R¹¹ is the same or different from each other,
when m12 is 2 or 3, each R¹² is the same or different from each other,
when m13 is 2 or 3, each R¹³ is the same or different from each other,
when m14 is 2, 3, or 4, each R¹⁴ is the same or different from each other, and
when m15 is 2, 3, or 4, each R¹⁵ is the same or different from each other; 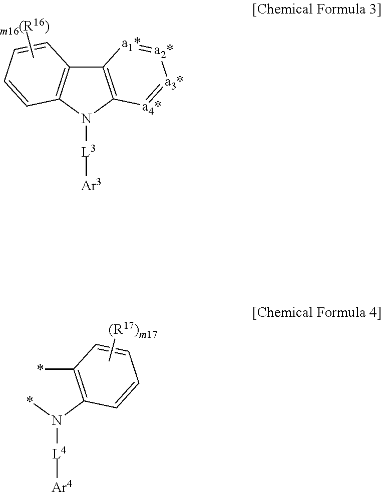
in Chemical Formula 3 and Chemical Formula 4,
two adjacent ones of a₁* to a₄* of Chemical Formula 3 are each independently linking carbons linked at * of Chemical Formula 4,
the remaining two of a₁* to a₄* of Chemical Formula 3, not linked at * of Chemical Formula 4, are each independently C-L^{a}-R^{a},
L^{a}, L³, and L⁴ are each independently a single bond or a substituted or unsubstituted C6 to C20 arylene group,
R^{a}, R¹⁶, and R¹⁷ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar³ and Ar⁴ are each independently a substituted or unsubstituted C6 to C20 aryl group or a substituted or unsubstituted C2 to C30 heterocyclic group, and
m16 and m17 are each independently an integer of 1 to 4,
when m16 is 2, 3, or 4, each R¹⁶ is the same or different from each other, and
when m17 2, 3, or 4, each R¹⁷ is the same or different from each other; 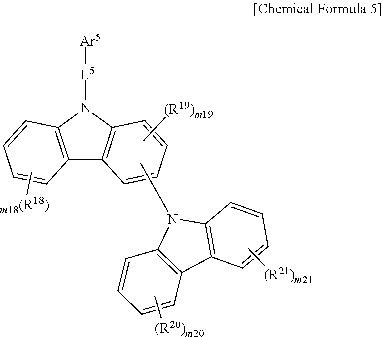
in Chemical Formula 5,
L⁵ is a single bond or a substituted or unsubstituted C6 to C20 arylene group,
R¹⁸ to R²¹ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁵ is a substituted or unsubstituted C6 to C20 aryl group or a substituted or unsubstituted C2 to C30 heterocyclic group,
m18, m20, and m21 are each independently an integer of 1 to 4, and
m19 is an integer of 1 to 3, and
when m18 is 2, 3, or 4, each R¹⁸ is the same or different from each other,
when m19 is 2 or 3, each R¹⁹ is the same or different from each other,
when m20 is 2, 3, or 4, each R²⁰ is the same or different from each other, and
when m21 is 2, 3, or 4, each R²¹ is the same or different from each other.

8. The composition for the organic optoelectronic device as claimed in claim 7, wherein:
Chemical Formula 2 is represented by Chemical Formula 2-8: 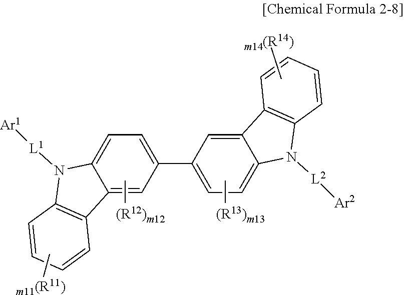
in Chemical Formula 2-8,
R¹¹ to R¹⁴ are each independently hydrogen, deuterium, or a substituted or unsubstituted C6 to C12 aryl group,
m11 and m14 are each independently an integer of 1 to 4,
m12 and m13 are each independently an integer of 1 to 3, and
moieties *-L¹-Ar¹ and *-L²-Ar² are each independently a moiety of Group II, 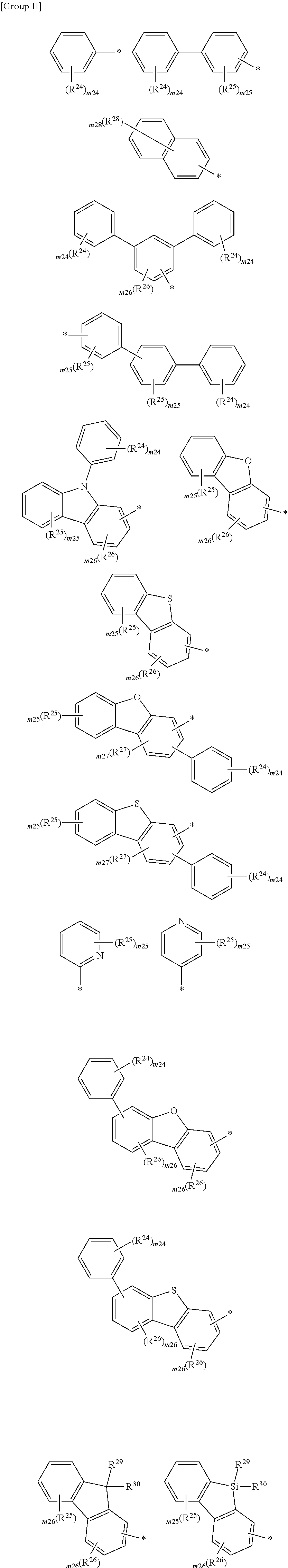 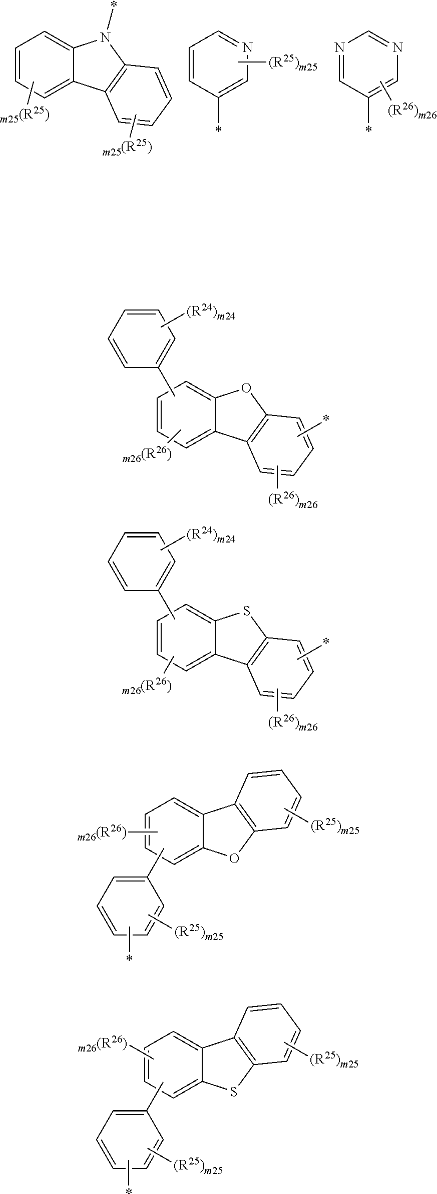 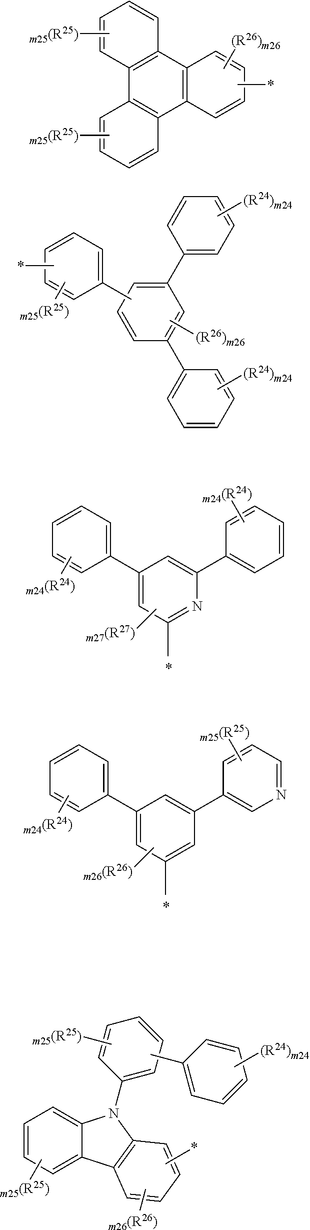 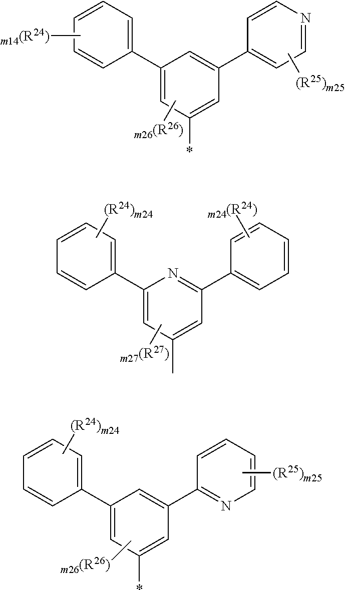
in Group II,
R²⁴ to R³⁰ are each independently hydrogen, deuterium, a cyano group, a substituted or unsubstituted C1 to C10 alkyl group, or a substituted or unsubstituted C6 to C12 aryl group,
m24 is an integer of 1 to 5,
m25 is an integer of 1 to 4,
m26 is an integer of 1 to 3,
m27 is an integer of 1 or 2,
m28 is an integer of 1 to 7, and
* is a linking point.

9. The composition for the organic optoelectronic device as claimed in claim 7, wherein:
the combination of Chemical Formula 3 and Chemical Formula 4 is represented by Chemical Formula 3C: 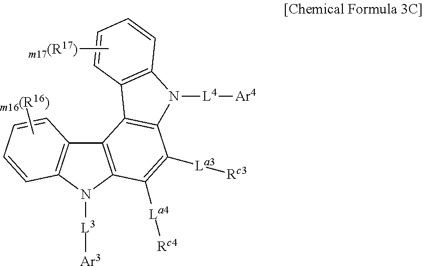
in Chemical Formula 3C,
L^{a3} and L^{a4} are each a single bond,
R¹⁶, R¹⁷, R^{a3}, and R^{a4} are each independently hydrogen, deuterium, or a C6 to C12 aryl group,
m16 and m17 are each independently an integer of 1 to 4, and
moieties *-L³-Ar³ and *-L⁴-Ar⁴ are each independently a moiety of Group II, 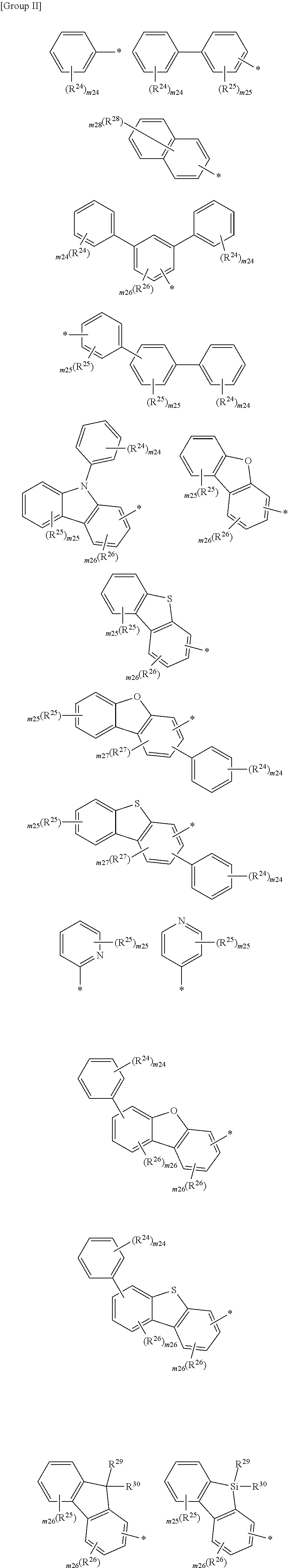 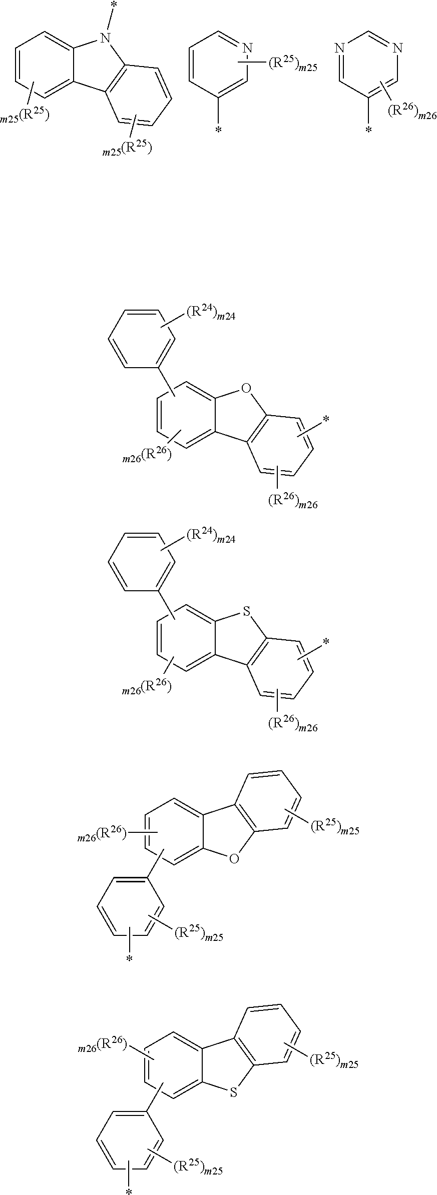 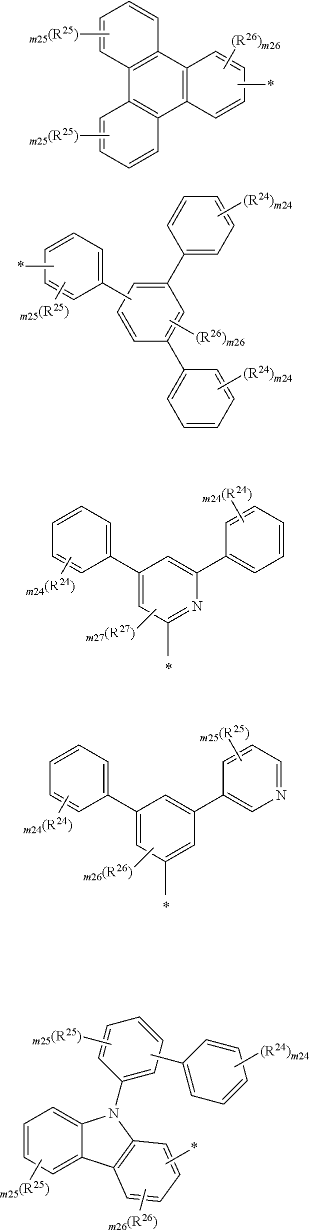 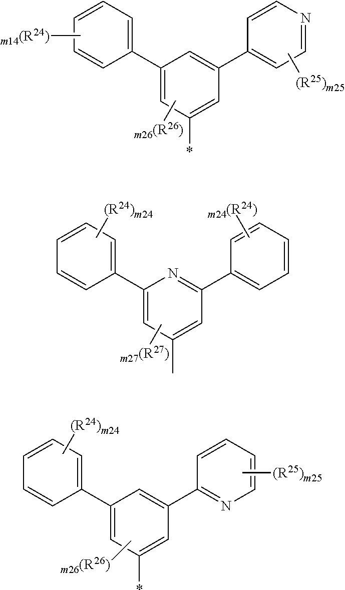
in Group II,
R²⁴ to R³⁰ are each independently hydrogen, deuterium, a cyano group, a substituted or unsubstituted C1 to C10 alkyl group, or a substituted or unsubstituted C6 to C12 aryl group,
m24 is an integer of 1 to 5,
m25 is an integer of 1 to 4,
m26 is an integer of 1 to 3,
m27 is an integer of 1 or 2,
m28 is an integer of 1 to 7, and
* is a linking point.

10. The composition for the organic optoelectronic device as claimed in claim 7, wherein:
the first compound is represented by Chemical Formula 1-2a to 1-4a, and
the second compound is represented by Chemical Formula 5-3: 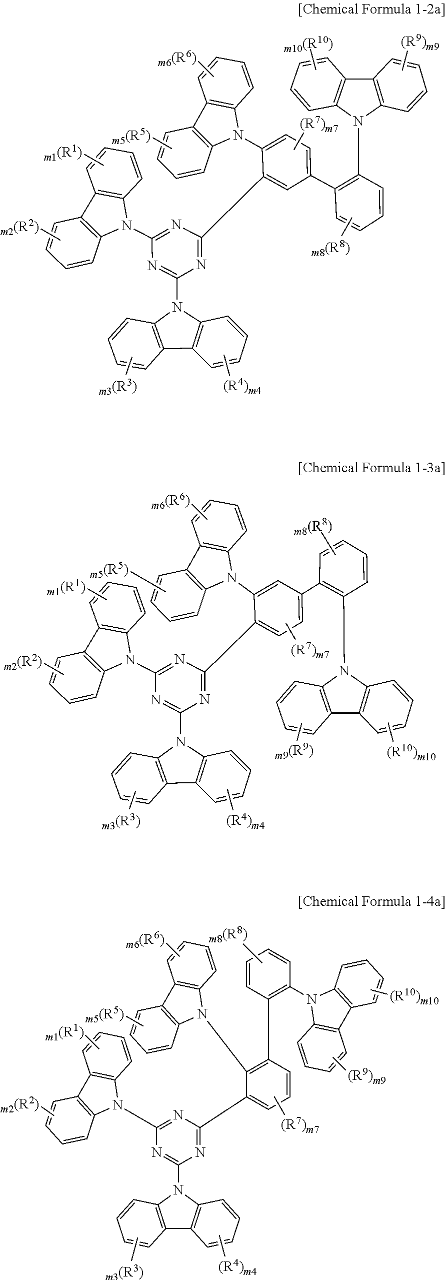
in Chemical Formula 1-2a to Chemical Formula 1-4a,
R¹ to R¹⁰ are each independently hydrogen, deuterium, a substituted or unsubstituted C1 to C10 alkyl group, a substituted or unsubstituted C3 to C10 cycloalkyl group, a substituted or unsubstituted phenyl group, or a substituted or unsubstituted biphenyl group,
m1 to m6 and m8 to m10 are each independently an integer of 1 to 4, and
m7 is an integer of 1 to 3, 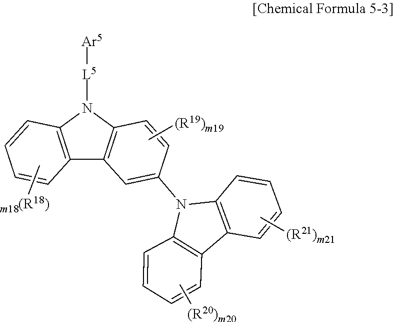
in Chemical Formula 5-3,
L⁵ is a substituted or unsubstituted C6 to C12 arylene group,
R¹⁸ to R²¹ are each independently hydrogen, deuterium, or a substituted or unsubstituted phenyl group,
Ar⁵ is a substituted or unsubstituted C6 to C120 aryl group,
m18, m20, and m21 are each independently an integer of 1 to 4, and
m19 is an integer of 1 to 3.

11. An organic optoelectronic device, comprising:
an anode and a cathode facing each other,
at least one organic layer between the anode and the cathode,
wherein the at least one organic layer includes the compound for the organic optoelectronic device as claimed in claim 1.

12. The organic optoelectronic device as claimed in claim 11, wherein:
the organic layer includes a light emitting layer, and
the light emitting layer includes the compound.

13. A display device comprising the organic optoelectronic device as claimed in claim 11.

14. An organic optoelectronic device, comprising:
an anode and a cathode facing each other,
at least one organic layer between the anode and the cathode,
wherein the at least one organic layer includes the composition for the organic optoelectronic device as claimed in claim 7.

15. The organic optoelectronic device as claimed in claim 14, wherein:
the organic layer includes a light emitting layer, and
the light emitting layer includes the composition.

16. A display device comprising the organic optoelectronic device as claimed in claim 14.
